(19) United States
(12) Patent Application Publication
(10) Pub. No.: US 2004/0142979 A1
(43) Pub. Date: Jul. 22, 2004

(54) Heteroarylsulfonylmethyl hydroxamic acids and amides and their use as protease inhibitors

(76) Inventors: Daniel P. Becker, Glenview, IL (US); Jeffery N. Carroll, St. Louis, MO (US); Yvette M. Fobian, Wildwood, MO (US); Margaret L. Grapperhaus, Troy, IL (US); Donald W. Hansen JR., Skokie, IL (US); Robert M. Heintz, Bourbon, MO (US); Darren J. Kassab, O'Fallon, MO (US); Mark A. Massa, Ballwin, MO (US); Joseph J. McDonald, Wildwood, MO (US); Mark A. Nagy, Chesterfield, MO (US); Barnett S. Pitzele, Skokie, IL (US); Joseph G. Rico, O'Fallon, MO (US); Michelle A. Schmidt, Belleville, IL (US); Dale P. Spangler, San Diego, CA (US)

Correspondence Address: HARNESS, DICKEY, & PIERCE, P.L.C, 7700 BONHOMME, STE 400, ST. LOUIS, MO, 63105, US

(21) Appl. No.: 10/722,104

(22) Filed: Nov. 25, 2003

Related U.S. Application Data
(60) Provisional application No. 60/429,068, filed on Nov. 25, 2002.
(60) Provisional application No. 60/504,281, filed on Sep. 19, 2003.

(51) Int. Cl. A61K 31/44; A61K 31/4015; A61K 31/165

(52) U.S. Cl. 514/346; 514/424; 514/618; 546/291; 548/546; 564/162

(57) ABSTRACT

This invention is directed generally to heteroarylsulfonylmethyl hydroxamic acids and amides that, inter alia, tend to inhibit protease activity, particularly matrix metalloproteinase (also known as “matrix metalloprotease” or “MMP”) activity and/or aggrecanase activity. This invention also is directed to compositions of such compounds; intermediates for the syntheses of such compounds; methods for making such compounds; and methods for treating conditions associated with MMP, tumor necrosis factors (or “TNFs”), and/or aggrecanase activity, particularly pathological conditions.

### PRIORITY CLAIM TO RELATED PATENT APPLICATIONS

This patent claims priority to U.S. Provisional Patent Application Serial Nos. 60/429,068 (filed Nov. 25, 2002) and 60/504,281 (filed Sep. 19, 2003). The entire text of each of the above-referenced applications is incorporated by reference into this patent.

### FIELD OF THE INVENTION

This invention is directed generally to heteroarylsulfonylmethyl hydroxamic acids and amides that, inter alia, tend to inhibit protease activity, particularly matrix metalloproteinase (also known as “matrix metalloprotease” or “MMP”) activity and/or aggrecanase activity. This invention also is directed to compositions of such compounds; intermediates for the syntheses of such compounds; methods for making such compounds; and methods for treating conditions associated with MMP, tumor necrosis factors (or “TNFs”), and/or aggrecanase activity, particularly pathological conditions.

### BACKGROUND OF THE INVENTION

Connective tissue is a required component of all mammals. It provides rigidity, differentiation, attachments, and, in some cases, elasticity. Connective tissue components include, for example, collagen, elastin, proteoglycans, fibronectin, and laminin. These biochemicals make up (or are components of) structures, such as skin, bone, teeth, tendon, cartilage, basement membrane, blood vessels, cornea, and vitreous humor.

Under normal conditions, connective tissue turnover and/or repair processes are in equilibrium with connective tissue production. Degradation of connective tissue is carried out by the action of proteinases released from resident tissue cells and/or invading inflammatory or tumor cells.

Matrix metalloproteinases, a family of zinc-dependent proteinases, make up a major class of enzymes involved in degrading connective tissue. Matrix metalloproteinases are divided into classes, with some members having several different names in common use. Examples are: MMP-1 (also known as collagenase 1, fibroblast collagenase, or EC 3.4.24.3); MMP-2 (also known as gelatinase A, 72 kDa gelatinase, basement membrane collagenase, or EC 3.4.24.24), MMP-3 (also known as stromelysin 1 or EC 3.4.24.17), proteoglycanase, MMP-7 (also known as matrilysin), MMP-8 (also known as collagenase II, neutrophil collagenase, or EC 3.4.24.34), MMP-9 (also known as gelatinase B, 92 kDa gelatinase, or EC 3.4.24.35), MMP-10 (also known as stromelysin 2 or EC 3.4.24.22), MMP-11 (also known as stromelysin 3), MMP-12 (also known as metalloelastase, human macrophage elastase or HME), MMP-13 (also known as collagenase 111), and MMP-14 (also known as MT1-MMP or membrane MMP). See, generally, Woessner, J. F., “The Matrix Metalloprotease Family” in Matrix Metalloproteinases, pp.1-14 (Edited by Parks, W. C. & Mecham, R. P., Academic Press, San Diego, Calif. 1998).

Excessive breakdown of connective tissue by MMPs is a feature of many pathological conditions. Inhibition of MMPs therefore provides a control mechanism for tissue decomposition to treat these pathological conditions. Such pathological conditions generally include, for example, tissue destruction, fibrotic diseases, pathological matrix weakening, defective injury repair, cardiovascular diseases, pulmonary diseases, kidney diseases, liver diseases, ophthalmologic diseases, and diseases of the central nervous system. Specific examples of such conditions include rheumatoid arthritis, osteoarthritis, septic arthritis, multiple sclerosis, a decubitis ulcer, corneal ulceration, epidermal ulceration, gastric ulceration, tumor metastasis, tumor invasion, tumor angiogenesis, periodontal disease, liver cirrhosis, fibrotic lung disease, emphysema, otosclerosis, atherosclerosis, proteinuria, coronary thrombosis, dilated cardiomyopathy, congestive heart failure, aortic aneurysm, epidermolysis bullosa, bone disease, Alzheimer's disease, defective injury repair (e.g., weak repairs, adhesions such as post-surgical adhesions, and scarring), post-myocardial infarction, bone disease, and chronic obstructive pulmonary disease. MMPs (particularly MMP-9) also have been reported to be associated with pathological conditions related to nitrosative and oxidative stress. See Gu, Zezong et al., “S-Nitrosylation of Matrix Metalloproteinases: Signaling Pathway to Neuronal Cell Death,” Science, vol. 297, pp. 1186-90 (2002).

Matrix metalloproteinases also are involved in the biosynthesis of tumor necrosis factors (TNFs). Tumor necrosis factors are implicated in many pathological conditions. TNF-α, for example, is a cytokine that is believed to be produced initially as a 28 kD cell-associated molecule. It is released as an active, 17 kD form that can mediate a large number of deleterious effects in vitro and in vivo. TNF-α can cause and/or contribute to the effects of inflammation (e.g., rheumatoid arthritis), autoimmune disease, graft rejection, multiple sclerosis, fibrotic diseases, cancer, infectious diseases (e.g., malaria, mycobacterial infection, meningitis, etc.), fever, psoriasis, cardiovascular diseases (e.g., post-ischemic reperfusion injury and congestive heart failure), pulmonary diseases, hemorrhage, coagulation, hyperoxic alveolar injury, radiation damage, and acute phase responses like those seen with infections and sepsis and during shock (e.g., septic shock and hemodynamic shock). Chronic release of active TNF-α can cause cachexia and anorexia. TNF-α also can be lethal.

Inhibiting TNF (and related compounds) production and action is an important clinical disease treatment. Matrix metalloproteinase inhibition is one mechanism that can be used. MMP (e.g., collagenase, stromelysin, and gelatinase) inhibitors, for example, have been reported to inhibit TNF-α release. See, e.g., Gearing et al. Nature, 370, 555-557 (1994). See also, McGeehan et al., Nature, 370, 558-561 (1994). MMP inhibitors also have been reported to inhibit TNF-α convertase, a metalloproteinase involved in forming active TNF-α. See, e.g., WIPO Int'l Pub. No. WO 94/24140. See also, WIPO Int'l Pub. No. WO 94/02466. See also, WIPO Int'l Pub. No. WO 97/20824.

Matrix metalloproteinases also are involved in other biochemical processes in mammals. These include control of ovulation, post-partum uterine involution, possibly implantation, cleavage of APP (β-amyloid precursor protein) to the amyloid plaque, and inactivation of (α₁-protease inhibitor (α₁-PI). Inhibiting MMPs therefore may be a mechanism that may be used to control of fertility. In addition, increasing and maintaining the levels of an endogenous or administered serine protease inhibitor (e.g., α₁-PI) supports the treatment of pathological conditions such as emphysema, pulmonary diseases, inflammatory diseases, and diseases of aging (e.g., loss of skin or organ stretch and resiliency).

Numerous metalloproteinase inhibitors are known. See, generally, Brown, P. D., “Synthetic Inhibitors of Matrix Metalloproteinases,” in Matrix Metalloproteinases, pp. 243-61 (Edited by Parks, W. C. & Mecham, R. P., Academic Press, San Diego, Calif. 1998).

Metalloproteinase inhibitors include, for example, natural biochemicals, such as tissue inhibitor of metalloproteinase (TIMP), α2-macroglobulin, and their analogs and derivatives. These are high-molecular-weight protein molecules that form inactive complexes with metalloproteinases.

A number of smaller peptide-like compounds also have been reported to inhibit metalloproteinases. Mercaptoamide peptidyl derivatives, for example, have been reported to inhibit angiotensin converting enzyme (also known as ACE) in vitro and in vivo. ACE aids in the production of angiotensin II, a potent pressor substance in mammals. Inhibiting ACE leads to lowering of blood pressure.

A wide variety of thiol compounds have been reported to inhibit MMPs. See, e.g., WIPO Int'l Pub. No. WO 95/13289. See also, WIPO Int'l Pub. No. WO 96/11209. See also, U.S. Pat. No. 4,595,700. See also, U.S. Pat. No. 6,013,649.

Various hydroxamic acid compounds also have been reported to inhibit MMPs. Such compounds reportedly include compounds having a carbon backbone. See, e.g., WIPO Int'l Pub. No. WO 95/29892. See also, WIPO Int'l Pub. No. WO 97/24117. See also, WIPO Int'l Pub. No. WO 97/49679 or U.S. Pat. No. 6,300,514. See also, European Patent No. EP 0 780 386. Such compounds also reportedly include compounds having peptidyl backbones or peptidomimetic backbones. See, e.g, WIPO Int'l Pub. No. WO 90/05719. See also, WIPO Int'l Pub. No. WO 93/20047. See also, WIPO Int'l Pub. No. WO 95/09841. See also, WIPO Int'l Pub. No. WO 96/06074. See also, Schwartz et al., Progr. Med. Chem., 29:271-334(1992). See also, Rasmussen et al., Pharmacol. Ther., 75(1): 69-75 (1997). See also, Denis et al., Invest New Drugs, 15: 175-185 (1997). Various piperazinylsulfonylmethyl and piperidinylsulfonylmethyl hydroxamic acid compounds also have been reported to inhibit MMPs. See, WIPO Int'l Pub. No. WO 00/46221. See also, U.S. Pat. Nos. 6,448,250; 6,372,758; and 6,492,367. See also, WIPO PCT Appl. No. PCT/US03/13123. And various aryl or heteroaryl sulfone hydroxamic acid compounds have been reported to inhibit MMPs. See, WIPO Int'l Pub. No. WO 99/25687 (which issued as U.S. Pat. No. 6,541,489 on Apr. 1, 2003). See also, WIPO Int'l Pub. No. WO 00/50396. See also, WIPO Int'l Pub. No. WO 00/69821. See also, WIPO Int'l Pub. No. WO 02/092588. See also, U.S. Appl. Publ. No. US-2003-0073718. See also, WIPO PCT Appl. No. PCT/US03/20028.

Various amide compounds also have been reported to inhibit MMPs. Such compounds include, for example, various aryl and heteroaryl sulfone compounds. See, e.g., WIPO Int'l Pub. No. WO 00/50396. See also, WIPO Int'l Pub. No. WO 00/69821. See also, WIPO PCT Appl. No. PCT/US03/20028.

It is generally advantageous for an MMP inhibitor drug to target a certain MMP(s) over another MMP(s). For example, it is typically preferred to inhibit MMP-2, MMP-3, MMP-9, and/or MMP-1 3 when treating cancer, inhibiting of metastasis, and inhibiting angiogenesis. It also is typically preferred to inhibit MMP-13 when treating osteoarthritis. See, e.g., Mitchell et al., J. Clin. Invest., 97(3):761-768 (1996). See also, Reboul et al., J. Clin. Invest., 97(9):2011-2019 (1996). Normally, however, it is preferred to use a drug that has little or no inhibitory effect on MMP-1 and MMP-14. This preference stems from the fact that both MMP-1 and MMP-14 are involved in several homeostatic processes, and inhibition of MMP-1 and/or MMP-14 consequently tends to interfere with such processes.

Many known MMP inhibitors exhibit the same or similar inhibitory effects against each of the MMPs. For example, batimastat (a peptidomimetic hydroxamic acid) has been reported to exhibit IC₅₀ values of from about 1 to about 20 nM against each of MMP-1, MMP-2, MMP-3, MMP-7, and MMP-9. Marimastat (another peptidomimetic hydroxamic acid) has been reported to be another broad-spectrum MMP inhibitor with an enzyme inhibitory spectrum similar to batimastat, except that Marimastat reportedly exhibited an IC₅₀ value against MMP-3 of 230 nM. See Rasmussen et al., Pharmacol. Ther., 75(1): 69-75 (1997).

Meta analysis of data from Phase I/II studies using Marimastat in patients with advanced, rapidly progressive, treatment-refractory solid tumor cancers (colorectal, pancreatic, ovarian, and prostate) indicated a dose-related reduction in the rise of cancer-specific antigens used as surrogate markers for biological activity. Although Marimastat exhibited some measure of efficacy via these markers, toxic side effects reportedly were observed. The most common drug-related toxicity of Marimastat in those clinical trials was musculoskeletal pain and stiffness, often commencing in the small joints in the hands, and then spreading to the arms and shoulder. A short dosing holiday of 1-3 weeks followed by dosage reduction reportedly permits treatment to continue. See Rasmussen et al., Pharmacol. Ther., 75(1): 69-75 (1997). It is believed that the lack of specificity of inhibitory effect among the MMPs may be a cause of that effect.

Another enzyme implicated in pathological conditions associated with excessive degradation of connective tissue is aggrecanase, particularly aggrecanase-1 (also known as ADAMTS-4). Specifically, articular cartilage contains large amounts of the proteoglycan aggrecan. Proteoglycan aggrecan provides mechanical properties that help articular cartilage in withstanding compressive deformation during joint articulation. The loss of aggrecan fragments and their release into synovial fluid caused by proteolytic cleavages is a central pathophysiological event in osteoarthritis and rheumatoid arthritis. It has been reported that two major cleavage sites exist in the proteolytically sensitive interglobular domains at the N-terminal region of the aggrecan core protein. One of those sites has been reported to be cleaved by several matrix metalloproteases. The other site, however, has been reported to be cleaved by aggrecanase-1. Thus, inhibiting excessive aggrecanase activity provides an additional and/or alternative treatment method for inflammatory conditions. See generally, Tang, B. L., “ADAMTS: A Novel Family of Extracellular Matrix Proteases,” Int'l Journal of Biochemistry & Cell Biology, 33, pp. 33-44 (2001). Such diseases reportedly include, for example, osteoarthritis, rheumatoid arthritis, joint injury, reactive arthritis, acute pyrophosphate arthritis, and psoriatic arthritis. See, e.g., European Patent Application Publ. No. EP 1 081 137 A1.

In addition to inflammatory conditions, there also is evidence that inhibiting aggrecanase may be used for treating cancer. For example, excessive levels of aggrecanase-1 reportedly have been observed with a ghoma cell line. It also has been postulated that the enzymatic nature of aggrecanase and its similarities with the MMPs would support tumor invasion, metastasis, and angiogenesis. See Tang, Int'l Journal of Biochemistry & Cell Biology, 33, pp. 33-44 (2001).

Various hydroxamic acid and amide compounds have been reported to inhibit aggrecanase-1. Such compounds include, for example, those described in European Patent Application Publ. No. EP 1 081 137 A1. Such compounds also include, for example, those described in WIPO PCT Int'l Publ. No. WO 99/09000. Such compounds also include, for example, those described in WIPO PCT Int'l Publ. No. WO 00/59874. Such compounds also include, for example, those described in WIPO Int'l Pub. No. WO 02/092588. Such compounds also include, for example, those described in U.S. Appl. Publ. No. US-2003-0073718. Such compounds also include, for example, those described in WIPO PCT Int'l Publ. No. WO 03/007930. Such compounds also include, for example, those described in WIPO PCT Appl. No. PCT/US03/13123. Such compounds also include, for example, those described in WIPO PCT Appl. No. PCT/US03/20028.

In view of the importance of hydroxamic acid and amide compounds in the treatment of several pathological conditions and the lack of enzyme specificity exhibited by two of the more potent MMP-inhibitor drugs that have been in clinical trials, there continues to be a need for hydroxamic acid and amide compounds having greater enzyme specificity (preferably toward MMP-2, MMP-9, MMP-13, and/or aggrecanase (particularly toward MMP-13 in some instances; toward both MMP-2 and MMP-9 in other instances; toward all of MMP-2, MMP-9, and MMP-13 in other instances; and aggrecanase in other instances) ), while exhibiting little or no inhibition of MMP-1 and/or MMP-14 (preferably both in many instances). The following disclosure describes hydroxamic acid and amide compounds that tend to exhibit such desirable activities.

### SUMMARY OF THE INVENTION

This invention is directed to hydroxamic acid and amide compounds (and salts thereof) that, for example, tend to inhibit pathological protease activity (particularly MMP-2, MMP-9, MMP-13, and/or aggrecanase activity), while generally exhibiting relatively little or no inhibition against MMP-1 and/or MMP-14 activity. This invention also is directed to a method for inhibiting MMP and/or aggrecanase activity, particularly pathological MMP and/or aggrecanase activity. Such a method is particularly suitable to be used with mammals, such as humans, other primates (e.g., monkeys, chimpanzees. etc.), companion animals (e.g., dogs, cats, horses, etc.), farm animals (e.g., goats, sheep, pigs, cattle, etc.), laboratory animals (e.g., mice, rats, etc.), and wild and zoo animals (e.g., wolves, bears, deer, etc.).

Briefly, therefore, this invention is directed, in part, to a compound or salt thereof. The compound corresponds in structure to Formula (I):

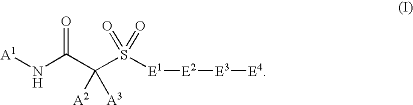

Here:

A¹ is hydrogen, hydroxyl, carbocyclyloxy, or heterocyclyloxy.

In some embodiments, A² and A³ are independently selected from the group consisting of hydrogen, alkyl, alkoxyalkyl, alkylthioalkyl, alkenyl, alkynyl, carbocyclyl, carbocyclylalkyl, carbocyclylalkenyl, carbocyclylalkynyl, carbocyclyloxyalkyl, carbocyclylalkoxyalkyl, carbocyclylalkylthio, carbocyclylthioalkyl, carbocyclylalkylthioalkyl, heterocyclyl, heterocyclylalkyl, heterocyclylalkenyl, heterocyclylalkynyl, heterocyclyloxyalkyl, heterocyclylalkoxyalkyl, heterocyclylalkylthio, heterocyclylthioalkyl, and heterocyclylalkylthioalkyl. Any such substituent optionally is substituted with:

up to three independently selected R^{X} substituents; and

two substituents such that the two substituents, together with the atom(s) to which they are bonded, form a carbocyclyl or heterocyclyl, wherein the optional heterocyclyl or carbocyclyl is, in turn, optionally substituted with up to three independently selected R^{X} substituents.

In some embodiments, A² and A³, together with the carbon to which they are both bonded, form heterocyclyl or carbocyclyl. The heterocyclyl or carbocyclyl optionally is substituted with:

up to three independently selected R^{X} substituents; and

two substituents such that the two substituents, together with the atom(s) to which they are bonded, form a carbocyclyl or heterocyclyl, wherein the optional heterocyclyl or carbocyclyl is, in turn, optionally substituted with up to three independently selected R^{X} substituents.

E¹ is heteroaryl. This heteroaryl is substituted by -E²-E³-E⁴. In addition to being substituted with -E²-E³-E⁴, the heteroaryl optionally is substituted with one or more independently selected R^{x} substituents.

E² is carbocyclyl or heterocyclyl. The carbocyclyl or heterocyclyl is substituted with -E³-E⁴, except when -E³-E⁴ is absent (e.g., when E² is oxatriazolyl). In addition to any such substitution by -E³-E⁴, the carbocyclyl or heterocyclyl optionally is substituted with one or more independently selected R^{x} substituents.

E³ is absent or is selected from the group consisting of —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, and a bond. Any alkyl or alkenyl portion of any such substituent optionally is substituted with one or more independently selected R^{c} substituents.

E⁴ is absent or selected from the group consisting of hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, and heterocyclylalkoxyalkyl. Any member of such group optionally is substituted with one or more independently selected R^{d} substituents.

Each R^{X} is independently selected from the group consisting of halogen, cyano, hydroxy, nitro, nitroso, oxo, alkyl, alkenyl, alkynyl, alkoxy, alkoxyalkoxy, R^{b}-oxyalkyl, alkenyloxy, alkynyloxy, alkylthio, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, carbocyclylthio, heterocyclyl, heterocyclylalkyl, heterocyclyloxy, heterocyclyloxyalkoxy, heterocyclylthio, alkyliminocarbonyl, alkylthioalkyl, alkylsulfonylalkyl, alkylsulfoxidoalkyl, alkylthioalkenyl, alkylsulfoxidoalkenyl, alkylsulfonylalkenyl, carbocyclylalkoxyalkyl, carbocyclyliminocarbonyl, carbocyclylthioalkyl, carbocyclylsulfoxidoalkyl, carbocyclylsulfonylalkyl, carbocyclylthioalkenyl, carbocyclylsulfoxidoalkenyl, carbocyclylsulfonylalkenyl, heterocyclylalkoxyalkyl, heterocyclylthioalkyl, heterocyclylsulfoxidoalkyl, heterocyclylsulfonylalkyl, heterocyclylthioalkenyl, heterocyclylsulfoxidoalkenyl, heterocyclylsulfonylalkenyl, heterocyclyliminocarbonyl, aminosulfonylalkyl, and —R^{x1}—R². Any member of such group optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkoxyalkyl, and alkoxyalkoxy. Any such optional substituent is, in turn, optionally substituted with one or more substituents independently selected from the group consisting of halogen, hydroxy, and alkyl.

Each R^{x1} is —C(O)—, —C(S)—, —C(NR^{y})—, —S(O)—, or —S(O)₂—. Here, each R^{y} is hydrogen or hydroxy.

Each R^{x2} is hydrogen, hydroxy, alkyl, alkenyl, alkynyl, alkoxy, alkoxyalkyl, alkoxyalkoxy, R^{b}-oxyalkyl, alkenyloxy, alkynyloxy, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, heterocyclyl, heterocyclylalkyl, heterocyclyloxy, or heterocyclyloxyalkoxy. Any member of such group optionally is substituted with one or more substituents independently selected from the group consisting of halogen, hydroxy, cyano, carboxy, thiol, sulfo, nitro, nitroso, oxo, thioxo, imino, alkyl, alkoxy, alkoxyalkyl, and alkoxyalkoxy. Any such optional substituent is, in turn, optionally substituted with one or more substituents independently selected from the group consisting of halogen and hydroxy.

Each R^{b} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl. Any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl.

Each R^{c} is independently selected from the group consisting of halogen, hydroxy, cyano, carboxy, —C(H)(NH), —C(H)(NOH), thiol, sulfo, nitro, nitroso, oxo, thioxo, imino, amino, alkyl, alkoxy, alkenyl, alkynyl, alkoxyalkyl, mono-alkylamino, di-alkylamino, alkylthio, carbocyclyl, carbocyclylalkyl, carbocyclyloxy, heterocyclyl, and heterocyclylalkyl. Any member of such group optionally is substituted with one or more substituents independently selected from the group consisting of halogen, hydroxy, cyano, carboxy, thiol, sulfo, nitro, nitroso, oxo, thioxo, imino, aminocarbonyl, amino, alkyl, and carbocyclylalkyl.

Each R^{d} is independently selected from the group consisting of halogen, hydroxy, cyano, sulfo, nitro, nitroso, oxo, thioxo, imino, alkyl, alkoxy, alkoxyalkyl, —N(R^{e})₂, —C(O)(R^{f}), —S—R^{e}, —S(O)₂—R^{e}, carbocyclyl, alkylcarbocyclyl, alkoxycarbocyclyl, carbocyclylalkyl, heterocyclyl, alkylheterocyclyl, alkoxyheterocyclyl, and heterocyclylalkyl. Any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino.

Each R^{e} is independently selected from the group consisting of hydrogen alkyl, carbocyclyl, carbocyclylalkyl, heterocyclyl, and heterocyclylalkyl. Any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino.

Each R^{f} is independently selected from the group consisting of hydrogen, alkyl, —O—R^{e}, —N(R^{e})₂, carbocyclylalkyl, and heterocyclylalkyl. Any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino.

This invention also is directed, in part, to a method for treating a condition (typically a pathological condition) in a mammal, wherein the condition comprises a condition associated with pathologically excessive matrix metalloprotease, TNF-α convertase, or aggrecanase activity. The method comprises administering an above-described compound (or a pharmaceutically acceptable salt thereof) to the mammal in an amount that is therapeutically effective to treat the condition.

This invention also is directed, in part, to a method for treating a condition in a mammal, wherein the condition comprises tissue destruction, a fibrotic disease, matrix weakening, defective injury repair, a cardiovascular disease, a pulmonary disease, a kidney disease, a liver disease, an ophthalmologic disease, or a central nervous system disease. The method comprises administering an above-described compound (or a pharmaceutically acceptable salt thereof) to the mammal in an amount that is therapeutically effective to treat the condition.

This invention also is directed, in part, to a method for treating a condition in a mammal, wherein the condition comprises osteoarthritis, rheumatoid arthritis, septic arthritis, tumor invasion, tumor metastasis, tumor angiogenesis, a decubitis ulcer, a gastric ulcer, a corneal ulcer, periodontal disease, liver cirrhosis, fibrotic lung disease, otosclerosis, atherosclerosis, multiple sclerosis, dilated cardiomyopathy, epidermal ulceration, epidermolysis bullosa, aortic aneurysm, defective injury repair, an adhesion, scarring, congestive heart failure, post myocardial infarction, coronary thrombosis, emphysema, proteinuria, Alzheimer's disease, bone disease, or chronic obstructive pulmonary disease. The method comprises administering an above-described compound (or a pharmaceutically acceptable salt thereof) to the mammal in an amount that is therapeutically effective to treat the condition.

This invention also is directed, in part, to a method for treating a condition in a mammal, wherein the condition comprises a pathological condition of the central nervous system. The method comprises administering an above-described compound (or a pharmaceutically acceptable salt thereof) to the mammal in an amount that is therapeutically effective to treat the condition.

This invention also is directed, in part, to a pharmaceutical composition comprising a therapeutically-effective amount of an above-described compound or a pharmaceutically acceptable salt thereof. Generally, such a composition further comprises one or more pharmaceutically-acceptable adjuvants.

This invention also is directed, in part, to a use of a therapeutically-effective amount of an above-described compound (or a pharmaceutically acceptable salt thereof) to prepare a medicament.

This invention also is directed, in part, to compounds or salts thereof that are, for example, useful as intermediates in processes for making the above-described compounds and salts. Such intermediate compounds correspond in structure to Formula (II):

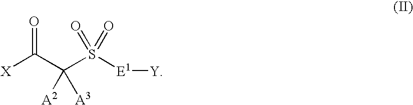

Here:

X is —O—R¹, —NH—O—R², —NH—O—R³, or —NR⁴R⁵.

R¹ is hydrogen, C₁-C₆-alkyl, aryl, or aryl-C₁-C₆-alkyl.

R² is a selectively removable protecting group.

R³ is hydrogen or C(W)R⁶.

W is O or S.

R⁶ is C₁-C₆-alkyl, aryl, heteroaryl-C₁-C₆-alkyl, C₃-C₈-cycloalkyl-C₁-C₆-alkyl, aryl-C₁-C₆-alkyl, heteroaryl, or amino-C₁-C₆-alkyl. The amino-C₁-C₆-alkyl nitrogen optionally is substituted with:

up to two substituents independently selected from the group consisting of C₁-C₆-alkyl, aryl, aryl-C₁-C₆-alkyl, C₃-C₈-cycloalkyl-C₁-C₆-alkyl, aryl-C₁-C₆-alkoxycarbonyl, C₁-C₆-alkoxycarbonyl, and C₁-C₆-alkylcarbonyl, or

two substituents such that the amino-C₁-C₆-alkyl nitrogen and two substituents form a 5- to 8-member heterocyclyl.

R⁴ is hydrogen, C₁-C₆-alkyl, C₁-C₆-alkoxy, amino-C₁-C₆-alkyl, hydroxy-C₁-C₆-alkyl, aryl, aryloxy, or aryl-C₁-C₆-alkyl; and R⁵ is hydrogen, C₁-C₆-alkyl, amino-C₁-C₆-alkyl, hydroxy-C₁-C₆-alkyl, aryl, or aryl-C₁-C₆-alkyl. Alternatively, R⁴ and R⁵, together with the nitrogen atom to which they are both bonded, form a 5- to 8-member ring optionally comprising up to one additional heteroatom (i.e., a heteroatom in addition to the nitrogen to which both R⁴ and R⁵ are bonded) selected from the group consisting of oxygen, nitrogen, and sulfur.

In some embodiments, A² and A³ are independently selected from the group consisting of hydrogen, alkyl, alkoxyalkyl, alkylthioalkyl, alkenyl, alkynyl, carbocyclyl, carbocyclylalkyl, carbocyclylalkenyl, carbocyclylalkynyl, carbocyclyloxyalkyl, carbocyclylalkoxyalkyl, carbocyclylalkylthio, carbocyclylthioalkyl, carbocyclylalkylthioalkyl, heterocyclyl, heterocyclylalkyl, heterocyclylalkenyl, heterocyclylalkynyl, heterocyclyloxyalkyl, heterocyclylalkoxyalkyl, heterocyclylalkylthio, heterocyclylthioalkyl, and heterocyclylalkylthioalkyl. Any member of such group optionally is substituted with:

up to three independently selected R^{x} substituents; and

two substituents such that the two substituents, together with the atom(s) to which they are bonded, form a carbocyclyl or heterocyclyl, wherein the optional heterocyclyl or carbocyclyl is, in turn, optionally substituted with up to three independently selected R^{X} substituents.

In some embodiments, A² and A³, together with the carbon to which they are both bonded, form heterocyclyl or carbocyclyl. The heterocyclyl or carbocyclyl optionally is substituted with:

up to three independently selected R^{x} substituents; and

two substituents such that the two substituents, together with the atom(s) to which they are bonded, form a carbocyclyl or heterocyclyl, wherein the optional heterocyclyl or carbocyclyl is, in turn, optionally substituted with up to three independently selected R^{X} substituents.

E¹ is heteroaryl. This heteroaryl is substituted with Y. In addition to being substituted with Y, the heteroaryl optionally is substituted with one or more independently selected R^{x} substituents.

Y is halogen, nitro, azido, phenylsulfoxido, aryloxy, C₂-C₆-alkoxy, C₁-C₆-alkylsulfonate, arylsulfonate, or trisubstituted ammonium. The trisubstituted ammonium substituents are independently selected from the group consisting of aryl, aryl-C₁-C₆-alkyl, and C₁-C₆-alkyl.

Each R^{X} is independently selected from the group consisting of halogen, cyano, hydroxy, nitro, nitroso, oxo, alkyl, alkenyl, alkynyl, alkoxy, alkoxyalkoxy, R^{b}-oxyalkyl, alkenyloxy, alkynyloxy, alkylthio, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, carbocyclylthio, heterocyclyl, heterocyclylalkyl, heterocyclyloxy, heterocyclyloxyalkoxy, heterocyclylthio, alkyliminocarbonyl, alkylthioalkyl, alkylsulfonylalkyl, alkylsulfoxidoalkyl, alkylthioalkenyl, alkylsulfoxidoalkenyl, alkylsulfonylalkenyl, carbocyclylalkoxyalkyl, carbocyclyliminocarbonyl, carbocyclylthioalkyl, carbocyclylsulfoxidoalkyl, carbocyclylsulfonylalkyl, carbocyclylthioalkenyl, carbocyclylsulfoxidoalkenyl, carbocyclylsulfonylalkenyl, heterocyclylalkoxyalkyl, heterocyclylthioalkyl, heterocyclylsulfoxidoalkyl, heterocyclylsulfonylalkyl, heterocyclylthioalkenyl, heterocyclylsulfoxidoalkenyl, heterocyclylsulfonylalkenyl, heterocyclyliminocarbonyl, aminosulfonylalkyl, and —R^{x1}—R^{x2}. Any member of such group optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkoxyalkyl, and alkoxyalkoxy. Any such optional substituent is, in turn, optionally substituted with one or more substituents independently selected from the group consisting of halogen, hydroxy, and alkyl.

Each R^{x1} is —C(O)—, —C(S)—, —C(NR^{y})—, —S(O)—, or —S(O)₂—. Each R^{y}, in turn, is hydrogen or hydroxy.

Each R^{x2} is hydrogen, hydroxy, alkyl, alkenyl, alkynyl, alkoxy, alkoxyalkyl, alkoxyalkoxy, R^{b}-oxyalkyl, alkenyloxy, alkynyloxy, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, heterocyclyl, heterocyclylalkyl, heterocyclyloxy, or heterocyclyloxyalkoxy. Any member of such group optionally is substituted with one or more substituents independently selected from the group consisting of halogen, hydroxy, cyano, carboxy, thiol, sulfo, nitro, nitroso, oxo, thioxo, imino, alkyl, alkoxy, alkoxyalkyl, and alkoxyalkoxy. Any such optional substituent is, in turn, optionally substituted with one or more substituents independently selected from the group consisting of halogen and hydroxy.

Each R^{b} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl. Any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl.

Further benefits of Applicants' invention will be apparent to one skilled in the art from reading this specification.

### DETAILED DESCRIPTION OF THE PREFERRED EMBODIMENTS

This detailed description of preferred embodiments is intended only to acquaint others skilled in the art with Applicants' invention, its principles, and its practical application so that others skilled in the art may adapt and apply the invention in its numerous forms, as they may be best suited to the requirements of a particular use. This detailed description and its specific examples, while indicating preferred embodiments of this invention, are intended for purposes of illustration only. This invention, therefore, is not limited to the preferred embodiments described in this specification, and may be variously modified.

### A. Compounds of This Invention

In accordance with this invention, it has been found that certain heteroarylsulfonylmethyl hydroxamic acid and amide compounds (and salts thereof) tend to be effective for inhibiting proteases, particularly those associated with excessive (or otherwise pathological) breakdown of connective tissue. Specifically, Applicants have found that these compounds and salts tend to be effective for inhibiting proteases (particularly MMP-2, MMP-9, MMP-13, other MMP's associated with pathological conditions, and/or aggrecanase) that are often particularly destructive to tissue if present or generated in abnormally excessive quantities or concentrations. Moreover, Applicants have discovered that these compounds and salts tend to be selective toward inhibiting pathological protease activity, while avoiding excessive inhibition of other proteases (particularly MMP-1 and/or MMP-14) that are typically essential to normal bodily function (e.g., tissue turnover and repair).

### A-1. Preferred Compound Structures

The compounds of this invention generally correspond in structure to Formula (I):

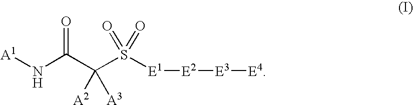

### General Description of Preferred A¹ Substituents

A¹ is hydrogen, hydroxyl, carbocyclyloxy, or heterocyclyloxy.

In some preferred embodiments, A¹ is hydrogen.

In some preferred embodiments, A¹ is hydroxy.

In some preferred embodiments, A¹ is tetrahydropyranyloxy.

### General Description of Preferred A² and A³ Substituents

In some embodiments, A² and A³ are independently selected from the group consisting of hydrogen, alkyl, alkoxyalkyl, alkylthioalkyl, alkenyl, alkynyl, carbocyclyl, carbocyclylalkyl, carbocyclylalkenyl, carbocyclylalkynyl, carbocyclyloxyalkyl, carbocyclylalkoxyalkyl, carbocyclylalkylthio, carbocyclylthioalkyl, carbocyclylalkylthioalkyl, heterocyclyl, heterocyclylalkyl, heterocyclylalkenyl, heterocyclylalkynyl, heterocyclyloxyalkyl, heterocyclylalkoxyalkyl, heterocyclylalkylthio, heterocyclylthioalkyl, and heterocyclylalkylthioalkyl. Any such substituent optionally is substituted with:

up to three independently selected R^{X} substituents; and

two substituents such that the two substituents, together with the atom(s) to which they are bonded, form a carbocyclyl or heterocyclyl, wherein the optional heterocyclyl or carbocyclyl is, in turn, optionally substituted with up to 3 independently selected R^{X} substituents.

In some preferred embodiments, A² and A³ are independently selected from the group consisting of hydrogen, alkoxyalkyl, alkylthioalkyl, alkenyl, alkynyl, carbocyclyl, carbocyclylalkyl, carbocyclylalkenyl, carbocyclylalkynyl, carbocyclyloxyalkyl, carbocyclylalkoxyalkyl, carbocyclylalkylthio, carbocyclylthioalkyl, carbocyclylalkylthioalkyl, heterocyclyl, heterocyclylalkyl, heterocyclylalkenyl, heterocyclylalkynyl, heterocyclyloxyalkyl, heterocyclylalkoxyalkyl, heterocyclylalkylthio, heterocyclylthioalkyl, and heterocyclylalkylthioalkyl. Any member of such group optionally is substituted with:

up to three independently selected R^{X} substituents; and

two substituents such that the two substituents, together with the atom(s) to which they are bonded, form a carbocyclyl or heterocyclyl, wherein the heterocyclyl or carbocyclyl optionally is substituted with up to three independently selected R^{x} substituents.

In some embodiments, A² and A³, together with the carbon to which they are both bonded, form heterocyclyl or carbocyclyl. The heterocyclyl or carbocyclyl optionally is substituted with:

up to three independently selected R^{X} substituents; and

two substituents such that the two substituents, together with the atom(s) to which they are bonded, form a carbocyclyl or heterocyclyl, wherein the optional heterocyclyl or carbocyclyl is, in turn, optionally substituted with up to three independently selected R^{X} substituents.

In some preferred embodiments,

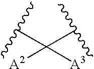

corresponds in structure to one of the following formulas:

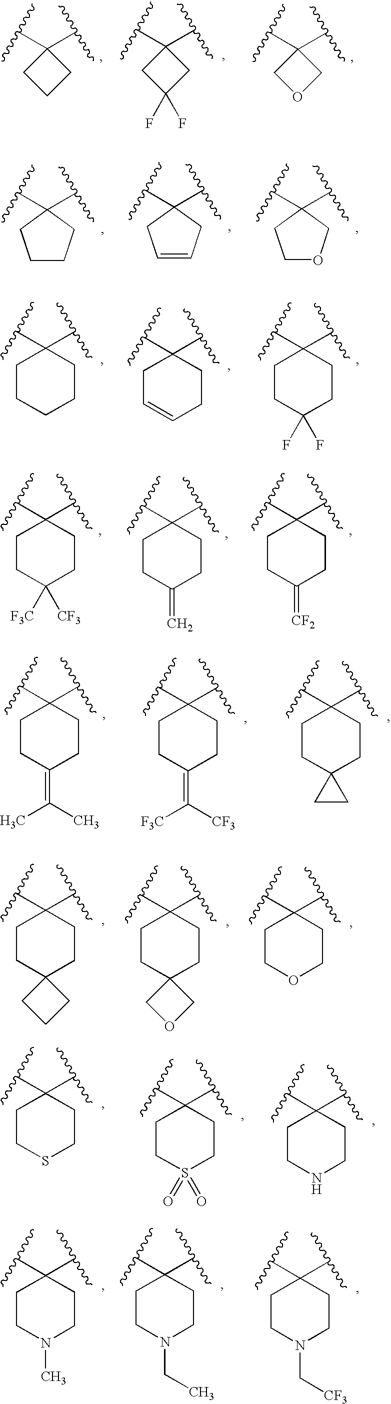

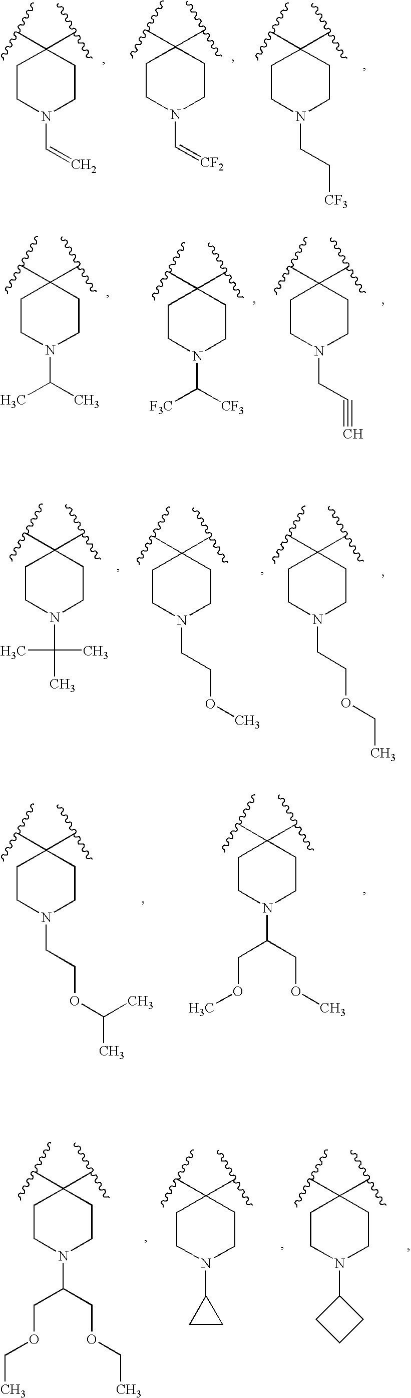

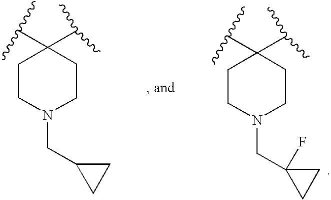

Where wavy lines are used in a chemical structure in this patent (such as in the structures above), each wavy line represents a moiety to which the depicted moiety is bonded.

In some preferred embodiments,

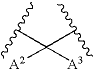

corresponds in structure to one of the following formulas:

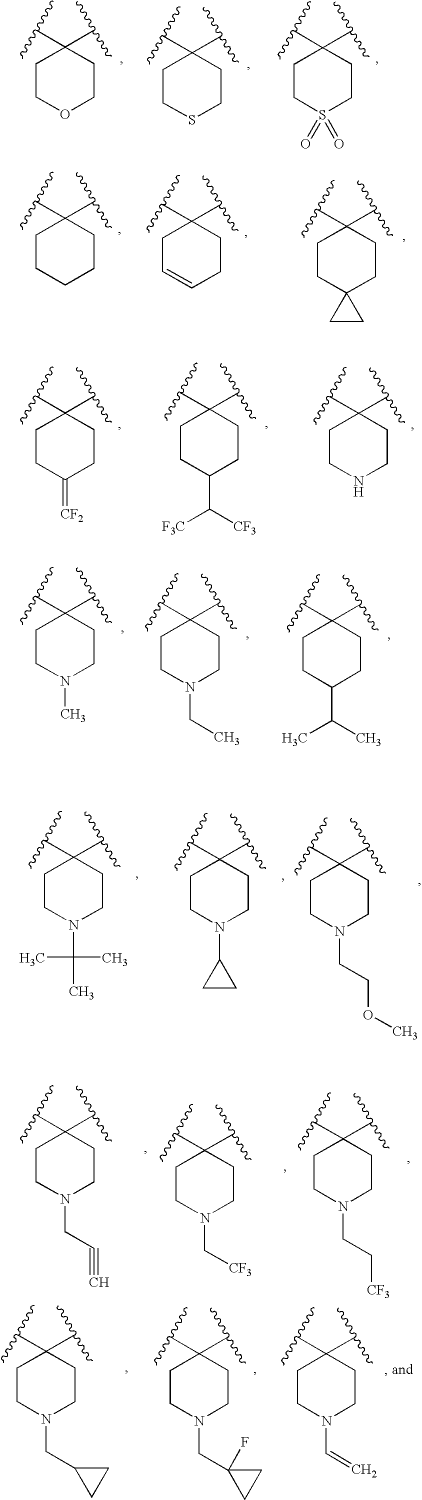

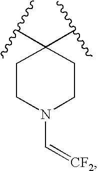

In some embodiments, A² and A³, together with the carbon to which they are both bonded, form a cyclic structure such that the compound corresponds in structure to Formula (I-1):

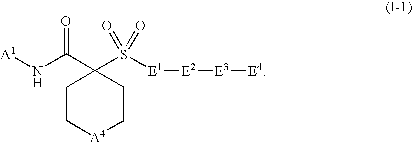

Here, A⁴ is —C(H)₂—, —C(R^{x})(H)—, —C(R^{x})₂—, —O—, —N(H)—, —N(R^{x})—, —S—, —S(O)—, or —S(O)₂—. In many such embodiments, A⁴ preferably is —O—, —N(H)—, —N(R^{x})—, —S—, —S(O)—, or —S(O)₂—.

In some particularly preferred embodiments, A⁴ is —O—. In those embodiments, the compound corresponds in structure to Formula (I-2):

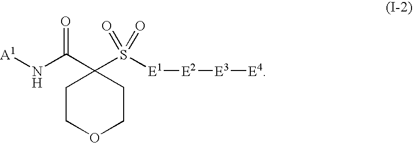

In other particularly preferred embodiments, A⁴ is —N(H)—. In those instances, the compound corresponds in structure to Formula (I-3):

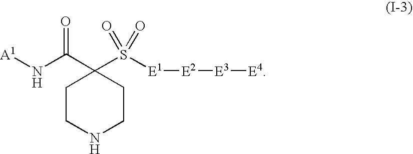

In other particularly preferred embodiments, A⁴ is —N(R^{x})—. In those instances, the compound corresponds in structure to Formula (I-4):

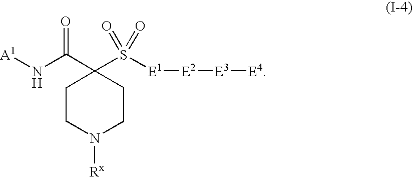

In other particularly preferred embodiments, A¹ is 2-tetrahydropyranyloxy, and the compound corresponds in structure to Formula (I-5):

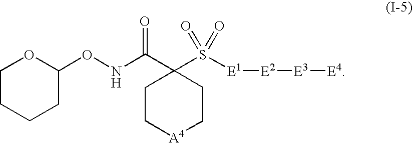

In other particularly preferred embodiments, A¹ is hydrogen, and the compound corresponds in structure to Formula (I-6):

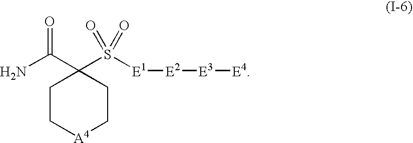

In other particularly preferred embodiments, A¹ is hydroxy, and the compound corresponds in structure to Formula (I-7):

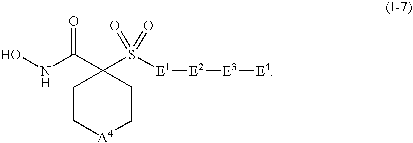

In some such particularly preferred embodiments, A⁴ is —O— such that the compound corresponds in structure to Formula (I-8):

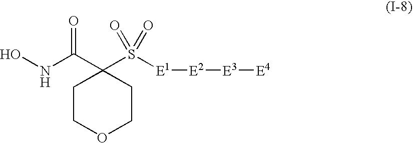

In other such particularly preferred embodiments, A⁴ is —N(R^{x})— such that the compound corresponds in structure to Formula (I-9):

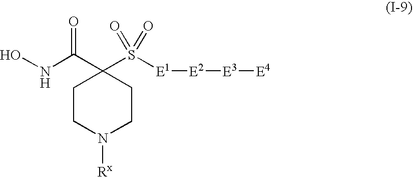

### General Description of Preferred E¹, E², E³, and E⁴ Substituents

E¹ is heteroaryl. This heteroaryl optionally is substituted with one or more independently selected R^{x} substituents. In some preferred embodiments, the heteroaryl has no such optional substituents.

In some preferred embodiments, E¹ is furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some preferred embodiments, E¹ is furanyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some preferred embodiments, E¹ is furanyl, thienyl, oxazolyl, isoxazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some preferred embodiments, E¹ is furanyl, thienyl, oxazolyl, isoxazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some preferred embodiments, E¹ is oxazolyl, isoxazolyl, isothiazolyl, thiodiazolyl, oxadiazolyl, pyrrolyl, pyrazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoisoxazolyl, anthranilyl, benzothienyl, isobenzothienyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some preferred embodiments, E¹ is oxazolyl, isoxazolyl, thiodiazolyl, oxadiazolyl, pyrrolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzothienyl, isobenzothienyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some preferred embodiments, E¹ is pyrazinyl, pyrimidinyl, pyridazinyl, furanyl, thienyl, pyrrolyl, imidazolyl, pyrazolyl, triazolyl, oxazolyl, isoxazolyl, thiazolyl, isothiazolyl, thiodiazolyl, oxathiazolyl, oxadiazolyl, pyridinyl, triazinyl, tetrazolyl, oxathiazinyl, oxepinyl, or thiepinyl. Any member of such group optionally is substituted with one or more independently selected R^{x} substituents. In many particularly preferred embodiments, however, there is no such optional substitution.

In some preferred embodiments, E¹ is pyrazinyl, pyrimidinyl, pyridazinyl, furanyl, pyrrolyl, imidazolyl, pyrazolyl, triazolyl, oxazolyl, isoxazolyl, thiazolyl, isothiazolyl, thiodiazolyl, oxathiazolyl, oxadiazolyl, pyridinyl, triazinyl, tetrazolyl, oxathiazinyl, oxepinyl, or thiepinyl. Any member of such group optionally is substituted with one or more independently selected R^{x} substituents. In many particularly preferred embodiments, however, there is no such optional substitution.

In some preferred embodiments, E¹ is pyrazinyl, pyrimidinyl, pyridazinyl, furanyl, pyrrolyl, imidazolyl, pyrazolyl, triazolyl, oxazolyl, isoxazolyl, thiazolyl, isothiazolyl, thiodiazolyl, oxathiazolyl, oxadiazolyl, triazinyl, tetrazolyl, oxathiazinyl, oxepinyl, or thiepinyl. Any member of such group optionally is substituted with one or more independently selected R^{x} substituents. In many particularly preferred embodiments, however, there is no such optional substitution.

In some preferred embodiments, E¹ is a 5-member ring. This ring optionally is substituted with one or more independently selected R^{x} substituents. In some particularly preferred embodiments, the ring has no such optional substituents. In some embodiments where E¹ is a 5-member ring, E¹ is thienyl. This thienyl optionally is substituted with one or more independently selected R^{x} substituents. In some particularly preferred embodiments, the thienyl has no such optional substituents. In such embodiments, -E¹-E²-E³-E⁴ may, for example, correspond in structure to the following formula:

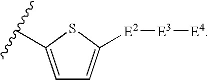

In some preferred embodiments, E¹ is a 6-member ring. This ring optionally is substituted with one or more independently selected R^{x} substituents. In some particularly preferred embodiments, the ring has no such optional substituents.

In some embodiments where E¹ is a 6-member ring, E¹ is pyrazinyl. This pyrazinyl optionally is substituted with one or more R^{x} substituents. In some particularly preferred embodiments, the pyrazinyl has no such optional substituents. In such embodiments, -E¹-E 2-E³-E⁴ may, for example, correspond in structure to the following formula:

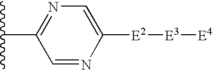

In other embodiments where E¹ is a 6-member ring, E¹ is pyrimidinyl. This pyrimidinyl optionally is substituted with one or more R^{x} substituents. In some particularly preferred embodiments, the pyrimidinyl has no such optional substituents. In such embodiments, -E¹-E²-E³-E⁴ may, for example, correspond in structure to one of the following formulas:

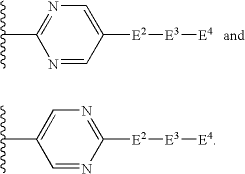

In other embodiments where E¹ is a 6-member ring, E¹ is pyridinyl. This pyridinyl optionally is substituted with one or more R^{x} substituents. In some particularly preferred embodiments, the pyridinyl has no such optional substituents. Here, the compound may, for example, correspond in structure to Formula (I-10):

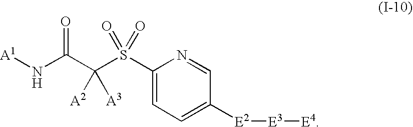

In some particularly preferred embodiments, the compound corresponds in structure to Formula (I-11):

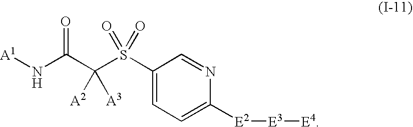

In some preferred embodiments, E¹ is a 9-member fused-ring structure. This ring structure optionally is substituted with one or more independently selected R^{x} substituents. In some particularly preferred embodiments, the ring structure has no such optional substituents. In some such embodiments, for example, the compound corresponds in structure to Formula (I-12):

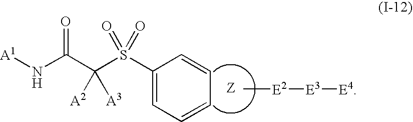

Here, the Z-ring is a 5-member ring. To illustrate, in some preferred embodiments, the compound corresponds in structure to Formula (I-13):

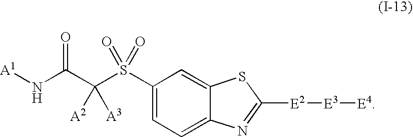

In some preferred embodiments, E¹ is a 12-member fused-ring structure. This ring structure optionally is substituted with one or more independently selected Rx substituents. In some particularly preferred embodiments, the ring structure has no such optional substituents. In some such embodiments, for example, the compound corresponds in structure to Formula (I-14):

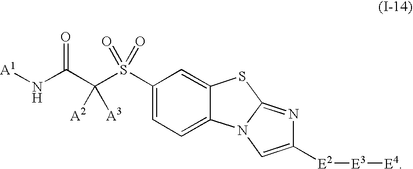

E² is carbocyclyl or heterocyclyl. The carbocyclyl or heterocyclyl optionally is substituted with one or more independently selected R^{x} substituents.

In some preferred embodiments, E² is carbocyclyl. This carbocyclyl optionally is substituted with one or more independently selected R^{x} substituents. In some particularly preferred embodiments, the carbocyclyl has no such optional substituents.

In some preferred embodiments, E² is cycloalkyl (typically single-ring cycloalkyl). This cycloalkyl optionally is substituted with one or more independently selected R^{x} substituents. In some particularly preferred embodiments, E² is single-ring cycloalkyl, wherein the cycloalkyl has no optional substituents.

In some preferred embodiments, E² is aryl (typically phenyl). This aryl optionally is substituted with one or more independently selected R^{x} substituents. In some preferred embodiments, E² is phenyl, wherein the phenyl has no such optional substituents. In some such embodiments, for example, the compound corresponds in structure to Formula (I-15):

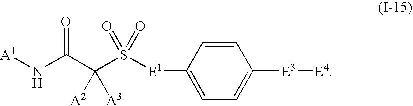

In some preferred embodiments, E² is heterocyclyl. This heterocyclyl optionally is substituted with one or more independently selected R^{x} substituents. In some particularly preferred embodiments, the heterocyclyl has no such optional substituents.

In some preferred embodiments, E² is furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, acridinyl, oxatriazolyl, dihydrofuranyl, tetrahydrofuranyl, dihydrothienyl, tetrahydrothienyl, isopyrrolyl, pyrrolinyl, pyrrolidinyl, isoimidazolyl, imidazolinyl, imidazolidinyl, pyrazolinyl, pyrazolidinyl, dithiolyl, oxathiolyl, oxathiolanyl, oxazolidinyl, isoxazolidinyl, thiazolinyl, isothiazolinyl, thiazolidinyl, isothiazolidinyl, dioxazolyl, pyranyl, dihydropyranyl, tetrahydropyranyl, piperidinyl, piperazinyl, oxazinyl, isoxazinyl, oxadiazinyl, morpholinyl, azepinyl, diazepinyl, pyrindinyl, isoindolyl, indoleninyl, pyrazolopyrimidinyl, pyrazolopyrazinyl, pyrazolopyridazyl, benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, 4H-quinolizinyl, benzoxazinyl, benzoisoxazinyl, benzoxadiazinyl, or xanthenyl. Any member of such group optionally is substituted with one or more independently selected R^{x} substituents. In many particularly preferred embodiments, however, there is no such optional substitution.

In some preferred embodiments, E² is furanyl, thienyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, acridinyl, oxatriazolyl, dihydrofuranyl, tetrahydrofuranyl, dihydrothienyl, tetrahydrothienyl, isopyrrolyl, pyrrolinyl, pyrrolidinyl, isoimidazolyl, imidazolinyl, imidazolidinyl, pyrazolinyl, pyrazolidinyl, dithiolyl, oxathiolyl, oxathiolanyl, oxazolidinyl, isoxazolidinyl, thiazolinyl, isothiazolinyl, thiazolidinyl, isothiazolidinyl, dioxazolyl, pyranyl, dihydropyranyl, tetrahydropyranyl, piperidinyl, piperazinyl, oxazinyl, isoxazinyl, oxadiazinyl, morpholinyl, azepinyl, diazepinyl, pyrindinyl, isoindolyl, indoleninyl, pyrazolopyrimidinyl, pyrazolopyrazinyl, pyrazolopyridazyl, benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, 4H-quinolizinyl, benzoxazinyl, benzoisoxazinyl, benzoxadiazinyl, or xanthenyl. Any member of such group optionally is substituted with one or more independently selected R^{x} substituents. In many particularly preferred embodiments, however, there is no such optional substitution.

In some preferred embodiments, E² is furanyl, thienyl, thiazolyl, isothiazolyl, thiodiazolyl, oxadiazolyl, pyrrolyl, pyrazolyl, imidazolyl, triazolyl, tetrazolyl, oxathiazolyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, acridinyl, oxatriazolyl, dihydrofuranyl, tetrahydrofuranyl, dihydrothienyl, tetrahydrothienyl, isopyrrolyl, pyrrolinyl, pyrrolidinyl, isoimidazolyl, imidazolinyl, imidazolidinyl, pyrazolinyl, pyrazolidinyl, dithiolyl, oxathiolyl, oxathiolanyl, oxazolidinyl, isoxazolidinyl, thiazolinyl, isothiazolinyl, thiazolidinyl, isothiazolidinyl, dioxazolyl, pyranyl, dihydropyranyl, tetrahydropyranyl, piperidinyl, piperazinyl, oxazinyl, isoxazinyl, oxadiazinyl, morpholinyl, azepinyl, diazepinyl, pyrindinyl, isoindolyl, indoleninyl, pyrazolopyrimidinyl, pyrazolopyrazinyl, pyrazolopyridazyl, benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, 4H-quinolizinyl, benzoxazinyl, benzoisoxazinyl, benzoxadiazinyl, or xanthenyl. Any member of such group optionally is substituted with one or more independently selected R^{x} substituents. In many particularly preferred embodiments, however, there is no such optional substitution.

In some preferred embodiments, E² is furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzothiadiazolyl, indolizinyl, pyranopyrrolyl, benzoxadiazolyl, indolyl, isoindazolyl, benzoimidazolyl, benzotriazolyl, purinyl, quinolinyl, isoquinolinyl, pyridopyridinyl, phthalazinyl, quinoxalinyl, benzodiazinyl, pteridinyl, carbazolyl, acridinyl, oxatriazolyl, dihydrofuranyl, tetrahydrofuranyl, dihydrothienyl, tetrahydrothienyl, isopyrrolyl, pyrrolinyl, pyrrolidinyl, isoimidazolyl, imidazolinyl, imidazolidinyl, pyrazolinyl, pyrazolidinyl, dithiolyl, oxathiolyl, oxathiolanyl, oxazolidinyl, isoxazolidinyl, thiazolinyl, isothiazolinyl, thiazolidinyl, isothiazolidinyl, dioxazolyl, pyranyl, dihydropyranyl, tetrahydropyranyl, piperidinyl, piperazinyl, oxazinyl, isoxazinyl, oxadiazinyl, morpholinyl, azepinyl, diazepinyl, pyrindinyl, isoindolyl, indoleninyl, benzodioxolyl, benzopyranyl, benzothiopyranyl, benzodioxanyl, tetrahydroisoquinolinyl, 4H-quinolizinyl, benzoxazinyl, benzoisoxazinyl, benzoxadiazinyl, or xanthenyl. Any member of such group optionally is substituted with one or more independently selected R^{x} substituents. In many particularly preferred embodiments, however, there is no such optional substitution.

In some preferred embodiments, E² is tetrazolyl, oxadiazolyl, pyrazolyl, pyridinyl, pyrimidinyl, or pyrazinyl. In some such preferred embodiments, for example, -E²-E³-E⁴ corresponds in structure to one of the following formulas:

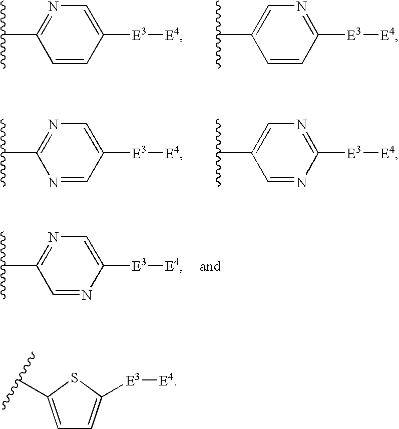

Here, -E²-E³-E⁴ may, for example, correspond in structure to one of the following formulas:

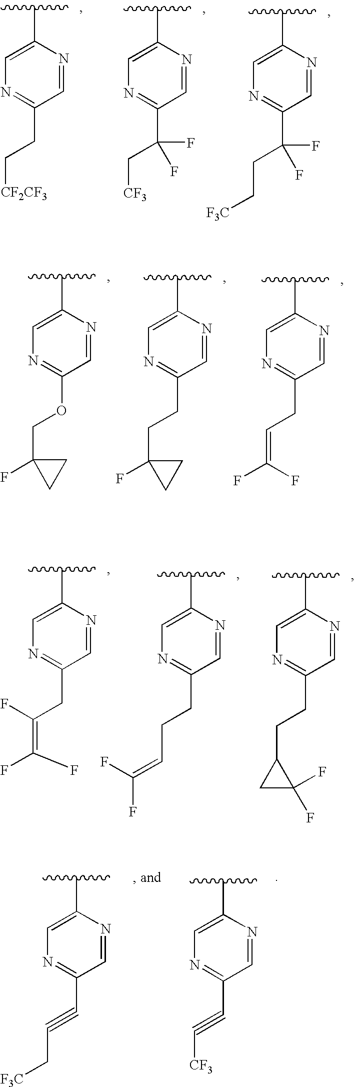

In still other such preferred embodiments, -E²-E³-E⁴ is tetrazolyl, oxadiazolyl, pyrazolyl, pyridinyl, pyrimidinyl, or pyrazinyl, wherein any member of such group optionally is substituted with alkyl, alkoxy, fluoroalkyl, or fluoroalkoxy.

In some preferred embodiments, E² is pyridinyl, pyrimidinyl, pyrazinyl, thienyl, pyrazolyl, triazolyl, oxazolyl, isoxazolyl, oxadiazolyl, thiazolyl, isothiazolyl, thiadiazolyl, or tetrazolyl. In some such embodiments, for example, -E²-E³-E⁴ corresponds in structure to one of the following formulas:

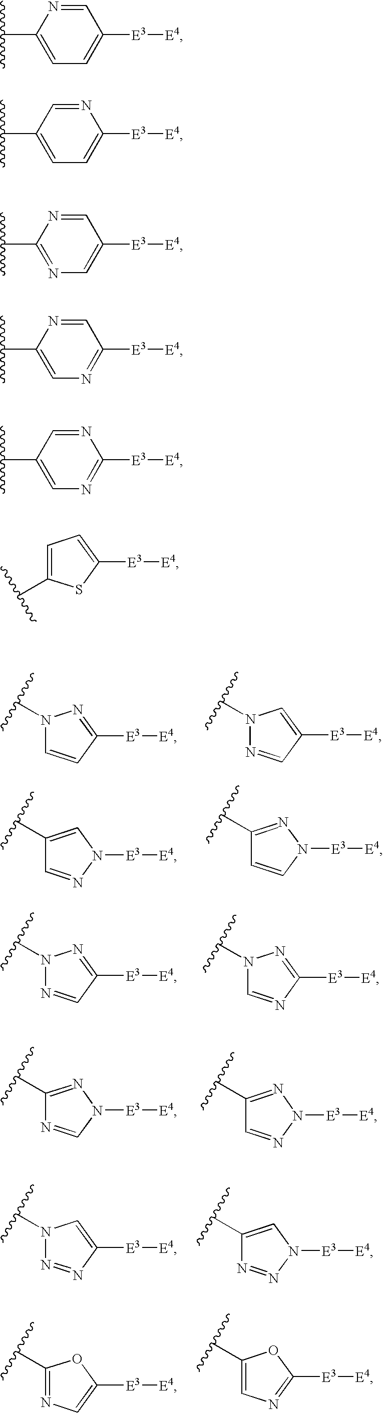

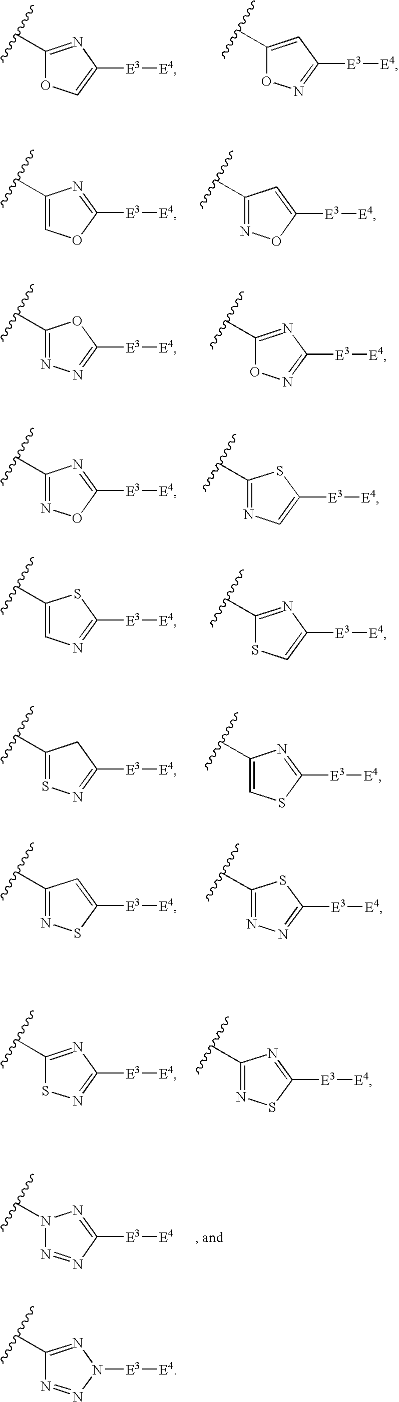

In some preferred embodiments, E² is pyridinyl, pyrimidinyl, or thienyl.

In some preferred embodiments, E² is thienyl, pyrazolyl, triazolyl, isoxazolyl, oxazolyl, isothiazolyl, thiazolyl, oxadiazolyl, thiadiazolyl, or tetrazolyl. In some such embodiments, for example, -E²-E³-E⁴ corresponds in structure to one of the following formulas:

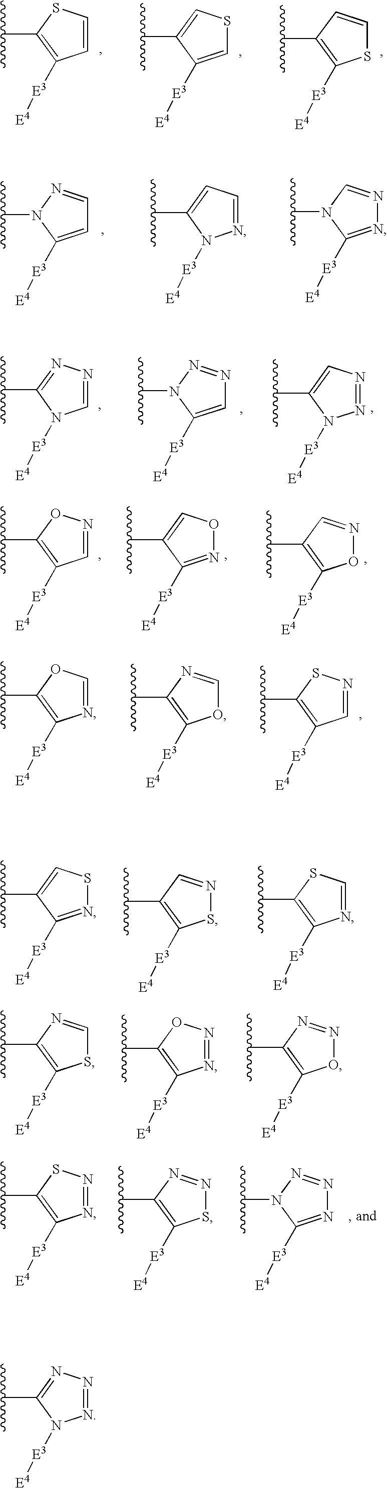

In some preferred embodiments, E² is 5-member heterocyclyl. This heterocyclyl optionally is substituted with one or more independently selected R^{x} substituents. In some particularly preferred embodiments, the heterocyclyl has no such optional R^{x} substituents.

In some preferred embodiments, E² is 5-member, saturated heterocyclyl.

In some preferred embodiments, E² is 5-member, partially-unsaturated heterocyclyl.

In some preferred embodiments, E² is 5-member heteroaryl.

In some preferred embodiments, E² is 6-member heterocyclyl. This heterocyclyl optionally is substituted with one or more independently selected Rx substituents. In some particularly preferred embodiments, the heterocyclyl has no such optional R^{x} substituents.

In some preferred embodiments, E² is 6-member, saturated heterocyclyl.

In some preferred embodiments, E² is 6-member, partially-unsaturated heterocyclyl.

In some preferred embodiments, E² is 6-member heteroaryl.

E³ is absent or selected from the group consisting of —O—, —C(O)—, —C(O)—O—, —O—C(O)—, —N(R^{b})—, —C(O)—N(R^{b})—, —N(R )—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, and a bond. Any alkyl or alkenyl portion of any such substituent optionally is substituted with one or more independently selected R^{c} substituents.

In some preferred embodiments, E³ is —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, or a bond. Any alkyl or alkenyl portion of any such substituent optionally is substituted with one or more independently selected R^{c} substituents.

In some preferred embodiments, E³ is a bond, —S—, —O—, —C(O)—, —C(O)—N(H)—, —C(O)—N(CH₃)—, —C(O)—N(CH₂CH₃)—, or —CH₂—C(O)—.

In some preferred embodiments, E³ is —C(O)—, —C(O)—N(CH₃)—, or —CH₂—C(O)—.

In some preferred embodiments, E³ is —C(O)—N(H)—, —C(O)—N(CH₃)—, or —C(O)—N(CH₂CH₃)—.

In some preferred embodiments, E³ is a bond, alkyl, —O—, —S—, or —S(O)₂—.

In some preferred embodiments, E³ is a bond, —O—, or —C(O)—.

In some preferred embodiments, E³ is —O—.

In some preferred embodiments, E³ is —S—.

In some preferred embodiments, E³ is a bond.

E⁴ is absent or selected from the group consisting of hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, and heterocyclylalkoxyalkyl. Any member of such group optionally is substituted with one or more independently selected R^{d} substituents.

In some preferred embodiments, E⁴ is hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. Any member of such group optionally is substituted with one or more independently selected R^{d} substituents.

In some preferred embodiments, E⁴ is halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. Any member of such group optionally is substituted with one or more independently selected R^{d} substituents.

In some preferred embodiments, E⁴ is alkyl, haloalkyl, alkenyl, haloalkenyl, alkynyl, haloalkynyl, cycloalkyl, halocycloalkyl, cycloalkylalkyl, or halocycloalkylalkyl. Any member of such group optionally is substituted with hydroxy.

In some preferred embodiments, E⁴ is methyl, ethyl, n-propyl, n-butyl, isopropyl, isobutyl, trifluoromethylmethyl, trifluoromethylethyl, trifluoromethylpropyl, cyclopropyl, cyclobutyl, cyclopentyl, cyclohexyl, or chloropropyl.

In some preferred embodiments, E⁴ corresponds in structure to one of the following formulas:

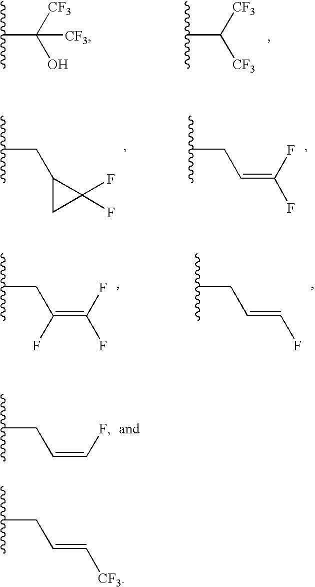

In some preferred embodiments, E⁴ corresponds in structure to one of the following formulas:

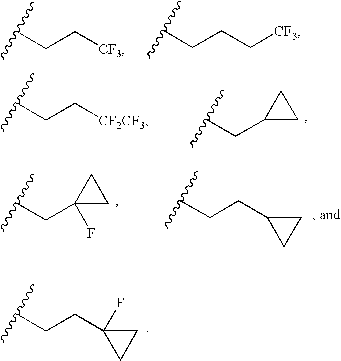

In some preferred embodiments, E⁴ is hydrogen. In some such embodiments, for example, -E³-E⁴ is hydrogen (i.e., E³ is a bond, and E⁴ is hydrogen).

In some preferred embodiments, E⁴ is alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, or aminoalkyl. Any member of such group optionally is substituted with one or more independently selected R^{d} substituents (often preferably halogen).

In some preferred embodiments, E⁴ is aminoalkyl optionally substituted with one or more independently selected R^{d} substituents. In some such embodiments, for example, E⁴ is aminocarbonylmethyl, wherein the amino is optionally substituted with up to two independently selected R^{d} substituents.

In some preferred embodiments, E⁴ is C₁-C₆-alkyl.

In some preferred embodiments, E⁴ is C₁-C₆-alkyl substituted with one or more independently selected halogen (preferably chloro or fluoro, with fluoro often being more preferred).

In some preferred embodiments, E⁴ is trifluoromethyl, or C₁-C₅-alkyl substituted with trifluoromethyl.

In some preferred embodiments, E⁴ is pentafluoroethyl, or C₁-C₄-alkyl substituted with pentafluoroethyl.

In some preferred embodiments, E⁴ is C₁-C₆-alkyl partially substituted with one or more independently selected halogen. In some such embodiments, for example, E⁴ is C₁-C₆-alkyl comprising a carbon atom bonded to at least one hydrogen and at least one halogen (often preferably fluoro).

In some preferred embodiments, E⁴ is halogen. In some such embodiments, for example, -E³-E⁴ is halogen (i.e., E³ is a bond, and E⁴ is halogen).

In some preferred embodiments, E⁴ is halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. Any member of such group optionally is substituted with one or more independently selected R^{d} substituents.

In some preferred embodiments, E⁴ corresponds in structure to one of the following formulas:

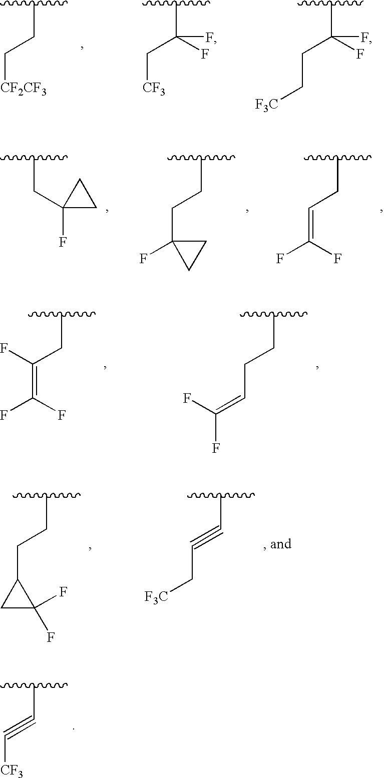

In some preferred embodiments, E⁴ is carbocyclyl, carbocyclylalkyl, carbocyclylalkoxyalkyl, heterocyclyl, heterocyclylalkyl, or heterocyclylalkoxyalkyl. Any member of such group optionally is substituted with one or more independently selected R^{d} substituents.

In some preferred embodiments, E⁴ is carbocyclyl optionally substituted with one or more independently selected R^{d} substituents.

In some preferred embodiments, E⁴ is heterocyclyl optionally substituted with one or more independently selected R^{d} substituents.

In some preferred embodiments, E⁴ is halogen, alkyl, or carbocyclyl. The alkyl or carbocyclyl optionally is substituted with one or more substituents independently selected from the group consisting of halogen, alkyl, and alkoxy. The optional alkyl and alkoxy is, in turn, optionally substituted with one or more independently selected halogen.

In some preferred embodiments, -E²-E³-E⁴ is phenyl substituted with alkyl, alkoxy, fluoroalkyl, or fluoroalkoxy.

In some preferred embodiments, -E³-E⁴ is absent. Such embodiments include, for example, compounds wherein E² is oxatriazolyl.

### General Description of Preferred R^{x} Substituents

Each R^{x} is independently selected from the group consisting of halogen, cyano, hydroxy, nitro, nitroso, oxo, alkyl, alkenyl, alkynyl, alkoxy, alkoxyalkoxy, R^{b}-oxyalkyl, alkenyloxy, alkynyloxy, alkylthio, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, carbocyclylthio, heterocyclyl, heterocyclylalkyl, heterocyclyloxy, heterocyclyloxyalkoxy, heterocyclylthio, alkyliminocarbonyl, alkylthioalkyl, alkylsulfonylalkyl, alkylsulfoxidoalkyl, alkylthioalkenyl, alkylsulfoxidoalkenyl, alkylsulfonylalkenyl, carbocyclylalkoxyalkyl, carbocyclyliminocarbonyl, carbocyclylthioalkyl, carbocyclylsulfoxidoalkyl, carbocyclylsulfonylalkyl, carbocyclylthioalkenyl, carbocyclylsulfoxidoalkenyl, carbocyclylsulfonylalkenyl, heterocyclylalkoxyalkyl, heterocyclylthioalkyl, heterocyclylsulfoxidoalkyl, heterocyclylsulfonylalkyl, heterocyclylthioalkenyl, heterocyclylsulfoxidoalkenyl, heterocyclylsulfonylalkenyl, heterocyclyliminocarbonyl, aminosulfonylalkyl, and —R^{x1}—R^{x2}. Any member of such group optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkoxyalkyl, and alkoxyalkoxy. Any such optional substituent is, in turn, optionally substituted with one or more substituents independently selected from the group consisting of halogen, hydroxy, and alkyl. In some particularly preferred embodiments, the optional alkyl, alkoxy, alkoxyalkyl, and alkoxyalkoxy are optionally substituted with one or more substituents independently selected from the group consisting of halogen and alkyl; and the optional amino is optionally substituted with up to two independently selected alkyl substituents.

Each R^{x1} is —C(O)—, —C(S)—, —C(NR^{y})—, —S(O)—, or —S(O)₂—. Here, each R^{y} is hydrogen or hydroxy.

In some preferred embodiments, each R^{x1} is —C(O)—, —C(S)—, —C(NR^{y})—, or —S(O)₂—.

Each R^{x2} is hydrogen, hydroxy, alkyl, alkenyl, alkynyl, alkoxy, alkoxyalkyl, alkoxyalkoxy, R^{b}-oxyalkyl, alkenyloxy, alkynyloxy, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, heterocyclyl, heterocyclylalkyl, heterocyclyloxy, or heterocyclyloxyalkoxy. Any member of such group optionally is substituted with one or more substituents independently selected from the group consisting of halogen, hydroxy, cyano, carboxy, thiol, sulfo, nitro, nitroso, oxo, thioxo, imino, alkyl, alkoxy, alkoxyalkyl, and alkoxyalkoxy. Any such optional substituent is, in turn, optionally substituted with one or more substituents independently selected from the group consisting of halogen and hydroxy.

### General Description of Preferred R^{b}, R^{c}, R^{d}, R^{e}, and R^{f} Substituents

Each R^{b} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl. Any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl.

Each R^{c} is independently selected from the group consisting of halogen, hydroxy, cyano, carboxy, —C(H)(NH), —C(H)(NOH), thiol, sulfo, nitro, nitroso, oxo, thioxo, imino, amino, alkyl, alkoxy, alkenyl, alkynyl, alkoxyalkyl, mono-alkylamino, di-alkylamino, alkylthio, carbocyclyl, carbocyclylalkyl, carbocyclyloxy, heterocyclyl, and heterocyclylalkyl. Any member of such group optionally is substituted with one or more substituents independently selected from the group consisting of halogen, hydroxy, cyano, carboxy, thiol, sulfo, nitro, nitroso, oxo, thioxo, imino, aminocarbonyl, amino, alkyl, and carbocyclylalkyl.

In some preferred embodiments, each R^{c} is independently selected from the group consisting of halogen, hydroxy, cyano, carboxy, —C(H)(NH), —C(H)(NOH), thiol, sulfo, nitro, nitroso, oxo, thioxo, imino, amino, alkyl, alkoxy, alkenyl, alkynyl, alkoxyalkyl, mono-alkylamino, di-alkylamino, alkylthio, carbocyclyl, carbocyclylalkyl, heterocyclyl, and heterocyclylalkyl. Any member of such group optionally is substituted with one or more substituents independently selected from the group consisting of halogen, hydroxy, cyano, carboxy, thiol, sulfo, nitro, nitroso, oxo, thioxo, imino, aminocarbonyl, amino, alkyl, and carbocyclylalkyl.

Each R^{d} is independently selected from the group consisting of halogen, hydroxy, cyano, sulfo, nitro, nitroso, oxo, thioxo, imino, alkyl, alkoxy, alkoxyalkyl, —N(R^{e})₂, —C(O)(R^{f}), —S—R^{e}, —S(O)₂—R^{e}, carbocyclyl, alkylcarbocyclyl, alkoxycarbocyclyl, carbocyclylalkyl, heterocyclyl, alkylheterocyclyl, alkoxyheterocyclyl, and heterocyclylalkyl. Any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino.

In some preferred embodiments, each R^{d} is independently selected from the group consisting of halogen, hydroxy, cyano, sulfo, nitro, nitroso, oxo, thioxo, imino, alkyl, alkoxy, alkoxyalkyl, —N(R^{e})₂, —C(O)(R^{f}), —S—R^{e}, —S(O)₂—R^{e}, carbocyclyl, alkylcarbocyclyl, carbocyclylalkyl, heterocyclyl, alkylheterocyclyl, and heterocyclylalkyl. Any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino.

Each R^{e} is independently selected from the group consisting of hydrogen alkyl, carbocyclyl, carbocyclylalkyl, heterocyclyl, and heterocyclylalkyl. Any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino.

Each R^{f} is independently selected from the group consisting of hydrogen, alkyl, —O—Re, —N(Re)₂, carbocyclylalkyl, and heterocyclylalkyl. Any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino.

### DETAILED DESCRIPTION OF PREFERRED EMBODIMENTS

The above discussion describes the compounds and salts of this invention in general terms. The following discussion, in turn, describes in detail several preferred embodiments.

### Preferred Embodiment No. 1

In some preferred embodiments:

A² and A³, together with the carbon to which they are both bonded, form heterocyclyl or carbocyclyl. The heterocyclyl or carbocyclyl optionally is substituted with:

up to three independently selected R^{x} substituents; and

two substituents such that the two substituents, together with the atom(s) to which they are bonded, form a carbocyclyl or heterocyclyl, wherein the optional heterocyclyl or carbocyclyl is, in turn, optionally substituted with up to three independently selected R^{x} substituents.

Alternatively, A² and A³ are independently selected from the group consisting of hydrogen, alkoxyalkyl, alkylthioalkyl, alkenyl, alkynyl, carbocyclyl, carbocyclylalkyl, carbocyclylalkenyl, carbocyclylalkynyl, carbocyclyloxyalkyl, carbocyclylalkoxyalkyl, carbocyclylalkylthio, carbocyclylthioalkyl, carbocyclylalkylthioalkyl, heterocyclyl, heterocyclylalkyl, heterocyclylalkenyl, heterocyclylalkynyl, heterocyclyloxyalkyl, heterocyclylalkoxyalkyl, heterocyclylalkylthio, heterocyclylthioalkyl, and heterocyclylalkylthioalkyl. Any member of such group optionally is substituted with:

up to three independently selected R^{x} substituents; and

two substituents such that the two substituents, together with the atom(s) to which they are bonded, form a carbocyclyl or heterocyclyl, wherein the heterocyclyl or carbocyclyl optionally is substituted with up to three independently selected R^{x} substituents.

E is carbocyclyl. This carbocyclyl optionally is substituted with one or more independently selected R^{x} substituents.

E³ is —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, or a bond. The alkyl or alkenyl portion of a substituent in such group optionally is substituted with one or more independently selected R^{c} substituents.

E⁴ is hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. Any member of such group optionally is substituted with one or more independently selected R^{d} substituents.

### Particularly Preferred Embodiments of Embodiment No. 1

In some particularly preferred embodiments, E¹ is furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some particularly preferred embodiments, E¹ is furanyl, thienyl, oxazolyl, isoxazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some particularly preferred embodiments, E¹ is furanyl, thienyl, oxazolyl, isoxazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some particularly preferred embodiments, E¹ is oxazolyl, isoxazolyl, isothiazolyl, thiodiazolyl, oxadiazolyl, pyrrolyl, pyrazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoisoxazolyl, anthranilyl, benzothienyl, isobenzothienyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some particularly preferred embodiments E¹, is oxazolyl, isoxazolyl, thiodiazolyl, oxadiazolyl, pyrrolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzothienyl, isobenzothienyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some particularly preferred embodiments, E¹ is thienyl, pyridinyl, pyrimidinyl, or pyrazinyl. In some such embodiments, for example, -E¹-E 2-E³-E⁴ corresponds in structure to one of the following formulas:

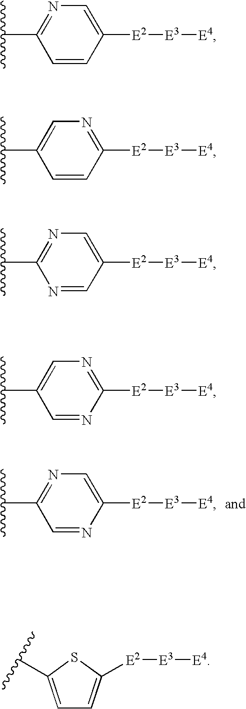

In some particularly preferred embodiments, E¹ is a 5-member ring. In some such embodiments, for example, E¹ is thienyl.

In some particularly preferred embodiments, E¹ is a 6-member ring. In some such embodiments, for example, A¹ is hydroxy, E¹ is pyridinyl, and the compound corresponds in structure to Formula (14-1):

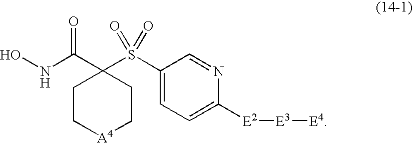

In some particularly preferred embodiments, E¹ is a 9-member fused-ring structure. In some such embodiments, for example, A¹ is hydroxy and the compound corresponds in structure to Formula (16-1):

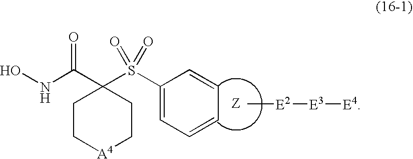

Here, the Z-ring is a 5-member ring. To illustrate, in some particularly preferred embodiments, the compound corresponds in structure to Formula (I-13A):

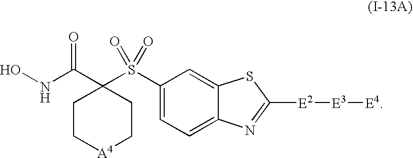

In some particularly preferred embodiments, E¹ is a 12-member fused-ring structure. In some such embodiments, for example, A¹ is hydroxy and the compound corresponds in structure to Formula (I-14A):

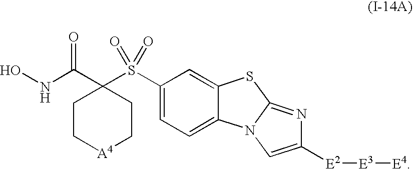

In some particularly preferred embodiments, E² is cycloalkyl (typically single-ring cycloalkyl). This cycloalkyl optionally is substituted with one or more independently selected R^{x} substituents. In many such embodiments, E² is single-ring cycloalkyl, wherein the cycloalkyl has no such optional substituents.

In some particularly preferred embodiments, E² is aryl (typically phenyl). This aryl optionally is substituted with one or more independently selected R^{x} substituents. In many embodiments, the aryl has no such optional substituents.

In some particularly preferred embodiments, E³ is a bond, —S—, —O—, —C(O)—, —C(O)—N(H)—, —C(O)—N(CH₃)—, —C(O)—N(CH₂CH₃)—, or —CH₂—C(O)—.

In some particularly preferred embodiments, E³ is —C(O)—, —C(O)—N(CH₃)—, or —CH₂—C(O)—.

In some particularly preferred embodiments, E³ is —C(O)—N(H)—, —C(O)—N(CH₃)—, or —C(O)—N(CH₂CH₃)—.

In some particularly preferred embodiments, E³ is alkyl, —O—, —S—, —S(O)₂—, or a bond.

In some particularly preferred embodiments, E³ is —O—.

In some particularly preferred embodiments, E³ is —S—.

In some particularly preferred embodiments, E³ is a bond. In some such embodiments, for example, A¹ is hydroxy, E² is phenyl, and the compound corresponds in structure to Formula I-15A:

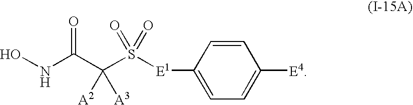

In some particularly preferred embodiments, E⁴ is hydrogen. In some such embodiments, for example, -E³-E⁴ is hydrogen (i.e., E³ is a bond, and E⁴ is hydrogen). Compounds falling within these embodiments include, for example, the compound corresponding in structure to Formula (19-1):

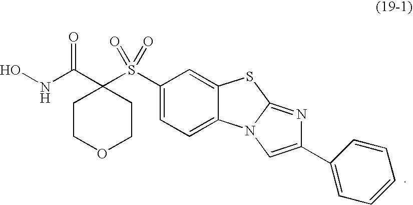

In some particularly preferred embodiments, E⁴ is halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. Any member of such group optionally is substituted with one or more independently selected R^{d} substituents.

In some particularly preferred embodiments, E⁴ is halogen. In some such embodiments, for example, -E³-E⁴ is halogen (i.e., E³ is a bond, and E⁴ is halogen). Compounds falling within these embodiments include, for example, the compounds corresponding in structure to the following formulas:

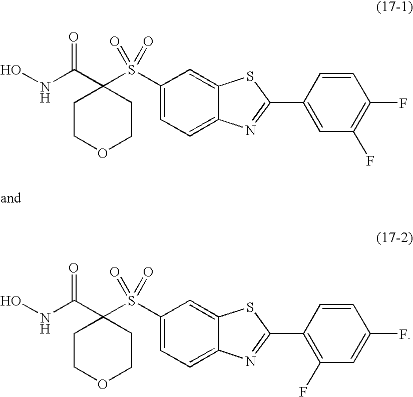

In some particularly preferred embodiments, E⁴ is carbocyclyl, carbocyclylalkyl, carbocyclylalkoxyalkyl, heterocyclyl, heterocyclylalkyl, or heterocyclylalkoxyalkyl. Any member of such group optionally is substituted with one or more independently selected R^{d} substituents.

In some particularly preferred embodiments, E⁴ is carbocyclyl optionally substituted with one or more independently selected R^{d} substituents. In some such embodiments, for example, E³ is —C(O)—, —C(O)—N(CH₃)—, or —CH₂—C(O)—. Compounds falling within such embodiments include, for example, the compounds corresponding to the following formulas:

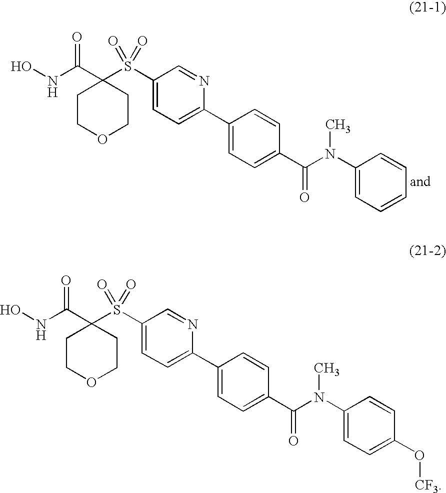

In some particularly preferred embodiments, E⁴ is heterocyclyl optionally substituted with one or more independently selected R^{d} substituents. In some such embodiments, for example, E³ is —C(O)—, —C(O)—N(CH₃)—, or —CH₂—C(O)—. Compounds falling within such embodiments include, for example, those corresponding to the following formulas:

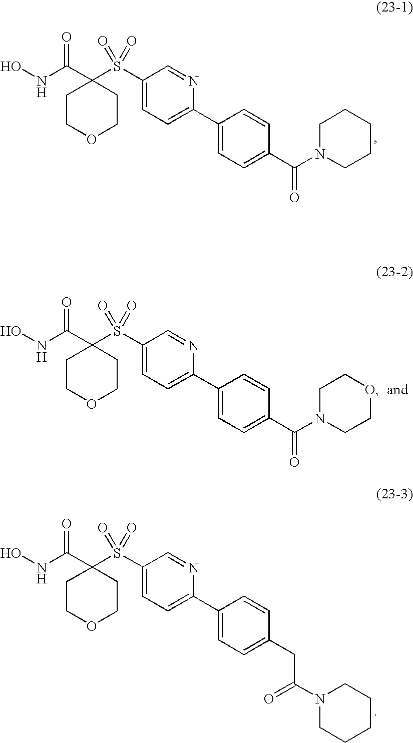

In some particularly preferred embodiments, E⁴ is alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, or aminoalkyl. Any member of such group optionally is substituted with one or more independently selected R^{d} substituents.

In some particularly preferred embodiments, E⁴ is aminoalkyl optionally substituted with one or more independently selected R^{d} substituents. In some such embodiments, for example, E⁴ is aminocarbonylmethyl, wherein the amino is optionally substituted with up to two independently selected R^{d} substituents. Compounds falling within these embodiments include, for example, the compounds corresponding to the following formulas:

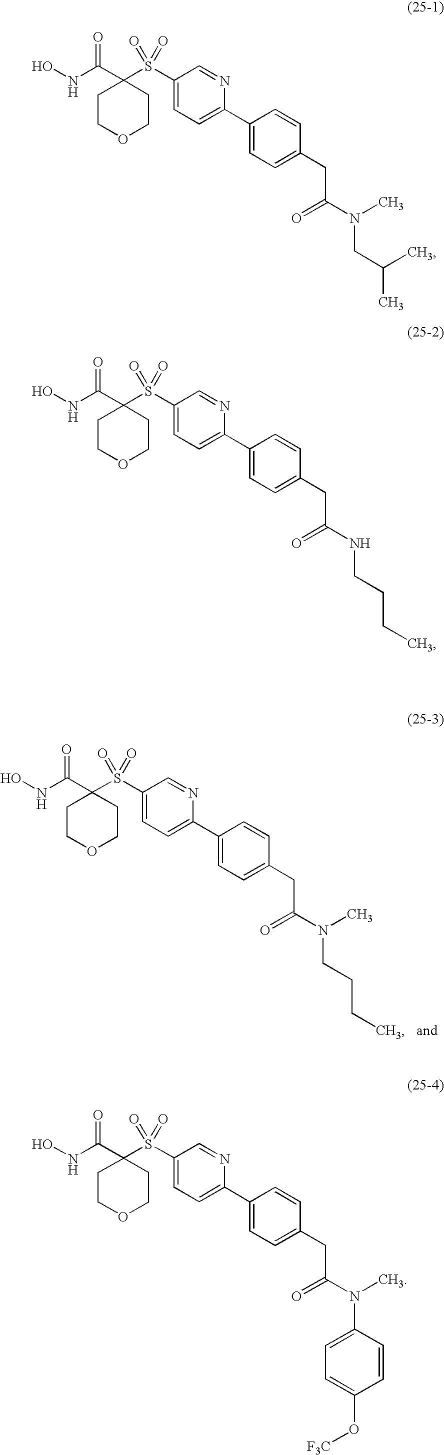

In some particularly preferred embodiments, E⁴ is alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, or aminoalkyl. Any member of such group optionally is substituted with one or more independently selected halogen.

In some particularly preferred embodiments, E⁴ is C₁-C₆-alkyl. In some such embodiments, for example, E³ is a bond. Compounds falling within such embodiments include, for example, compounds corresponding in structure to the following formulas:

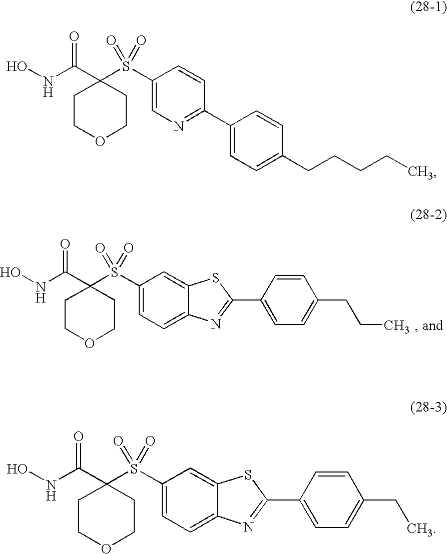

In other embodiments, E³ is —O—. Compounds falling within such embodiments include, for example, those corresponding in structure to the following formulas:

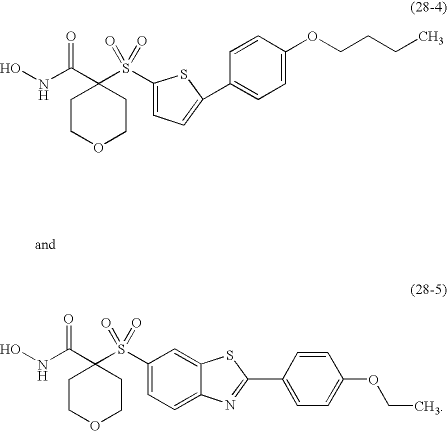

In still other embodiments, E³ is —C(O)—N(H)—, —C(O)—N(CH₃)—, or —C(O)—N(CH₂CH₃)—. Compounds falling within such embodiments include, for example, those corresponding in structure to the following formulas:

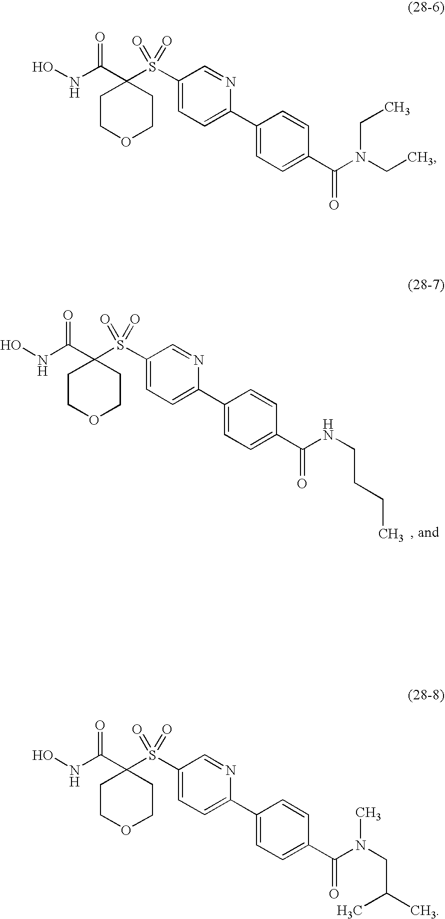

In some particularly preferred embodiments, E⁴ is C₁-C₆-alkyl substituted with one or more independently selected halogen. Such halogen are preferably chloro or fluoro, with fluoro often being more preferred.

In some particularly preferred embodiments, E⁴ is trifluoromethyl, or C₁-C₅-alkyl substituted with trifluoromethyl. In some such embodiments, for example, E³ is a bond. Compounds falling within such embodiments include, for example, those corresponding in structure to the following formulas:

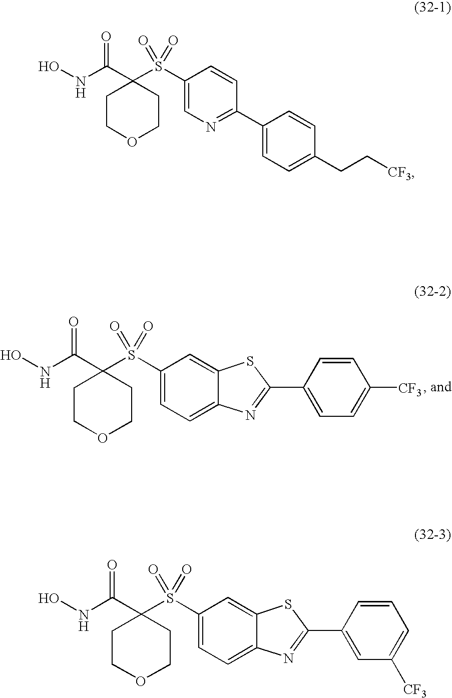

In other embodiments, E³ is —O—. Compounds falling within such embodiments include, for example, those corresponding in structure to the following formulas:

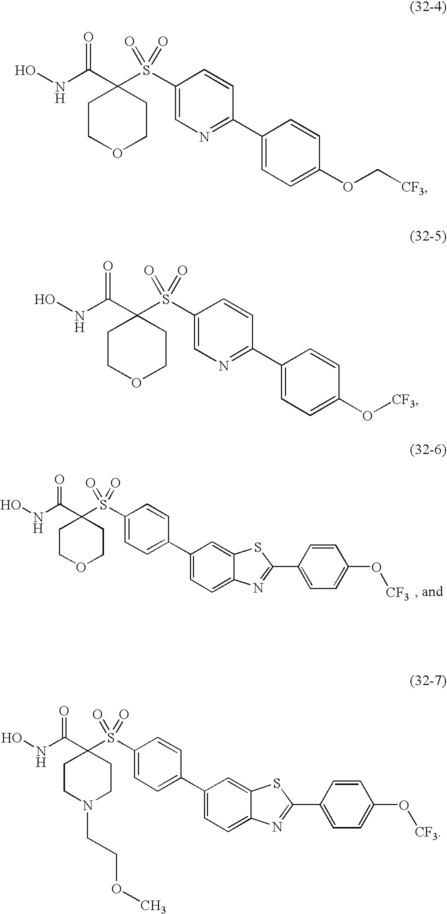

In still other embodiments, E³ is —S—. Compounds falling within such embodiments include, for example, the compound corresponding in structure to Formula (39-8):

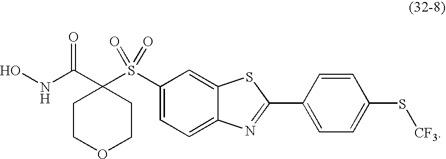

In some particularly preferred embodiments, E⁴ is pentafluoroethyl, or C₁-C₄-alkyl substituted with pentafluoroethyl. Compounds falling within such embodiments include, for example, the compound corresponding in structure to Formula (34-1):

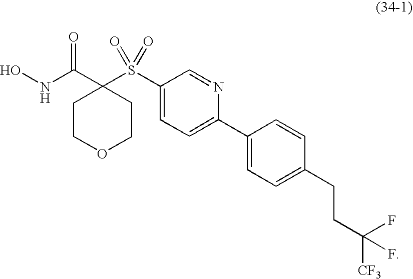

In some particularly preferred embodiments, E⁴ is C₁-C₆-alkyl partially substituted with one or more independently selected halogen. In some such embodiments, for example, E⁴ is C₁-C₆-alkyl comprising a carbon atom bonded to at least one hydrogen and at least one halogen (often preferably fluoro). Compounds falling within such embodiments include, for example, those corresponding in structure to the following formulas:

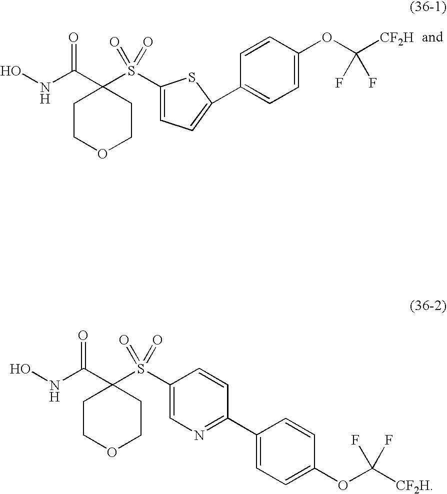

In some particularly preferred embodiments, -E²-E³-E⁴ is phenyl substituted with alkyl, alkoxy, fluoroalkyl, or fluoroalkoxy.

### Preferred Embodiment No. 2

In some preferred embodiments:

E¹ is furanyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents.

E² is heterocyclyl. This heterocyclyl optionally is substituted with one or more independently selected R^{x} substituents.

### Particularly Preferred Embodiments of Embodiment No. 2

In some particularly preferred embodiments, E¹ is oxazolyl, isoxazolyl, isothiazolyl, thiodiazolyl, oxadiazolyl, pyrrolyl, pyrazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoisoxazolyl, anthranilyl, benzothienyl, isobenzothienyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some particularly preferred embodiments, E¹ is oxazolyl, isoxazolyl, thiodiazolyl, oxadiazolyl, pyrrolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzothienyl, isobenzothienyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some particularly preferred embodiments, E¹ is 5-member heteroaryl. This heteroaryl optionally is substituted with one or more independently selected R^{x} substituents. In many preferred embodiments, the heteroaryl has no such optional substituents.

In some particularly preferred embodiments, E¹ is 6-member heteroaryl. This heteroaryl optionally substituted with one or more independently selected R^{x} substituents. In many preferred embodiments, the heteroaryl has no such optional substituents.

In some embodiments where E¹ is 6-member heteroaryl, E¹ is pyrimidinyl, pyridinyl, or pyrazinyl. In some such embodiments, for example, -E¹-E²-E³-E⁴ corresponds in structure to a formula selected from the group consisting of:

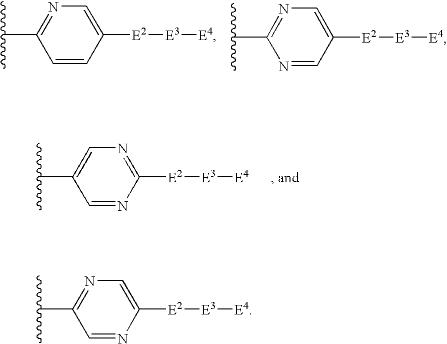

In some particularly preferred embodiments wherein E¹ is pyridinyl, -E¹-E²-E³-E⁴ corresponds in structure to the following formula:

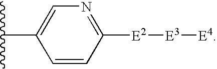

Compounds falling within such embodiments include, for example, the compound corresponding in structure to Formula (43-1):

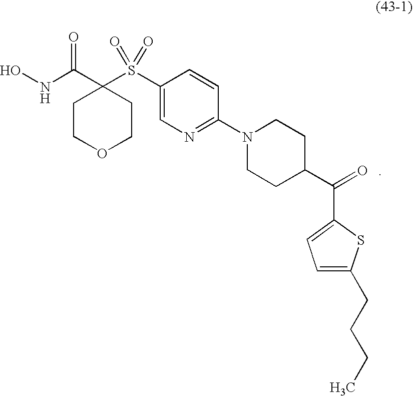

In some particularly preferred embodiments, E¹ is 9-member heteroaryl. This heteroaryl optionally is substituted with one or more independently selected R^{x} substituents. In many embodiments, the heteroaryl has no such optional substituents. In some such embodiments, for example, -E¹-E 2-E³-E⁴ corresponds in structure to the following formula:

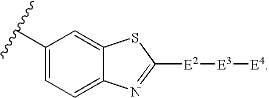

Such embodiments include, for example, compounds wherein E² is thienyl, thiazolyl, pyrazinyl, imidazolyl, piperidinyl, or benzodioxolyl. Compounds falling within such embodiments include, for example, those corresponding in structure to the following formulas:

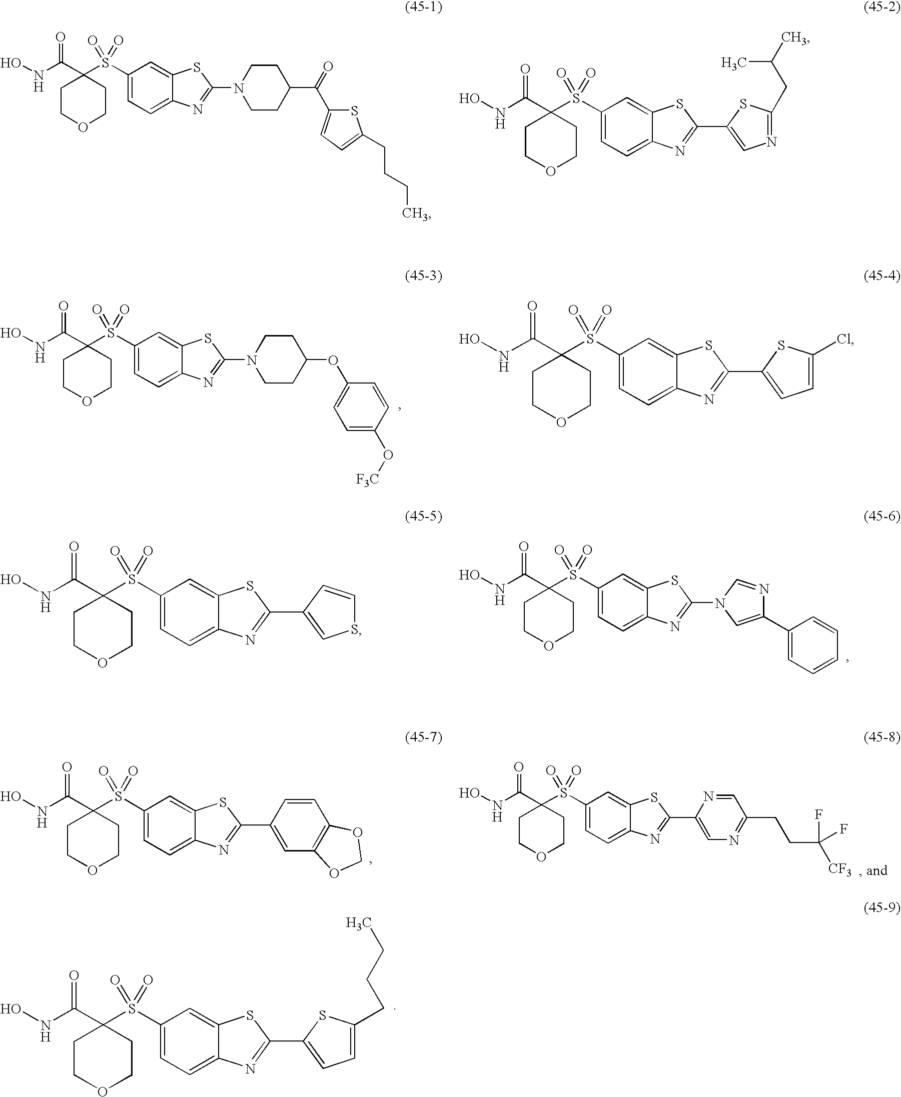

In some particularly preferred embodiments, E¹ is 12-member heteroaryl. This heteroaryl optionally is substituted with one or more independently selected R^{x} substituents. In many embodiments, the heteroaryl has no such optional substituents. In some such embodiments, for example, -E¹-E²-E³-E⁴ corresponds in structure to the following formula:

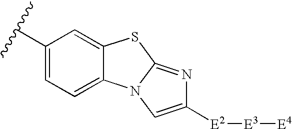

In some particularly preferred embodiments, E² is furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, acridinyl, oxatriazolyl, dihydrofuranyl, tetrahydrofuranyl, dihydrothienyl, tetrahydrothienyl, isopyrrolyl, pyrrolinyl, pyrrolidinyl, isoimidazolyl, imidazolinyl, imidazolidinyl, pyrazolinyl, pyrazolidinyl, dithiolyl, oxathiolyl, oxathiolanyl, oxazolidinyl, isoxazolidinyl, thiazolinyl, isothiazolinyl, thiazolidinyl, isothiazolidinyl, dioxazolyl, pyranyl, dihydropyranyl, tetrahydropyranyl, piperidinyl, piperazinyl, oxazinyl, isoxazinyl, oxadiazinyl, morpholinyl, azepinyl, diazepinyl, pyrindinyl, isoindolyl, indoleninyl, pyrazolopyrimidinyl, pyrazolopyrazinyl, pyrazolopyridazyl, benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, 4H-quinolizinyl, benzoxazinyl, benzoisoxazinyl, benzoxadiazinyl, or xanthenyl. Any member of such group optionally is substituted with one or more independently selected R^{x} substituents. In many particularly preferred embodiments, however, there is no such optional substitution.

In some particularly preferred embodiments, E² is furanyl, thienyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, acridinyl, oxatriazolyl, dihydrofuranyl, tetrahydrofuranyl, dihydrothienyl, tetrahydrothienyl, isopyrrolyl, pyrrolinyl, pyrrolidinyl, isoimidazolyl, imidazolinyl, imidazolidinyl, pyrazolinyl, pyrazolidinyl, dithiolyl, oxathiolyl, oxathiolanyl, oxazolidinyl, isoxazolidinyl, thiazolinyl, isothiazolinyl, thiazolidinyl, isothiazolidinyl, dioxazolyl, pyranyl, dihydropyranyl, tetrahydropyranyl, piperidinyl, piperazinyl, oxazinyl, isoxazinyl, oxadiazinyl, morpholinyl, azepinyl, diazepinyl, pyrindinyl, isoindolyl, indoleninyl, pyrazolopyrimidinyl, pyrazolopyrazinyl, pyrazolopyridazyl, benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, 4H-quinolizinyl, benzoxazinyl, benzoisoxazinyl, benzoxadiazinyl, or xanthenyl. Any member of such group optionally is substituted with one or more independently selected R^{x} substituents. In many particularly preferred embodiments, however, there is no such optional substitution.

In some particularly preferred embodiments, E² is furanyl, thienyl, thiazolyl, isothiazolyl, thiodiazolyl, oxadiazolyl, pyrrolyl, pyrazolyl, imidazolyl, triazolyl, tetrazolyl, oxathiazolyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, acridinyl, oxatriazolyl, dihydrofuranyl, tetrahydrofuranyl, dihydrothienyl, tetrahydrothienyl, isopyrrolyl, pyrrolinyl, pyrrolidinyl, isoimidazolyl, imidazolinyl, imidazolidinyl, pyrazolinyl, pyrazolidinyl, dithiolyl, oxathiolyl, oxathiolanyl, oxazolidinyl, isoxazolidinyl, thiazolinyl, isothiazolinyl, thiazolidinyl, isothiazolidinyl, dioxazolyl, pyranyl, dihydropyranyl, tetrahydropyranyl, piperidinyl, piperazinyl, oxazinyl, isoxazinyl, oxadiazinyl, morpholinyl, azepinyl, diazepinyl, pyrindinyl, isoindolyl, indoleninyl, pyrazolopyrimidinyl, pyrazolopyrazinyl, pyrazolopyridazyl, benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, 4H-quinolizinyl, benzoxazinyl, benzoisoxazinyl, benzoxadiazinyl, or xanthenyl. Any member of such group optionally is substituted with one or more independently selected R^{x} substituents. In many particularly preferred embodiments, however, there is no such optional substitution.

In some particularly preferred embodiments, E² is thienyl, thiazolyl, pyrazinyl, imidazolyl, piperidinyl, or benzodioxolyl.

In some particularly preferred embodiments, E² is tetrazolyl, oxadiazolyl, pyrazolyl, pyridinyl, pyrimidinyl, or pyrazinyl. In some such particularly preferred embodiments, for example, -E²-E³-E⁴ corresponds in structure to one of the following formulas:

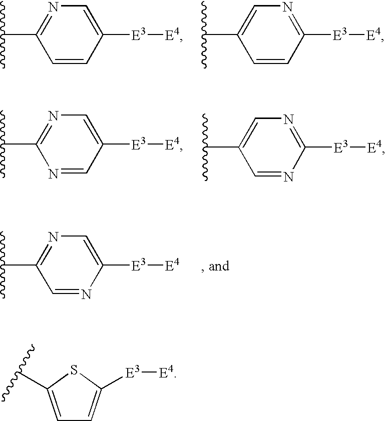

In other such particularly preferred embodiments, for example, -E²-E³-E⁴ corresponds in structure to one of the following formulas:

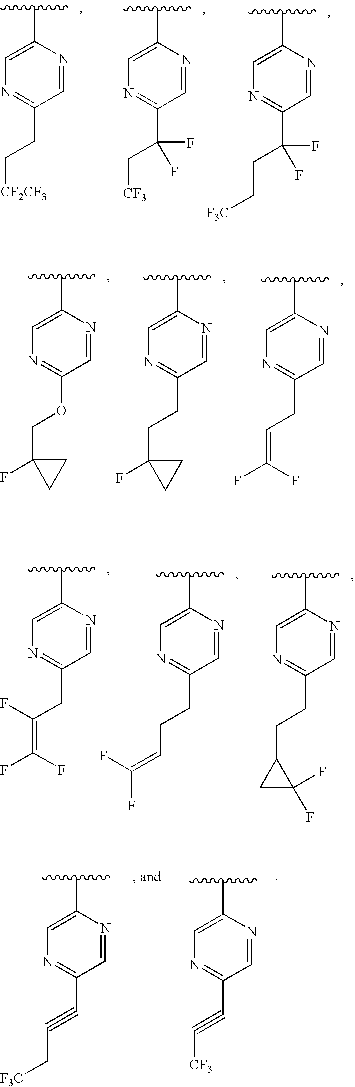

In still other such particularly preferred embodiments, -E²-E³-E⁴ is tetrazolyl, oxadiazolyl, pyrazolyl, pyridinyl, pyrimidinyl, or pyrazinyl, wherein any member of such group optionally is substituted with alkyl, alkoxy, fluoroalkyl, or fluoroalkoxy.

In some particularly preferred embodiments, E² is pyridinyl, pyrimidinyl, pyrazinyl, thienyl, pyrazolyl, triazolyl, oxazolyl, isoxazolyl, oxadiazolyl, thiazolyl, isothiazolyl, thiadiazolyl, or tetrazolyl.

In some particularly preferred embodiments, E² is pyridinyl, pyrimidinyl, or thienyl.

In some particularly preferred embodiments, E² is thienyl, pyrazolyl, triazolyl, isoxazolyl, oxazolyl, isothiazolyl, thiazolyl, oxadiazolyl, thiadiazolyl, or tetrazolyl. In some such embodiments, for example, -E²-E³-E⁴ corresponds in structure to one of the following formulas:

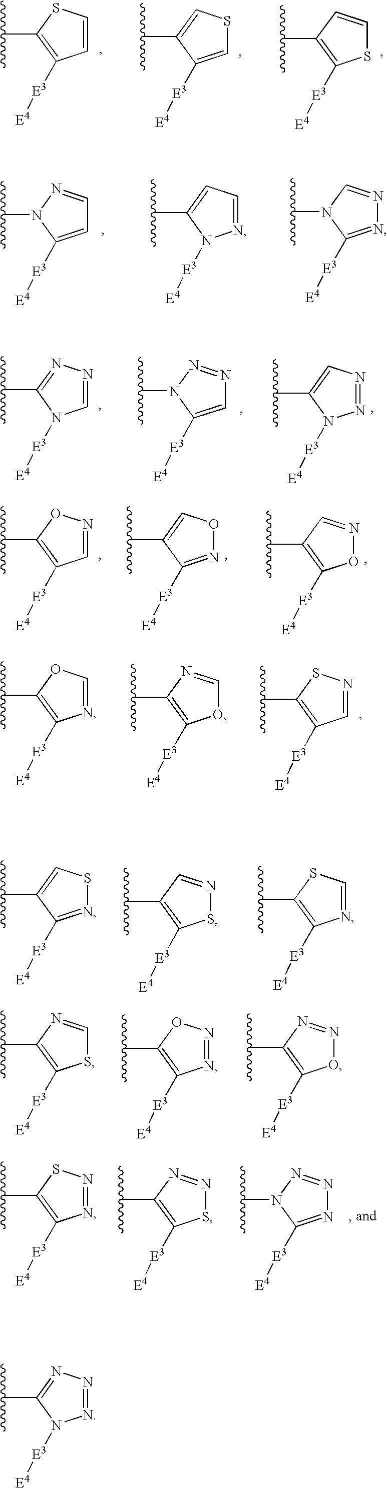

In some particularly preferred embodiments, -E²-E³-E⁴ is selected from the group consisting of:

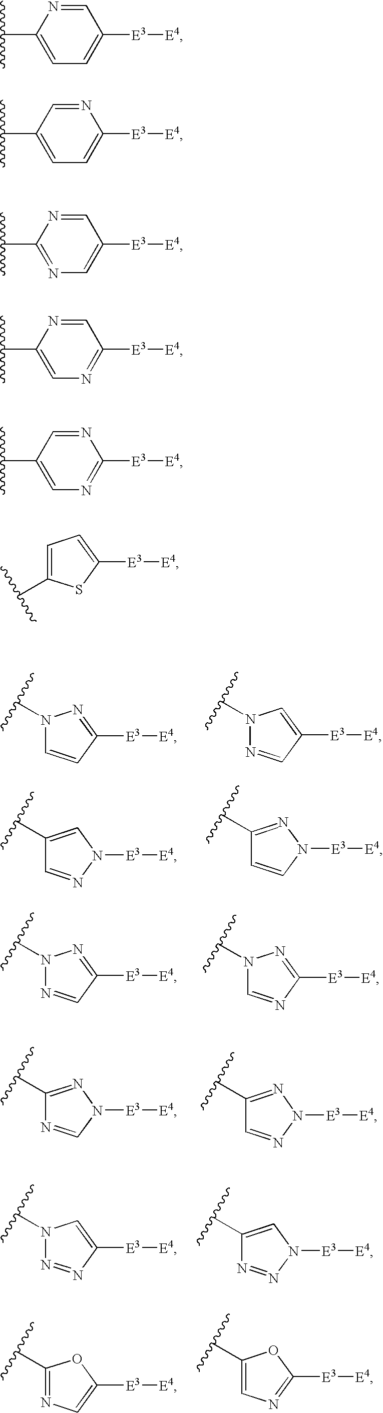

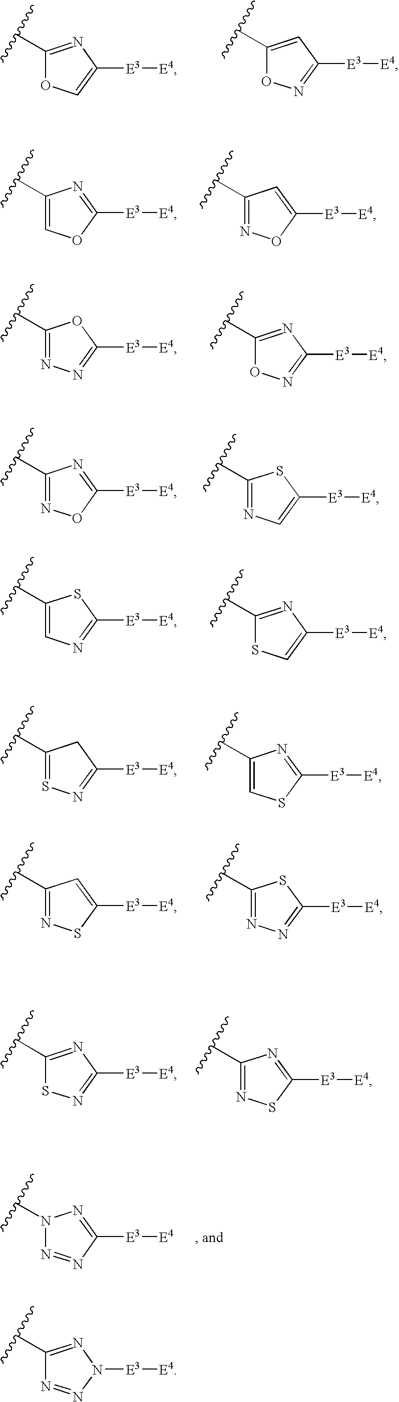

In some particularly preferred embodiments, E² is 5-member heterocyclyl. This heterocyclyl optionally is substituted with one or more independently selected R^{x} substituents. In many such embodiments, the heterocyclyl has no such optional substituents.

In some particularly preferred embodiments, E² is 5-member, saturated heterocyclyl.

In some particularly preferred embodiments, E² is 5-member, partially-unsaturated heterocyclyl.

In some particularly preferred embodiments, E² is 5-member heteroaryl.

In some particularly preferred embodiments, E² is 6-member heterocyclyl. This heterocyclyl optionally is substituted with one or more independently selected R^{x} substituents. In many such embodiments, the heterocyclyl has no such optional R^{x} substituents.

In some particularly preferred embodiments, E² is 6-member, saturated heterocyclyl.

In some particularly preferred embodiments, E² is 6-member, partially-unsaturated heterocyclyl.

In some particularly preferred embodiments, E² is 6-member heteroaryl.

In some particularly preferred embodiments, -E³-E⁴ is absent.

In some particularly preferred embodiments, E³ is —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, or a bond. Any alkyl or alkenyl portion of any such substituent optionally is substituted with one or more independently selected R^{c} substituents.

In some particularly preferred embodiments, E⁴ is hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. Any such substituent optionally is substituted with one or more independently selected R^{d} substituents.

### Preferred Embodiment No. 3

In some preferred embodiments:

E³ is —O—, —C(O)—, —C(O)—O—, —O—C(O)—, —N(R^{b}), —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, or a bond. Any alkyl or alkenyl portion of a substituent in such group optionally is substituted with one or more independently selected R^{c} substituents.

E⁴ is halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. Any member of such group optionally is substituted with one or more independently selected R^{d} substituents.

### Particularly Preferred Embodiments of Embodiment No. 3

In some particularly preferred embodiments, E¹ is furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some particularly preferred embodiments, E¹ is oxazolyl, isoxazolyl, isothiazolyl, thiodiazolyl, oxadiazolyl, pyrrolyl, pyrazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoisoxazolyl, anthranilyl, benzothienyl, isobenzothienyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some particularly preferred embodiments E¹, is oxazolyl, isoxazolyl, thiodiazolyl, oxadiazolyl, pyrrolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzothienyl, isobenzothienyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some particularly preferred embodiments, E¹ is pyridinyl, pyrimidinyl, or pyrazinyl. In some such embodiments, for example, -E¹-E²-E³-E⁴ corresponds in structure to one of the following formulas:

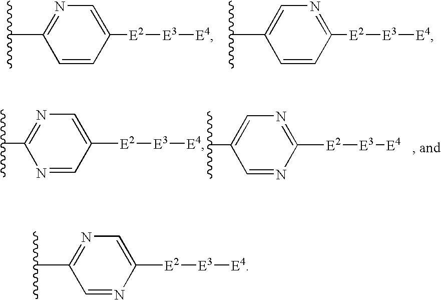

In some particularly preferred embodiments, E¹ is thienyl. In some such embodiments, for example, -E¹-E²-E³-E⁴ corresponds in structure to the following formula:

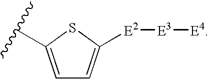

Compounds falling within such embodiments include, for example, compounds corresponding in structure to one of the following formulas:

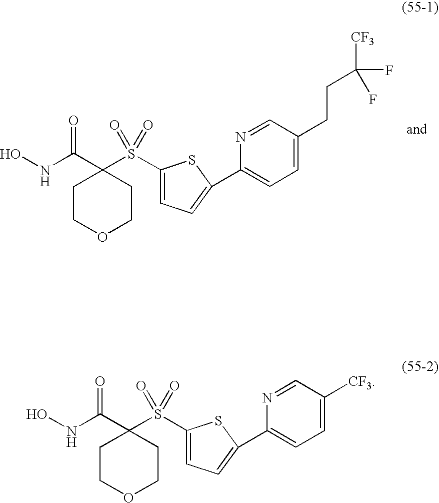

(55-1)

In some particularly preferred embodiments, E² is furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, acridinyl, dihydrofuranyl, tetrahydrofuranyl, dihydrothienyl, tetrahydrothienyl, isopyrrolyl, pyrrolinyl, pyrrolidinyl, isoimidazolyl, imidazolinyl, imidazolidinyl, pyrazolinyl, pyrazolidinyl, dithiolyl, oxathiolyl, oxathiolanyl, oxazolidinyl, isoxazolidinyl, thiazolinyl, isothiazolinyl, thiazolidinyl, isothiazolidinyl, dioxazolyl, pyranyl, dihydropyranyl, tetrahydropyranyl, piperidinyl, piperazinyl, oxazinyl, isoxazinyl, oxadiazinyl, morpholinyl, azepinyl, diazepinyl, pyrindinyl, isoindolyl, indoleninyl, pyrazolopyrimidinyl, pyrazolopyrazinyl, pyrazolopyridazyl, benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, 4H-quinolizinyl, benzoxazinyl, benzoisoxazinyl, benzoxadiazinyl, or xanthenyl. Any member of such group optionally is substituted with one or more independently selected R^{x} substituents. In many particularly preferred embodiments, however, there is no such optional substitution.

In some particularly preferred embodiments, E² is furanyl, thienyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, acridinyl, dihydrofuranyl, tetrahydrofuranyl, dihydrothienyl, tetrahydrothienyl, isopyrrolyl, pyrrolinyl, pyrrolidinyl, isoimidazolyl, imidazolinyl, imidazolidinyl, pyrazolinyl, pyrazolidinyl, dithiolyl, oxathiolyl, oxathiolanyl, oxazolidinyl, isoxazolidinyl, thiazolinyl, isothiazolinyl, thiazolidinyl, isothiazolidinyl, dioxazolyl, pyranyl, dihydropyranyl, tetrahydropyranyl, piperidinyl, piperazinyl, oxazinyl, isoxazinyl, oxadiazinyl, morpholinyl, azepinyl, diazepinyl, pyrindinyl, isoindolyl, indoleninyl, pyrazolopyrimidinyl, pyrazolopyrazinyl, pyrazolopyridazyl, benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, 4H-quinolizinyl, benzoxazinyl, benzoisoxazinyl, benzoxadiazinyl, or xanthenyl. Any member of such group optionally is substituted with one or more independently selected R^{x} substituents. In many particularly preferred embodiments, however, there is no such optional substitution.

In some particularly preferred embodiments, E² is furanyl, thienyl, thiazolyl, isothiazolyl, thiodiazolyl, oxadiazolyl, pyrrolyl, pyrazolyl, imidazolyl, triazolyl, tetrazolyl, oxathiazolyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, acridinyl, dihydrofuranyl, tetrahydrofuranyl, dihydrothienyl, tetrahydrothienyl, isopyrrolyl, pyrrolinyl, pyrrolidinyl, isoimidazolyl, imidazolinyl, imidazolidinyl, pyrazolinyl, pyrazolidinyl, dithiolyl, oxathiolyl, oxathiolanyl, oxazolidinyl, isoxazolidinyl, thiazolinyl, isothiazolinyl, thiazolidinyl, isothiazolidinyl, dioxazolyl, pyranyl, dihydropyranyl, tetrahydropyranyl, piperidinyl, piperazinyl, oxazinyl, isoxazinyl, oxadiazinyl, morpholinyl, azepinyl, diazepinyl, pyrindinyl, isoindolyl, indoleninyl, pyrazolopyrimidinyl, pyrazolopyrazinyl, pyrazolopyridazyl, benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, 4H-quinolizinyl, benzoxazinyl, benzoisoxazinyl, benzoxadiazinyl, or xanthenyl. Any member of such group optionally is substituted with one or more independently selected R^{x} substituents. In many particularly preferred embodiments, however, there is no such optional substitution.

In some particularly preferred embodiments, E³ is a bond, —S—, —O—, —C(O)—, —C(O)—N(H)—, —C(O)—N(CH₃)—, —C(O)—N(CH₂CH₃)—, or —CH₂—C(O)—.

In some particularly preferred embodiments, E³ is a bond, —O—, or —C(O)—.

In some particularly preferred embodiments, E⁴ is halogen, alkyl, or carbocyclyl. The alkyl or carbocyclyl optionally is substituted with one or more substituents independently selected from the group consisting of halogen, alkyl, and alkoxy. The optional alkyl and alkoxy are, in turn, optionally substituted with one or more independently selected halogen.

### A-2. Preferred MMP Selectivities

When a compound or salt of this invention is used to treat conditions associated with MMP activity, the compound or salt preferably has an inhibitory activity against MMP-1 or MMP-14 that is substantially less than its inhibitory activity against MMP-2, MMP-9, or MMP-13. In other words, the compound or salt preferably has an in inhibition constant (Kᵢ) against at least one of MMP-2, MMP-9, and MMP-13 that is no greater than about 0.1 times its inhibition constant(s) against at least one of MMP-1 and MMP-14. The inhibition constant of a compound or salt may be determined using an in vitro inhibition assay, such as the Kᵢ assay described in the Examples below.

In some particularly preferred embodiments, the compound or salt preferably has a Kᵢ against MMP-2 that is no greater than about 0.1 (more preferably no greater than about 0.01, even more preferably no greater than about 0.001, still more preferably no greater than about 0.0001, and still even more preferably no greater than about 0.00001) times its Kᵢ(s) against one or both of MMP-1 and MMP-14 (often preferably both).

In some particularly preferred embodiments, the compound or salt preferably has a Kᵢ against MMP-9 that is no greater than about 0.1 (more preferably no greater than about 0.01, even more preferably no greater than about 0.001, still more preferably no greater than about 0.0001, and still even more preferably no greater than about 0.00001) times its Kᵢ(s) against one or both of MMP-1 and MMP-14 (often preferably both). It is believed that such a selectivity profile is often particularly preferred when treating, for example, a pathological condition of the central nervous system associated with nitrosative or oxidative stress. Such a pathological condition may be, for example, cerebral ischemia, stroke, or other neurodegenerative disease.

In some particularly preferred embodiments, the compound or salt preferably has a Kᵢ against MMP-13 that is no greater than about 0.1 (more preferably no greater than about 0.01, even more preferably no greater than about 0.001, still more preferably no greater than about 0.0001, and still even more preferably no greater than about 0.00001) times its Kᵢ(s) against one or both of MMP-1 and MMP-14 (often preferably both). It is believed that such a selectivity profile is often particularly preferred when treating, for example, a cardiovascular condition or arthritis.

In some particularly preferred embodiments, the compound or salt preferably has Kᵢ's against both MMP-2 and MMP-9 that are no greater than about 0.1 (more preferably no greater than about 0.01, even more preferably no greater than about 0.001, still more preferably no greater than about 0.0001, and still even more preferably no greater than about 0.00001) times its Kᵢ(s) against one or both of MMP-1 and MMP-14 (often preferably both). It is believed that such a selectivity profile is often particularly preferred when treating, for example, cancer, a cardiovascular condition, or an ophthalmologic condition.

In some particularly preferred embodiments, the compound or salt preferably has Kᵢ's against all of MMP-2, MMP-9, and MMP-13 that are no greater than about 0.1 (more preferably no greater than about 0.01, even more preferably no greater than about 0.001, still more preferably no greater than about 0.0001, and still even more preferably no greater than about 0.00001) times its Kᵢ(s) against one or both of MMP-1 and MMP-14 (often preferably both). It is believed that such a selectivity profile is often particularly preferred when treating, for example, cancer, a cardiovascular condition, arthritis, or an ophthalmologic condition.

The activity and selectivity of a compound or salt of this invention may alternatively be determined using an in vitro IC₅₀ assay, such as the IC₅₀ assay described in WIPO Publ. No. WO 02/092588 (Appl. No. PCT/US02/15257; filed May 10, 2002; published Nov. 21, 2002) (incorporated by reference into this patent). In that instance, the compound or salt preferably has an IC₅₀ value against at least one of MMP-2, MMP-9, and MMP-13 that is no greater than about 0.1 times its IC₅₀ value(s) against at least one of MMP-1 and MMP-14.

In some particularly preferred embodiments, the compound or salt preferably has an IC₅₀ value against MMP-2 that is no greater than about 0.1 (more preferably no greater than about 0.01, even more preferably no greater than about 0.001, still more preferably no greater than about 0.0001, and still even more preferably no greater than about 0.00001) times its IC₅₀ value(s) against one or both of MMP-1 and MMP-14 (often preferably both).

In some particularly preferred embodiments, the compound or salt preferably has an IC₅₀ value against MMP-9 that is no greater than about 0.1 (more preferably no greater than about 0.01, even more preferably no greater than about 0.001, still more preferably no greater than about 0.0001, and still even more preferably no greater than about 0.00001) times its IC₅₀ value(s) against one or both of MMP-1 and MMP-14 (often preferably both). It is believed that such a selectivity profile is often particularly preferred when treating, for example, a pathological condition of the central nervous system associated with nitrosative or oxidative stress. Such a pathological condition may be, for example, cerebral ischemia, stroke, or other neurodegenerative disease.

In some particularly preferred embodiments, the compound or salt preferably has an IC₅₀ value against MMP-13 that is no greater than about 0.1 (more preferably no greater than about 0.01, even more preferably no greater than about 0.001, still more preferably no greater than about 0.0001, and still even more preferably no greater than about 0.00001) times its IC₅₀ value(s) against one or both of MMP-1 and MMP-14 (often preferably both). It is believed that such a selectivity profile is often particularly preferred when treating, for example, a cardiovascular condition or arthritis.

In some particularly preferred embodiments, the compound or salt preferably has IC₅₀ values against both MMP-2 and MMP-9 that are no greater than about 0.1 (more preferably no greater than about 0.01, even more preferably no greater than about 0.001, still more preferably no greater than about 0.0001, and still even more preferably no greater than about 0.00001) times its IC₅₀ value(s) against one or both of MMP-1 and MMP-14 (often preferably both). It is believed that such a selectivity profile is often particularly preferred when treating, for example, cancer, a cardiovascular condition, or an ophthalmologic condition.

In some particularly preferred embodiments, the compound or salt preferably has IC₅₀ values against all of MMP-2, MMP-9, and MMP-13 that are no greater than about 0.1 (more preferably no greater than about 0.01, even more preferably no greater than about 0.001, still more preferably no greater than about 0.0001, and still even more preferably no greater than about 0.00001) times its IC₅₀ value(s) against one or both of MMP-1 and MMP-14 (often preferably both). It is believed that such a selectivity profile is often particularly preferred when treating, for example, cancer, a cardiovascular condition, arthritis, or an ophthalmologic condition.

### B. Salts of the Compounds of this Invention

The compounds of this invention can be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. Pharmaceutically acceptable salts include salts commonly used to form alkali metal salts and to form addition salts of free acids or free bases. In general, these salts typically may be prepared by conventional means with a compound of this invention by reacting, for example, the appropriate acid or base with the compound.

Pharmaceutically acceptable acid addition salts of the compounds of this invention may be prepared from an inorganic or organic acid. Examples of suitable inorganic acids include hydrochloric, hydrobromic acid, hydroiodic, nitric, carbonic, sulfuric, and phosphoric acid. Suitable organic acids generally include, for example, aliphatic, cycloaliphatic, aromatic, araliphatic, heterocyclic, carboxylic, and sulfonic classes of organic acids. Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), ethanesulfonate, benzenesulfonate, pantothenate, 2-hydroxyethanesulfonate, sulfanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate.

Pharmaceutically acceptable base addition salts of the compounds of this invention include, for example, metallic salts and organic salts. Preferred metallic salts include alkali metal (group Ia) salts, alkaline earth metal (group Ia) salts, and other physiologically acceptable metal salts. Such salts may be made from aluminum, calcium, lithium, magnesium, potassium, sodium, and zinc. Preferred organic salts can be made from amines, such as tromethamine, diethylamine, N,N′-dibenzylethylenediamine, chloroprocaine, diethanolamine, ethylenediamine, meglumine (N-methylglucamine), and procaine. Basic nitrogen-containing groups can be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibuytl, and diamyl sulfates), long chain halides (e.g., decyl, lauryl, myristyl, and stearyl chlorides, bromides, and iodides), aralkyl halides (e.g., benzyl and phenethyl bromides), and others.

In some particularly preferred embodiments, the salt comprises a hydrochloric acid (HCl) salt.

In other particularly preferred embodiments, the salt comprises a trifluoroacetate (CF₃COOH or “TFA”) salt.

### C. Treating Conditions Using the Compounds and Salts of this Invention

One embodiment of this invention is directed to a process for treating a pathological condition associated with pathologically-excessive MMP, TNF, and/or aggrecanase activity in a mammal (e.g., a human, companion animal, farm animal, laboratory animal, zoo animal, or wild animal) having or disposed to having such a condition. Such a condition may be, for example, tissue destruction, a fibrotic disease, pathological matrix weakening, defective injury repair, a cardiovascular disease, a pulmonary disease, a kidney disease, a liver disease, an ophthalmologic disease, or a central nervous system disease. Specific examples of such conditions include osteoarthritis, rheumatoid arthritis, septic arthritis, tumor invasion, tumor metastasis, tumor angiogenesis, a decubitis ulcer, a gastric ulcer, a corneal ulcer, periodontal disease, liver cirrhosis, fibrotic lung disease, otosclerosis, atherosclerosis, multiple sclerosis, dilated cardiomyopathy, epidermal ulceration, epidermolysis bullosa, aortic aneurysm, weak injury repair, an adhesion, scarring, congestive heart failure, post myocardial infarction, coronary thrombosis, emphysema, proteinuria, bone disease, chronic obstructive pulmonary diseases, Alzheimer's disease, and diseases of the central nervous system (particularly those associated with nitrosative or oxidative stress).

In some particularly contemplated embodiments, the condition comprises arthritis.

In some particularly contemplated embodiments, the condition comprises tumor invasion, tumor metastasis, or tumor angiogenesis.

In some particularly contemplated embodiments, the condition comprises periodontal disease.

In some particularly contemplated embodiments, the condition comprises atherosclerosis.

In some particularly contemplated embodiments, the condition comprises multiple sclerosis.

In some particularly contemplated embodiments, the condition comprises dilated cardiomyopathy.

In some particularly contemplated embodiments, the condition comprises post myocardial infarction.

In some particularly contemplated embodiments, the condition comprises congestive heart failure.

In some particularly contemplated embodiments, the condition comprises chronic obstructive pulmonary disease.

In some particularly contemplated embodiments, the condition comprises an ophthalmologic disease.

In some particularly contemplated embodiments, the condition comprises a disease of the central nervous system, particularly a disease associated with nitrosative or oxidative stress. Such a disease may be, for example, stroke, cerebral ischemia, and other neurodegenerative diseases.

The condition may alternatively (or additionally) be associated with TNF-α convertase activity. Examples of such a condition include inflammation (e.g., rheumatoid arthritis), autoimmune disease, graft rejection, multiple sclerosis, a fibrotic disease, cancer, an infectious disease (e.g., malaria, mycobacterial infection, meningitis, etc.), fever, psoriasis, a cardiovascular disease (e.g., post-ischemic reperfusion injury, congestive heart failure, etc.), a pulmonary disease (e.g., hyperoxic alveolar injury), hemorrhage, coagulation, radiation damage, acute phase responses like those seen with infections and sepsis and during shock (e.g., septic shock, hemodynamic shock, etc.), cachexia, and anorexia.

The condition may alternatively (or additionally) be associated with aggrecanase activity. Examples of such a condition include inflammation diseases (e.g., osteoarthritis, rheumatoid arthritis, joint injury, reactive arthritis, acute pyrophosphate arthritis, and psoriatic arthritis) and cancer.

In this specification, the phrase “treating a condition” means ameliorating, suppressing, eradicating, preventing, reducing the risk of, or delaying the onset of the condition. The pathological condition may be (a) the result of pathological aggrecanase and/or MMP activity itself, and/or (b) affected by aggrecanase and/or MMP activity (e.g., diseases associated with TNF-α).

A wide variety of methods may be used alone or in combination to administer the compounds and salt thereof described above. For example, the compounds or salts thereof may be administered orally, parenterally, by inhalation spray, rectally, or topically.

Typically, a compound (or pharmaceutically acceptable salt thereof) described in this patent is administered in an amount effective to inhibit a target MMP(s), TNF, and/or aggrecanase. The target MMP(s) is/are typically MMP-2, MMP-9, and/or MMP-13.

In some preferred embodiments, the A¹ substituent of the compound or salt is hydrogen, i.e., the compound is an amide. In other preferred embodiments, the A¹ substituent of the compound or salt is hydroxy, i.e., the compound is a hydroxamic acid.

The preferred total daily dose of the compound or salt (administered in single or divided doses) is typically from about 0.001 to about 100 mg/kg, more preferably from about 0.001 to about 30 mg/kg, and even more preferably from about 0.01 to about 10 mg/kg (i.e., mg of compound or salt of this invention per kg body weight). Dosage unit compositions can contain such amounts or submultiples thereof to make up the daily dose. In many instances, the administration of the compound or salt will be repeated a plurality of times. Multiple doses per day typically may be used to increase the total daily dose, if desired.

Factors affecting the preferred dosage regimen include the type, age, weight, sex, diet, and condition of the patient; the severity of the pathological condition; the route of administration; pharmacological considerations, such as the activity, efficacy, pharmacokinetic, and toxicology profiles of the particular compound or salt used; whether a drug delivery system is utilized; and whether the compound or salt is administered as part of a drug combination. Thus, the dosage regimen actually employed can vary widely, and, therefore, can deviate from the preferred dosage regimen set forth above.

### D. Pharmaceutical Compositions Containing the Compounds and Salts of this Invention

This invention also is directed to pharmaceutical compositions comprising a compound or salt thereof described above, and to methods for making pharmaceutical compositions (or medicaments) comprising a compound or salt thereof described above.

The preferred composition depends on the method of administration, and typically comprises one or more conventional pharmaceutically acceptable carriers, adjuvants, and/or vehicles. Formulation of drugs is generally discussed in, for example, Hoover, John E., Remington's Pharmaceutical Sciences (Mack Publishing Co., Easton, Pa.: 1975). See also, Liberman, H. A. See also, Lachman, L., eds., Pharmaceutical Dosage Forms (Marcel Decker, New York, N.Y., 1980).

Solid dosage forms for oral administration include, for example, capsules, tablets, pills, powders, and granules. In such solid dosage forms, the compounds or salts are ordinarily combined with one or more adjuvants. If administered per os, the compounds or salts can be mixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation, as can be provided in a dispersion of the compound or salt in hydroxypropylmethyl cellulose. In the case of capsules, tablets, and pills, the dosage forms also can comprise buffering agents, such as sodium citrate, or magnesium or calcium carbonate or bicarbonate. Tablets and pills additionally can be prepared with enteric coatings.

Liquid dosage forms for oral administration include, for example, pharmaceutically acceptable emulsions, solutions, suspensions, syrups, and elixirs containing inert diluents commonly used in the art (e.g., water). Such compositions also can comprise adjuvants, such as wetting, emulsifying, suspending, flavoring (e.g., sweetening), and/or perfuming agents.

“Parenteral administration” includes subcutaneous injections, intravenous injections, intramuscular injections, intrasternal injections, and infusion. Injectable preparations (e.g., sterile injectable aqueous or oleaginous suspensions) can be formulated according to the known art using suitable dispersing, wetting agents, and/or suspending agents. Acceptable vehicles and solvents include, for example, water, 1,3-butanediol, Ringer's solution, isotonic sodium chloride solution, bland fixed oils (e.g., synthetic mono- or diglycerides), fatty acids (e.g., oleic acid), dimethyl acetamide, surfactants (e.g., ionic and non-ionic detergents), and/or polyethylene glycols.

Formulations for parenteral administration may, for example, be prepared from sterile powders or granules having one or more of the carriers or diluents mentioned for use in the formulations for oral administration. The compounds or salts of this invention can be dissolved in water, polyethylene glycol, propylene glycol, ethanol, corn oil, cottonseed oil, peanut oil, sesame oil, benzyl alcohol, sodium chloride, and/or various buffers.

Suppositories for rectal administration can be prepared by, for example, mixing the drug with a suitable nonirritating excipient that is solid at ordinary temperatures, but liquid at the rectal temperature and will therefore melt in the rectum to release the drug. Suitable excipients include, for example, such as cocoa butter; synthetic mono-, di-, or triglycerides; fatty acids; and/or polyethylene glycols

“Topical administration” includes the use of transdermal administration, such as transdermal patches or iontophoresis devices.

Other adjuvants and modes of administration well-known in the pharmaceutical art may also be used.

### E. Intermediates

This invention is further directed to compounds that are, for example, useful as intermediates in processes (such as those illustrated below in Section G) for making the above-described compounds and salts. Such intermediate compounds correspond in structure to Formula (63-1):

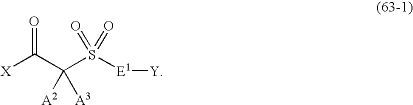

The following discussion describes preferred substituents for this structure.

### Preferred X Substituents

In some embodiments, X is —O—R¹. Here, R¹ is hydrogen, C₁-C₆-alkyl, aryl, or aryl-C₁-C₆-alkyl. In some preferred embodiments, R¹ is t-butyl.

In some embodiments, X is —NH—O—R². Here, R² is a selectively removable protecting group. In some preferred embodiments, R² is 2-tetrahydropyranyl.

In some embodiments, X is —NH—O—R³. Here, R³ is hydrogen or C(W)R⁶, and W is O or S. R⁶ is C₁-C₆-alkyl, aryl, heteroaryl-C₁-C₆-alkyl, C₃-C₈-cycloalkyl-C₁-C₆-alkyl, aryl-C₁-C₆-alkyl, heteroaryl, or amino-C₁-C₆-alkyl. The amino-C₁-C₆-alkyl nitrogen optionally is substituted with:

up to two substituents independently selected from the group consisting of C₁-C₆-alkyl, aryl, aryl-C₁-C₆-alkyl, C₃-C₈-cycloalkyl-C₁-C₆-alkyl, aryl-C₁-C₆-alkoxycarbonyl, C₁-C₆-alkoxycarbonyl, and C₁-C₆-alkylcarbonyl, or

two substituents such that the amino-C₁-C₆-alkyl nitrogen and two substituents form a 5- to 8-member heterocyclyl.

In some embodiments, X is —N R⁴R⁵. Here, R⁴ is hydrogen, C₁-C₆-alkyl, C₁-C₆-alkoxy, amino-C₁-C₆-alkyl, hydroxy-C₁-C₆-alkyl, aryl, aryloxy, or aryl-C₁-C₆-alkyl; and R⁵ is hydrogen, C₁-C₆-alkyl, amino-C₁-C₆-alkyl, hydroxy-C₁-C₆-alkyl, aryl, or aryl-C₁-C₆-alkyl. Alternatively, R⁴ and R⁵, together with the nitrogen atom to which they are both bonded, form a 5- to 8-member ring optionally comprising up to one additional heteroatom (i.e., a heteroatom in addition to the nitrogen atom to which both R⁴ and R⁵ are bonded) selected from the group consisting of oxygen, nitrogen, and sulfur.

In some preferred embodiments, R⁴ and R⁵ are independently selected from the group consisting of hydrogen, C₁-C₆-alkyl, amino-C₁-C₆-alkyl, hydroxy-C₁-C₆-alkyl, aryl, and aryl-C₁-C₆-alkyl.

In some preferred embodiments, R⁴ is C₁-C₆-alkyl, amino-C₁-C₆-alkyl, hydroxy-C₁-C₆-alkyl, aryl, or aryl-C₁-C₆-alkyl; and R⁵ is hydrogen, C₁-C₆-alkyl, amino-C₁-C₆-alkyl, hydroxy-C₁-C₆-alkyl, aryl, or aryl-C₁-C₆-alkyl.

### Preferred A² and A³ Substituents

In some embodiments, A² and A³ are independently selected from the group consisting of hydrogen, alkyl, alkoxyalkyl, alkylthioalkyl, alkenyl, alkynyl, carbocyclyl, carbocyclylalkyl, carbocyclylalkenyl, carbocyclylalkynyl, carbocyclyloxyalkyl, carbocyclylalkoxyalkyl, carbocyclylalkylthio, carbocyclylthioalkyl, carbocyclylalkylthioalkyl, heterocyclyl, heterocyclylalkyl, heterocyclylalkenyl, heterocyclylalkynyl, heterocyclyloxyalkyl, heterocyclylalkoxyalkyl, heterocyclylalkylthio, heterocyclylthioalkyl, and heterocyclylalkylthioalkyl. Any member of such group optionally is substituted with:

up to three independently selected R^{x} substituents; and

two substituents such that the two substituents, together with the atom(s) to which they are bonded, form a carbocyclyl or heterocyclyl, wherein the optional heterocyclyl or carbocyclyl is, in turn, optionally substituted with up to three independently selected R^{x} substituents.

In some embodiments, A² and A³, together with the carbon to which they are both bonded, form heterocyclyl or carbocyclyl. The heterocyclyl or carbocyclyl optionally is substituted with:

up to three independently selected R^{X} substituents; and

two substituents such that the two substituents, together with the atom(s) to which they are bonded, form a carbocyclyl or heterocyclyl, wherein the optional heterocyclyl or carbocyclyl is, in turn, optionally substituted with up to three independently selected R^{X} substituents.

In some preferred embodiments,

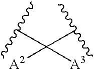

is selected from one of the following formulas:

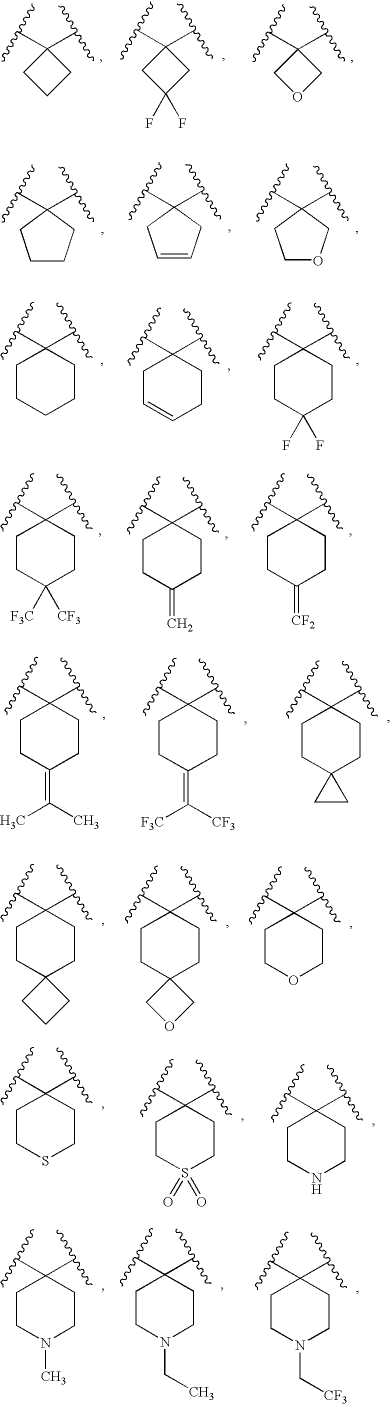

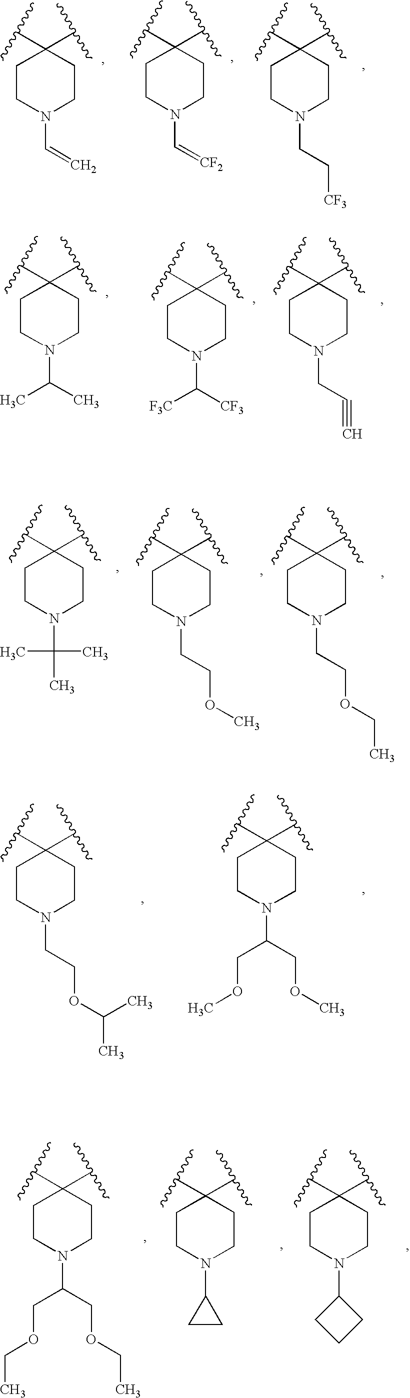

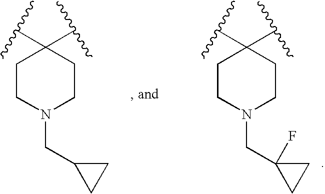

In some preferred embodiments, the compound corresponds in structure to Formula (II):

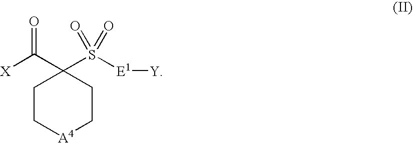

Here, A⁴ is —C(H)₂—, —C(R^{x})(H)—, —C(R^{x})₂—, —O—, —N(H)—, —N(R^{x})—, —S—, —S(O)—, or —S(O)₂—. In some particularly preferred embodiments, A⁴ is —O—, —N(H)—, —N(R^{x})—, —S—, —S(O)—, or —S(O)₂.

In some preferred embodiments, the compound corresponds in structure to Formula (II-1A):

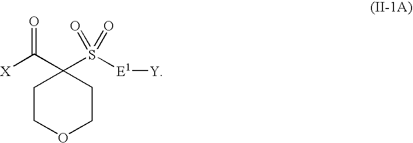

In some preferred embodiments, the compound corresponds in structure to Formula (II-2A):

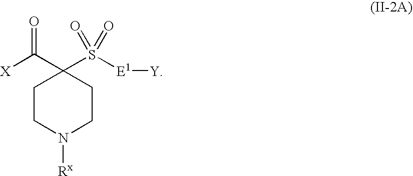

### Preferred E¹ Substituents

E¹ is heteroaryl. This heteroaryl optionally is substituted with one or more independently selected R^{x} substituents. In some particularly preferred embodiments, this heteroaryl has no such optional substituents.

In some preferred embodiments, E¹ is furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some preferred embodiments, E¹ is oxazolyl, isoxazolyl, isothiazolyl, thiodiazolyl, oxadiazolyl, pyrrolyl, pyrazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoisoxazolyl, anthranilyl, benzothienyl, isobenzothienyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected RX substituents. In many particularly preferred embodiments, however, there is no such optional substitution.

In some preferred embodiments, E¹ is oxazolyl, isoxazolyl, thiodiazolyl, oxadiazolyl, pyrrolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzothienyl, isobenzothienyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, or acridinyl. Any member of such group optionally is substituted with one or more independently selected R^{x} substituents. In many particularly preferred embodiments, however, there is no such optional substitution.

In some preferred embodiments, E¹ is thienyl. This thienyl optionally is substituted with one or more independently selected R^{x} substituents. In some particularly preferred embodiments, the thienyl has no such optional substituents.

In some preferred embodiments, E¹ is pyridinyl. This pyridinyl optionally is substituted with one or more independently selected R^{x} substituents. In some particularly preferred embodiments, the pyridinyl has no such optional substituents.

In some preferred embodiments, E¹ is benzothiazolyl. This benzothiazolyl optionally is substituted with one or more independently selected R^{x} substituents. In some preferred embodiments, the benzothiazolyl has no such optional substituents.

In some preferred embodiments, E¹ is benzoimidazothiazolyl. This benzoimidazothiazolyl optionally is substituted with one or more independently selected R^{x} substituents. In some particularly preferred embodiments, the benzoimidazothiazolyl has no such optional substituents.

### Preferred Y Substituents

Y is a nucleophilically displaceable leaving group. Generally, Y may be, for example, halogen, nitro, azido, phenylsulfoxido, aryloxy, C₂-C₆-alkoxy, C₁-C₆-alkylsulfonate, arylsulfonate, or trisubstituted ammonium. Here, the trisubstituted ammonium substituents are independently selected from the group consisting of aryl, aryl-C₁-C₆-alkyl, and C₁-C₆-alkyl.

In some preferred embodiments, Y is halogen, nitro, azido, phenylsulfoxido, aryloxy, C₁-C₆-alkylsulfonate, arylsulfonate, or trisubstituted ammonium. The trisubstituted ammonium substituents are independently selected from the group consisting of aryl, aryl-C₁-C₆-alkyl, and C₁-C₆-alkyl.

In some preferred embodiments, Y is bromo. Compounds falling within such embodiments include, for example, the compound corresponding in structure to Formula (68-1):

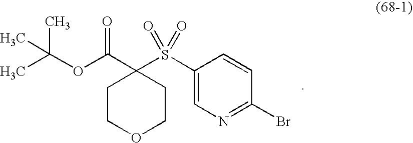

### Preferred R^{x} Substituents

Each R^{x} is independently selected from the group consisting of halogen, cyano, hydroxy, nitro, nitroso, oxo, alkyl, alkenyl, alkynyl, alkoxy, alkoxyalkoxy, R^{b}-oxyalkyl, alkenyloxy, alkynyloxy, alkylthio, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, carbocyclylthio, heterocyclyl, heterocyclylalkyl, heterocyclyloxy, heterocyclyloxyalkoxy, heterocyclylthio, alkyliminocarbonyl, alkylthioalkyl, alkylsulfonylalkyl, alkylsulfoxidoalkyl, alkylthioalkenyl, alkylsulfoxidoalkenyl, alkylsulfonylalkenyl, carbocyclylalkoxyalkyl, carbocyclyliminocarbonyl, carbocyclylthioalkyl, carbocyclylsulfoxidoalkyl, carbocyclylsulfonylalkyl, carbocyclylthioalkenyl, carbocyclylsulfoxidoalkenyl, carbocyclylsulfonylalkenyl, heterocyclylalkoxyalkyl, heterocyclylthioalkyl, heterocyclylsulfoxidoalkyl, heterocyclylsulfonylalkyl, heterocyclylthioalkenyl, heterocyclylsulfoxidoalkenyl, heterocyclylsulfonylalkenyl, heterocyclyliminocarbonyl, aminosulfonylalkyl, and —R^{x1}—R^{x2}. Any member of such group optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkoxyalkyl, and alkoxyalkoxy. Any such optional substituent is, in turn, optionally substituted with one or more substituents independently selected from the group consisting of halogen, hydroxy, and alkyl.

Each R^{x1} is —C(O)—, —C(S)—, —C(NR^{y})—, —S(O)—, or —S(O)₂—. Each R^{y}, in turn, is hydrogen or hydroxy.

Each R^{x2} is hydrogen, hydroxy, alkyl, alkenyl, alkynyl, alkoxy, alkoxyalkyl, alkoxyalkoxy, R^{b}-oxyalkyl, alkenyloxy, alkynyloxy, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, heterocyclyl, heterocyclylalkyl, heterocyclyloxy, or heterocyclyloxyalkoxy. Any member of such group optionally is substituted with one or more substituents independently selected from the group consisting of halogen, hydroxy, cyano, carboxy, thiol, sulfo, nitro, nitroso, oxo, thioxo, imino, alkyl, alkoxy, alkoxyalkyl, and alkoxyalkoxy. Any such optional substituent is, in turn, optionally substituted with one or more substituents independently selected from the group consisting of halogen and hydroxy.

### Preferred R^{b} Substituents

Each R^{b} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl. Any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl.

### F. Definitions

The term “alkyl” (alone or in combination with another term(s)) means a straight- or branched-chain saturated hydrocarbyl substituent typically containing from 1 to about 20 carbon atoms, more typically from 1 to about 8 carbon atoms, and even more typically from 1 to about 6 carbon atoms. Examples of such substituents include methyl, ethyl, n-propyl, isopropyl, n-butyl, isobutyl, sec-butyl, tert-butyl, pentyl, iso-amyl, hexyl, octyl, and the like.

The term “alkenyl” (alone or in combination with another term(s)) means a straight- or branched-chain hydrocarbyl substituent containing one or more double bonds and typically from 1 to about 20 carbon atoms, more typically from about 2 to about 20 carbon atoms, even more typically from about 2 to about 8 carbon atoms, and still even more typically from about 2 to about 6 carbon atoms. Examples of such substituents include ═CH₂, ethenyl (vinyl); 2-propenyl; 3-propenyl; 1,4-pentadienyl; 1,4-butadienyl; 1-butenyl; 2-butenyl; 3-butenyl; decenyl; and the like.

The term “alkynyl” (alone or in combination with another term(s)) means a straight- or branched-chain hydrocarbyl substituent containing one or more triple bonds and typically from 2 to about 20 carbon atoms, more typically from about 2 to about 8 carbon atoms, and even more typically from about 2 to about 6 carbon atoms. Examples of such substituents include ethynyl, 2-propynyl, 3-propynyl, decynyl, 1-butynyl, 2-butynyl, 3-butynyl, and the like.

The term “carbocyclyl” (alone or in combination with another term(s)) means a saturated cyclic (i.e., “cycloalkyl”), partially saturated cyclic (i.e., “cycloalkenyl”), or completely unsaturated (i.e., “aryl”) hydrocarbyl substituent typically containing from 3 to 14 carbon ring atoms (“ring atoms” are the atoms bound together to form the ring or rings of a cyclic substituent). A carbocyclyl may be a single ring, which typically contains from 3 to 6 ring atoms. Examples of such single-ring carbocyclyls include cyclopropanyl, cyclobutanyl, cyclopentyl, cyclopentenyl, cyclopentadienyl, cyclohexyl, cyclohexenyl, cyclohexadienyl, and phenyl. A carbocyclyl alternatively may be multiple (typically 2 or 3) rings fused together, such as naphthalenyl, tetrahydronaphthalenyl (also known as “tetralinyl”), indenyl, isoindenyl, indanyl, bicyclodecanyl, anthracenyl, phenanthrene, benzonaphthenyl (also known as “phenalenyl”), fluoreneyl, decalinyl, and norpinanyl.

The term “cycloalkyl” (alone or in combination with another term(s)) means a saturated cyclic hydrocarbyl substituent typically containing from 3 to 14 carbon ring atoms. A cycloalkyl may be a single carbon ring, which typically contains from 3 to 6 carbon ring atoms. Examples of single-ring cycloalkyls include cyclopropyl (or “cyclopropanyl”), cyclobutyl (or “cyclobutanyl”), cyclopentyl (or “cyclopentanyl”), and cyclohexyl (or “cyclohexanyl”). A cycloalkyl alternatively may be multiple (typically 2 or 3) carbon rings fused together, such as, decalinyl or norpinanyl.

The term “aryl” (alone or in combination with another term(s)) means an aromatic carbocyclyl typically containing from 6 to 14 carbon ring atoms. Examples of aryls include phenyl, naphthalenyl, and indenyl.

In some instances, the number of carbon atoms in a hydrocarbyl substituent (e.g., alkyl, alkenyl, alkynyl, or cycloalkyl) is indicated by the prefix “Cₓ-C_{y}”, wherein x is the minimum and y is the maximum number of carbon atoms in the substituent. Thus, for example, “C₁-C₆-alkyl” refers to an alkyl substituent containing from 1 to 6 carbon atoms. Illustrating further, C₃-C₆-cycloalkyl means a saturated hydrocarbyl ring containing from 3 to 6 carbon ring atoms.

The term “hydrogen” (alone or in combination with another term(s)) means a hydrogen radical (or “hydrido”), and may be depicted as —H.

The term “hydroxy” (alone or in combination with another term(s)) means —OH.

The term “nitro” (alone or in combination with another term(s)) means —NO₂.

The term “cyano” (alone or in combination with another term(s)) means —CN, which also may be depicted:

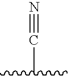

The term “keto” (alone or in combination with another term(s)) means an oxo radical, and may be depicted as ═O.

The term “carboxy” (alone or in combination with another term(s)) means —C(O)—OH, which also may be depicted as:

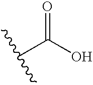

The term “amino” (alone or in combination with another term(s)) means —NH². The term “monosubstituted amino” (alone or in combination with another term(s)) means an amino substituent wherein a non-hydrogen substituent is in the place of one of the hydrogens. The term “disubstituted amino” (alone or in combination with another term(s)) means an amino substituent wherein non-hydrogen substituents (which may be identical or different) are in the place of both of the hydrogens.

The term “halogen” (alone or in combination with another term(s)) means a fluorine radical (“fluoro”, which may be depicted as —F), chlorine radical (“chloro”, which may be depicted as —Cl), bromine radical (“bromo”, which may be depicted as —Br), or iodine radical (“iodo”, which may be depicted as —I). Typically, fluoro or chloro is preferred, with fluoro often being particularly preferred.

A substituent is “substitutable” if it comprises at least one carbon, nitrogen, oxygen, or sulfur atom that is bonded to one or more hydrogen atoms. Thus, for example, hydrogen, halogen, and cyano do not fall within this definition.

If a substituent is described as being “substituted”, a non-hydrogen substituent is in the place of a hydrogen on a carbon, nitrogen, oxygen, or sulfur of the substituent. Thus, for example, a substituted alkyl substituent is an alkyl substituent wherein at least one non-hydrogen substituent is in the place of a hydrogen on the alkyl substituent. To illustrate, monofluoroalkyl is alkyl substituted with a fluoro, and difluoroalkyl is alkyl substituted with two fluoros. It should be recognized that if there are more than one substitutions on a substituent, each non-hydrogen substituent may be identical or different (unless otherwise stated).

If a substituent is described as being “optionally substituted”, the substituent may be either (1) not substituted or (2) substituted. If a substituent is described as being optionally substituted with up to a particular number of non-hydrogen substituents, that substituent may be either (1) not substituted; or (2) substituted by up to that particular number of non-hydrogen substituents or by up to the maximum number of substitutable positions on the substituent, whichever is less. Thus, for example, if a substituent is described as a heteroaryl optionally substituted with up to 3 non-hydrogen substituents, then any heteroaryl with less than 3 substitutable positions would be optionally substituted by up to only as many non-hydrogen substituents as the heteroaryl has substitutable positions. To illustrate, tetrazolyl (which has only one substitutable position when it is bonded to a single non-hydrogen moiety by a single bond) would be optionally substituted with up to one non-hydrogen substituent. To illustrate further, if an amino nitrogen is described as being optionally substituted with up to 2 non-hydrogen substituents, then a primary amino nitrogen will be optionally substituted with up to 2 non-hydrogen substituents, whereas a secondary amino nitrogen will be optionally substituted with up to only one non-hydrogen substituent. Further illustrations of this definition may be found above at, for example, the sub-section entitled “General Description of Preferred A² and A³ Substituents.”

This specification uses the terms “substituent” and “radical” interchangeably.

The prefix “halo” indicates that the substituent to which the prefix is attached is substituted with one or more independently selected halogens. For example, haloalkyl means an alkyl substituent having a halogen in the place of a hydrogen, or multiple halogens in the place of the same number of hydrogens. Examples of haloalkyls include chloromethyl, 1-bromoethyl, fluoromethyl, difluoromethyl, trifluoromethyl, 1,1,1-trifluoroethyl, and the like. Illustrating further, “haloalkoxy” means an alkoxy substituent wherein a halogen is in the place of a hydrogen, or multiple halogens are in the place of the same number of hydrogens. Examples of haloalkoxy substituents include chloromethoxy, 1-bromoethoxy, fluoromethoxy, difluoromethoxy, trifluoromethoxy (also known as “perfluoromethyloxy”), 1,1,1trifluoroethoxy, and the like. It should be recognized that if a substituent is substituted by more than one halogen, those halogens may be identical or different (unless otherwise stated).

The prefix “perhalo” indicates that a halogen is in the place of each hydrogen on the substituent to which the prefix is attached. If all the halogens are identical, the prefix typically will identify the halogen. Thus, for example, the term “perfluoro” means that a fluoro is in the place of each hydrogen on the substituent to which the prefix is attached. To illustrate, the term “perfluoroalkyl” means an alkyl substituent wherein a fluoro is in the place of each hydrogen. Examples of perfluoroalkyl substituents include trifluoromethyl (—CF₃), perfluorobutyl, perfluoroisopropyl, perfluorododecyl, perfluorodecyl, and the like. To illustrate further, the term “perfluoroalkoxy” means an alkoxy substituent wherein a fluoro is in the place of each hydrogen. Examples of perfluoroalkoxy substituents include trifluoromethoxy (—O—CF₃), perfluorobutoxy, perfluoroisopropoxy, perfluorododecoxy, perfluorodecoxy, and the like.

The term “carbonyl” (alone or in combination with another term(s)) means —C(O)—, which also may be depicted as:

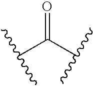

This term also is intended to encompass a hydrated carbonyl substituent, i.e., —C(OH)₂—.

The term “aminocarbonyl” (alone or in combination with another term(s)) means —C(O)—NH₂, which also may be depicted as:

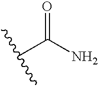

The term “oxy” (alone or in combination with another term(s)) means an ether substituent, and may be depicted as —O—.

The term “alkoxy” (alone or in combination with another term(s)) means an alkylether substituent, i.e., —O-alkyl. Examples of such a substituent include methoxy (—O—CH₃), ethoxy, n-propoxy, isopropoxy, n-butoxy, iso-butoxy, sec-butoxy, tert-butoxy, and the like.

The term “alkylcarbonyl” (alone or in combination with another term(s)) means —C(O)-alkyl. For example, “ethylcarbonyl” may be depicted as:

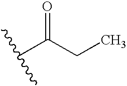

The term “aminoalkylcarbonyl” (alone or in combination with another term(s)) means —C(O)-alkyl-NH₂. For example, “aminomethylcarbonyl” may be depicted as:

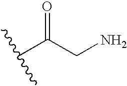

The term “alkoxycarbonyl” (alone or in combination with another term(s)) means —C(O)—O-alkyl. For example, “ethoxycarbonyl” may be depicted as:

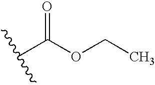

The term “carbocyclylcarbonyl” (alone or in combination with another terrn(s)) means —C(O)-carbocyclyl. For example, “phenylcarbonyl” may be depicted as:

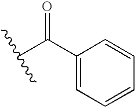

Similarly, the term “heterocyclylcarbonyl” (alone or in combination with another term(s)) means —C(O)-heterocyclyl.

The term “carbocyclylalkylcarbonyl” (alone or in combination with another term(s)) means —C(O)-alkyl-carbocyclyl. For example, “phenylethylcarbonyl” may be depicted as:

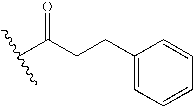

Similarly, the term “heterocyclylalkylcarbonyl” (alone or in combination with another term(s)) means —C(O)-alkyl-heterocyclyl.

The term “carbocyclyloxycarbonyl” (alone or in combination with another term(s)) means —C(O)—O-carbocyclyl. For example, “phenyloxycarbonyl” may be depicted as:

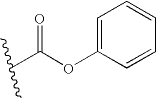

The term “carbocyclylalkoxycarbonyl” (alone or in combination with another term(s)) means —C(O)—O-alkyl-carbocyclyl. For example, “phenylethoxycarbonyl” may be depicted as:

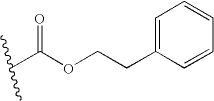

The term “thio” or “thia” (alone or in combination with another term(s)) means a thiaether substituent, i.e., an ether substituent wherein a divalent sulfur atom is in the place of the ether oxygen atom. Such a substituent may be depicted as —S—. This, for example, “alkyl-thio-alkyl” means alkyl-S-alkyl.

The term “thiol” or “mercapto” (alone or in combination with another term(s)) means a sulfhydryl substituent, and may be depicted as —SH.

The term “(thiocarbonyl)” (alone or in combination with another term(s)) means a carbonyl wherein a sulfur is in the place of the oxygen. Such a substituent may be depicted as —C(S)—, and also may be depicted as:

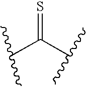

The term “sulfonyl” (alone or in combination with another term(s)) means —S(O)₂—, which also may be depicted as:

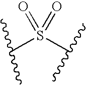

Thus, for example, “alkyl-sulfonyl-alkyl” means alkyl-S(O)₂-alkyl.

The term “aminosulfonyl” (alone or in combination with another term(s)) means —S(O)₂—NH², which also may be depicted as:

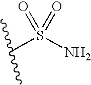

The term “sulfoxido” (alone or in combination with another term(s)) means —S(O)—, which also may be depicted as:

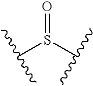

Thus, for example, “alkyl-sulfoxido-alkyl” means alkyl-S(O)-alkyl.

The term “heterocyclyl” (alone or in combination with another term(s)) means a saturated (i.e., “heterocycloalkyl”), non-aromatic partially-saturated (i.e., “heterocycloalkenyl”), or heterocyclic aromatic (i.e., “heteroaryl”) ring structure typically containing a total of 3 to 14 ring atoms. At least one of the ring atoms is a heteroatom (typically oxygen, nitrogen, or sulfur), with the remaining ring atoms being independently selected from the group typically consisting of carbon, oxygen, nitrogen, and sulfur.

A heterocyclyl may be a single ring, which typically contains from 3 to 7 ring atoms, more typically from 3 to 6 ring atoms, and even more typically 5 to 6 ring atoms. Examples of single-ring heterocyclyls include furanyl, thienyl (also known as “thiophenyl” and “thiofuranyl”), oxazolyl, isoxazolyl, thiazolyl, isothiazolyl, thiodiazolyl, oxadiazolyl (including 1,2,3-oxadiazolyl, 1,2,4-oxadiazolyl (also known as “azoximyl”), 1,2,5-oxadiazolyl (also known as “furazanyl”), and 1,3,4-oxadiazolyl), pyrrolyl, pyrazolyl, imidazolyl, triazolyl, tetrazolyl, oxathiazolyl, oxatriazolyl (including 1,2,3,4-oxatriazolyl and 1,2,3,5-oxatriazolyl), pyridinyl, diazinyl (including pyridazinyl (also known as “1,2-diazinyl”), pyrimidinyl (also known as “1,3-diazinyl”), and pyrazinyl (also known as “1,4-diazinyl”)), triazinyl (including s-triazinyl (also known as “1,3,5-triazinyl”), as-triazinyl (also known 1,2,4-triazinyl), and v-triazinyl (also known as “1,2,3-triazinyl”)), oxathiazinyl (including 1,2,5-oxathiazinyl and 1,2,6-oxathiazinyl), oxepinyl, thiepinyl, dihydrofuranyl, tetrahydrofuranyl, dihydrothienyl (also known as “dihydrothiophenyl”), tetrahydrothienyl (also known as “tetrahydrothiophenyl”), isopyrrolyl, pyrrolinyl, pyrrolidinyl, isoimidazolyl, imidazolinyl, imidazolidinyl, pyrazolinyl, pyrazolidinyl, dithiolyl, oxathiolyl, oxathiolanyl, oxazolidinyl, isoxazolidinyl, thiazolinyl, isothiazolinyl, thiazolidinyl, isothiazolidinyl, dioxazolyl (including 1,2,3-dioxazolyl, 1,2,4-dioxazolyl, 1,3,2-dioxazolyl, and 1,3,4-dioxazolyl), pyranyl (including 1,2-pyranyl and 1,4-pyranyl), dihydropyranyl, tetrahydropyranyl, piperidinyl, piperazinyl, oxazinyl (including 1,2,3-oxazinyl, 1,3,2-oxazinyl, 1,3,6-oxazinyl (also known as “pentoxazolyl”), 1,2,6-oxazinyl, and 1,4-oxazinyl), isoxazinyl (including o-isoxazinyl and p-isoxazinyl), oxadiazinyl (including 1,4,2-oxadiazinyl and 1,3,5,2-oxadiazinyl), morpholinyl, azepinyl, and diazepinyl.

A heterocyclyl alternatively may be 2 or 3 rings fused together, such as, for example, indolizinyl, pyranopyrrolyl, purinyl, imidazopyrazinyl, imidazolopyridazyl, pyridopyridinyl (including pyrido[3,4-b]-pyridinyl, pyrido[3,2-b]-pyridinyl, pyrido[4,3-b]-pyridinyl, and naphthyridinyl), pteridinyl, pyridazinotetrazinyl, pyrazinotetrazinyl, pyrimidinotetrazinyl, pyrindinyl, pyrazolopyrimidinyl, pyrazolopyrazinyl, pyrazolopyridazyl, or 4H-quinolizinyl. In some embodiments, the preferred multi-ring heterocyclyls are indolizinyl, pyranopyrrolyl, purinyl, pyridopyridinyl, pyrindinyl, and 4H-quinolizinyl.

Other examples of fused-ring heterocyclyls include benzo-fused heterocyclyls, such as, for example, benzofuranyl (also known as “coumaronyl”), isobenzofuranyl, benzoxazolyl, benzoisoxazolyl (also known as “indoxazinyl”), anthranilyl, benzothienyl (also known as “benzothiophenyl”, “thionaphthenyl”, and “benzothiofuranyl”), isobenzothienyl (also known as “isobenzothiophenyl”, “isothionaphthenyl”, and “isobenzothiofuranyl”), benzothiazolyl, benzoisothiazolyl, benzothiadiazolyl, benzoxadiazolyl, indolyl, isoindazolyl (also known as “benzpyrazolyl”), benzoimidazolyl, benzotriazolyl, benzazinyl (including quinolinyl (also known as “1-benzazinyl”) and isoquinolinyl (also known as “2-benzazinyl”)), phthalazinyl, quinoxalinyl, benzodiazinyl (including cinnolinyl (also known as “1,2-benzodiazinyl”) and quinazolinyl (also known as “1,3-benzodiazinyl”)), benzoimidazothiazolyl, carbazolyl, acridinyl, isoindolyl, indoleninyl (also known as “pseudoindolyl”), benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, benzoxazinyl (including 1,3,2-benzoxazinyl, 1,4,2-benzoxazinyl, 2,3,1-benzoxazinyl, and 3,1,4-benzoxazinyl), benzoisoxazinyl (including 1,2-benzisoxazinyl and 1,4-benzisoxazinyl), benzoxadiazinyl, and xanthenyl. In some embodiments, the preferred benzo-fused heterocyclyls are benzofuranyl, isobenzofuranyl, benzoxazolyl, benzoisoxazolyl, anthranilyl, benzothienyl, isobenzothienyl, benzothiazolyl, benzothiadiazolyl, benzoxadiazolyl, indolyl, isoindazolyl, benzoimidazolyl, benzotriazolyl, benzazinyl, phthalazinyl, quinoxalinyl, benzodiazinyl, carbazolyl, acridinyl, isoindolyl, indoleninyl, benzodioxolyl, chromanyl, isochromanyl, thiochromanyl, benzodioxanyl, tetrahydroisoquinolinyl, benzoxazinyl, benzoisoxazinyl, and xanthenyl.

The term “2-fused-ring” heterocyclyl (alone or in combination with another term(s)) means a saturated, non-aromatic partially-saturated, or heteroaryl containing two fused rings. Such heterocyclyls include, for example, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, pyrindinyl, isoindolyl, indoleninyl, pyrazolopyrimidinyl, pyrazolopyrazinyl, pyrazolopyridazyl, benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, 4H-quinolizinyl, benzoxazinyl, and benzoisoxazinyl. In some embodiments, preferred 2-fused-ring heterocyclyls include benzofuranyl, isobenzofuranyl, benzoxazolyl, benzoisoxazolyl, anthranilyl, benzothienyl, isobenzothienyl, benzothiazolyl, benzothiadiazolyl, indolizinyl, pyranopyrrolyl, benzoxadiazolyl, indolyl, isoindazolyl, benzoimidazolyl, benzotriazolyl, purinyl, quinolinyl, isoquinolinyl, pyridopyridinyl, phthalazinyl, quinoxalinyl, benzodiazinyl, pteridinyl, pyrindinyl, isoindolyl, indoleninyl, benzodioxolyl, benzodioxanyl, tetrahydroisoquinolinyl, 4H-quinolizinyl, benzoxazinyl, and benzoisoxazinyl.

The term “heteroaryl” (alone or in combination with another term(s)) means an aromatic heterocyclyl typically containing from 5 to 14 ring atoms. A heteroaryl may be a single ring or multiple (typically 2 or 3) fused rings. Such moieties include, for example, 5-membered rings such as furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, and oxatriazolyl; 6-membered rings such as pyridinyl, pyrazinyl, pyrimidinyl, pyridazinyl, triazinyl, and oxathiazinyl; 7-membered rings such as oxepinyl and thiepinyl; 6/5-membered fused-ring systems such as benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, and imidazolopyridazyl; and 6/6-membered fused-ring systems such as quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, and acridinyl. In some embodiments, the preferred 5-membered rings include furanyl, thienyl, oxazolyl, isoxazolyl, thiazolyl, isothiazolyl, oxadiazolyl, pyrazolyl, and imidazolyl; the preferred 6-membered rings include pyridinyl, pyrazinyl, pyrimidinyl, pyridazinyl, and triazinyl; the preferred 6/5-membered fused-ring systems include benzoxazolyl, benzoisoxazolyl, anthranilyl, benzothienyl, isobenzothienyl, and purinyl; and the preferred 6/6-membered fused-ring systems include quinolinyl, isoquinolinyl, and benzodiazinyl.

A carbocyclyl or heterocyclyl can optionally be substituted with, for example, one or more substituents independently selected from the group consisting of halogen, hydroxy, carboxy, keto, alkyl, alkoxy, alkoxyalkyl, alkylcarbonyl (also known as “alkanoyl”), aryl, arylalkyl, arylalkoxy, arylalkoxyalkyl, arylalkoxycarbonyl, cycloalkyl, cycloalkylalkyl, cycloalkylalkoxy, cycloalkylalkoxyalkyl, and cycloalkylalkoxycarbonyl. More typically, a carbocyclyl or heterocyclyl may optionally be substituted with, for example, one or more substituents independently selected from the group consisting of halogen, —OH, —C(O)—OH, keto, C₁-C₆-alkyl, C₁-C₆-alkoxy, C₁-C₆-alkoxy-C₁-C₆-alkyl, C₁-C₆-alkylcarbonyl, aryl, aryl-C₁-C₆-alkyl, aryl-C₁-C₆-alkoxy, aryl-C₁-C₆-alkoxy-C₁-C₆-alkyl, aryl-C₁-C₆-alkoxycarbonyl, cycloalkyl, cycloalkyl-C₁-C₆-alkyl, cycloalkyl-C₁-C₆-alkoxy, cycloalkyl-C₁-C₆-alkoxy-C₁-C₆-alkyl, and cycloalkyl-C₁-C₆-alkoxycarbonyl. The alkyl, alkoxy, alkoxyalkyl, alkylcarbonyl, aryl, arylalkyl, arylalkoxy, arylalkoxyalkyl, or arylalkoxycarbonyl substituent(s) may further be substituted with, for example, one or more halogen. The aryl and cycloalkyl portions of such optional substituents are typically single-rings containing from 3 to 6 ring atoms, and more typically from 5 to 6 ring atoms.

An aryl or heteroaryl can optionally be substituted with, for example, one or more substituents independently selected from the group consisting of halogen, —OH, —CN, —NO₂, —SH, —C(O)—OH, amino, aminoalkyl, alkyl, alkylthio, carboxyalkylthio, alkylcarbonyloxy, alkoxy, alkoxyalkyl, alkoxycarbonylalkoxy, alkoxyalkylthio, alkoxycarbonylalkylthio, carboxyalkoxy, alkoxycarbonylalkoxy, carbocyclyl, carbocyclylalkyl, carbocyclyloxy, carbocyclylthio, carbocyclylalkylthio, carbocyclylamino, carbocyclylalkylamino, carbocyclylcarbonylamino, carbocyclylalkyl, carbocyclylcarbonyloxy, carbocyclyloxyalkoxycarbocyclyl, carbocyclylthioalkylthiocarbocyclyl, carbocyclylthioalkoxycarbocyclyl, carbocyclyloxyalkylthiocarbocyclyl, heterocyclyl, heterocyclylalkyl, heterocyclyloxy, heterocyclylthio, heterocyclylalkylthio, heterocyclylamino, heterocyclylalkylamino, heterocyclylcarbonylamino, heterocyclylcarbonyloxy, heterocyclyloxyalkoxyheterocyclyl, heterocyclylthioalkylthioheterocyclyl, heterocyclylthioalkoxyheterocyclyl, and heterocyclyloxyalkylthioheterocyclyl. More typically, an aryl or heteroaryl may, for example, optionally be substituted with one or more substituents independently selected from the group consisting of halogen, —OH, —CN, —NO₂, —SH, —C(O)—OH, amino, amino-C₁-C₆-alkyl, C₁-C₆-alkyl, C₁-C₆-alkylthio, carboxy-C₁-C₆-alkylthio, C₁-C₆-alkylcarbonyloxy, C₁-C₆-alkoxy, C₁-C₆-alkoxy-C₁-C₆-alkyl, C₁-C₆-alkoxycarbonyl-C₁-C₆-alkoxy, C₁-C₆-alkoxy-C₁-C₆-alkylthio, C₁-C₆-alkoxycarbonyl-C₁-C₆-alkylthio, carboxy-C₁-C₆-alkoxy, C₁-C₆-alkoxycarbonyl-C₁-C₆-alkoxy, aryl, aryl-C₁-C₆-alkyl, aryloxy, arylthio, aryl-C₁-C₆-alkylthio, arylamino, aryl-C₁-C₆-alkylamino, arylcarbonylamino, arylcarbonyloxy, aryloxy-C₁-C₆-alkoxyaryl, arylthio-C₁-C₆-alkylthioaryl, arylthio-C₁-C₆-alkoxyaryl, aryloxy-C₁-C₆-alkylthioaryl, cycloalkyl, cycloalkyl-C₁-C₆-alkyl, cycloalkyloxy, cycloalkylthio, cycloalkyl-C₁-C₆-alkylthio, cycloalkylamino, cycloalkyl-C₁-C₆-alkylamino, cycloalkylcarbonylamino, cycloalkylcarbonyloxy, heteroaryl, heteroaryl-C₁-C₆-alkyl, heteroaryloxy, heteroarylthio, heteroaryl-C₁-C₆-alkylthio, heteroarylamino, heteroaryl-C₁-C₆-alkylamino, heteroarylcarbonylamino, and heteroarylcarbonyloxy. Here, one or more hydrogens bound to a carbon in any such substituent may, for example, optionally be replaced with halogen. In addition, any cycloalkyl, aryl, and heteroaryl portions of such optional substituents are typically single-rings containing 3 to 6 ring atoms, and more typically 5 or 6 ring atoms.

A prefix attached to a multi-component substituent only applies to the first component. To illustrate, the term “alkylcycloalkyl” contains two components: alkyl and cycloalkyl. Thus, the C₁-C₆— prefix on C₁-C₆-alkylcycloalkyl means that the alkyl component of the alkylcycloalkyl contains from 1 to 6 carbon atoms; the C₁-C₆— prefix does not describe the cycloalkyl component. To illustrate further, the prefix “halo” on haloalkoxyalkyl indicates that only the alkoxy component of the alkoxyalkyl substituent is substituted with one or more halogens. If halogen substitution may alternatively or additionally occur on the alkyl component, the substituent would instead be described as “halogen-substituted alkoxyalkyl” rather than “haloalkoxyalkyl.” And finally, if the halogen substitution may only occur on the alkyl component, the substituent would instead be described as “alkoxyhaloalkyl.”

If substituents are described as being “independently selected” from a group, each substituent is selected independent of the other. Each substituent therefore may be identical to or different from the other selected substituent(s).

When words are used to describe a substituent, the rightmost-described component of the substituent is the component that has the free valence. To illustrate, benzene substituted with methoxyethyl has the following structure:

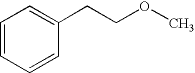

As can be seen, the ethyl is bound to the benzene, and the methoxy is the component of the substituent that is the component furthest from the benzene. As further illustration, benzene substituted with cyclohexanylthiobutoxy has the following structure:

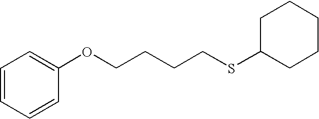

When words are used to describe a linking element between two other elements of a depicted chemical structure, the rightmost-described component of the substituent is the component that is bound to the left element in the depicted structure. To illustrate, if the chemical structure is X-L-Y and L is described as methylcyclohexanylethyl, then the chemical would be X-ethyl-cyclohexanyl-methyl-Y.

When a chemical formula is used to describe a mono-valent substituent, the dash on the left side of the formula indicates the portion of the substituent that has the free valence. To illustrate, benzene substituted with —C(O)—OH has the following structure:

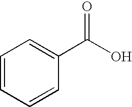

When a chemical formula is used to describe a di-valent (or “linking”) element between two other elements of a depicted chemical structure, the leftmost dash of the substituent indicates the portion of the substituent that is bound to the left element in the depicted structure. The rightmost dash, on the other hand, indicates the portion of the substituent that is bound to the right element in the depicted structure. To illustrate, if the depicted chemical structure is X-L-Y and L is described as —C(O)—N(H)—, then the chemical would be:

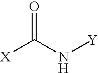

The term “pharmaceutically acceptable” is used adjectivally in this patent to mean that the modified noun is appropriate for use as a pharmaceutical product or as a part of a pharmaceutical product.

With reference to the use of the words “comprise” or “comprises” or “comprising” in this patent (including the claims), Applicants note that unless the context requires otherwise, those words are used on the basis and clear understanding that they are to be interpreted inclusively, rather than exclusively, and that Applicants intend each of those words to be so interpreted in construing this patent.

### G. Compound Preparation

The detailed examples below illustrate preparation of compounds and salts of this invention. Other compounds and salts of this invention may be prepared using the methods illustrated in these examples (either alone or in combination with techniques generally known in the art). Such known techniques include, for example, those disclosed in Int'l Publ. No. WO 99/25687 (PCT Patent Application No. PCT/US98/23242 published on May 27, 1999), which issued as U.S. Pat. No. 6,541,489 on Apr. 1, 2003 (incorporated herein by reference). Such known techniques also include, for example, those disclosed in Int'l Publ. No. WO 00/50396 (PCT Patent Application No. PCT/US00/02518 published on Aug. 31, 2000) (incorporated herein by reference). Such known techniques further include, for example, those disclosed in Int'l Publ. No. WO 00/69821 (PCT Patent Application No. PCT/US00/06719 published on Nov. 23, 2000) (incorporated herein by reference). Such known techniques also include, for example, those disclosed in Int'l Publ. No. WO 02/092588 (PCT Application No. PCT/US02/15257 published Nov. 21, 2002) (incorporated herein by reference). Such known techniques further include, for example, those disclosed in U.S. Appl. Publ. No. US-2003-0073718 published Apr. 17, 2003 (incorporated herein by reference). Such known techniques also include, for example, those disclosed in WIPO PCT Appl. No. PCT/IUSO3/20028 filed Jun. 25, 2003 (incorporated herein by reference).

### EXAMPLES

The following examples are merely illustrative, and not limiting to the remainder of this disclosure in any way.

### Example 1

### Preparation of 4-{[5-(4-butoxyphenyl)thien-2-yl]sulfonyl}-N-hydroxytetrahydro-2H-pyran-4-carboxamide

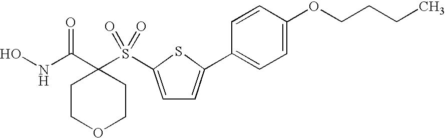

Part A. Preparation of 2-(4-butoxyphenyl)thiophene (3):

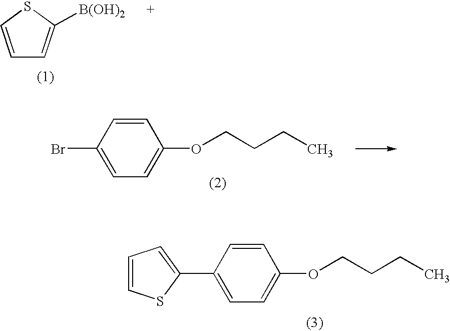

2-Thiophene boronic acid (1) (from Aldrich, 5.0 g, MW 127.96), 4-butoxybromobenzene (2) (from Maybridge, 9.4 g, MW 229.12, 1.05 eq), tetrakis(triphenylphosphine)palladium (from Aldrich, 2.2 g, MW 1155.58, 0.05 eq), and 2 M sodium carbonate (aqueous) (25.4 ml, 1.3 eq) were slurried in ethylene glycol dimethylether (80 ml). The resulting mixture was stirred at 80° C. for 5 hr under N₂. The reaction vessel was then cooled to −40° C. Afterward, a mixture of dichloromethane (150 ml) and ice (200 g) were introduced into the mixture. The mixture was allowed to increase to room temperature, and then the layers were separated. The organics were washed with water (2×), washed with brine (1×), dried over Na₂SO₄, and concentrated to afford a brown oil that was chromatographed (ethylacetate:hexanes , 1:49) to afford 2-(4-butoxyphenyl)thiophene (3) as a pale yellow solid (5.3 g, 58% yield). ¹H NMR confirmed the presence of the desired compound (3). The “equivalents” above indicate equivalents relative to the charged amount of 2-thiophene boronic acid.

Part B. Preparation of 2-(4-butoxyphenyl)-5-(methylsulfonyl)thiophene:

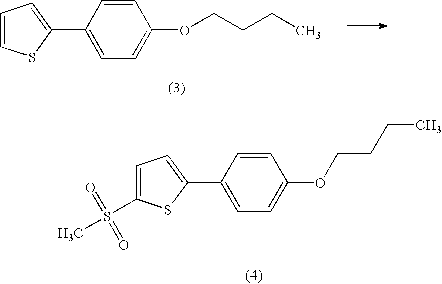

A solution of 2-(4-butoxyphenyl)thiophene (3) from Part A (3.4 g, MW 232.34) in tetrahydrofuran (20 ml) was cooled to 0° C. under N₂. Once cooled, a solution of n-butyllithium (from Aldrich, 1.6 M hexanes, 11.0 ml, 1.2 eq) was slowly added. The reaction stirred for 1 hr at 0° C. Afterward, a solution of methyldisulfide (from Aldrich, 1.4 g, MW 94.2, 1.05 eq) in tetrahydrofuran (10 ml) was added. The ice bath was removed, and the reaction stirred for 2 hr at room temperature. After complete lithiation, the following were slowly added in order: water (25 ml), tetrahydrofuran (50 ml), and Oxone (from Aldrich, 50.8 g, MW 614, 5.7 eq). After 3 hr, the mixture was filter through a Celite pad. The filtrate was then separated, and the organic was washed with water (3×), washed with brine (1×), dried over sodium sulfate, and concentrated to afford a dark purple solid. The resulting solid was dissolved in ethyl acetate, and a solid was then precipitated out with hexanes to afford 2-(4-butoxyphenyl)-5-(methylsulfonyl)thiophene (4) as a light purple solid. This solid was collected and dried to afford 2.65 g (58% yield). ¹H NMR confirmed the presence of the desired compound (4). The “equivalents” above indicate equivalents relative to the charged amount of 2-(4-butoxyphenyl)thiophene.

Part C. Preparation of tert-butyl {[5-(4-butoxyphenyl)thien-2-yl]sulfonyl}acetate (5):

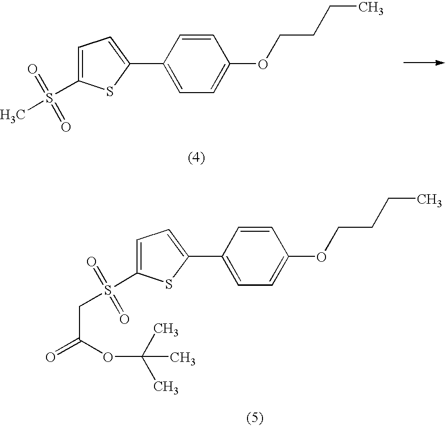

A solution of 2-(4-butoxyphenyl)-5-(methylsulfonyl)thiophene (4) from Part B (3.8 g, MW 310.43, 1.0 eq) and t-butylcarboxlyate anhydride (from Aldrich, 3.2 g, MW 218.25, 1.2 eq) in tetrahydrofuran (from Aldrich, 20 ml) was cooled to −75° C. A solution of lithium bis(trimethylsilyl)amide (from Aldrich, 1.0 M in tetrahydrofuran, 36.6 ml, 3.0 eq) was slowly added while maintaining the temperature at less than −65° C. Afterward, the mixture was warmed to 0° C. and stirred 1 hr. The mixture was then cooled back to −75° C. and quenched with a saturated solution of ammonium chloride (aqueous). The mixture was then warmed to room temperature, and the layers were separated. The aqueous layer was extracted with ethylacetate (2×). The organics were then combined and washed with water (2×), washed with brine (2×), dried over Na₂SO₄, and concentrated to afford a crude black oil. This oil was chromatographed (ethyl acetate:hexanes, 2:10) to afford tert-butyl {[5-(4-butoxyphenyl)thien-2-yl]sulfonyl}acetate (5) as brown solid (4.47 g 89% yield). ¹H NMR confirmed the presence of the desired compound (5). The “equivalents” above indicate equivalents relative to the charged amount of 2-(4-butoxyphenyl)-5-(methylsulfonyl)thiophene (4).

Part D. Preparation of tert-butyl-4-{[5-(4-butoxyphenyl)thien-2-yl]sulfonyl}tetrahydro-2H-pyran-4-carboxylate (6):

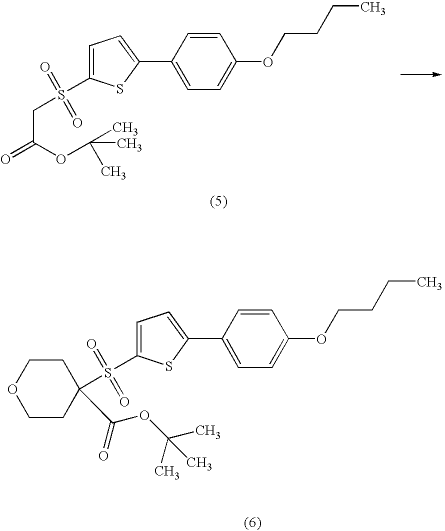

Tert-butyl {[5-(4-butoxyphenyl)thien-2-yl]sulfonyl} acetate (5) from Part C (4.0 g, MW 410.55), 18-crown-6 (from Aldrich, 0.5 g, catalytic amount), potassium carbonate (from Aldrich, 5.4 g, MW 138.21, 4.0 eq), and bis(bromoethyl)ether (from Aldrich, 3.4 g, MW 231.93, 1.5 eq) were slurried in N,N-dimethylformamide (20 ml). The resulting mixture was stirred at 65° C. for 15 hr. Afterward, the mixture was diluted with water (50 ml) and extracted with ethyl acetate (3×-100 ml). The organics were combined and washed with water (2×), washed with brine (1×), dried over Na₂SO₄, and concentrated for a tan oil. The oil was washed with hexanes and dried to afford tert-butyl-4-{[5-(4-butoxyphenyl)thien-2-yl]sulfonyl}tetrahydro-2H-pyran-4-carboxylate (6) as a tan oil (4.3 g, 91% yield). ¹H NMR and LCMS confirmed the presence of the desired compound (6). The “equivalents” above indicate equivalents relative to the charged amount of tert-butyl {[5-(4-butoxyphenyl)thien-2-yl]sulfonyl} acetate.

Part E. Preparation of 4-{[5-(4-butoxyphenyl)thien-2-yl]sulfonyl}tetrahydro-2H-pyran-4-carboxylic Acid (7):

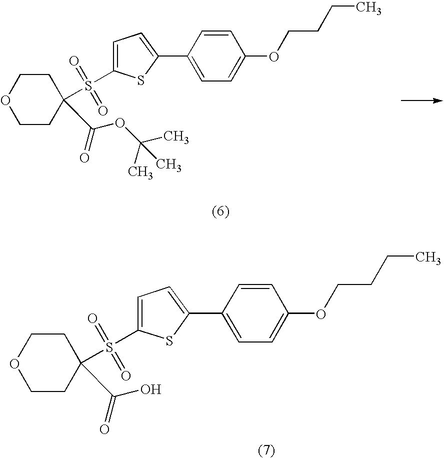

To a solution of tert-butyl-4-{[5-(4-butoxyphenyl)thien-2-yl]sulfonyl}tetrahydro-2H-pyran-4-carboxylate (6) from Part D (4.3 g, MW 480.64) in dichloromethane (10 ml) was added trifluoroacetic acid (from Aldrich, 20 ml). The resulting mixture was stirred overnight at room temperature. The mixture was then concentrated to one-third volume. The concentrated residue was dripped into stirring diethylether (500 ml). The resulting solid was collected, washed with diethylether, and dried to afford 4-{[5-(4-butoxyphenyl)thien-2-yl]sulfonyl}tetrahydro-2H-pyran-4-carboxylic acid (7) as a gray-green solid (325 g, 85% yield). ¹H NMR confirmed the presence of the desired compound (7).

Part F. Preparation of 4-{[5-(4-butoxyphenyl)thien-2-yl]sulfonyl}-N-(tetrahydro-2H-pyran-2-yloxy)tetrahydro-2H-pyran-4-carboxamide (8):

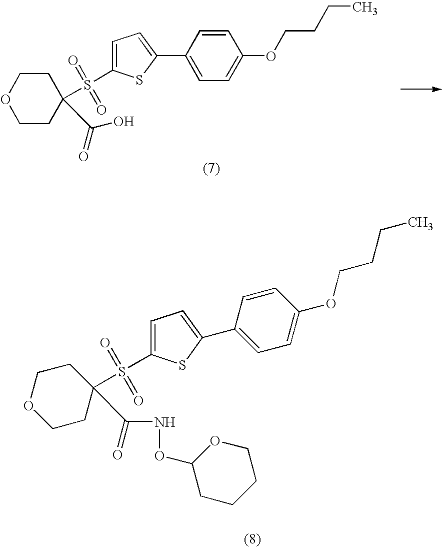

To the solid of 4-{[5-(4-butoxyphenyl)thien-2-yl]sulfonyl}tetrahydro-2H-pyran-4-carboxylic acid (7) from Part E (1.6 g, MW 424.53) in N,N-dimethylformamide (10 ml) was added triethylamine (from Aldrich, 0.64 ml, MW 101.19, 2.0 eq) followed by N-hydroxybenzotriazole hydrate (from Aldrich, 1.0 g, MW 135.13, 2.0 eq), 0- (tetrahydro-2H-pyran-2-yl)hydroxylamine (0.88 g, MW 117.16, 2.0 eq), and, lastly, 1-(3-dimethylaminopropyl)-3-ethylcarbodiimide hydrochloride (from Sigma, 1.6 g, MW 191.76, 2.2 eq)). The mixture stirred at room temperature for 5 hr. Workup consisted of diluting with water (15 ml) and ethylacetate (100 ml). The organic was separated and the aqueous was further extracted with ethylacetate (2×75 ml). The organics were combined and washed with sat. NaHCO₃(aq) (2×150 ml), water (2×-100 ml), and brine (1×200 ml). After drying over sodium sulfate, the organics were concentrated to afford 4-{[5-(4-butoxyphenyl)thien-2-yl]sulfonyl}-N-(tetrahydro-2H-pyran-2-yloxy)tetrahydro-2H-pyran-4-carboxamide (8) as a tan oil (2.0 g, 100% crude yield). ¹H NMR confirmed the presence of the desired compound (8). The “equivalents” above indicate equivalents relative to the charged amount of 4-{[5-(4-butoxyphenyl)thien-2-yl]sulfonyl} tetrahydro-2H-pyran-4-carboxylic acid.

Part G. Preparation of 4-{[5-(4-butoxyphenyl)thien-2-yl]sulfonyl}-N-hydroxytetrahydro-2H-pyran-4-carboxamide (9):

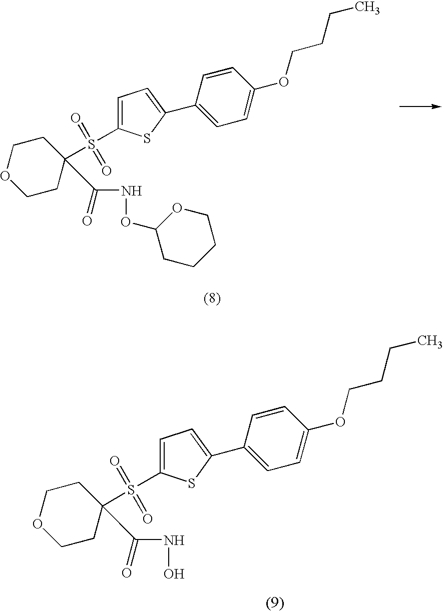

To 4-{[5-(4-butoxyphenyl)thien-2-yl]sulfonyl}-N-(tetrahydro-2H-pyran-2-yloxy)tetrahydro-2H-pyran-4-carboxamide (8) from Part F (2.0 g, MW 523.66) was added methanol (1 ml) and 4 N HCl in dioxane (8 ml) over 1 hr. The mixture was then concentrated to one-third volume. Afterward, diethylether was added. The resulting solid was filtered, washed with diethylether, and dried to afford 4-{[5-(4-butoxyphenyl)thien-2-yl]sulfonyl}-N-hydroxytetrahydro-2H-pyran-4-carboxamide (9) as a greenish solid (1.24 g, 74% yield). ¹H NMR confirmed the presence of the desired compound (9). HRMS for C₂₀H₂₅NO₆S₂ showed M^{+H}_{found}=440.1232 (M^{+H}_{calc}=440.1201).

### Example 2

### Preparation of N-hydroxy-4-({5-[4-(1,1,2,2-tetrafluoroethoxy)phenyl]thien-2-yl}sulfonyl)tetrahydro-2H-pyran-4-carboxamide

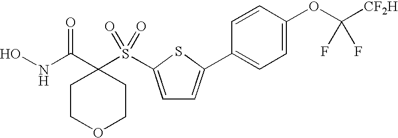

Part A. Preparation of tert-butyl (thien-2-ylthio)acetate (3):

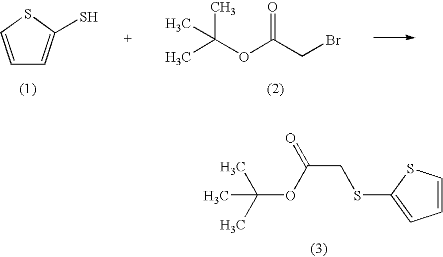

2-Mercapto thiophene (1) (Lancaster, 5.0 g, MW 116.21), t-butylbromoacetate (2) (from Aldrich, 6.4 ml, MW 195.05, 1.0 eq), and potassium carbonate (from Aldrich, 6.2 g, MW 138.21, 1.05 eq) were slurried in N,N-dimethylformamide (80 ml). The mixture stirred at room temperature for 15 hr under N₂. After completion, the mixture was diluted with water (100 ml) then extracted with ethyl acetate (3×100 ml). The organics were washed with water (2×) and brine (1×) then dried over Na₂SO₄ and concentrated to afford tert-butyl (thien-2-ylthio)acetate (3) as a brown oil that was used directly in the next step. ¹H NMR confirmed the presence of the desired compound (3). The “equivalents” above indicate equivalents relative to the charged amount of 2-mercapto thiophene.

Part B. Preparation of Tert-Butyl (thien-2-ylsulfonyl)acetate (4):

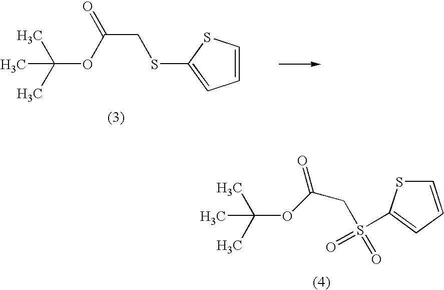

To a solution of tert-butyl (thien-2-ylthio)acetate (3) from Part A (9.9 g, MW 230.35) in tetrahydrofuran (45 ml) and water (30 ml) was slowly added Oxone (from Aldrich, 52.9 g, MW 614, 2.0 eq). After stirring for 15 hr at room temperature, the mixture was filtered through a pad of Celite. The filtrate was stripped of organics. The aqueous was extracted with ethyl acetate (3×-100 ml). The organics were then combined and washed with water (3×), washed with brine (1×), dried over sodium sulfate, and concentrated to afford tert-butyl-(thien-2-ylsulfonyl)acetate (4) as a tan oil (100% crude yield). ¹H NMR confirmed the presence of the desired compound (4). The “equivalents” above indicate equivalents relative to the charged amount of tert-butyl (thien-2-ylthio)acetate.

Part C. Preparation of tert-butyl-4-(thien-2-ylsulfonyl)tetrahydro-2H-pyran-4-carboxylate (5):

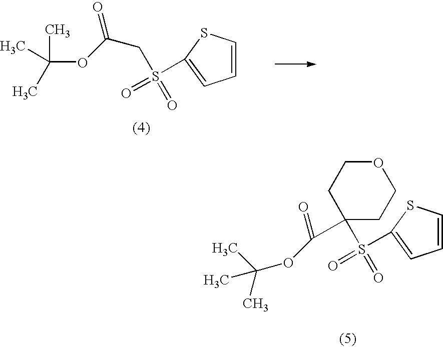

Tert-butyl (thien-2-ylsulfonyl)acetate (4) from Part B (11.3 g, MW 262.35), 18-crown-6 (from Aldrich, 0.5 g, catalytic amount), potassium carbonate (from Aldrich, 17.9 g, MW 138.21, 3.0 eq), and bis(bromoethyl)ether (from Aldrich, 15.0 g, MW 231.93, 1.5 eq) were slurried in N,N-dimethylformamide (20 ml) and stirred at 65° C. for 15 hr. Afterward, the mixture was diluted with water (50 ml) and extracted with ethyl acetate (3×-100 ml). The organics were combined and washed with water (2×), washed with brine (1×), dried over Na₂SO₄, and concentrated for a tan oil. The oil was chromatographed (silica gel, 1:5, Ethyl acetate:hexanes ) to afford tert-butyl-4-(thien-2-ylsulfonyl)tetrahydro-2H-pyran-4-carboxylate (5) as a white solid (10.9 g, 76% yield). ¹H NMR and LCMS confirmed the presence of the desired compound (5). The “equivalents” above indicate equivalents relative to the charged amount of tert-butyl (thien-2-ylsulfonyl)acetate.

Part D. Preparation of tert-butyl-4-({5-[4-(1,1,2,2-tetrafluoroethoxy)phenyl]thien-2-yl}sulfonyl)tetrahydro-2H-pyran-4-carboxylate (6):

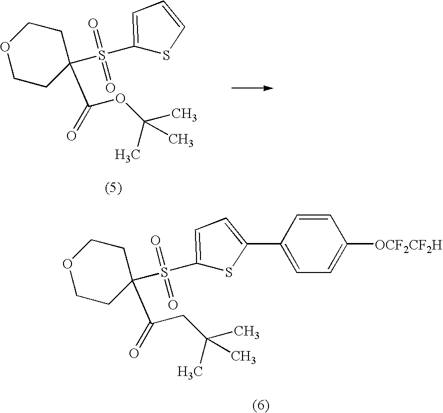

Tert-butyl-4-(thien-2-ylsulfonyl)tetrahydro-2H-pyran-4-carboxylate (5) from Part C (2.0 g, MW 332.44), tetrakis(triphenylphosphine)palladium (from Aldrich, 0.35 g, MW 1155.58, 0.05 eq), potassium acetate (from Aldrich, 1.5 g, MW 98.14, 2.5 eq), and 4-bromo-tetrafluorethoxybenzene (Indofine, 1.8 g, MW 273.03, 1.1 eq) were slurried in N,N-dimethylacetamide (15 ml) and stirred at 80° C. for 5 hr. Afterward, the mixture was filtered through a Celite pad and washed with ethyl acetate. The filtrate was washed with water (3×-50 ml), washed with brine (1x-100 ml), dried over Na₂SO₄, and concentrated to form a black oil. The oil was chromatographed (silica gel, 1:10, ethyl acetate:hexanes ) to afford tert-butyl-4-({5-[4-(1,1,2,2-tetrafluoroethoxy)phenyl]thien-2-yl}sulfonyl)tetrahydro-2H-pyran-4-carboxylate (6) as a tan solid (1.1 g, 35% yield). ¹H NMR and LCMS confirmed the presence of the desired compound (6). The “equivalents” above indicate equivalents relative to the charged amount of tert-butyl-4-(thien-2-ylsulfonyl)tetrahydro-2H-pyran-4-carboxylate.

Part E. Preparation of 4-({5-[4-(1,1,2,2-tetrafluoroethoxy)phenyl]thien-2-yl}sulfonyl)tetrahydro-2H-pyran-4-carboxylic Acid (7):

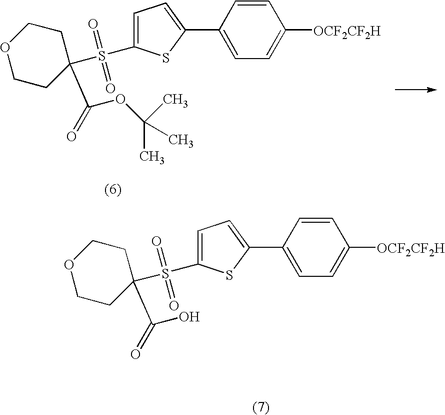

To a solution of tert-butyl-4-({5-[4-(1,1,2,2-tetrafluoroethoxy)phenyl]thien-2-yl}sulfonyl)tetrahydro-2H-pyran-4-carboxylate (6) from Part D (1.1 g, MW 524.55) in dichloromethane (5 ml) was added trifluoroacetic acid (from Aldrich, 10 ml). The reaction was stirred for 4 hr at room temperature. Afterward, the mixture was concentrated to one-third volume. The residue was then dripped into stirring diethylether (500 ml). The resulting solid was collected, washed with diethylether, and dried to afford 4-({5-[4-(1,1,2,2-tetrafluoroethoxy)phenyl]thien-2-yl}sulfonyl)tetrahydro-2H-pyran-4-carboxylic acid (7) as a white solid (1.0 g, 100% yield). LCMS confirmed the presence of the desired compound (7).

Part F. Preparation of 4-({5-[4-(1,1,2,2-tetrafluoroethoxy)phenyl]thien-2-yl}sulfonyl)-N-(tetrahydro-2H-pyran-2-yloxy)tetrahydro-2H-pyran-4-carboxamide (8):

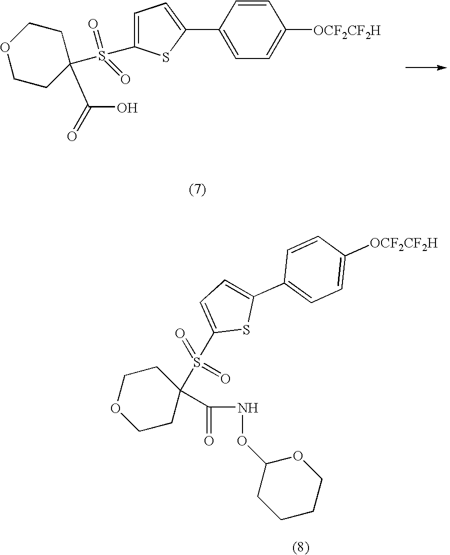

To the solid of 4-({5-[4-(1,1,2,2-tetrafluoroethoxy)phenyl]thien-2-yl}sulfonyl)tetrahydro-2H-pyran-4-carboxylic acid (7) from Part E (1.0 g, MW 468.44) in N,N-dimethylformamide (10 ml) was added triethylamine (from Aldrich, 0.58 ml, MW 101.19, 2.0 eq), followed by N-hydroxybenzotriazole hydrate (from Aldrich, 0.57 g, MW 135.13, 2.0 eq), O-(tetrahydro-2H-pyran-2-yl) hydroxylamine (0.37 g, MW 117.16, 1.5 eq), and, lastly, 1-(3-dimethylaminopropyl)-3-ethylcarbodiimide hydrochloride (from Sigma, 1.0 g, MW 191.76, 2.5 eq)). The mixture was then stirred at room temperature for 15 hr. Afterward, the mixture was diluted with water (15 ml) and ethylacetate (100 ml). The organic phase was separated, and the aqueous was further extracted with ethylacetate (2×75 ml). The organics were then combined and washed with saturated NaHCO₃aq (2×150 ml), washed with water (2×-100 ml), washed with brine (1×200 ml), dried over sodium sulfate, and concentrated to afford 4-({5-[4-(1,1,2,2-tetrafluoroethoxy)phenyl]thien-2-yl}sulfonyl)-N-(tetrahydro-2H-pyran-2-yloxy)tetrahydro-2H-pyran-4-carboxamide (8) as a tan oil (1.5 g, 100% crude yield). ¹H NMR confirmed the presence of the desired compound (8). The “equivalents” above indicate equivalents relative to the charged amount of 4-({5-[4-(1,1,2,2-tetrafluoroethoxy)phenyl]thien-2-yl}sulfonyl)tetrahydro-2H-pyran-4-carboxylic acid.

Part G. Preparation of N-hydroxy-4-({5-[4-(1,1,2,2-tetrafluoroethoxy)phenyl]thien-2-yl}sulfonyl)tetrahydro-2H-pyran-4-carboxamide (9):

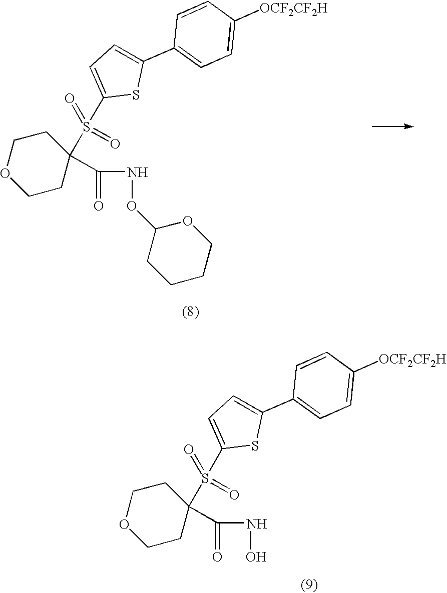

To 4-({5-[4-(1,1,2,2-tetrafluoroethoxy)phenyl]thien-2-yl}sulfonyl)-N-(tetrahydro-2H-pyran-2-yloxy)tetrahydro-2H-pyran-4-carboxamide (8) from Part F (1.5 g, MW 567.57) was added methanol (1 ml) and 4 N HCl in dioxane (8 ml) over 1 hr. The mixture was then concentrated to one-third volume. Diethylether was then added. The resulting oil was dissolved in methanol, and then a solid was precipitated out with water. The solid was dried to afford N-hydroxy-4-({5-[4-(1,1,2,2-tetrafluoroethoxy)phenyl]thien-2-yl}sulfonyl)tetrahydro-2H-pyran-4-carboxamide (9) as a white solid (0.53 g, 53% yield). ¹H NMR confirmed the presence of the desired compound (9). HRMS for C₁₈H₁₇F₄NO₆S₂ showed M^{+H}_{found}=484.0506 (M^{+H}_{calc}=484.0536).

### Example 3

### Preparation of tert-butyl 4-[(6-bromopyridin-3-yl)sulfonyl]tetrahydro-2H-pyran-4-carboxylate

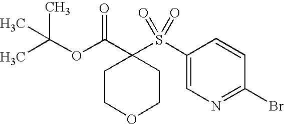

Part A. Preparation of 2-bromo-5-(methylsulfonyl)pyridine (2):

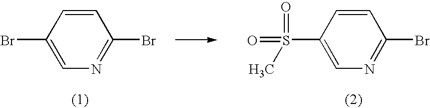

2,5-Dibromopyridine (1) (from Aldrich, 10.0 g, MW 236.89) was dissolved in anhydrous diethyl ether (from Aldrich, 200 ml) and cooled to −78° C. Anhydrous N-Butyllithium (1.6 M in hexanes, 28 ml, 1.05 eq) was then slowly dripped into the mixture while maintaining the temperature at less than −60° C. After complete lithium-bromide exchange, a solution of methyl disulfide (from Aldrich, 4.0 ml, MW 94.2, 1.05 eq) in diethyl ether (80 ml) was added, again maintaining temperature at less than −60° C. After stirring for 1 hr at −78° C., the reaction mixture was quenched with water (100 ml) and diluted with tetrahydrofuran (from Aldrich, 100 ml). Oxone (from Aldrich, 77 g, MW 614 g, 3 eq) was then added while vigorously stirring the mixture. Afterward, the ice bath was removed, and the mixture was stirred for an additional 15 hr at room temperature. The mixture was then filtered through a Celite pad, and the filtrate was separated. The organics were concentrated to a residue, and then dissolved in ethyl acetate. The ethyl acetate was washed with water (3×), washed with brine (1×), dried over Na₂SO₄, and concentrated to afford 2-bromo-5-(methylsulfonyl)pyridine (2) as a tan solid (9.2 g, 93% yield). ¹H, NOE, and HMBC NMR and LCMS confirmed the presence of the desired compound (2). The “equivalents” above indicate equivalents relative to the charged amount of 2,5-dibromopyridine.

Part B. Preparation of tert-butyl [(6-bromopyridin-3-yl)sulfonyl]acetate (3):

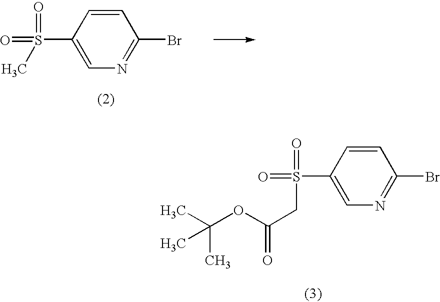

To a solution of 2-bromo-5-(methylsulfonyl)pyridine (2) from Part A (9.2 g, MW 236.09) and t-butylcarboxlyate anhydride (from Aldrich, 10.5 g, MW 218.25, 1.2 eq) in tetrahydrofuran (from Aldrich, 80 ml) was cooled to −78° C. A solution of lithium bis(trimethylsilyl)amide (from Aldrich, 1.0 M in tetrahydrofuran, 116.9 ml, 3.0 eq) was slowly added, keeping the temperature at less than −65° C. Afterward, the mixture was warmed to 0° C. and stirred for 1 hr. The mixture was then cooled back to −75° C., and then quenched with a saturated solution of ammonium chloride (aqueous). The mixture was subsequently warmed to room temperature and then separated. The aqueous layer was further extracted with ethylacetate (2×). The organics were then combined and washed with water (2×), washed with brine (2×), dried over Na₂SO₄, and concentrated to a crude black oil, which was chromatographed (ethyl acetate:hexanes, 2:10) to afford tert-butyl [(6-bromopyridin-3-yl)sulfonyl]acetate (3) as a tan oil (7.9 g 59% yield). ¹H NMR confirmed the presence of the desired compound (3). The “equivalents” above indicate equivalents relative to the charged amount of 2-bromo-5-(methylsulfonyl)pyridine.

Part C. Preparation of tert-butyl 4-[(6-bromopyridin-3-yl)sulfonyl]tetrahydro-2H-pyran-4-carboxylate (4):

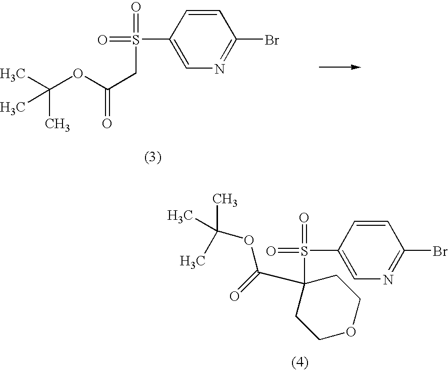

Tert-butyl [(6-bromopyridin-3-yl)sulfonyl]acetate (3) from Part B (4.37 g, MW 262.35), 18-crown-6 (from Aldrich, 0.5 g, catalytic amount), potassium carbonate (from Aldrich, 7.39 g, MW 138.21, 5.3 eq), and bis(bromoethyl)ether (from Aldrich, 3.4 ml, MW 231.93, 2.1 eq) were slurried in N,N-dimethylformamide (25 ml) and stirred at 65° C. for 15 hr. Afterward, the mixture was diluted with water (50 ml) and extracted with ethyl acetate (3×100 ml). The organics were combined and washed with water (2×), washed with brine (1×), dried over Na₂SO₄, and concentrated to form an orangish oily solid. This oil was slurried with hexanes, filtered, and dried to afford tert-butyl 4-[(6-bromopyridin-3-yl)sulfonyl]tetrahydro-2H-pyran-4-carboxylate (4) as a yellow solid (3.8 g, 72% yield). ¹H NMR and LCMS confirmed the presence of the desired compound (4). The “equivalents” above indicate equivalents relative to the charged amount of tert-butyl [(6-bromopyridin-3-yl)sulfonyl] acetate.

### Example 4

### Preparation of N-hydroxy-4-{[6-(4-pentylphenyl)pyridin-3-yl]sulfonyl}tetrahydro-2H-pyran-4-carboxamide hydrochloride

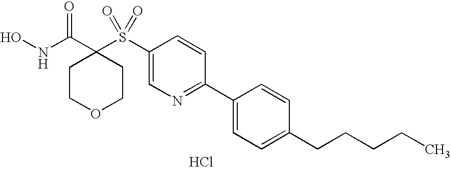

Part A. Preparation of tert-butyl-4-{[6-(4-pentylphenyl)pyridin-3-yl]sulfonyl}tetrahydro-2H-pyran-4-carboxylate (2):

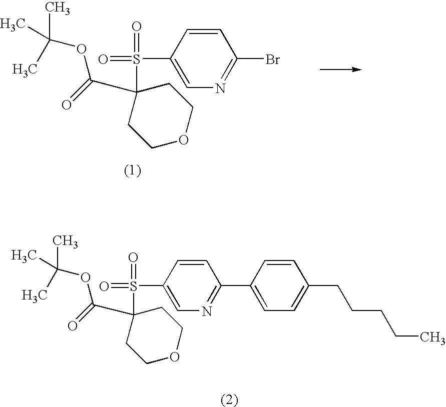

Tert-butyl 4-[(6-bromopyridin-3-yl)sulfonyl]tetrahydro-2H-pyran-4-carboxylate (1) from Example 3 (1.0 g, MW 406.29), tetrakis(triphenylphosphine)palladium (from Aldrich, 0.14 g, MW 1155.58, 0.05 eq), sodium carbonate (from Aldrich, 2 M aqueous, 1.6 ml, 1.3 eq), and 4-n-pentylphenyl boronic acid (Lancaster, 0.53 g, MW 192.06, 1.1 eq) were slurried in ethylene glycol dimethylether (10 ml) and stirred at 80° C. for 3 hr. Afterward, the mixture was filtered through a Celite pad and washed with ethyl acetate. The filtrate was then washed with water (3×50 ml), washed with brine (1x-100 ml), dried over Na₂SO₄, and concentrated to form an orange solid. This solid was chromatographed (silica gel, 3:20, ethyl acetate:hexanes ) to afford tert-butyl-4-{[6-(4-pentylphenyl)pyridin-3-yl]sulfonyl}tetrahydro-2H-pyran-4-carboxylate (2) as a tan solid (1.1 g, 92% yield). ¹H NMR and LCMS confirmed the presence of the desired compound (2). The “equivalents” above indicate equivalents relative to the charged amount of tert-butyl 4-[(6-bromopyridin-3-yl)sulfonyl]tetrahydro-2H-pyran-4-carboxylate.

Part B. Preparation of 4-{[6-(4-pentylphenyl)pyridin-3-yl]sulfonyl}tetrahydro-2H-pyran-4-carboxylic Acid Trifluoroacetate (3):

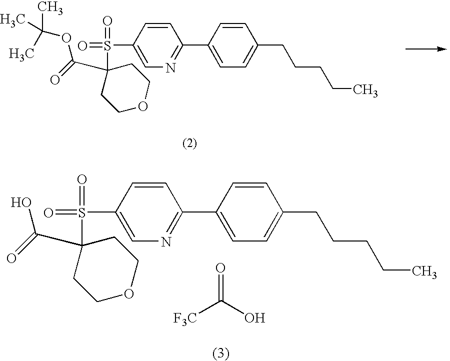

To a solution of tert-butyl-4-{[6-(4-pentylphenyl)pyridin-3-yl]sulfonyl}tetrahydro-2H-pyran-4-carboxylate (2) from Part A (1.1 g, MW 473.63) in dichloromethane (10 ml) was added trifluoroacetic acid (from Aldrich, 5 ml). The resulting mixture was stirred for 4 hr at room temperature. The mixture was then concentrated to one-third volume. Afterward, the residue was slowly dripped into stirring diethylether (5 ml). The resulting solid was collected, washed with diethylether, and dried to afford 4-{[6-(4-pentylphenyl)pyridin-3-yl]sulfonyl}tetrahydro-2H-pyran-4-carboxylic acid trifluoroacetate (3) as a white solid (0.93 g, 97% yield). LCMS confirmed the presence of the desired compound (3).

Part C. Preparation of 4-{[6-(4-pentylphenyl)pyridin-3-yl]sulfonyl}-N-(tetrahydro-2H-pyran-2-yloxy)tetrahydro-2H-pyran-4-carboxamide (4):

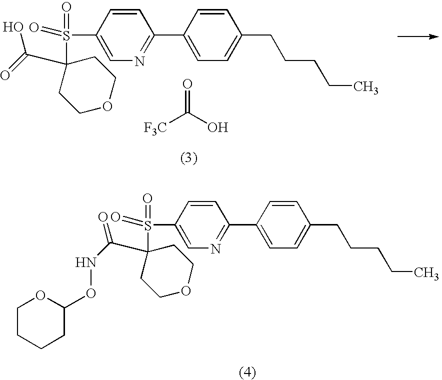

To the solid of 4-{[6-(4-pentylphenyl)pyridin-3-yl]sulfonyl} tetrahydro-2H-pyran-4-carboxylic acid trifluoroacetate (3) from Part B (0.9 g, FW 531.54) in N,N-dimethylformamide (5 ml) was added triethylamine (from Aldrich, 0.47 ml, MW 101.19, 2.0 eq), followed by N-hydroxybenzotriazole hydrate (from Aldrich, 0.46 g, MW 135.13, 2.0 eq), O-(tetrahydro-2H-pyran-2-yl)hydroxylamine (0.31 g, MW 117.16, 1.5 eq), and, lastly, 1-(3-dimethylaminopropyl)-3-ethylcarbodiimide hydrochloride (from Sigma, 0.81 g, MW 191.76, 2.5 eq)). The resulting mixture was stirred at room temperature for 15 hr. The mixture was then diluted with water (15 ml) and ethylacetate (100 ml). The organic layer was separated, and the aqueous was further extracted with ethylacetate (2×75 ml). The organics were then combined and washed with saturated NaHCO_{3(aq)} (2×-150 ml), washed with water (2×-100 ml), washed with brine (1×-200 ml), dried over sodium sulfate, and concentrated to afford 4-{[6-(4-pentylphenyl)pyridin-3-yl]sulfonyl}-N-(tetrahydro-2H-pyran-2-yloxy)tetrahydro-2H-pyran-4-carboxamide (4) as a foamy orange solid (0.83 g, 94% yield). ¹H NMR and LCMS confirmed the presence of the desired compound (4). The “equivalents” above indicate equivalents relative to the charged amount of 4-{[6-(4-pentylphenyl)pyridin-3-yl]sulfonyl}tetrahydro-2H-pyran-4-carboxylic acid trifluoroacetate.

Part D. Preparation of N-hydroxy-4-{[6-(4-pentylphenyl)pyridin-3-yl]sulfonyl}tetrahydro-2H-pyran-4-carboxamide Hydrochloride (5):

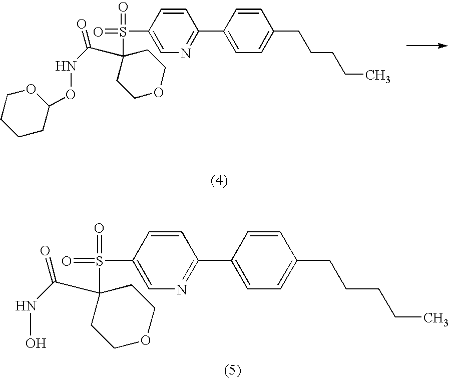

To 4-{[6-(4-pentylphenyl)pyridin-3-yl]sulfonyl}-N-(tetrahydro-2H-pyran-2-yloxy)tetrahydro-2H-pyran-4-carboxamide (4) from Part C (0.83 g, MW 516.65) was added methanol (1 ml) and 4 N HCl in dioxane (5 ml) for 1 hr. The mixture was concentrated to one-third volume, and then diethylether was added. The resulting oil was dissolved in methanol, and a solid was then precipitated out with water. The solid was dried to afford N-hydroxy-4-{[6-(4-pentylphenyl)pyridin-3-yl]sulfonyl} tetrahydro-2H-pyran-4-carboxamide hydrochloride (5) as a tan solid (0.57 g, 76% yield). ¹H NMR confirmed the presence of the desired compound (5). HRMS for C₂₂H₂₈N₂O₅S showed M^{+H}_{found}=433.1759 (M^{+H}_{calc}=433.1792).

### Example 5

### Preparation of N-hydroxy-4-([6-[4-(2,2,2-trifluoroethoxy)phenyl]pyridin-3-yl]sulfonyl)tetrahydro-2H-pyran-4-carboxamide Trifluoroacetate

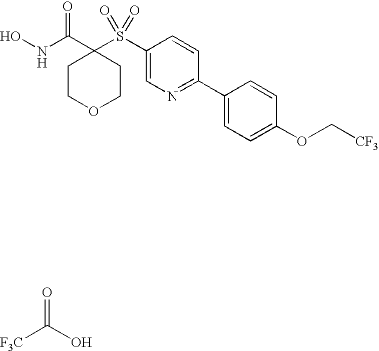

Part A. Preparation of tert-butyl-4-({6-[4-(2,2,2-trifluoroethoxy)phenyl]pyridin-3-yl}sulfonyl)tetrahydro-2H-pyran-4-carboxylate (3):

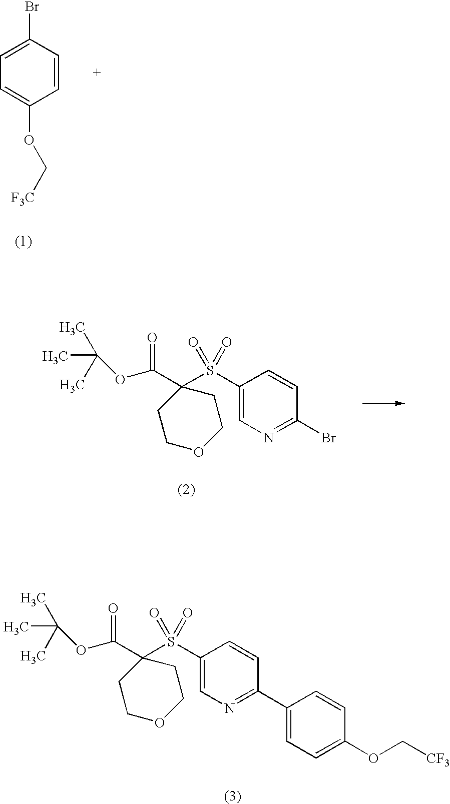

1-bromo-4-(2,2,2-trifluoroethoxy)benzene (1) (0.85 g, MW 255.03, 1.5 eq), pinacol diborane (from Aldrich, 0.89 g, MW 253.95, 1.6 eq), potassium acetate (from Aldrich, 0.86 g, MW 98.15, 4.0 eq), and (1,1′-bis(diphenylphosphino)-ferrocene)dichloropalladium(II) complex with dichloromethane (from Aldrich, 54 mg, MW 816.64, 0.03 eq) were charged in a round-bottom flask. The flask was purged with N₂. N,N-Dimethylformamide (from Aldrich, 8.0 ml) was then added, and the mixture was stirred at 80° C. for 2 hr. Tert-butyl 4-[(6-bromopyridin-3-yl)sulfonyl]tetrahydro-2H-pyran-4-carboxylate (2) (0.90 g, MW 406.29) was then added, along with sodium carbonate solution (2 M aqueous, 5.5 ml, 5 eq) and additional palladium complex (above, 54 mg, 0.03 eq). The reaction was continued at 80° C. for 3 hr. Afterward, the mixture was cooled to room temperature and filtered through a Celite pad. The filter cake was washed with ethyl acetate (2×50 ml). The filtrate and washes were then combined and washed with water (3×100 ml) and brine (1×100 ml). The organics were then dried over sodium sulfate and concentrated to form a black residue. The residue was chromatographed (silica gel, ethyl acetate:hexanes , 1:5) to afford tert-butyl-4-({6-[4-(2,2,2-trifluoroethoxy)phenyl]pyridin-3-yl}sulfonyl)tetrahydro-2H-pyran-4-carboxylate (3) as a white solid (0.26 g, 24% yield). The product (3) was confirmed by LCMS. The “equivalents” above indicate equivalents relative to the charged amount of tert-butyl 4-[(6-bromopyridin-3-yl)sulfonyl]tetrahydro-2H-pyran-4-carboxylate.

Part B. Preparation of 4-({6-[4-(2,2,2-trifluoroethoxy)phenyl]pyridin-3-yl}sulfonyl)tetrahydro-2H-pyran-4-carboxylic Acid Trifluoroacetate (4):

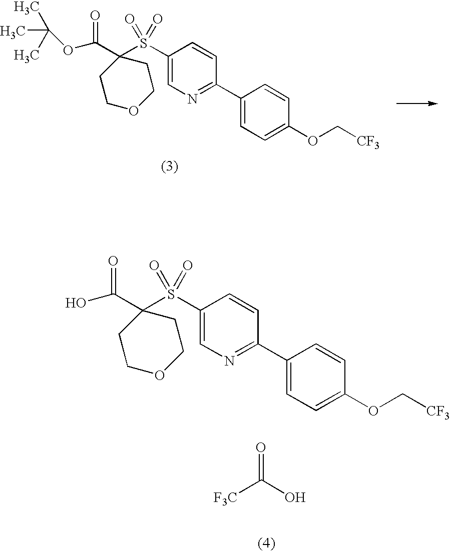

To a solution of tert-butyl-4-({6-[4-(2,2,2-trifluoroethoxy)phenyl]pyridin-3-yl}sulfonyl)tetrahydro-2H-pyran-4-carboxylate (3) from Part A (0.24 g, MW 501.52) in dichloromethane (5 ml) was added trifluoroacetic acid (from Aldrich, 5 ml). The mixture was stirred for 4 hr at room temperature. The mixture was concentrated to one-third volume. Afterward, the residue was dripped into stirring diethyl ether (5 ml). The resulting solid was collected, washed with diethyl ether, and dried to afford 4-({6-[4-(2,2,2-trifluoroethoxy)phenyl]pyridin-3-yl}sulfonyl)tetrahydro-2H-pyran-4-carboxylic acid trifluoroacetate (4) as a white solid (0.25 g, 96% yield). LCMS confirmed the presence of the desired compound (4).

Part C. Preparation of N-(tetrahydro-2H-pyran-2-yloxy)-4-({6-[4-(2,2,2-trifluoroethoxy)phenyl]pyridin-3-yl}sulfonyl)tetrahydro-2H-pyran-4-carboxamide (5):

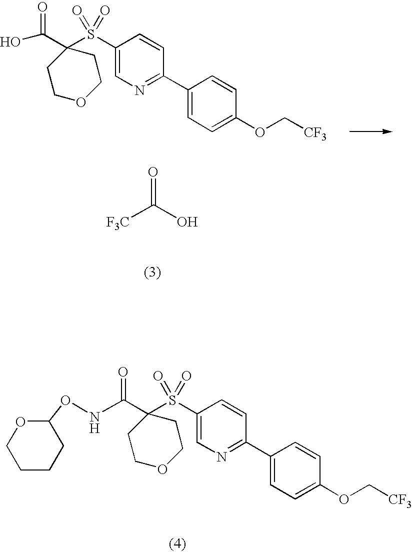

To the solid of 4-({6-[4-(2,2,2-trifluoroethoxy)phenyl]pyridin-3-yl}sulfonyl)tetrahydro-2H-pyran-4-carboxylic acid trifluoroacetate (4) from Part B (0.24 g, FW 559.43) in N,N-dimethylformamide (3 ml) was added triethylamine (from Aldrich, 0.17 ml, MW 101.19, 3.0 eq), followed by N-hydroxybenzotriazole hydrate (from Aldrich, 0.11 g, MW 135.13, 2.0 eq), 0- (tetrahydro-2H-pyran-2-yl) hydroxylamine (0.07 g, MW 117.16, 1.5 eq), and, lastly, 1-(3-dimethylaminopropyl)-3-ethylcarbodiimide hydrochloride (from Sigma, 0.19 g, MW 191.76, 2.5 eq)). The mixture was stirred at room temperature for 15 hr. Afterward, the mixture was diluted with water (15 ml) and ethylacetate (50 ml). The organic layer was separated, and the aqueous was further extracted with ethylacetate (2×-50 ml). The organics were then combined and washed with saturated NaHCO_{3(aq)} (2×100 ml), washed with water (2×100 ml), washed with brine (1×200 ml), dried over sodium sulfate, and concentrated to afford N-(tetrahydro-2H-pyran-2-yloxy)-4-({6-[4-(2,2,2-trifluoroethoxy)phenyl]pyridin-3-yl}sulfonyl)tetrahydro-2H-pyran-4-carboxamide (5) as a foamy orange solid (0.31 g, 100% crude yield). ¹H NMR and LCMS confirmed the presence of the desired compound (5). The “equivalents” above indicate equivalents relative to the charged amount of 4-({6-[4-(2,2,2-trifluoroethoxy)phenyl]pyridin-3-yl}sulfonyl)tetrahydro-2H-pyran-4-carboxylic acid trifluoroacetate.

Part D. Preparation of N-hydroxy-4-({6-[4-(2,2,2-trifluoroethoxy)phenyl]pyridin-3-yl}sulfonyl)tetrahydro-2H-pyran-4-carboxamide trifluoroacetate (6):

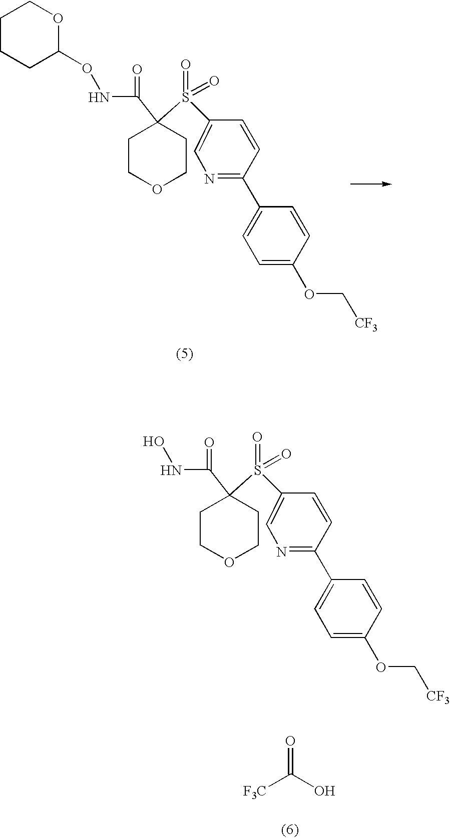

To N-(tetrahydro-2H-pyran-2-yloxy)-4-({6-[4-(2,2,2-trifluoroethoxy)phenyl]pyridin-3-yl}sulfonyl)tetrahydro-2H-pyran-4-carboxamide (5) from Part C (0.83 g, MW 516.65) was added methanol (1 ml) and 4 N HCl in dioxane (5 ml) over 1 hr. The mixture was then concentrated to one-third volume. Afterward, diethyl ether was added. The resulting oil was chromatographed on reverse phase (C-1 8, acetonitrile:water) to afford N-hydroxy-4-({6-[4-(2,2,2-trifluoroethoxy)phenyl]pyridin-3-yl}sulfonyl)tetrahydro-2H-pyran-4-carboxamide trifluoroacetate (6) as a white solid (0.05 g, 28% yield). ¹H NMR confirmed the presence of the desired compound (6). HRMS for C₁₉H₁₉F₃ N₂O₆S showed M^{+H}_{found}=461.0965 (M^{+H}_{calc}=461.0989).

### Example 6

### Preparation of N-hydroxy-4-({6-[4-(1,1,2,2-tetrafluoroethoxy)phenyl]pyridin-3-yl}sulfonyl)tetrahydro-2H-pyran-4-carboxamide Trifluoroacetate

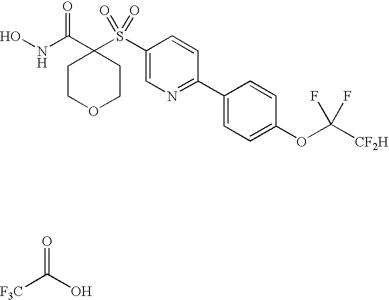

Part A. Preparation of tert-butyl-4-({6-[4-(1,1,2,2-tetrafluoroethoxy)phenyl]pyridin-3-yl}sulfonyl)tetrahydro-2H-pyran-4-carboxylate (3):

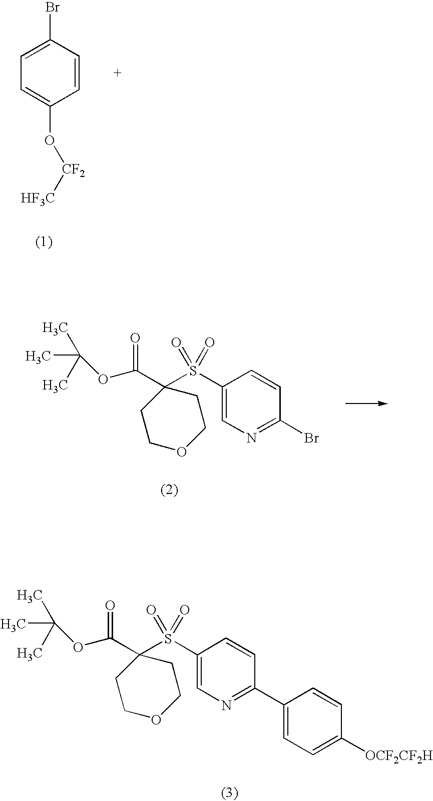

4-Bromo-tetrafluorethoxybenzene (1) (Indofine, 0.50 g, MW 273.03, 1.5 eq), pinacol diborane (from Aldrich, 0.49 g, MW 253.95, 1.6 eq), potassium acetate (from Aldrich, 0.47 g, MW 98.15, 4.0 eq), and (1,1′-bis(diphenylphosphino)-ferrocene)dichloropalladium(II) complex with dichloromethane (from Aldrich, 29 mg, MW 816.64, 0.03 eq) were charged to a round bottom flask. The flask was purged with N₂. N,N-Dimethylformamide (from Aldrich, 5.0 ml) was then added, and the mixture was stirred at 80° C. for 2 hr. Afterward, tert-butyl 4-[(6-bromopyridin-3-yl)sulfonyl]tetrahydro-2H-pyran-4-carboxylate (2) (0.50 g, MW 406.29) was added, along with sodium carbonate solution (2 M aqueous, 5.5 ml, 5 eq) and additional palladium complex (above, 29 mg, 0.03 eq). The reaction continued at 80° C. for 3 hr. The mixture was then cooled to room temperature and filtered through a Celite pad. The filter cake was washed with ethyl acetate (2×50 ml). The filtrate and washes were then combined and washed with water (3×-100 ml) and brine (1x-100 ml). The organics were then dried over sodium sulfate and concentrated to form a black residue. The residue was chromatographed (silica gel, ethyl acetate:hexanes , 1:5) to afford tert-butyl-4-({6-[4-(1,1,2,2-tetrafluoroethoxy)phenyl]pyridin-3-yl}sulfonyl)tetrahydro-2H-pyran-4-carboxylate (3) as a white solid (0.25 g, 40% yield). The product (3) was confirmed by LCMS. The “equivalents” above indicate equivalents relative to the charged amount of tert-butyl 4-[(6-bromopyridin-3-yl)sulfonyl]tetrahydro-2H-pyran-4-carboxylate.

Part B. Preparation of 4-({6-[4-(1,1,2,2-tetrafluoroethoxy)phenyl]pyridin-3-yl}sulfonyl)tetrahydro-2H-pyran-4-carboxylic acid trifluoroacetate (4):

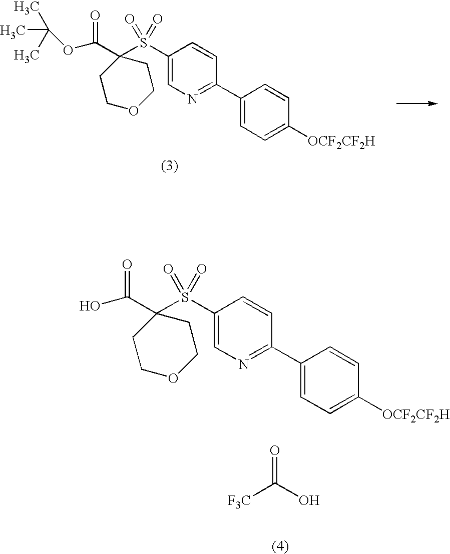

To a solution of tert-butyl-4-({6-[4-(1,1,2,2-tetrafluoroethoxy)phenyl]pyridin-3-yl}sulfonyl)tetrahydro-2H-pyran-4-carboxylate (4) (0.22 g, MW 519.21) in dichloromethane (2 ml) was added trifluoroacetic acid (from Aldrich, 3 ml). The mixture was then stirred for 4 hr at room temperature. Afterward, the mixture was concentrated to an oil and triturated with diethyl ether (5×). The resulting semi-solid was dried to afford 4-({6-[4-(1,1,2,2-tetrafluoroethoxy)phenyl]pyridin-3-yl}sulfonyl)tetrahydro-2H-pyran-4-carboxylic acid trifluoroacetate (5) as a white solid (0.24 g, 100% yield). LCMS confirmed the presence of the desired compound (5).

Part C. Preparation of 4-({6-[4-(1,1,2,2-tetrafluoroethoxy)phenyl]pyridin-3-yl}sulfonyl)tetrahydro-2H-pyran-4-carboxylic acid trifluoroacetate (6):

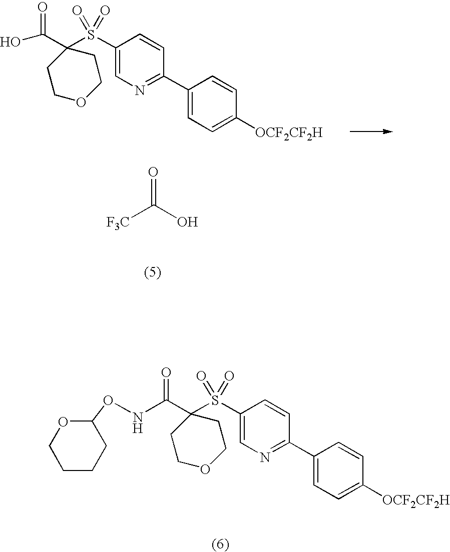

To the solid of 4-({6-[4-(1,1,2,2-tetrafluoroethoxy)phenyl]pyridin-3-yl}sulfonyl)tetrahydro-2H-pyran-4-carboxylic acid trifluoroacetate (5) from Part B (0.24 g, FW 577.43) in N,N-dimethylformamide (3 ml) was added triethylamine (from Aldrich, 0.17 ml, MW 101.19, 3.0 eq), followed by N-hydroxybenzotriazole hydrate (from Aldrich, 0.11 g, MW 135.13, 2.0 eq), O-(tetrahydro-2H-pyran-2-yl) hydroxylamine (0.07 g, MW 117.16, 1.5 eq), and, lastly, 1-(3-dimethylaminopropyl)-3-ethylcarbodiimide hydrochloride (from Sigma, 0.20 g, MW 191.76, 2.5 eq)). The resulting mixture was stirred at room temperature for 15 hr. Afterward, the mixture was diluted with water (15 ml) and ethylacetate (50 ml). The organic layer was separated, and the aqueous layer was further extracted with ethylacetate (2×-50 ml). The organics were then combined and washed with saturated NaHCO_{3(aq)} (2×100 ml), washed with water (2×100 ml), washed with brine (1×200 ml), dried over sodium sulfate, and concentrated to afford 4-({6-[4-(1,1,2,2-tetrafluoroethoxy)phenyl]pyridin-3-yl}sulfonyl)tetrahydro-2H-pyran-4-carboxylic acid trifluoroacetate (6) as a foamy orange solid (0.21 g, 88% yield). LCMS confirmed the presence of the desired compound (6). The “equivalents” above indicate equivalents relative to the charged amount of 4-({6-[4-(1,1,2,2-tetrafluoroethoxy)phenyl]pyridin-3-yl}sulfonyl)tetrahydro-2H-pyran-4-carboxylic acid trifluoroacetate.

Part D. Preparation of N-hydroxy-4-({6-[4-(1,1,2,2-tetrafluoroethoxy)phenyl]pyridin-3-yl}sulfonyl)tetrahydro-2H-pyran-4-carboxamide trifluoroacetate (7):

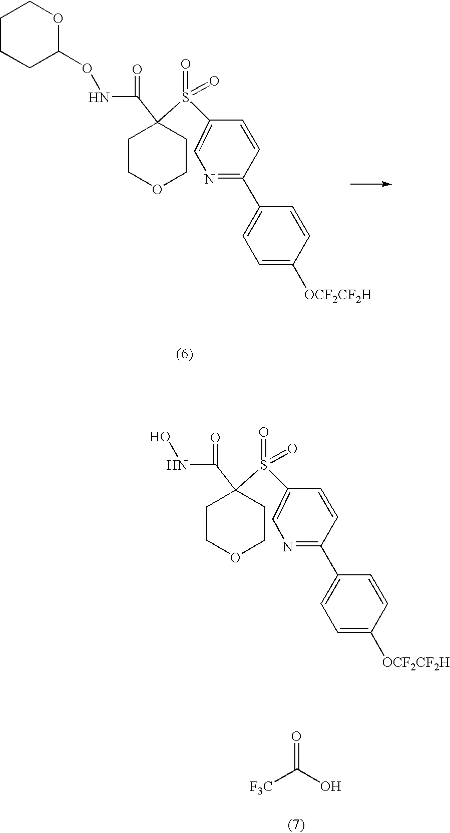

To 4-({6-[4-(1,1,2,2-tetrafluoroethoxy)phenyl]pyridin-3-yl}sulfonyl)tetrahydro-2H-pyran-4-carboxylic acid trifluoroacetate (6) from Part C (0.21 g, MW 562.53) was added methanol (1 ml) and 4 N HCl in dioxane (5 ml) over 1 hr. The mixture was then concentrated to one-third volume. Afterward, diethyl ether was added. The resulting oil was chromatographed on reverse phase (C-18, acetonitrile:water) to afford N-hydroxy-4-({6-[4-(1,1,2,2-tetrafluoroethoxy)phenyl]pyridin-3-yl}sulfonyl)tetrahydro-2H-pyran-4-carboxamide trifluoroacetate (7) as a white solid (0.05 g, 26% yield). ¹H NMR confirmed the presence of the desired compound (7). HRMS for C₁₉H₁₈F₄N₂O₆S showed M found 479.0863 (M^{+H}_{calc}=479.0894).

### Example 7

### Preparation of N-hydroxy-4-({5-[5-(3,3,4,4,4-pentafluorobutyl)pyridin-2-yl]thien-2-yl}sulfonyl) tetrahydro-2H-pyran-4-carboxamide Hydrochloride

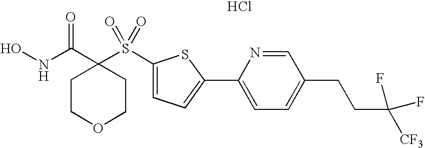

Part A. Preparation of 2-bromo-5-(methylthio)thiophene:

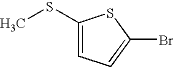

2,5-Dibromothiophene (from Aldrich, 40.0 g, MW 241.93) was dissolved in diethyl ether (300 ml) and then cooled to −78° C. A solution of n-butyl lithium (from Aldrich, 1.6 M in hexanes, 118 ml, 1.15 eq) was slowly added while maintaining the temperature at less than −65° C. After complete mono-exchange, a solution of dimethyldisulfide (from Aldrich, 14.2 ml, MW 94.20, 1.0 eq) in diethyl ether (20 ml) was added and the ice bath was removed while stirring, allowing the mixture to warm to ambient temperature. After the addition was complete, the mixture was diluted with water (500 ml) and then separated. The organic layer was washed with water (2×200 ml), washed with brine (1×200 ml), dried over sodium sulfate, and concentrated to form a black residue. The residue was passed through a silica gel plug and eluted with hexanes. Evaporation of the organics afforded the desired compound as a tan oil (34.3 g, 100+% yield). Some non-substituted thiophene was produced during the reaction and co-eluted with the product. ¹H NMR confirmed the presence of the desired compound. The “equivalents” above indicate equivalents relative to the charged amount of 2,5-dibromothiophene.

Part B. Preparation of 5-bromo-2-[5-(methylsulfonyl)thien-2-yl]pyridine:

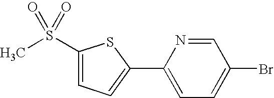

A dried round bottom flask was charged with magnesium turnings (from Aldrich, 1.26 g, MW 24.0 g) and iodide (from Aldrich, 20 mg, cat amt). The flask was heated with a heat gun until purple vapors were evident. The flask was then cooled to room temperature. Afterward, a solution of 2-bromo-5-(methylthio)thiophene from Part A (10 g, MW 209.13) in THF (50 ml) was added to form a Grignard reagent. The reaction mixture was heated at reflux until complete exchange was observed via HPLC. The mixture was then cooled to 0° C. In another dried round bottom flask, 2,5-dibromopyridine (from Aldrich, 11.3 g, MW 236.89, 1.0 eq) was slurried in THF (50 ml) along with (1,1′bis-(diphenylphosphino)-ferrocene)palladium dichloride (from Aldrich, 1.17 g, MW 816.64, 0.03 eq). This pyridine mixture was then cooled to 0° C. Subsequently, the Grignard mixture was poured into the pyridine mixture in a single shot. The ice bath was removed, and the resulting mixture stirred for 24 hr. The mixture was then filtered through a Celite plug to remove the palladium catalyst. Afterward, the mixture was diluted with water (100 ml). Oxone (from Aldrich, 88.1 g, MW 614, 3.0 eq) was then slowly added. The resulting mixture was stirred at room temperature for 15 hr (the reaction was complete at the end of the 15 hr). Afterward, the mixture was filtered through a pad of Celite. The filtrate was stripped of organics, and the resulting aqueous layer was extracted with ethyl acetate (3×100 ml). The organics were combined and washed with water (3×), washed with brine (1×), dried over sodium sulfate, and concentrated to afford the desired compound as an orange solid (4.1 g, 27% yield). ¹H NMR confirmed the presence of the desired compound. The “equivalents” above indicate equivalents relative to the charged amount of 2-bromo-5-(methylthio)thiophene.

Part C. Preparation of tert-butyl {[5-(5-bromopyridin-2-yl)thien-2-yl]sulfonyl}acetate:

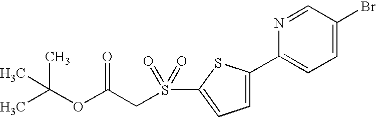

A solution of the product from Part B (4.1 g, MW 318.21) and t-butyl-dicarboxylate (from Aldrich, 3.3 g, MW 218.75, 1.2 eq) in THF (24 ml) was cooled to −78° C. A lithium hexamethyldisylisane solution in THF (1.0 M, 39 ml, 3.0 eq) was then slowly added while maintaining the temperature at less than −65° C. After the addition, the mixture was stirred for 1 hr, and then dripped into a saturated ammonium chloride aqueous solution (50 ml) to quench the reaction. The resulting mixture was warmed to room temperature. The organic layer was separated, and the aqueous layer was extracted with ethyl acetate (2×100 ml). The organics were then combined and washed with water, washed with brine, dried over sodium sulfate, and concentrated to form a black solid. The solid was chromatographed on silica gel (ethyl acetate/hexanes) to afford the desired compound as a yellow solid (2.0 g, 37% yield). ¹H NMR and LCMS confirmed the presence of the desired compound. The “equivalents” above indicate equivalents relative to the charged amount of product from Part B.

Part D. Preparation of tert-butyl 4-{[5-(5-bromopyridin-2-yl)thien-2-yl]sulfonyl}tetrahydro-2H-pyran-4-carboxylate:

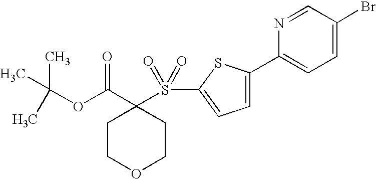

The product from Part C (1.75 g, MW 418.33), potassium carbonate (from Aldrich, 2.26 g, MW 138.21, 4.0 eq), and bis(bromoethyl)ether (from Aldrich, 1.16 g, MW 231.93, 1.2 eq) were slurried in N,N-dimethylformamide (10 ml). The resulting mixture was stirred at 65° C. for 15 hr. Afterward, the mixture was diluted with water (10 ml). The diluted mixture was extracted with ethyl acetate (3×50 ml). The organics were combined and washed with water (2×), washed with brine (1×), dried over Na₂SO₄, and concentrated to form an orangish, oily solid. The solid was washed with hexanes, and then dried to afford the desired compound as a yellow solid (0.9 g, 45% yield). ¹H NMR and LCMS confirmed the presence of the desired compound. The “equivalents” above indicate equivalents relative to the charged amount of product from Part C.

Part E. Preparation of tert-butyl 4-({5-[5-(3,3,4,4,4-pentafluorobutyl)pyridin-2-yl]thien-2-yl}sulfonyl)tetrahydro-2H-pyran-4-carboxylate:

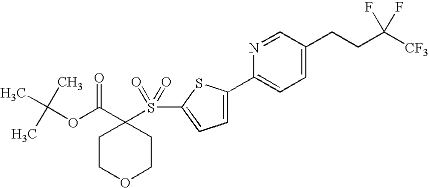

The product from Part D (0.5 g, MW 488.42), dichlorobis(benzonitrile)palladium (from Strem Chemical, 25 mg, MW 383.57, 0.064 eq), 2-(dicyclohexylphosphino)-2′-methyl-biphenyl (from Strem Chemical, 40 mg, MW 364.51, 0.107 eq) were slurried in N,N-dimethylacetamide (1.5 ml) for 20 min. A stock solution of 4,4,4,3,3-pentafluoro-iodozincbutane (0.7 M in THF, 2 ml, 1.4 eq) was then added. The resulting mixture was stirred at 55° C. for 2 hr. Once the reaction was complete, the mixture was quenched with 1N aqueous ammonium chloride, extracted with diethyl ether, filtered through filter syringe, and concentrated to form the crude solid. Recrystallization from ethanol afforded the desired compound as an orange solid (0.41 g, 72% yield). ¹H NMR and LCMS confirmed the presence of the desired compound. The “equivalents” above indicate equivalents relative to the charged amount of product from Part D.

Part F. Preparation of 4-({5-[5-(3,3,4,4,4-pentafluorobutyl)pyridin-2-yl]thien-2-yl}sulfonyl)tetrahydro-2H-pyran-4-carboxylic Acid:

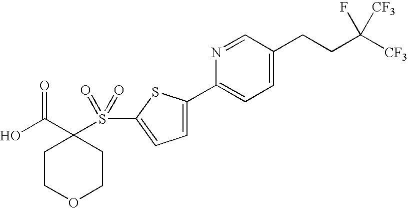

To a solution of the product from Part E (0.41 g, MW 499.47) in dichloromethane (3 ml) was added trifluoroacetic acid (from Aldrich, 5 ml). Afterward, the mixture was stirred for 4 hr at room temperature. The mixture was then concentrated to one-third volume to form a residue, which, in turn, was dripped into stirring diethylether (500 ml). The resulting solid was collected, washed with diethylether, and dried to afford the desired carboxylic acid as a tan solid (0.31 g, 84% yield). LCMS confirmed the presence of the desired compound.

Part G. Preparation of 4-({5-[5-(3,3,4,4,4-pentafluorobutyl)pyridin-2-yl]thien-2-yl}sulfonyl)tetrahydro-2H-pyran-4-carboxylic Acid:

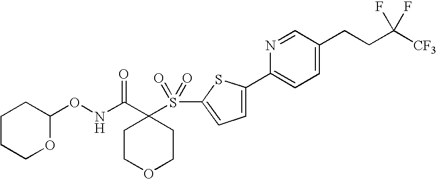

To the carboxylic solid from Part F (0.31 g, MW 499.47) in N,N-dimethylacetamide (3 ml) was added triethylamine (from Aldrich, 0.26 ml, MW 101.19, 3.0 eq), followed by N-hydroxybenzotriazole hydrate (from Aldrich, 0.17 g, MW 135.13, 2.0 eq), O-(tetrahydro-2H-pyran-2-yl) hydroxylamine (0.11 g, MW 117.16, 1.5 eq), and, lastly, 1-(3-dimethylaminopropyl)-3-ethylcarbodiimide hydrochloride (from Sigma, 0.30 g, MW 191.76, 2.5 eq). The resulting mixture was stirred at room temperature for 15 hr. Afterward, the mixture was diluted with water (15 ml) and ethylacetate (100 ml). The organic was separated, and the aqueous was further extracted with ethylacetate (2×75 ml). The organics were combined and then washed with saturated aqueous NaHCO₃ (2×150 ml), washed with water (2×100 ml), washed with brine (1×200 ml), dried over sodium sulfate, and concentrated to afford the desired THP-hydroxamate as a tan foam (0.31 g, 84% crude yield). ¹H NMR and LCMS confirmed the presence of the desired THP-hydroxamate. The “equivalents” above indicate equivalents relative to the charged amount of product from Part F.

Part H. Preparation of N-hydroxy-4-({5-[5-(3,3,4,4,4-pentafluorobutyl)pyridin-2-yl]thien-2-yl}sulfonyl)tetrahydro-2H-pyran-4-carboxamide Hydrochloride:

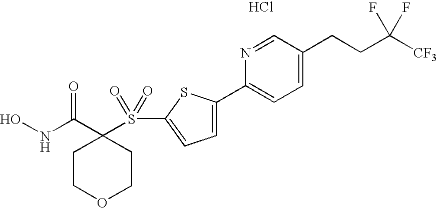

To the THP-hydroxamate product from Part G (0.31 g, MW 598.61) was added methanol (0.5 ml) and 4 N HCl in dioxane (3 ml). The resulting mixture was stirred for 1 hr at room temperature. The solvent was then concentrated to one-third volume, and diethylether was added. The resulting solid was dried to afford the desired hydroxamic acid as a yellow solid (0.27 g, ¹⁰⁰% yield). ¹H NMR confirmed the presence of the desired compound. HRMS for C₁₉H₁₉F₅N₂O₅S₂ showed M^{+H}_{found}=515.0729 (M^{+H}_{calc}=515.0728).

### Example 8

### Preparation of N-hydroxy-4-({5-[5-(trifuoromethyl)pyridin-2-yl]thien-2-yl-sulfonyl}tetrahydro-2H-pyran-4-carboxamide Hydrochloride

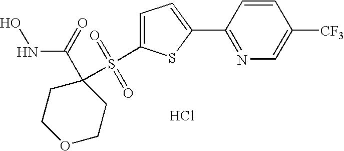

Part A. Preparation of 2-bromo-5-(methylsulfonyl)thiophene:

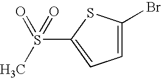

2-Bromo-5-(methylthio)thiophene (10.0 g; MW 209.13; prepared in accordance with Part A, Example 7) was dissolved in THF (100 ml) and water (50 ml). Oxone (from Aldrich, 88.1 g, MW 614, 3.0 eq) was then slowly added portion-wise. The resulting mixture was stirred at room temperature until the reaction was complete. After stirring for 15 hr at room temperature, the mixture was filtered through a pad of Celite. The filtrate was stripped of organics, and the resulting aqueous layer was extracted with ethyl acetate (3×100 ml). The organics were combined and washed with water (3×), washed with brine (1×), dried over sodium sulfate, and concentrated to the desired compound as a light amber oil (4.1 g, 27% yield). ¹H NMR confirmed the presence of desired compound. The “equivalents” above indicate equivalents relative to the charged amount of 2-bromo-5-(methylthio)thiophene.

Part B. Preparation of tert-butyl [(5-bromothien-2-yl)sulfonyl]acetate:

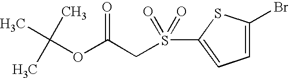

A solution of the product from Part A (12.1 g, MW 241.13) and t-butyl-dicarboxylate (from Aldrich, 2.6 g, MW 218.75, 1.2 eq) in THF (100 ml) was cooled to −78° C. A lithium hexamethyldisylisane solution in THF (1.0 M, 144 ml, 3.0 eq) was slowly added, keeping temperature at less than −65° C. After the addition, the mixture was stirred for 1 hr, and then dripped into a saturated ammonium chloride aqueous solution (50 ml) to quench the reaction. The resulting mixture was warmed to room temperature. Afterward, the organic layer was separated off. The aqueous layer was extracted with ethyl acetate (2×100 ml). The organics were then combined and washed with water and brine, dried over sodium sulfate, and concentrated to form a black solid. The solid was chromatographed on silica gel (ethyl acetate/hexanes) to afford the desired compound as a tan oil (18.6 g, 100+% crude yield). ¹H NMR and LCMS confirmed the presence of desired compound. The “equivalents” above indicate equivalents relative to the charged amount of product from Part A.

Part C. Preparation of tert-butyl 4-[(5-bromothien-2-yl)sulfonyl]tetrahydro-2H-pyran-4-carboxylate:

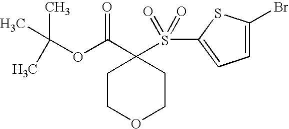

The product from Part B (16.4 g, MW 418.33), potassium carbonate (from Aldrich, 19.5 g, MW 138.21, 3.0 eq), and bis(bromoethyl)ether (from Aldrich, 16.8 g, MW 231.93, 1.5 eq) were slurried in N,N-dimethylformamide (100 ml). The resulting mixture was stirred at 65° C. for 15 hr (the reaction was complete at the end of the 15 hr). Afterward, the mixture was diluted with water (100 ml). The diluted mixture was extracted with ethyl acetate (3×100 ml). The organics were combined and washed with water (2×), washed with brine (1×), dried over Na₂SO₄, and concentrated to form an orangish, oily solid. The solid was washed with hexanes and then chromatographed on silica gel (ethyl acetate/hexanes) to afford the desired compound as a white solid (7.0 g, 36% yield). ¹H NMR and LCMS confirmed the presence of the desired compound. The “equivalents” above indicate equivalents relative to the charged amount of product from Part B.

Part D. Preparation of tert-butyl 4-({5-[5-(trifluoromethyl)pyridin-2-yl]thien-2-yl}sulfonyl)tetrahydro-2H-pyran-4-carboxylate:

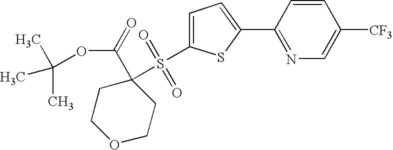

The product from Part C (1.0 g, MW 411.33), bis-pinacol diborane (from Aldrich, 0.80 g, MW 253.95, 1.3 eq), potassium acetate (from Aldrich, 0.95 g, MW 98.14, 4.0 eq), and (1,1′bis-(diphenylphosphino)-ferrocene) palladium dichloride (from Aldrich, 0.06 g, MW 816.64, 0.03 eq) were slurried in N,N-dimethylacetamide (5 ml). The resulting mixture was heated at 80° C. for 2 hr. At the end of the 2 hr period, no bromide was detected by HPLC. Additional (1,1′bis-(diphenylphosphino)-ferrocene) palladium dichloride (from Aldrich, 0.06 g, MW 816.64, 0.03 eq) was added, along with aqueous sodium carbonate (2 M, 3.6 ml, 3.0 eq) and 2-chloro-5-trifluoromethylpyridine (from Lancaster, 0.53 g, MW 181.54, 1.2 eq). Stirring was continued at 80° C. for 2 hr. The reaction was then quenched with water (5 ml). Subsequently, the mixture was filtered through a Celite pad. The filtrate was extracted with ethyl acetate (3×15 ml). The organics were then combined and washed with water (2×30 ml), washed with brine (1×30 ml), dried over sodium sulfate, filtered, and concentrated to form a black residue. The residue was chromatographed on silica gel (ethyl acetate/hexanes) to afford the desired compound as a tan oil (0.34 g, 29% yield). ¹H NMR and LCMS confirmed the presence of the desired compound. The “equivalents” above indicate equivalents relative to the charged amount of product from Part C.

Part E. Preparation of 4-({5-[5-(trifluoromethyl)pyridin-2-yl]thien-2-yl}sulfonyl)tetrahydro-2H-pyran-4-carboxylic Acid:

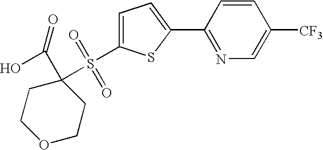

To a solution of the product from Part D (0.30 g, MW 477.52) in dichloromethane (1 ml) was added trifluoroacetic acid (from Aldrich, 3 ml). The resulting mixture was stirred for 4 hr at room temperature. The mixture was then concentrated to one-third volume to form a residue, which, in turn, was dripped into stirring diethylether (10 ml). The resulting solid was collected, washed with diethylether, and dried to afford the desired carboxylic acid as a yellow solid (0.11 g, 42% yield). LCMS confirmed the presence of the desired carboxylic acid.

Part F. Preparation of N-(tetrahydro-2H-pyran-2-yloxy)-4-({5-[5-(trifluoromethyl)pyridin-2-yl]thien-2-yl}sulfonyl)tetrahydro-2H-pyran-4-carboxamide:

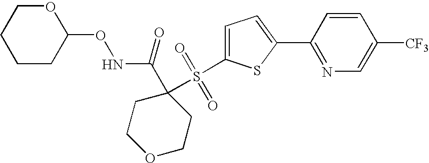

To the carboxylic acid product from Part E (0.11 g, MW 421.41) in N,N-dimethylacetamide (3 ml) was added triethylamine (from Aldrich, 0.07 ml, MW 101.19, 3.0 eq), followed by N-hydroxybenzotriazole hydrate (from Aldrich, 0.05 g, MW 135.13, 2.0 eq), O-(tetrahydro-2H-pyran-2-yl) hydroxylamine (0.04 g, MW 117.16, 1.5 eq), and, lastly, 1-(3-dimethylaminopropyl)-3-ethylcarbodiimide hydrochloride (from Sigma, 0.10 g, MW 191.76, 2.5 eq). The resulting mixture stirred at room temperature for 15 hr. The mixture was then diluted with water (1 ml) and ethylacetate (10 ml). The organic layer was separated, and the aqueous was further extracted with ethylacetate (2×15 ml). The organics were combined and washed with saturated aqueous NaHCO₃ (2×15 ml), washed with water (2×10 ml), washed with brine (1×20 ml), dried over sodium sulfate, and concentrated to afford the desired THP-hydroxamate as a clear oil (0.1 g, 100% crude yield). ¹H NMR and LCMS confirmed the presence of the desired THP-hydroxamate. The “equivalents” above indicate equivalents relative to the charged amount of product from Part E.

Part G. Preparation of N-hydroxy-4-({5-[5-(trifluoromethyl)pyridin-2-yl]thien-2-yl}sulfonyl)tetrahydro-2H-pyran-4-carboxamide Hydrochloride:

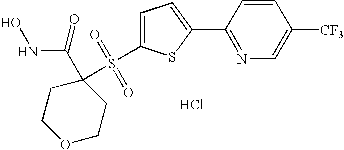

To the THP-hydroxamate product from Part F (0.20 g, MW 520.44) was added methanol (0.5 ml) and 4 N HCl in dioxane (4 ml). The resulting mixture was stirred for 1 hr at room temperature. The solvent was then concentrated to one-third volume, and diethylether was added. The resulting solid was dried to afford the desired hydroxamic acid as a white solid (0.07 g, 39% yield). ¹H NMR confirmed the presence of the desired compound. HRMS for C₁₆H₁₅F₃N₂O₅S₂ showed M^{+H}_{found}=437.0475 (M^{+H}_{calc}=437.0447).

### Example 9

### Preparation of N-hydroxy-4-({2-[4-(trifluoromethoxy)phenyl]-1,3-benzothiazol-6-yl}sulfonyl)tetrahydro-2H-pyran-4-carboxamide

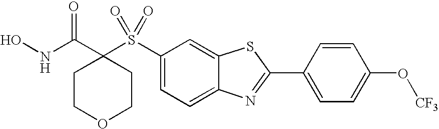

Part A. Preparation of 2-bromo-6-(methylsulfonyl)-1,3-benzothiazole:

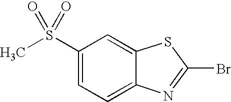

In dry glassware under N₂, a mixture of copper(II)bromide (11.7 g, 52.4 mmol) and tert-butyl nitrite (6.7 g, 65 mmol) was added to acetonitrile (87 mL) cooled to 0° C. To this mixture was added 2-amino-6-(methylsulfonyl)benzothiazole (from Aldrich, 10 g, 43 mmol) in portion, and the ice bath was removed. The reaction mixture was then stirred for an additional 2-3 hr (at the end of this period, the reaction was complete). Afterward, the slurry was slowly poured into water (100 mL). The resulting solid was filtered and washed with 10% aqueous HCl (50 mL) to afford the desired compound as a tan solid (10 g, 78% yield). LCMS m/z=293 [M+H]⁺.

Part B. Preparation of tert-butyl[(2-bromo-1,3-benzothiazol-6-yl)sulfonyl]acetate:

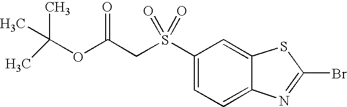

A tetrahydrofuran solution (17 mL) of the methyl sulfone prepared in Part A (5 g, 17 mmol) and di-tert-butyl dicarbonate (4.5 g, 19 mmol) was cooled to −78° C. under N₂. The resulting yellow suspension was treated with 1M lithium bis(trimethylsilyl)amide in tetrahydrofuran (52 mL, 51 mmol) over 15 min. After 1 hr, the resulting homogeneous solution was warmed to 0° C. After an additional 1 hr, the mixture was cooled to −78° C. Subsequently, the reaction was quenched with aqueous, saturated ammonium chloride (50.0 mL). The mixture was then warmed to ambient temperature, and then partitioned with ethyl acetate (100 mL) and water (50 mL). The organic layer was separated, washed with saturated NaHCO₃ (50 mL), washed with 1:1 brine/water (50 mL), washed with brine (2×25 mL), dried over Na₂SO₄, filtered, and concentrated in vacuo to afford the desired ester as a yellow solid (5 g, 75% yield). LC/MS m/z=392 [M+H].

Part C. Preparation of tert-butyl-4-[(2-bromo-1,3-benzothiazol-6-yl)sulfonyl]tetrahydro-2H-pyran-4-carboxylate:

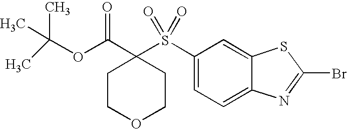

An N,N-dimethylformamide (25.0mL) solution of bis(2-chloroethyl)ether (3.5 g, 19 mmol, from Clariant), potassium carbonate (4.8 g, 57 mmol), and 18-crown-6 ether (0.34 g, 1.29 mmol) being stirred at 60° C. under N₂ was treated with the ester prepared in Part B (5.0 g, 13 mmol). After 23 hr at 60° C., the reaction mixture was diluted with ethyl acetate (30 mL) and partitioned with water (25 mL). The aqueous layer was separated, and extracted with ethyl acetate (2×20 mL). The organics were combined and then washed with saturated NaHCO₃ (20 mL), washed with 1:1 brine/water (20 mL), washed with brine (20 mL), dried over Na₂SO₄, filtered, and concentrated in vacuo. The resulting oil solidified and was purified by tritiation with methanol to afford the desired compound as a solid (6 g, 85% yield). LC/MS m/z=462 [M+H].

Part D. Preparation of tert-butyl-4-({2-[4-(trifluoromethoxy)phenyl]-1,3-benzothiazol-6-yl}sulfonyl)tetrahydro-2H-pyran-4-carboxylate:

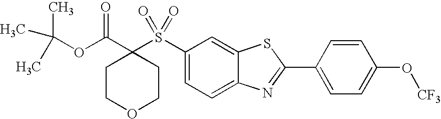

To a solution of the bromo-benzothiazole product from Part C (3.0 g, 6.5 mmol) in dimethoxyethane (13 ml) was added trifluoromethoxybenzene boronic acid (from Aldrich, 3.4 g, 14 mmol) and aqueous sodium carbonate (13 mL). This mixture was stirred at ambient temperature for 20 min while bubbling an N₂ stream below the surface of the solution. [1,1′Bis(diphenylphosphino)ferrocene)dichloropalladium(II) (from Aldrich, 1 g, 1.2 mmol) was then added, and the resulting mixture was stirred at 80° C. until analytical reverse phase high pressure liquid chromatography indicated complete reaction. The mixture was then cooled to ambient temperature and filtered through a Celite pad. The filtrate was concentrated, and the resulting residue was purified on silica gel (ethylacetate/hexanes) to afford the desired compound as a black oil (2.6 g, 75% yield). LC/MS m/z=531 [M+H]. ¹H NMR confirmed the presence of the desired compound.

Part E. Preparation of 4-({2-[4-(trifluoromethoxy)phenyl]-1,3-benzothiazol-6-yl}sulfonyl)tetrahydro-2H-pyran-4-carboxylic Acid:

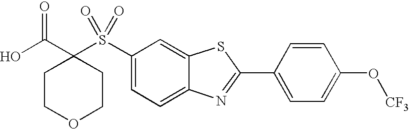

A methylene chloride solution (20 mL) of the product prepared in Part D (2.6 g, 4.9 mmol) was treated with trifluoroacetic acid (5.0 mL, 64.9 mmol) and stirred at ambient temperature. After 14 hr, the reaction mixture was concentrated in vacuo. The concentrated mixture was then treated with diethyl ether (25 mL) and concentrated in vacuo. This exchange was repeated once more. The resulting material was treated with diethyl ether (20 mL), and stirred at ambient temperature for 15 min. Afterward, the solid that separated from solution was filtered to afford the desired carboxylic acid compound as a white solid (2.2 g)

Part F. Preparation of N-(tetrahydro-2H-pyran-2-yloxy)-4-({2-[4-(trifluoromethoxy)phenyl]-1,3-benzothiazol-6-yl}sulfonyl)tetrahydro-2H-pyran-4-carboxamide:

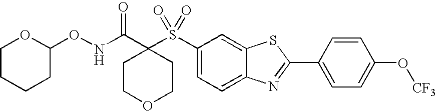

In dry glassware under N₂, the carboxylic acid product from Part C (2.1 g, 3.9 mmol) was dissolved in dry dimethylformamide (30 mL). The following reagents were then added to the solution in the following order: N-hydroxybenzotriazole hydrate (0.55 g, 3.9 mmol), triethylamine (1.2 mL, 12 mmol), O-(tetrahydro-2H-pyran-2-yl)hydroxylamine (0.5,6 mmol), and 1-(3-dimethylaminopropyl)-3-ethylcarbodiimide hydrochloride (1.1 g, 6 mmol). After 12 hr at ambient temperature, the mixture was poured into water. The THP-hydroxamate product was then extracted (using ethyl acetate), washed with water, washed with saturated NaHCO₃, dried over Na₂SO₄, filtered, and concentrated in vacuo. Chromatography (on silica, ethyl acetate/hexanes) provided the THP-hydroxamate as a white foam (1.9 g, 81% yield). LCMS m/z=587 [M+H]⁺.

Part G. Preparation of N-hydroxy-4-({2-[4-(trifluoromethoxy)phenyl]-1,3-benzothiazol-6-yl}sulfonyl)tetrahydro-2H-pyran-4-carboxamide:

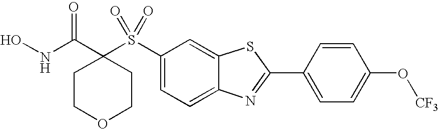

To the THP-hydroxamate product from Part F (1.9 g, 3.2 mmol) was added acetonitrile (20 mL) and aqueous 6 N HCl (4 mL). The solution was stirred for 1 hr at ambient temperature (after this period, the reaction was complete). A stream of N₂ was then placed over the surface of the solution. After 1 hr, enough acetonitrile had evaporated to cause the desired hydroxamic acid product to separate from the solution. This solid was filtered and dried to afford the hydroxamic acid product as a white solid (0.55 mg, 40% yield). HRMS (ES+) M+H⁺ calculated for C₂₀H₁₇N₂O₆S₂F₃: 503.2, found 503.1.

### Example 10

### Preparation of 4-[(2-{4-[(5-butylthien-2-yl)carbonyl]piperidin-1-yl}-1,3-benzothiazol-6-yl)sulfonyl]-N-hydroxytetrahydro-2H-pyran-4-carboxamide Hydrochloride

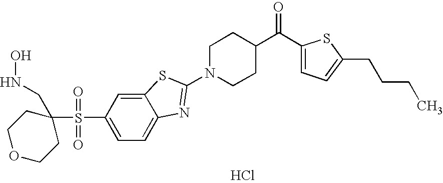

Part A. Preparation of tert-butyl 4-[(2-{4-[(5-butylthien-2-yl)carbonyl]piperidin-1-yl}-1,3-benzothiazol-6-yl)sulfonyl]tetrahydro-2H-pyran-4-carboxylate:

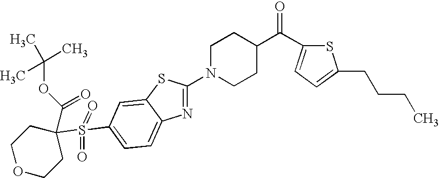

Tert-butyl-4-[(2-bromo-1,3-benzothiazol-6-yl)sulfonyl]tetrahydro-2H-pyran-4-carboxylate (0.70 g; MW 462.38; prepared in accordance with Part C, Example 9), (5-butyl-thiophen-2-yl)-piperidin-4-yl-methanone hydrochloride (0.52 g, MW 287.85, 1.2 eq), and potassium carbonate (from Aldrich, 0.63 g, MW 138.25, 3.0 eq) were slurried in N,N-dimethylformamide (5 ml). The resulting mixture was heated at 80° C. for 16 hr. The reaction was then quenched with water (5 ml). Afterward, the mixture was extracted with ethyl acetate (3×15 ml). The organics were combined and then washed with 1% aqueous HCl (1×20 ml), washed with water (2×30 ml), washed with brine (1×30 ml), dried over sodium sulfate, filtered, and concentrated to form a tan oil. The residue was chromatographed on silica gel (ethyl acetate/hexanes) to afford the desired compound as a tan oil (0.45 g, 47% yield). ¹H NMR and LCMS confirmed the presence of the desired compound. The “equivalents” above indicate equivalents relative to the charged amount of tert-butyl-carboxylate.

Part B. Preparation of 4-[(2-{4-[(5-butylthien-2-yl)carbonyl]piperidin-1-yl}-1,3-benzothiazol-6-yl)sulfonyl]tetrahydro-2H-pyran-4-carboxylic Acid:

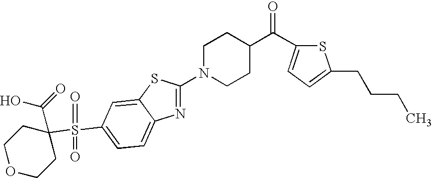

To a solution of the product from Part A (0.45 g, MW 632.85) in dichloromethane (4 ml) was added trifluoroacetic acid (from Aldrich, 6 ml). The mixture was then stirred for 4 hr at room temperature, and then concentrated to one-third volume to form a residue, which, in turn, was dripped into stirring diethyl ether (10 ml). The resulting solid was collected, washed with diethyl ether, and dried to afford the desired carboxylic acid compound as a tan solid (0.31 g, 63% yield). LCMS confirmed the presence of the desired compound.

Part C. Preparation of 4-[(2-{4-[(5-butylthien-2-yl)carbonyl]piperidin-1-yl}-1,3-benzothiazol-6-yl)sulfonyl]-N-(tetrahydro-2H-pyran-2-yloxy)tetrahydro-2H-pyran-4-carboxamide:

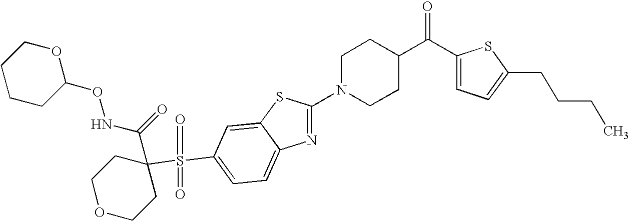

To the carboxylic acid product of Part B (0.31 g, MW 576.76) in N,N-dimethylacetamide (3 ml) was added triethylamine (from Aldrich, 0.15 ml, MW 101.19, 3.0 eq), followed by N-hydroxybenzotriazole hydrate (from Aldrich, 0.14 g, MW 135.13, 2.0 eq), O-(tetrahydro-2H-pyran-2-yl) hydroxylamine (0.10 g, MW 117.16, 1.5 eq), and, lastly, 1-(3-dimethylaminopropyl)-3-ethylcarbodiimide hydrochloride (from Sigma, 0.26 g, MW 191.76, 2.5 eq). The resulting mixture was stirred at room temperature for 15 hr. Subsequently, the mixture was diluted with water (1 ml) and ethyl acetate (10 ml). The organic layer was then separated, and the aqueous was further extracted with ethyl acetate (2×15 ml). The organics were combined and washed with saturated aqueous NaHCO₃ (2×15 ml), washed with water (2×10 ml), washed with brine (1×20 ml), dried over sodium sulfate, and concentrated to afford the desired THP-hydroxamate as an off-white solid (0.35 g, 97% crude yield). ¹H NMR and LCMS confirmed the presence of the desired THP-hydroxamate. The “equivalents” above indicate equivalents relative to the charged amount of product from Part B.

Part D. Preparation of 4-[(2-{4-[(5-butylthien-2-yl)carbonyl]piperidin-1-yl}-1,3-benzothiazol-6-yl)sulfonyl]-N-hydroxytetrahydro-2H-pyran-4-carboxamide hydrochloride:

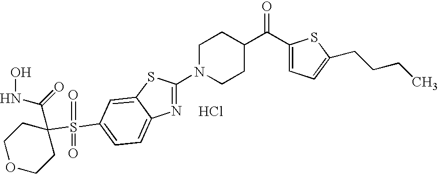

To the THP-hydroxamate product from Part C (0.35 g, MW 675.88) was added methanol (0.5 ml) and 4 N HCl in dioxane (6 ml). The resulting mixture was stirred for 1 hr at room temperature. The solvent was then concentrated to one-third volume, and diethyl ether was added. The resulting solid was dried to afford the desired hydroxamic acid as a white solid (0.26 g, 81% yield). ¹H NMR confirmed the presence of the desired hydroxamic acid. HRMS for C₂₇H₃₃N₃O₆S₃ showed M^{+H}_{found}=592.1618 (M^{+H}_{calc}=592.1604).

### Example 11

### Preparation of N-hydroxy-4-{[2-(4-propylphenyl)-1,3-benzothiazol-6-yl]sulfonyl}tetrahydro-2H-pyran-4-carboxamide

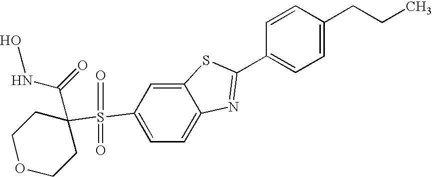

Part A. Preparation of tert-butyl 4-{[2-(4-propylphenyl)-1,3-benzothiazol-6-yl]sulfonyl}tetrahydro-2H-pyran-4-carboxylate:

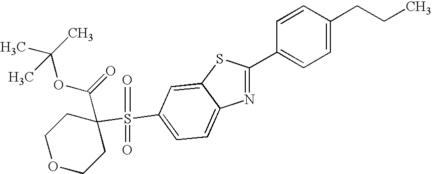

Tert-butyl-4-[(2-bromo-1,3-benzothiazol-6-yl)sulfonyl]tetrahydro-2H-pyran-4-carboxylate (1.5 g; MW 465.63; prepared in accordance with Part C, Example 9), n-propylphenyl boranic acid (from Aldrich, 0.58 g, MW 164.01, 1.1 eq), tetrakis(triphenylphosphine)palladium (from Strem Chemical, 185 mg, MW 1155.58, 0.05 eq), and 2 M sodium carbonate (aqueous, 2.1 ml, 1.3 eq) were slurried in ethylene glycol dimethylether (12 ml) and heated at 55° C. for 3 hr. Reaction mixture was cooled to room temperature then filtered through a Celite plug. The filtrate was diluted with water (20 ml). The diluted mixture was extracted with ethyl acetate (3×25 ml). The organics were combined and washed with water (2×30 ml) and brine (1×30 ml) then dried over sodium sulfate, filtered, and concentrated for a black oil. The residue was chromatographed on silica gel (ethyl acetate/hexanes) to afford the desired ester as an orange solid (0.61 g, 38% yield). ¹H NMR and LCMS confirmed the presence of the desired ester. The “equivalents” above indicate equivalents relative to the charged amount of tert-butyl-carboxylate.

Part B. Preparation of 4-{[2-(4-propylphenyl)-1,3-benzothiazol-6-yl]sulfonyl}tetrahydro-2H-pyran-4-carboxylic Acid:

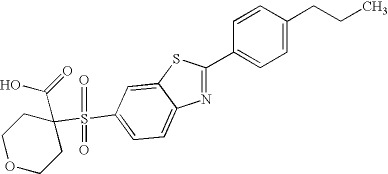

To a solution of the ester product from Part A (0.6 g, MW 501.66) in dichloromethane (4 ml) was added trifluoroacetic acid (from Aldrich, 6 ml). The resulting mixture was stirred for 4 hr at room temperature, and then concentrated to one-third volume to form a residue, which, in turn, was dripped into stirring diethyl ether (10 ml). The resulting solid was collected, washed with diethyl ether, and dried to afford the desired carboxylic acid as a brown solid (0.6 g, 100+% crude yield). LCMS confirmed the presence of the desired carboxylic acid.

Part C. Preparation of 4-{[2-(4-propylphenyl)-1,3-benzothiazol-6-yl]sulfonyl}-N-(tetrahydro-2H-pyran-2-yloxy)tetrahydro-2H-pyran-4-carboxamide:

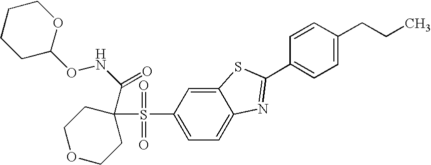

To the carboxylic acid product from Part B (0.60 g, MW 445.55) in N,N-dimethylacetamide (3 ml) was added triethylamine (from Aldrich, 0.28 ml, MW 101.19, 3.0 eq), followed by N-hydroxybenzotriazole hydrate (from Aldrich, 0.36 g, MW 135.13, 2.0 eq), O-(tetrahydro-2H-pyran-2-yl) hydroxylamine (0.23 g, MW 117.16, 1.5 eq), and, lastly, 1-(3-dimethylaminopropyl)-3-ethylcarbodiimide hydrochloride (from Sigma, 0.66 g, MW 191.76, 2.5 eq). The resulting mixture was stirred at room temperature for 15 hr. Afterward, the mixture was diluted with water (1 ml) and ethyl acetate (10 ml). The organic layer was separated, and the aqueous was further extracted with ethyl acetate (2×1 5 ml). The organics were then combined and washed with saturated aqueous NaHCO₃ (2×15 ml), washed with water (2×10 ml), washed with brine (1×20 ml), dried over sodium sulfate, and concentrated to form a crude product in the form of a beige solid. The solid was chromatographed (RP-Carbon 18, acetonitrile/water) to afford the desired THP-hydroxamate as a colorless oil (0.14 g, 19% yield). ¹H NMR and LCMS confirmed the presence of the desired THP-hydroxamate. The “equivalents” above indicate equivalents relative to the charged amount of product from Part B.

Part D. Preparation of N-hydroxy-4-{[2-(4-propylphenyl)-1,3-benzothiazol-6-yl]sulfonyl}tetrahydro-2H-pyran-4-carboxamide:

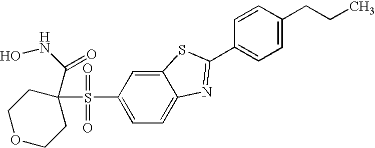

To the THP-hydroxamate product from Part C (0.14 g, MW 508.65) was added methanol (0.5 ml) and 4 N HCl in dioxane (5 ml). The resulting mixture was stirred for 1 hr at room temperature. The solvent was then concentrated to one-third volume, and diethyl ether was added. The resulting solid was dried to afford the desired hydroxamic acid as a white solid (0.09 g, 75% yield). ¹H NMR confirmed the presence of the desired hydroxamic acid. HRMS for C₂₂H₂₄N₂O₅S₂ showed M^{+H}_{found}=461.5698 (M^{+H}_{calc}=461.5684).

### Example 12

### Preparation of N-hydroxy-4-{[2-(2-isobutyl-1,3-thiazol-5-yl)-1,3-benzothiazol-6-yl]sulfonyl}tetrahydro-2H-pyran-4-carboxamide

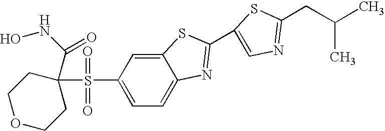

Part A. Preparation of tert-butyl 4-{[2-(4-ethoxyphenyl)-1,3-benzothiazol-6-yl]sulfonyl}tetrahydro-2H-pyran-4-carboxylate:

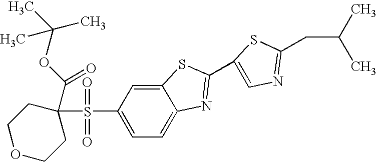

A solution of 2-isobutylthiazole (from Aldrich; 0.72 g; MW 141.25; 1.3 eq) in tetrahydrofuran (15 ml) was cooled to −78° C. Next, a solution of t-butyllithium (from Aldrich; 1.7M in pentane; 5.06 ml; 2.7 eq) was slowly added. The mixture was then stirred for 30 min at −78° C. Afterward, a solution of zinc(II)chloride (from Aldrich; 1.0 M in diethyl ether; 6.4 ml; 2.0 eq) was slowly added. The mixture was then warmed to ambient temperature and stirred for 30 min. Lastly, a solution of tert-butyl-4-[(2-bromo-1,3-benzothiazol-6-yl)sulfonyl]tetrahydro-2H-pyran-4-carboxylate (1.5 g; MW 462.38; prepared in accordance with Part C, Example 9) and bis(triphenylphosphine)dichloropalladium (from Aldrich, 0.11 g, MW 701.89, 0.05 eq added) in tetrahydrofuran (20 ml) was added. The resulting mixture was heated at reflux for 16 hr. The reaction was then quenched with a saturated ammonium chloride solution (20 ml). The aqueous layer was separated and extracted with ethyl acetate (2×25 ml). The resulting organics were combined, washed with brine (2×50 ml), dried over sodium sulfate, and concentrated to form a dark oil. The oil was chromatographed on silica get (ethyl acetate/hexanes) to afford the desired compound as a tan solid (0.55 g, 33% yield). ¹H NMR and LCMS confirmed the presence of the desired compound. The “equivalents” above indicate equivalents relative to the charged amount of tert-butyl-carboxylate.

Part B. Preparation of 4-{[2-(2-isobutyl-1,3-thiazol-5-yl)-1,3-benzothiazol-6-yl]sulfonyl}tetrahydro-2H-pyran-4-carboxylic acid:

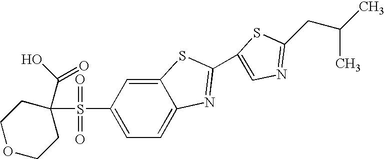

To a solution of the product from Part A (0.55 g, MW 522.70) in dichloromethane (2 ml) was added trifluoroacetic acid (from Aldrich, 4 ml). The resulting mixture stirred for 4 hr at room temperature, and concentrated to one-third volume to form a residue, which, in turn, was dripped into stirring diethyl ether (10 ml). The resulting solid was collected, washed with diethyl ether, and dried to afford the desired carboxylic acid as a yellow solid (0.39 g, 80% crude yield). LCMS confirmed the presence of the desired compound.

Part C. Preparation of 4-{[2-(2-isobutyl-1,3-thiazol-5-yl)-1,3-benzothiazol-6-yl]sulfonyl}-N-(tetrahydro-2H-pyran-2-yloxy)tetrahydro-2H-pyran-4-carboxamide:

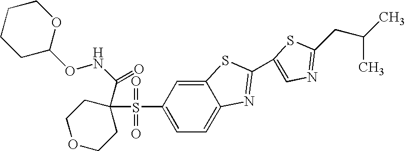

To the carboxylic acid product from Part B (0.55 g, MW 466.60) in N,N-dimethylacetamide (5 ml) was added triethylamine (from Aldrich, 0.17 ml, MW 101.19, 3.0 eq), followed by N-hydroxybenzotriazole hydrate (from Aldrich, 0.22 g, MW 135.13, 2.0 eq), O-(tetrahydro-2H-pyran-2-yl) hydroxylamine (0.14 g, MW 117.16, 1.5 eq), and, lastly, 1-(3-dimethylaminopropyl)-3-ethylcarbodiimide hydrochloride (from Sigma, 0.40 g, MW 191.76, 2.5 eq). The resulting mixture was stirred at room temperature for 15 hr. Afterward, the mixture was dilute with water (1 ml) and ethyl acetate (10 ml). The organic layer was separated, and the aqueous was further extracted with ethyl acetate (2×15 ml). The organics were combined and then washed with saturated aqueous NaHCO₃ (2×15 ml), washed with water (2×10 ml), washed with brine (1×20 ml), dried over sodium sulfate, and concentrated to form a crude product in the form of a beige solid. The solid was tritiated with diethyl ether, and then dried to afford the desired THP-hydroxamate as a tan oil (0.38 g, 83% yield). ¹H NMR and LCMS confirmed the presence of the desired THP-hydroxamate. The “equivalents” above indicate equivalents relative to the charged amount of product from Part B.

Part D. Preparation of N-hydroxy-4-{1[2-(2-isobutyl-1,3-thiazol-5-yl)-1,3-benzothiazol-6-yl]sulfonyl}tetrahydro-2H-pyran-4-carboxamide:

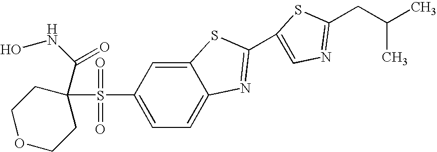

To the THP-hydroxamate product from Part C (0.38 g, MW 565.73) was added methanol (0.5 ml) and 4 N HCl in dioxane (5 ml). The resulting mixture was stirred for 1 hr at room temperature. The solvent was then concentrated to one-third volume, and diethyl ether was added. The resulting solid was dried to afford the desired hydroxamic acid as a pale yellow solid (0.19 g, 68% yield). ¹H NMR confirmed the presence of the desired hydroxamic acid. HRMS for C₂₀H₂₃N₃O₅S₃ showed M^{+H}_{found}=482.6206 (M^{+H}_{calc}=482.6198).

### Example 13

### Preparation of N-hydroxy-4-({2-[3-(trifluoromethyl)phenyl]-1,3-benzothiazol-6-yl}sulfonyl)tetrahydro-2H-pyran-4-carboxamide

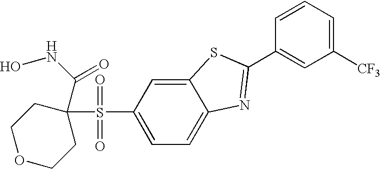

Part A. Preparation of tert-butyl 4-({2-[3-(trifluoromethyl)phenyl]-1,3-benzothiazol-6-yl}sulfonyl)tetrahydro-2H-pyran-4-carboxylate:

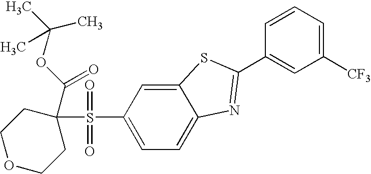

Tert-butyl-4-[(2-bromo-1,3-benzothiazol-6-yl)sulfonyl]tetrahydro-2H-pyran-4-carboxylate (2.0 g; MW 465.63; prepared in accordance with Part C, Example 9), 3-trifluoromethylphenyl boranic acid (from Aldrich, 0.90 g, MW 184.93, 1.1 eq), (1,1′bis-(diphenylphosphino)-ferrocene) palladium dichloride (from Aldrich, 0.18 g, MW 816.64, 0.05 eq), and 2 M sodium carbonate (aqueous, 6.5 ml, 1.3 eq) were slurried in ethylene glycol dimethylether (10 ml). The resulting mixture was heated at 55° C. for 3 hr. Afterward, the mixture was cooled to room temperature and filtered through a Celite plug. The filtrate was diluted with water (20 ml). The diluted mixture was extracted with ethyl acetate (3×25 ml). The organics were combined and then washed with water (2×30 ml), washed with brine (1×30 ml), dried over sodium sulfate, filtered, and concentrated to form a black oily solid. Recrystallization from methanol afforded the desired ester as a tan solid (1.3 g, 56% yield). ¹H NMR and LCMS confirmed the presence of the desired ester. The “equivalents” above indicate equivalents relative to the tert-butyl-carboxylate.

Part B. Preparation of 4-({2-[3-(trifluoromethyl)phenyl]-1,3-benzothiazol-6-yl}sulfonyl)tetrahydro-2H-pyran-4-carboxylic acid:

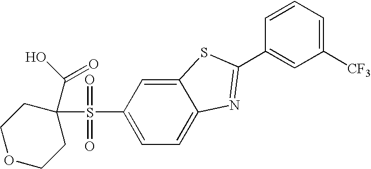

To a solution of the ester product from Part A (1.3 g, MW 527.59) in dichloromethane (4 ml) was added trifluoroacetic acid (from Aldrich, 8 ml). This mixture was stirred for 4 hr at room temperature, and then concentrated to one-third volume to form a residue, which, in turn, was dripped into stirring diethyl ether (10 ml). The resulting solid was collected, washed with diethyl ether, and dried to afford the desired carboxylic acid as a brown solid (0.95 g, 82% crude yield). LCMS confirmed the presence of the desired compound.

Part C. Preparation of N-(tetrahydro-2H-pyran-2-yloxy)-4-({2-[3-(trifluoromethyl)phenyl]-1,3-benzothiazol-6-yl}sulfonyl)tetrahydro-2H-pyran-4-carboxamide:

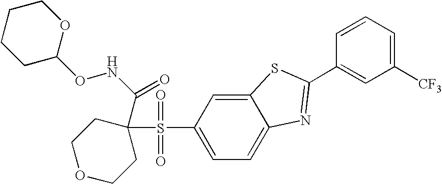

To the carboxylic acid product of Part B (0.98 g, MW 471.48) in N,N-dimethylacetamide (5 ml) was added triethylamine (from Aldrich, 0.40 ml, MW 101.19, 3.0 eq), followed by N-hydroxybenzotriazole hydrate (from Aldrich, 0.51 g, MW 135.13, 2.0 eq), O-(tetrahydro-2H-pyran-2-yl) hydroxylamine (0.34 g, MW 117.16, 1.5 eq), and, lastly, 1-(3-dimethylaminopropyl)-3-ethylcarbodiimide hydrochloride (from Sigma, 0.93 g, MW 191.76, 2.5 eq). The resulting mixture was stirred at room temperature for 15 hr. Afterward, the mixture was diluted with water (1 ml) and ethyl acetate (10 ml). The organic layer was separated, and the aqueous layer was further extracted with ethyl acetate (2×15 ml). The organics were combined and then washed with saturated aqueous NaHCO₃ (2×15 ml), washed with water (2×10 ml), washed with brine (1×20 ml), dried over sodium sulfate, and concentrated to form a crude product in the form of a beige solid. The resulting solid was chromatographed (RP-Carbon 18, acetonitrile/water) to afford the desired THP-hydroxamate as a colorless oil (0.50 g, 46% yield). ¹H NMR and LCMS confirmed the presence of the desired THP-hydroxamate. The “equivalents” above indicate equivalents relative to the charged amount of product from Part B.

Part D. Preparation of N-hydroxy-4-({2-[3-(trifluoromethyl)phenyl]-1,3-benzothiazol-6-yl}sulfonyl)tetrahydro-2H-pyran-4-carboxamide:

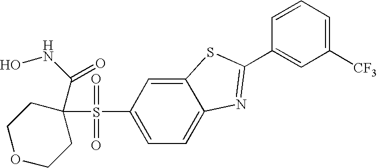

To the THP-hydroxamate product from Part C (0.50 g, MW 570.61) was added methanol (0.5 ml) and 4 N HCl in dioxane (5 ml). The resulting mixture was stirred for 1 hr at room temperature. The solvent was then concentrated to one-third volume, and diethyl ether was added. The resulting solid was dried to afford the desired hydroxamic acid as a pink solid (0.42 g, 98% yield). ¹H NMR confirmed the presence of the desired hydroxamic acid. HRMS for C₂₀H₁₇F₃N₂O₅S₂ showed M^{+H}_{found}=487.0628 (M^{+H}_{calc}=487.0604).

### Example 14

### Preparation of N-hydroxy-4-[(2-{4-[4(trifluoromethoxy)phenoxy]piperidin-1-yl}-1,3-benzothiazol-6-yl)sulfonyl]tetrahydro-2H-pyran-4-carboxamide

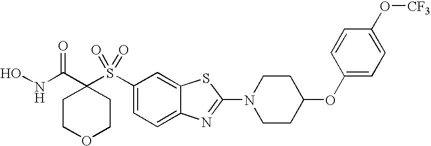

Part A. Preparation of tert-butyl 4-[(2-{4-[4-(trifluoromethoxy)phenoxy]piperidin-1-yl}-1,3-benzothiazol-6-yl)sulfonyl]tetrahydro-2H-pyran-4-carboxylate:

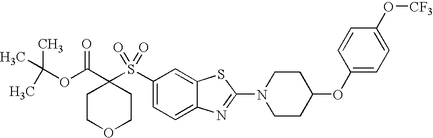

To a solution of tert-butyl-4-[(2-bromo-1,3-benzothiazol-6-yl)sulfonyl]tetrahydro-2H-pyran-4-carboxylate (3.0 g, 6.5 mmol, prepared as in Part C, Example 9) in dioxane (20 ml) was added 4-[4-(trifluoromethoxy)phenoxy]piperidine (2.1 g, 7 mmol) and potassium carbonate (2 g, 15 mmol). The resulting mixture was stirred at 80° C. until analytical reverse phase high pressure liquid chromatography indicated complete reaction. The mixture was then cooled to ambient temperature. After the mixture was concentrated using a rotary evaporator, water (100 ml) added. The mixture was then filtered, and the resulting residue was air dried to afford the desired ester as a white solid (3.5 g, 84% yield). LC/MS m/z=643 [M+H]. ¹H NMR confirmed the presence of the desired ester.

Part B. Preparation of 4-{2-[4-(4-trifluoromethoxy-phenoxy)-piperidin-1-yl]-benzothiazole-6-sulfonyl}-tetrahydro-pyran-4-carboxylic Acid:

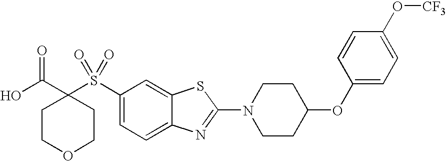

A methylene chloride solution (20 mL) of the ester product from Part A (3.5 g, 5.5 mmol) was treated with trifluoroacetic acid (5.0 mL, 64.9 mmol). This solution was stirred at ambient temperature for 14 hr. Afterward, the mixture was concentrated in vacuo. The concentrated mixture was treated with diethyl ether (25 mL), and then concentrated in vacuo. This exchange was repeated once more. The material was then treated with diethyl ether (20 mL). This mixture was stirred at ambient temperature for 15 min, and the solid that separated from solution was filtered to afford the desired carboxylic acid as a white solid (2.9 g)

Part C. Preparation of 4-{2-[4-(4-trifluoromethoxy-phenoxy)-piperidin-1-yl]-benzothiazole-6-sulfonyl}-tetrahydro-pyran-4-carboxylic Acid (tetrahydro-pyran-2-yloxy)-amide:

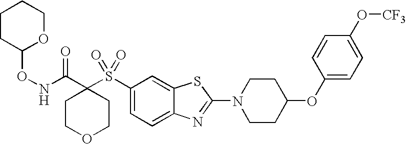

In dry glassware under N₂, the carboxylic acid product from Part B (2.8 g, 4.8 mmol) was dissolved in dry dimethylacetamide (25 mL). The following additional were then added to the solution in the following order: N-hydroxybenzotriazole hydrate (0.65 g, 4.8 mmol), triethylamine (1.2 mL, 12 mmol), O-(tetrahydro-2H-pyran-2-yl)hydroxylamine (0.5,6 mmol), and 1-(3-dimethylaminopropyl)-3-ethylcarbodiimide hydrochloride (1.1 g, 6 mmol). After 12 hr at ambient temperature, the mixture was poured into water. The THP-hydroxamate was then extracted using ethyl acetate, washed with water, washed with saturated NaHCO₃, dried over Na₂SO₄, filtered, and concentrated in vacuo. Chromatography (on silica, ethyl acetate/hexanes) afforded the THP-hydroxamate as a white foam (2.8 g, 85% yield). LCMS m/z=686 [M+H]⁺.

Part D. Preparation of 4-{2-[4-(4-trifluoromethoxy-phenoxy)-piperidin-1-yl]-benzothiazole-6-sulfonyl}-tetrahydro-pyran-4-carboxylic Acid Hydroxyamide:

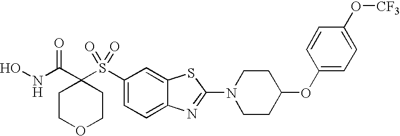

To the THP-hydroxamate product from Part C (2.8 g, 4 mmol) was added acetonitrile (20 mL) and aqueous 6N HCl (4 mL). The solution was stirred for 1 hr at ambient temperature. After the reaction was complete, a stream of N₂ was placed over the surface of the solution. Over the next hour, enough acetonitrile evaporated to cause the hydroxamic acid to separate from solution. This solid was filtered, dried, and purified on reverse-phase chromatography (C18) to afford the desired hydroxamic acid as an off-white solid (1 g, 40% yield). HRMS (ES+) M+H⁺ calculated for C₂₅H₂₅N₃O₇S₂F₃: 602, found 602.

### Example 15

### Preparation of N-hydroxy-4-({2-[4-(trifluoromethyl)phenyl]-1,3-benzothiazol-6-yl}sulfonyl)tetrahydro-2H-pyran-4-carboxamide

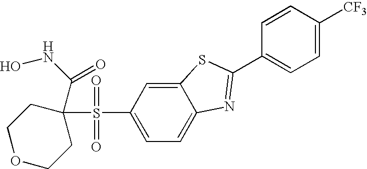

Part A. Preparation of tert-butyl 4-({2-[4-(trifluoromethyl)phenyl]-1,3-benzothiazol-6-yl}sulfonyl)tetrahydro-2H-pyran-4-carboxylate:

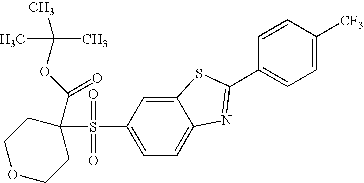

Tert-butyl-4-[(2-bromo-1,3-benzothiazol-6-yl)sulfonyl]tetrahydro-2H-pyran-4-carboxylate (1.0 g, MW 465.63, prepared in accordance with Part C, Example 9), 4-trifluoromethylphenyl boranic acid (from Aldrich, 0.49 g, MW 184.93, 1.2 eq), (1,1′bis-(diphenylphosphino)-ferrocene) palladium dichloride (from Aldrich, 0.09 g, MW 816.64, 0.05 eq), and 2 M sodium carbonate (aqueous, 3.3 ml, 3.0 eq) were slurried in ethylene glycol dimethylether (15 ml). The resulting mixture was heated at 55° C. for 3 hr. Afterward, the mixture was cooled to room temperature. The cooled mixture was filtered through a Celite plug. The filtrate was diluted with water (20 ml). The diluted mixture was extracted with ethyl acetate (3×25 ml). The organics were combined and then washed with water (2×30 ml), washed with brine (1×30 ml), dried over sodium sulfate, filtered, and concentrated to form a black oily solid. Recrystallization from methanol afforded the desired ester as a tan solid (1.0 g, 86% yield). ¹H NMR and LCMS confirmed the presence of the desired ester. The “equivalents” above indicate equivalents relative to the charged amount of tert-butyl-carboxylate.

Part B. Preparation of 4-({2-[4-(trifluoromethyl)phenyl]-1,3-benzothiazol-6-yl}sulfonyl)tetrahydro-2H-pyran-4-carboxylic Acid:

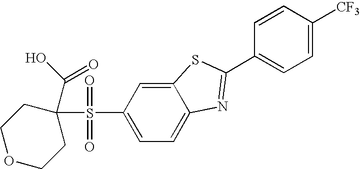

To a solution of the ester product from Part A (1.3 g, MW 527.59) in dichloromethane (4 ml) was added trifluoroacetic acid (from Aldrich, 8 ml). The resulting mixture was stirred for 4 hr at room temperature. The mixture was then concentrated to one-third volume to form a residue, which, in turn, was dripped into stirring diethyl ether (10 ml). The resulting solid was collected, washed with diethyl ether, and dried to afford the desired carboxylic acid as a brown solid (0.95 g, 82% crude yield). LCMS confirmed the presence of the desired carboxylic acid.

Part C. Preparation of N-(tetrahydro-2H-pyran-2-yloxy)-4-({2-[4-(trifluoromethyl)phenyl]-1,3-benzothiazol-6-yl}sulfonyl)tetrahydro-2H-pyran-4-carboxamide:

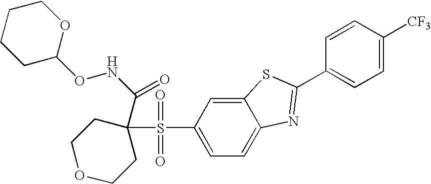

To the carboxylic acid product from Part B (0.40 g, MW 471.48) in N,N-dimethylacetamide (5 ml) was added triethylamine (from Aldrich, 0.24 ml, MW 101.19, 3.0 eq), followed by N-hydroxybenzotriazole hydrate (from Aldrich, 0.23 g, MW 135.13, 2.0 eq), O-(tetrahydro-2H-pyran-2-yl) hydroxylamine (0.15 g, MW 117.16, 1.5 eq), and, lastly, 1-(3-dimethylaminopropyl)-3-ethylcarbodiimide hydrochloride (from Sigma, 0.42 g, MW 191.76, 2.5 eq). The resulting mixture was stirred at room temperature for 15 hr. Afterward, the mixture was diluted with water (1 ml) and ethyl acetate (10 ml). The organic layer was separated, and the aqueous was further extracted with ethyl acetate (2×15 ml). The organics were combined and then washed with saturated aqueous NaHCO₃ (2×15 ml), washed with water (2×10 ml), washed with brine (1×20 ml), dried over sodium sulfate, and concentrated to form a crude product in the form of a beige solid. The solid was chromatographed (RP-Carbon 18, acetonitrile/water) to afford the desired THP-hydroxamate as a colorless oil (0.45 g, 94% yield). ¹H NMR and LCMS confirmed the presence of the desired THP-hydroxamate. The “equivalents” above indicate equivalents relative to the charged amount of product from Part B.

Part D. Preparation of N-hydroxy-4-({2-[4-(trifluoromethyl)phenyl]-1,3-benzothiazol-6-yl}sulfonyl)tetrahydro-2H-pyran-4-carboxamide:

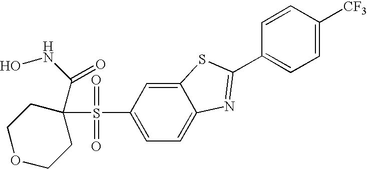

To the THP-hydroxamate product from Part C (0.45 g, MW 570.61) was added methanol (0.5 ml) and 4 N HCl in dioxane (5 ml). The resulting mixture was stirred for 1 hr at room temperature. The solvent was then concentrated to one-third volume, and diethyl ether was added. The resulting solid was dried to afford the desired hydroxamic acid as a white solid (0.35 g, 92% yield). ¹H NMR confirmed the presence of the desired hydroxamic acid. HRMS for C₂₀H₁₇F₃N₂O₅S₂ showed M^{+H}_{found}=487.0628 (M^{+H}_{calc}=487.0604).

### Example 16

### Preparation of 4-{[2-(4-ethylphenyl)-1,3-benzothiazol-6-yl]sulfonyl}-N-hydroxytetrahydro-2H-pyran-4-carboxamide

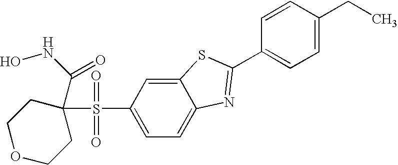

Part A. Preparation of tert-butyl 4-{[2-(4-ethylphenyl)-1,3-benzothiazol-6-yl]sulfonyl}tetrahydro-2H-pyran-4-carboxylate:

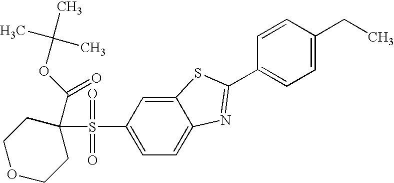

Tert-butyl-4-[(2-bromo-1,3-benzothiazol-6-yl)sulfonyl]tetrahydro-2H-pyran-4-carboxylate (1.0 g, MW 465.63, prepared in accordance with Part C, Example 9), 4-trifluoromethylphenyl boranic acid (from Aldrich, 0.39 g, MW 149.99, 1.2 eq), (1,1′bis-(diphenylphosphino)-ferrocene) palladium dichloride (from Aldrich, 0.09 g, MW 816.64, 0.05 eq), and 2 M sodium carbonate (aqueous, 3.3 ml, 3.0 eq) were slurried in ethylene glycol dimethylether (15 ml). The resulting mixture was heated at 55° C. for 3 hr. Subsequently, the mixture was cooled to room temperature. The cooled mixture was filtered through a Celite plug. The filtrate was diluted with water (20 ml). The diluted mixture was extracted with ethyl acetate (3×25 ml). The organics were combined and then washed with water (2×30 ml), washed with brine (1×30 ml), dried over sodium sulfate, filtered, and concentrated to form a black, oily solid. Recrystallization from methanol afforded the desired ester as a tan solid (0.5 g, 47% yield). ¹H NMR and LCMS confirmed the presence of the desired ester. The “equivalents” above indicate equivalents relative to the charged amount of tert-butyl-carboxylate.

Part B. Preparation of 4-{[2-(4-ethylphenyl)-1,3-benzothiazol-6-yl]sulfonyl}tetrahydro-2H-pyran-4-carboxylic Acid:

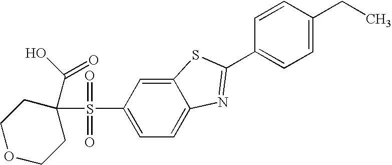

To a solution of the ester product from Part A (1.3 g, MW 487.64) in dichloromethane (4 ml) was added trifluoroacetic acid (from Aldrich, 8 ml). This mixture was stirred for 4 hr at room temperature. Afterward, the mixture was concentrated to one-third volume to form a residue, which, in turn, was dripped into stirring diethyl ether (10 ml). The resulting solid was collected, washed with diethyl ether, and dried to afford the desired carboxylic acid as a brown solid (0.39 g, 91% crude yield). LCMS confirmed the presence of the desired carboxylic acid.

Part C. Preparation of N-(tetrahydro-2H-pyran-2-yloxy)-4-({2-[4-(trifluoromethyl)phenyl]-1,3-benzothiazol-6-yl}sulfonyl)tetrahydro-2H-pyran-4-carboxamide:

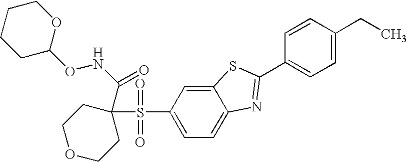

To the carboxylic acid product from Part B (0.39 g, MW 431.53) in N,N-dimethylacetamide (5 ml) was added triethylamine (from Aldrich, 0.25 ml, MW 101.19, 3.0 eq), followed by N-hydroxybenzotriazole hydrate (from Aldrich, 0.24 g, MW 135.13, 2.0 eq), O-(tetrahydro-2H-pyran-2-yl) hydroxylamine (0.15 g, MW 117.16, 1.5 eq), and, lastly, 1-(3-dimethylaminopropyl)-3-ethylcarbodiimide hydrochloride (from Sigma, 0.43 g, MW 191.76, 2.5 eq). The resulting mixture was stirred at room temperature for 15 hr. Afterward, the mixture was diluted with water (1 ml) and ethyl acetate (10 ml). The organic layer was separated, and the aqueous was further extracted with ethyl acetate (2×15 ml). The organics were combined and then washed with saturated aqueous NaHCO₃ (2×15 ml), washed with water (2×10 ml), washed with brine (1×20 ml), dried over sodium sulfate, and concentrated to form a crude product in the form of a beige solid. The solid was chromatographed (RP-Carbon 18, acetonitrile/water) to afford the desired THP-hydroxamate as a colorless oil (0.47 g, 98% yield). ¹H NMR and LCMS confirmed, the presence of the desired THP-hydroxamate. The “equivalents” above indicate equivalents relative to the charged amount of product from Part B.

Part D. Preparation of 4-{[2-(4-ethylphenyl)-1,3-benzothiazol-6-yl]sulfonyl}-N-hydroxytetrahydro-2H-pyran-4-carboxamide:

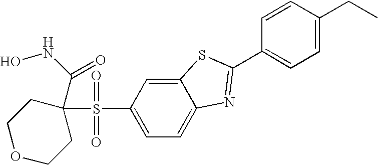

To the THP-hydroxamate product from Part C (0.47 g, MW 530.67) was added methanol (0.5 ml) and 4 N HCl in dioxane (5 ml). The resulting mixture was stirred for 1 hr at room temperature. The solvent was then concentrated to one-third volume, and diethyl ether was added. The resulting solid was dried to afford the desired hydroxamic acid as a white solid (0.37 g, 92% yield). ¹H NMR confirmed the presence of the desired hydroxamic acid. HRMS for C₂₁H₂₂N₂O₅S₂ showed M^{+H}_{found}=447.5507 (M^{+H}_{calc}=447.5499).

### Example 17

### Preparation of 4-{[2-(5-chlorothien-2-yl)-1,3-benzothiazol-6-yl]sulfonyl}-N-hydroxytetrahydro-2H-pyran-4-carboxamide

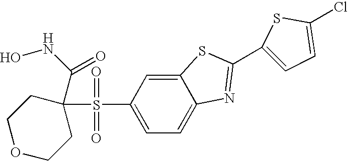

Part A. Preparation of tert-butyl 4-{[2-(5-chlorothien-2-yl)-1,3-benzothiazol-6-yl]sulfonyl}tetrahydro-2H-pyran-4-carboxylate:

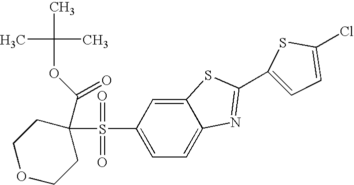

Tert-butyl-4-[(2-bromo-1,3-benzothiazol-6-yl)sulfonyl]tetrahydro-2H-pyran-4-carboxylate (1.0 g; MW 465.63; prepared in accordance with Part C, Example 9), 4-chlorothiophene boronic acid (from Aldrich, 0.42 g, MW 162.40, 1.2 eq), (1,1′bis-(diphenylphosphino)-ferrocene) palladium dichloride (from Aldrich, 0.09 g, MW 816.64, 0.05 eq), and 2 M sodium carbonate (aqueous, 3.3 ml, 3.0 eq) were slurried in ethylene glycol dimethylether (15 ml). The resulting mixture was heated at 55° C. for 3 hr. After cooling to room temperature, the mixture was filtered through a Celite plug. The filtrate was diluted with water (20 ml). The diluted mixture was extracted with ethyl acetate (3×25 ml). The organics were combined and then washed with water (2×30 ml), washed with brine (1×30 ml), dried over sodium sulfate, filtered, and concentrated to form a black oily solid. Recrystallization from methanol afforded the desired ester as a brown solid (0.90 g, 82% yield). ¹H NMR and LCMS confirmed the presence of the desired ester. The “equivalents” above indicate equivalents relative to the charged amount of tert-butyl-carboxylate.

Part B. Preparation of 4-{1[2-(5-chlorothien-2-yl)-1,3-benzothiazol-6-yl]sulfonyl}tetrahydro-2H-pyran-4-carboxylic Acid:

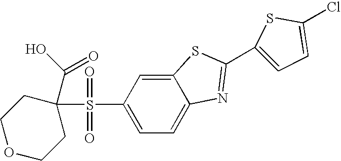

To a solution of the ester product from Part A (0.55 g, MW 522.70) in dichloromethane (2 ml) was added trifluoroacetic acid (from Aldrich, 4 ml). This mixture was stirred for 4 hr at room temperature. Afterward, the mixture was concentrated to one-third volume to form a residue, which, in turn, was dripped into stirring diethyl ether (10 ml). The resulting solid was collected, washed with diethyl ether, and dried to afford the desired carboxylic acid as a brown oil (0.94 g, 100+% crude yield). LCMS confirmed the presence of the desired carboxylic acid.

Part C. Preparation of 4-{[2-(5-chlorothien-2-yl)-1,3-benzothiazol-6-yl]sulfonyl}-N-(tetrahydro-2H-pyran-2-yloxy)tetrahydro-2H-pyran-4-carboxamide:

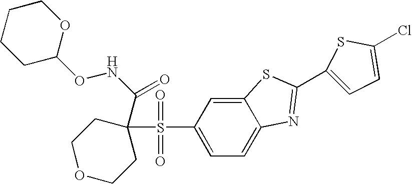

To the carboxylic acid product from Part B (0.80 g, MW 443.95) in N,N-dimethylacetamide (5 ml) was added triethylamine (from Aldrich, 0.59 ml, MW 101.19, 3.0 eq), followed by N-hydroxybenzotriazole hydrate (from Aldrich, 0.57 g, MW 135.13, 2.0 eq), O-(tetrahydro-2H-pyran-2-yl) hydroxylamine (0.37 g, MW 117.16, 1.5 eq), and, lastly, 1-(3-dimethylaminopropyl)-3-ethylcarbodiimide hydrochloride (from Sigma, 1.04 g, MW 191.76, 2.5 eq). The resulting mixture was stirred at room temperature for 15 hr. Afterward, the mixture was diluted with water (1 ml) and ethyl acetate (10 ml). The organic layer was separated, and the aqueous layer was further extracted with ethyl acetate (2×15 ml). The organics were combined and then washed with saturated aqueous NaHCO₃ (2×15 ml), washed with water (2×10 ml), washed with brine (1×20 ml), dried over sodium sulfate, and concentrated to form a crude product in the form of a beige solid. The solid was tritiated with diethyl ether and then dried to afford the desired THP-hydroxamate as a tan oil. The oil was chromatographed (RP-C¹⁸, acetonitrile/water) to afford the THP-hydroxamate as a clear oil (0.25 g, 22% yield). ¹H NMR and LCMS confirmed the presence of the desired compound. The “equivalents” above indicate equivalents relative to the charged amount of product from Part B.

Part D. Preparation of 4-{[2-(5-chlorothien-2-yl)-1,3-benzothiazol-6-yl]sulfonyl}-N-hydroxytetrahydro-2H-pyran-4-carboxamide:

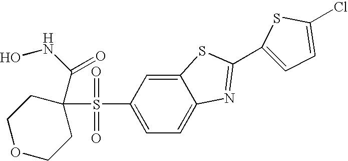

To the THP-hydroxamate product from Part C (0.25 g, MW 543.08) was added methanol (0.5 ml) and 4 N HCl in dioxane (5 ml). The resulting mixture was stirred for 1 hr at room temperature. The solvent was then concentrated to one-third volume, and diethyl ether was added. The resulting solid was dried to the desired hydroxamic acid as a yellow solid (0.10 g, 48% yield). ¹H NMR confirmed the presence of the desired hydroxamic acid. HRMS for C₁₇H₁₅C¹N₂O₅S₃ showed M^{+H}_{found}=459.9714 (M^{+H}_{calc}=459.9702).

### Example 18

### Preparation of 4-{[2-(2,4-difluorophenyl)-1,3-benzothiazol-6-yl]sulfonyl}-N-hydroxytetrahydro-2H-pyran-4-carboxamide

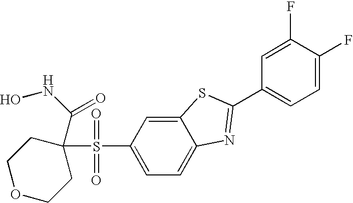

Part A. Preparation of tert-butyl 4-{[2-(3,4-difluorophenyl)-1,3-benzothiazol-6-yl]sulfonyl}tetrahydro-2H-pyran-4-carboxylate:

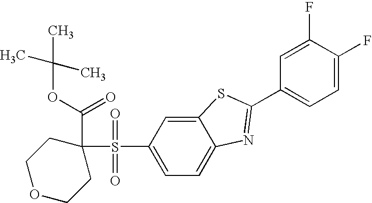

Tert-butyl-4-[(2-bromo-1,3-benzothiazol-6-yl)sulfonyl]tetrahydro-2H-pyran-4-carboxylate (1.0 g; MW 465.63; prepared in accordance with Part C, Example 9), 3,4-difluorophenyl boranic acid (from Aldrich, 0.41 g, MW 157.91, 1.2 eq), (1,1′bis-(diphenylphosphino)-ferrocene) palladium dichloride (from Aldrich, 0.09 g, MW 816.64, 0.05 eq), and 2 M sodium carbonate (aqueous, 3.3 ml, 3.0 eq) were slurried in ethylene glycol dimethylether (15 ml). The resulting mixture was heated at 55° C. for 3 hr. Afterward, the mixture was cooled to room temperature and then filtered through a Celite plug. The filtrate was diluted with water (20 ml). The diluted mixture was extracted with ethyl acetate (3×25 ml). The organics were combined and then washed with water (2×30 ml), washed with brine (1×30 ml), dried over sodium sulfate, filtered, and concentrated to form a black oily solid. Recrystallization from methanol afforded the desired ester as a tan solid (0.76 g, 71% yield). ¹H NMR and LCMS confirmed the presence of the desired ester. The “equivalents” above indicate equivalents relative to the charged amount of tert-butyl-carboxylate.

Part B. Preparation of 4-{[2-(3,4-difluorophenyl)-1,3-benzothiazol-6-yl]sulfonyl}tetrahydro-2H-pyran-4-carboxylic Acid:

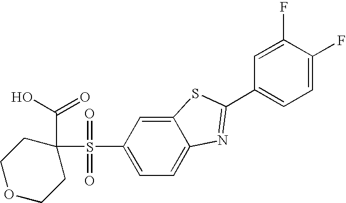

To a solution of the ester product from Part A (0.30 g, MW 495.57) in dichloromethane (4 ml) was added trifluoroacetic acid (from Aldrich, 8 ml). The reaction mixture stirred 4 hr at room temperature. Work up consisted of concentrating the mixture to one-third volume then dripping residue into stirring diethyl ether (10 ml). The resulting solid was collected, washed with diethyl ether, and dried to afford the desired carboxylic acid as a brown solid (0.70 g, 100+% crude yield). LCMS confirmed the presence of the desired carboxylic acid.

Part C. Preparation of 4-{[2-(3,4-difluorophenyl)-1,3-benzothiazol-6-yl]sulfonyl}-N-(tetrahydro-2H-pyran-2-yloxy)tetrahydro-2H-pyran-4-carboxamide:

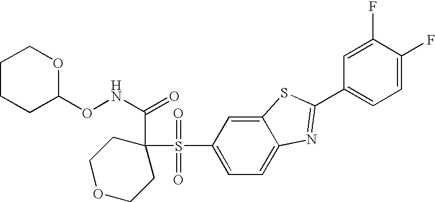

To the carboxylic acid product from Part B (0.70 g, MW 439.45) in N,N-dimethylacetamide (5 ml) was added triethylamine (from Aldrich, 0.33 ml, MW 101.19, 3.0 eq), followed by N-hydroxybenzotriazole hydrate (from Aldrich, 0.43 g, MW 135.13, 2.0 eq), O-(tetrahydro-2H-pyran-2-yl) hydroxylamine (0.27 g, MW 117.16, 1.5 eq), and, lastly, 1-(3-dimethylaminopropyl)-3-ethylcarbodiimide hydrochloride (from Sigma, 0.78 g, MW 191.76, 2.5 eq). The resulting mixture was stirred at room temperature for 15 hr. Afterward, the mixture was diluted with water (1 ml) and ethyl acetate (10 ml). The organic layer was separated, and the aqueous was further extracted with ethyl acetate (2×15 ml). The organics were combined and washed with saturated aqueous NaHCO₃ (2×15 ml), washed with water (2×10 ml), washed with brine (1×20 ml), dried over sodium sulfate, and concentrated to form a crude product in the form of a beige solid. The solid was tritiated with diethyl ether and then dried to afford the desired THP-hydroxamate as a colorless oil (0.83 g, 96% yield). ¹H NMR and LCMS confirmed the presence of the desired THP-hydroxamate. The “equivalents” above indicate equivalents relative to the charged amount of product from Part B.

Part D. Preparation of 4-{[2-(3,4-difluorophenyl)-1,3-benzothiazol-6-yl]sulfonyl}-N-hydroxytetrahydro-2H-pyran-4-carboxamide:

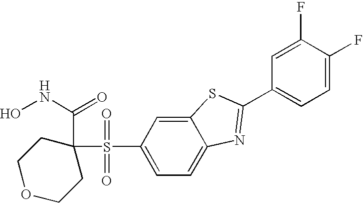

To the THP-hydroxamate product from Part C (0.83 g, MW 538.59) was added methanol (0.5 ml) and 4 N HCl in dioxane (5 ml). The resulting mixture was stirred for 1 hr at room temperature. The solvent was then concentrated to one-third volume, and diethyl ether was added. The resulting solid was dried to the desired hydroxamic acid as a white solid (0.61 g, 87% yield). ¹H NMR confirmed the presence of the desired hydroxamic acid. HRMS for C₁₇H₁₅F₂N₂O₅S₂ showed M^{+H}_{found}=455.4783 (M^{+H}_{calc}=455.4776).

### Example 19

### Preparation of 4-{[2-(2,4-difluorophenyl)-1,3-benzothiazol-6-yl]sulfonyl}-N-hydroxytetrahydro-2H-pyran-4-carboxamide

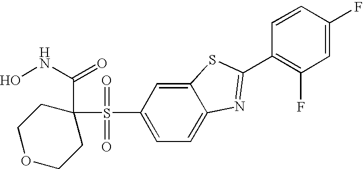

Part A. Preparation of tert-butyl 4-{[2-(2,4-difluorophenyl)-1,3-benzothiazol-6-yl]sulfonyl}tetrahydro-2H-pyran-4-carboxylate:

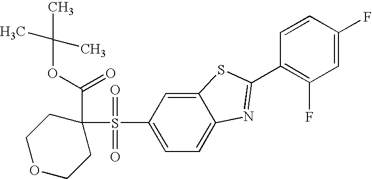

Tert-butyl-4-[(2-bromo-1,3-benzothiazol-6-yl)sulfonyl]tetrahydro-2H-pyran-4-carboxylate (1.0 g; MW 465.63; prepared in accordance with Part C, Example 9), 2,4-difluorophenyl boranic acid (from Aldrich, 0.41 g, MW 157.91, 1.2 eq), (1,1′bis-(diphenylphosphino)-ferrocene) palladium dichloride (from Aldrich, 0.09 g, MW 816.64, 0.05 eq), and 2 M sodium carbonate (aqueous, 3.3 ml, 3.0 eq) were slurried in ethylene glycol dimethylether (15 ml). The resulting mixture was heated at 55° C. for 3 hr. Afterward, the mixture was cooled to room temperature. The cooled mixture was filtered through a Celite plug. The filtrate was diluted with water (20 ml). The diluted mixture was extracted with ethyl acetate (3×25 ml). The organics were combined and then washed with water (2×30 ml), washed with brine (1×30 ml), dried over sodium sulfate, filtered, and concentrated to form a black oily solid. Recrystallization from methanol afforded the desired ester as a tan solid (0.34 g, 31% yield). ¹H NMR and LCMS confirmed the presence of the desired ester. The “equivalents” above indicate equivalents relative to the charged amount of tert-butyl-carboxylate.

Part B. Preparation of 4-{[2-(2,4-difluorophenyl)-1,3-benzothiazol-6-yl]sulfonyl}tetrahydro-2H-pyran-4-carboxylic Acid:

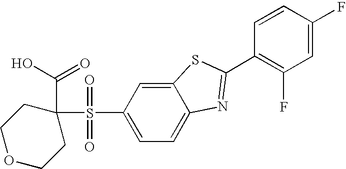

To a solution of the ester product from Part A (0.30 g, MW 495.57) in dichloromethane (4 ml) was added trifluoroacetic acid (from Aldrich, 8 ml). This mixture was stirred for 4 hr at room temperature. The mixture was then concentrated to one-third volume to form a residue, which, in turn, was dripped into stirring diethyl ether (10 ml). The resulting solid was collected, washed with diethyl ether, and dried to afford the desired carboxylic acid as a brown solid (0.30 g, 100+% crude yield). LCMS confirmed the presence of the desired carboxylic acid.

Part C. Preparation of 4-{[2-(2,4-difluorophenyl)-1,3-benzothiazol-6-yl]sulfonyl}-N-(tetrahydro-2H-pyran-2-yloxy)tetrahydro-2H-pyran-4-carboxamide:

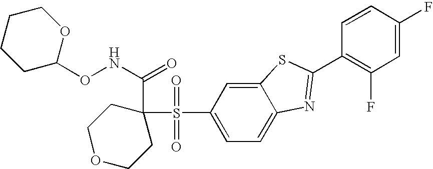

To the carboxylic acid product from Part B (0.30 g, MW 495.57) in N,N-dimethylacetamide (5 ml) was added triethylamine (from Aldrich, 0.33 ml, MW 101.19, 3.0 eq), followed by N-hydroxybenzotriazole hydrate (from Aldrich, 0.43 g, MW 135.13, 2.0 eq), O-(tetrahydro-2H-pyran-2-yl) hydroxylamine (0.27 g, MW 117.16, 1.5 eq), and, lastly, 1-(3-dimethylaminopropyl)-3-ethylcarbodiimide hydrochloride (from Sigma, 0.78 g, MW 191.76, 2.5 eq). The resulting mixture was stirred at room temperature for 15 hr. Afterward, the mixture was diluted with water (1 ml) and ethyl acetate (10 ml). The organic layer was separated, and the aqueous was further extracted with ethyl acetate (2×15 ml). The organics were combined and then washed with saturated aqueous NaHCO₃ (2×15 ml), washed with water (2×10 ml), washed with brine (1×20 ml), dried over sodium sulfate, and concentrated to form a crude product in the form of a beige solid. The solid was tritiated with diethyl ether and then dried to afford the desired THP-hydroxamate as a colorless oil (0.38 g, 100+% yield). ¹H NMR and LCMS confirmed the presence of the desired THP-hydroxamate. The “equivalents” above indicate equivalents relative to the charged amount of product from Part B.

Part D. Preparation of 4-{[2-(2,4-difluorophenyl)-1,3-benzothiazol-6-yl]sulfonyl}-N-hydroxytetrahydro-2H-pyran-4-carboxamide:

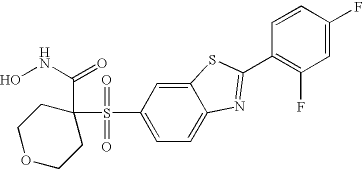

To the THP-hydroxamate product from Part C (0.38 g, MW 538.59) was added methanol (0.5 ml) and 4 N HCl in dioxane (5 ml). The resulting mixture was stirred for 1 hr at room temperature. The solvent was then concentrated to one-third volume, and diethyl ether was added. The resulting solid was dried to afford the desired hydroxamic acid as a white solid (0.23 g, 72% yield). ¹H NMR confirmed the presence of the desired hydroxamic acid. HRMS for C₁₇H₁₅F₂N₂O₅S₂ showed M^{+H}_{found}=455.4785 (M^{+H}_{calc}=455.4776).

### Example 20

### Preparation of N-hydroxy-4-[(2-thien-3-yl-1,3-benzothiazol-6-yl)sulfonyl]tetrahydro-2H-pyran-4-carboxamide

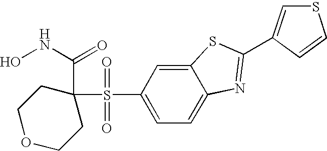

Part A. Preparation of tert-butyl 4-[(2-thien-3-yl-1,3-benzothiazol-6-yl)sulfonyl]tetrahydro-2H-pyran-4-carboxylate:

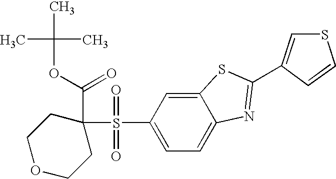

Tert-butyl-4-[(2-bromo-1,3-benzothiazol-6-yl)sulfonyl]tetrahydro-2H-pyran-4-carboxylate (1.0 g; MW 465.63; prepared in accordance with Part C, Example 9), 3-thiophene boronic acid (from Aldrich, 0.33 g, MW 127.96, 1.2 eq), (1,1′bis-(diphenylphosphino)-ferrocene) palladium dichloride (from Aldrich, 0.09 g, MW 816.64, 0.05 eq), and 2 M sodium carbonate (aqueous, 3.3 ml, 3.0 eq) were slurried in ethylene glycol dimethylether (15 ml). The resulting mixture was heated at 55° C. for 3 hr. The mixture was then cooled to room temperature. The cooled mixture was filtered through a Celite plug. The filtrate was diluted with water (20 ml) and then extracted with ethyl acetate (3×25 ml). The organics were combined and then washed with water (2×30 ml), washed with brine (1×30 ml), dried over sodium sulfate, filtered, and concentrated to form a black, oily solid. Recrystallization from methanol afforded the desired ester as a tan solid (0.69 g, 68% yield). ¹H NMR and LCMS confirmed the presence of the desired ester. The “equivalents” above indicate equivalents relative to the charged amount of tert-butyl-carboxylate.

Part B. Preparation of 4-{[2-(2-thien-3-yl)-1,3-benzothiazol-6-yl]sulfonyl}tetrahydro-2H-pyran-4-carboxylic Acid:

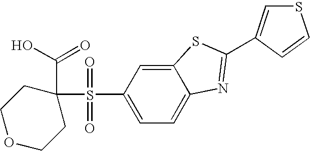

To a solution of the ester product from Part A (0.65 g, MW 465.61) in dichloromethane (4 ml) was added trifluoroacetic acid (from Aldrich, 8 ml). This mixture was stirred for 4 hr at room temperature. Afterward, the mixture was concentrated to one-third volume to form a residue, which, in turn, was dripped into stirring diethyl ether (10 ml). The resulting solid was collected, washed with diethyl ether, and dried to afford the desired carboxylic acid as a brown solid (0.60 g, 100+% crude yield). LCMS confirmed the presence of the desired carboxylic acid.

Part C. Preparation of 4-{[2-(2-thien-3-yl)-1,3-benzothiazol-6-yl]sulfonyl}-N-(tetrahydro-2H-pyran-2-yloxy)tetrahydro-2H-pyran-4-carboxamide:

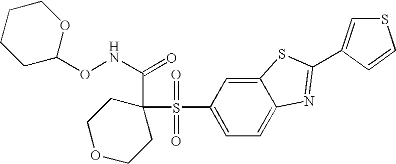

To the carboxylic acid product from Part B (0.60 g, MW 409.50) in N, N-dimethylacetamide (5 ml) was added triethylamine (from Aldrich, 0.31 ml, MW 101.19, 3.0 eq), followed by N-hydroxybenzotriazole hydrate (from Aldrich, 0.40 g, MW 135.13, 2.0 eq), O-(tetrahydro-2H-pyran-2-yl) hydroxylamine (0.26 g, MW 117.16, 1.5 eq), and, lastly, 1-(3-dimethylaminopropyl)-3-ethylcarbodiimide hydrochloride (from Sigma, 0.74 g, MW 191.76, 2.5 eq). The resulting mixture was stirred at room temperature for 15 hr. The mixture was then diluted with water (1 ml) and ethyl acetate (10 ml). The organic layer was separated and the aqueous layer was further extracted with ethyl acetate (2×15 ml). The organics were combined and washed with saturated aqueous NaHCO₃ (2×15 ml), washed with water (2×10 ml), washed with brine (1×20 ml), dried over sodium sulfate, and concentrated to form a crude product in the form of a beige solid. The solid was tritiated with diethyl ether and then dried to afford the desired THP-hydroxamate as a tan oil (0.71 g, 93% yield). ¹H NMR and LCMS confirmed the presence of the desired THP-hydroxamate. The “equivalents” above indicate equivalents relative to the charged amount of product from Part B.

Part D. Preparation of 4-{[2-(2-thien-3-yl)-1,3-benzothiazol-6-yl]sulfonyl}-N-hydroxytetrahydro-2H-pyran-4-carboxamide:

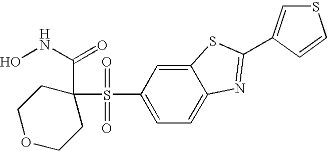

To the THP-hydroxamate product from Part C (0.71 g, MW 508.63) was added methanol (0.5 ml) and 4 N HCl in dioxane (5 ml). The resulting mixture was stirred for 1 hr at room temperature. The solvent was then concentrated to one-third volume, and diethyl ether was added. The resulting solid was dried to afford the desired hydroxamic acid as a white solid (0.49 g, 83% yield). ¹H NMR confirmed the presence of the desired hydroxamic acid. HRMS for C₁₇H₁₆N₂O₅S₃ showed M^{+H}_{found}=425.5259 (M^{+H}_{calc}=425.5254).

### Example 21

### Preparation of N-hydroxy-1-(2-methoxyethyl)-4-({2-[4-(trifluoromethoxy)phenyl]-1,3-benzothiazol-6-yl}sulfonyl)piperidine-4-carboxamide

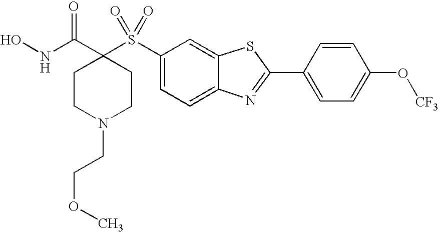

Part A. Preparation of tert-butyl({2-[4-(trifluoromethoxy)phenyl]-1,3-benzothiazol-6-yl}sulfonyl)acetate:

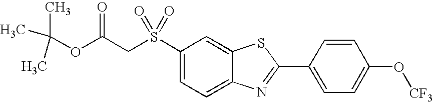

To a solution of tert-butyl[(2-bromo-1,3-benzothiazol-6-yl)sulfonyl]acetate (5.0 g, 12.8 mmol, prepared as in Part B, Example 9) in dimethoxyethane (25 ml) was added trifluoromethoxybenzene boronic acid (from Aldrich, 2.8 g, 14 mmol) and aqueous sodium carbonate (20 mL). This mixture was stirred at ambient temperature for 20 min while an N₂ stream was bubbled below the surface of the solution. [1,1′Bis(diphenylphosphino)ferrocene)dichloropalladium(II) (from Aldrich, 1 g, 1.2 mmol) was then added, and the resulting mixture was stirred at 80° C. until analytical reverse phase high pressure liquid chromatography indicated complete reaction. Afterward, the mixture was cooled to ambient temperature, and then filtered through a Celite pad. The filtrate was concentrated to form a residue, which, in turn, was purified on silica gel (ethylacetate/hexanes) to afford the desired tert-butyl ester as a black oil (4 g, 66% yield). LC/MS m/z=474 [M+H]. ¹H NMR confirmed the presence of the desired tert-butyl ester.

Part B. Preparation of tert-butyl]-(2-methoxyethyl)-4-({2-[4-(trifluoromethoxy)phenyl]-1,3-benzothiazol-6-yl}sulfonyl)piperidine-4-carboxylate:

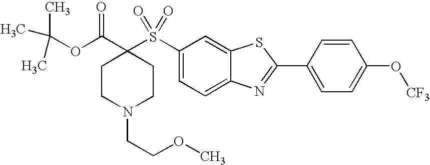

An N,N-dimethylformamide (25.0 mL) solution of bis(2-chloroethyl)-2-methoxyethylamine HCl (3.5 g, 19 mmol, from Clariant), potassium carbonate (4.8 g, 57 mmol), and 18-crown-6 ether (0.34 g, 1.29 mmol) being stirred at 60° C. under N₂ was treated with the ester prepared in Part A (5.0 g, 13 mmol). After 23 hr at 60° C., the mixture was diluted with ethyl acetate (30 mL) and then partitioned with water (25 mL). The aqueous layer was separated, extracted with ethyl acetate (2×20 mL). The combined organics were subsequently washed with saturated NaHCO₃ (20 mL), washed with 1:1 brine/water (20 mL), washed with brine (20 mL), dried over Na₂SO₄, filtered, and concentrated in vacuo. The resulting oil solidified and was purified by tritiation with methanol to afford the desired ester as a solid (6 g, 85% yield). LC/MS m/z=601 [M+H].

Part C. Preparation of 1-(2-methoxyethyl)-4-({2-[4-(trifluoromethoxy)phenyl]-1,3-benzothiazol-6-yl}sulfonyl)piperidine-4-carboxylic Acid:

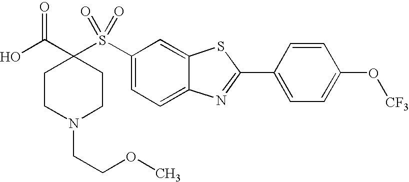

A methylene chloride solution (20 mL) of the ester prepared in Part B (2.6 g, 4.9 mmol) was treated with trifluoroacetic acid (5.0 mL, 64.9 mmol) and stirred at ambient temperature. After 14 hr, the mixture was concentrated in vacuo. The concentrated mixture was treated with diethyl ether (25 mL), and then concentrated in vacuo. This exchange was repeated once more. The resulting material was treated with diethyl ether (20 mL). After stirring this mixture at ambient temperature for 15 min, the solid that separated from solution was filtered. This afforded the desired carboxylic acid as a white solid (2.2 g)

Part D. Preparation of 4-[2-(4-trifluoromethoxy-phenyl)-benzothiazole-6-sulfonyl]-tetrahydro-pyran-4-carboxylic acid (tetrahydro-pyran-2-yloxy)-amide:

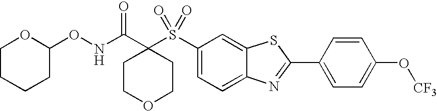

In dry glassware under N₂, the carboxylic acid from Part C (2.2 g, 4 mmol) was dissolved in dry dimethylformamide (30 mL). The following reagents were then added to the solution in the following order: N-hydroxybenzotriazole hydrate (0.65 g, 4 mmol), triethylamine (1.2 mL, 12 mmol), O-(tetrahydro-2H-pyran-2-yl)hydroxylamine (0.5,6 mmol), and 1-(3-dimethylaminopropyl)-3-ethylcarbodiimide hydrochloride (1.1 g, 6 mmol). After 12 hr at ambient temperature, the mixture was poured into water. A crude product was then extracted using ethyl acetate. The crude product, in turn, was washed with water, washed with saturated NaHCO₃, dried over Na₂SO₄, filtered, and concentrated in vacuo. Chromatography (on silica, ethyl acetate/hexanes) provided the desired THP-hydroxamate as a white foam (1.9 g, 80% yield). LCMS m/z=587 [M+H]⁺.

Part E. Preparation of N-hydroxy-1-(2-methoxyethyl)-4-({2-[4-(trifluoromethoxy)phenyl]-1,3-benzothiazol-6-yl}sulfonyl)piperidine-4-carboxamide:

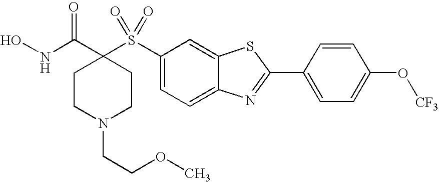

To the THP-hydroxamate product from Part D (1.9 g, 3.2 mmol) was added acetonitrile (20 mL) and aqueous 6N HCl (4 mL). This solution was stirred for 1 hr at ambient temperature (the reaction was complete at the end of this period). Afterward, a stream of N₂ was placed over the surface of the solution. After 1 hr, enough acetonitrile evaporated to cause the desired hydroxamic acid to separate from solution. This solid was filtered, dried, and purified on reverse phase column chromatography (C18) to afford the desired hydroxamic acid as an off-white solid (0.25 mg, 14% yield). HRMS (ES+) M+H⁺ calculated for C₂₃H₂₄N₃O₆S₂F₃: 560.4, found 560.

### Example 22

### Preparation of N-hydroxy-4-{[2-(4-phenyl-1H-imidazol-1-yl)-1,3-benzothiazol-6-yl]sulfonyl}tetrahydro-2H-pyran-4-carboxamide

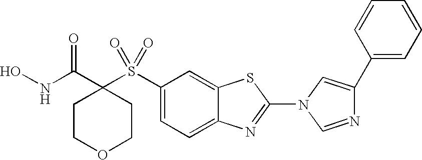

Part A. Preparation of tert-butyl 4-{[2-(4-phenyl-1H-imidazol-1-yl)-1,3-benzothiazol-6-yl]sulfonyl}tetrahydro-2H-pyran-4-carboxylate:

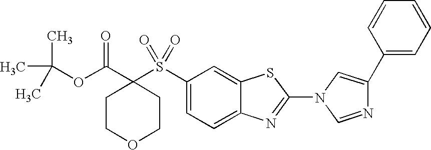

To a solution of tert-butyl-4-[(2-bromo-1,3-benzothiazol-6-yl)sulfonyl]tetrahydro-2H-pyran-4-carboxylate (2.5 g; 5.5 mmol; prepare as in Part C, Example 9) in dioxane (20 ml) was added phenyl imidazole (800 mg, 5.6 mmol) and potassium carbonate (1.5 g, 12 mmol). This mixture was stirred at 80° C. until analytical reverse phase high pressure liquid chromatography indicated complete reaction. Afterward, the mixture was cooled to ambient temperature and then concentrated using a rotary evaporator. After water (100 ml) was added, the mixture was filtered. The resulting residue was air dried to afford the desired ester as a white solid (3.5 g, 84% yield). LC/MS m/z=525 [M+H]. ¹H NMR confirmed the presence of the desired ester.

Part B. Preparation of 4-[2-(4-phenyl-imidazol-1-yl)-benzothiazole-6-sulfonyl]-tetrahydro-pyran-4-carboxylic Acid:

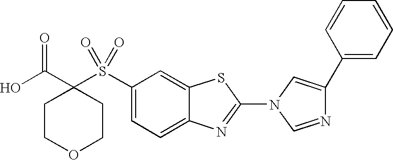

A methylene chloride solution (20 mL) of the ester from Part A (3.5 g, 5.5 mmol) was treated with trifluoroacetic acid (5.0 mL, 64.9 mmol). This mixture was stirred at ambient temperature for 14 hr. Afterward, the mixture was concentrated in vacuo. The concentrated mixture was treated with diethyl ether (50 mL), and then concentrated in vacuo. This exchange was repeated once more. The resulting material was treated with diethyl ether (20 mL). After stirring the mixture at ambient temperature for 15 min, the solid that separated from solution was filtered to afford the desired carboxylic acid as a white solid (2.5 g).

Part C. Preparation of 4-[2-(4-phenyl-imidazol-1-yl)-benzothiazole-6-sulfonyl]-tetrahydro-pyran-4-carboxylic acid (tetrahydro-pyran-2-yloxy)-amide:

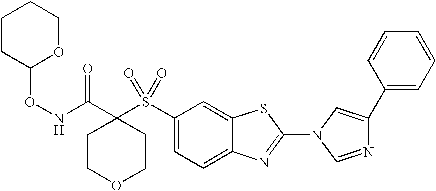

In dry glassware under N₂, the carboxylic acid from Part B (2.4 g, 5.1 mmol) was dissolved in dry dimethylacetamide (25 mL). The following reagents were then added to the solution in the following order: N-hydroxybenzotriazole hydrate (0.65 g, 4.8 mmol), triethylamine (1.2 mL, 12 mmol), O-(tetrahydro-2H-pyran-2-yl)hydroxylamine (0.5 g, 6 mmol), and 1-(3-dimethylaminopropyl)-3-ethylcarbodiimide hydrochloride (1.1 g, 6 mmol). After 12 hr at ambient temperature, the mixture was poured into water, and a crude THP-hydroxamate product was extracted using ethyl acetate. The extracted product was washed with water, washed with saturated NaHCO₃, dried over Na₂SO₄, filtered, and concentrated in vacuo. Chromatography (on silica, ethyl acetate/hexanes) provided the desired THP-hydroxamate as a white foam (2.1 g, 72% yield). LCMS m/z=568 [M+H]⁺.

Part D. Preparation of N-hydroxy-4-{[2-(4-phenyl-1H-imidazol-1-yl)-1,3-benzothiazol-6-yl]sulfonyl}tetrahydro-2H-pyran-4-carboxamide:

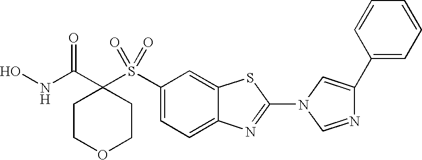

To the THP-hydroxamate product from Part C (2.1 g, 3.6 mmol) was added acetonitrile (20 mL) and aqueous 6N HCl (4 mL). This solution was stirred for 1 hr at ambient temperature (the reaction was complete at the end of this period). A stream of N₂ was then placed over the surface of the solution. After 1 hr, enough acetonitrile had evaporated to cause the desired hydroxamic acid to separate from solution. This solid was filtered, dried, and purified on reverse phase column chromatography (C¹⁸) to afford the desired hydroxamic acid as an off-white solid after (1 g, 40% yield). HRMS (ES+) M+H⁺ calculated for C₂₂H₂₀N₄O₅S₂: 485.6, found 485.1.

### Example 23

### Preparation of 4-{[2-(1,3-benzodioxol-5-yl)-1,3-benzothiazol-6-yl]sulfonyl}-N-hydroxytetrahydro-2H-pyran-4-carboxamide

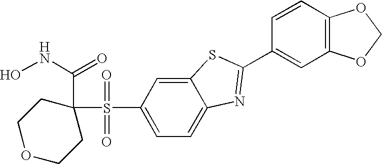

Part A. Preparation of tert-butyl 4-{[2-(1,3-benzodioxol-5-yl)-1,3-benzothiazol-6-yl]sulfonyl}tetrahydro-2H-pyran-4-carboxylate:

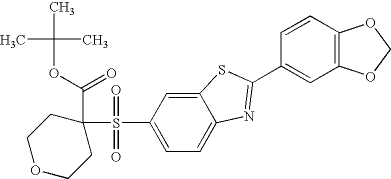

Tert-butyl-4-[(2-bromo-1,3-benzothiazol-6-yl)sulfonyl]tetrahydro-2H-pyran-4-carboxylate (1.0 g; MW 465.63; prepared in accordance with Part C, Example 9), 1,3-benzodioxol-5-ylboronic acid (from Lancaster, 0.43 g, MW 165.94, 1.2 eq), (1,1′bis-(diphenylphosphino)-ferrocene) palladium dichloride (from Aldrich, 0.09 g, MW 816.64, 0.05 eq), and 2 M sodium carbonate (aqueous, 3.3 ml, 3.0 eq) were slurried in ethylene glycol dimethylether (15 ml). The resulting mixture was heated at 55° C. for 3 hr. Afterward, the mixture was cooled to room temperature. The cooled mixture was filtered through a Celite plug. The filtrate was diluted with water (20 ml). The diluted mixture was extracted with ethyl acetate (3×25 ml). The organics were combined and then washed with water (2×30 ml), washed with brine (1×30 ml), dried over sodium sulfate, filtered, and concentrated to form a black oily solid. Recrystallization from methanol afforded the desired ester as a white solid (0.45 g, 44% yield). ¹H NMR and LCMS confirmed the presence of the desired ester. The “equivalents” above indicate equivalents relative to the charged amount of tert-butyl-carboxylate.

Part B. Preparation of 4-{[2-(1,3-benzodioxol-5-yl)-1,3-benzothiazol-6-yl]sulfonyl}tetrahydro-2H-pyran-4-carboxylic Acid:

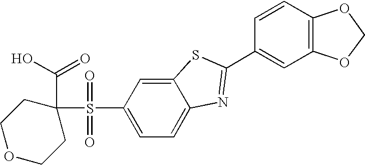

To a solution of the ester product from Part A (0.45 g, MW 503.59) in dichloromethane (4 ml) was added trifluoroacetic acid (from Aldrich, 8 ml). This mixture was stirred for 4 hr at room temperature. Afterward, the mixture was concentrated to one-third volume to form a residue, which, in turn, was dripped into stirring diethyl ether (10 ml). The resulting solid was collected, washed with diethyl ether, and dried to afford the desired carboxylic acid as a tan solid (0.45 g, 100+% crude yield). LCMS confirmed the presence of the desired carboxylic acid.

Part C. Preparation of 4-{[2-(1,3-benzodioxol-5-yl)-1,3-benzothiazol-6-yl]sulfonyl}-N-(tetrahydro-2H-pyran-2-yloxy)tetrahydro-2H-pyran-4-carboxamide:

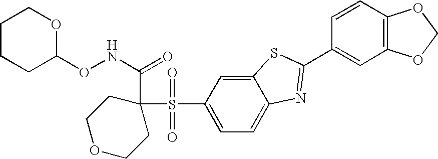

To the carboxylic acid product from Part B (0.44 g, MW 447.48, 1.0 eq) in N,N-dimethylacetamide (5 ml) was added triethylamine (from Aldrich, 0.19 ml, MW 101.19, 3.0 eq), followed by N-hydroxybenzotriazole hydrate (from Aldrich, 0.24 g, MW 135.13, 2.0 eq), O-(tetrahydro-2H-pyran-2-yl) hydroxylamine (0.16 g, MW 117.16, 1.5 eq), and, lastly, 1-(3-dimethylaminopropyl)-3-ethylcarbodiimide hydrochloride (from Sigma, 0.44 g, MW 191.76, 2.5 eq). The resulting mixture was stirred at room temperature for 15 hr. Afterward, the mixture was diluted with water (1 ml) and ethyl acetate (10 ml). The organic layer was separated, and the aqueous layer was further extracted with ethyl acetate (2×15 ml). The organics were combined and then washed with saturated aqueous NaHCO₃ (2×15 ml), washed with water (2×10 ml), washed with brine (1×20 ml), dried over sodium sulfate, and concentrated to form a crude product in the form of a beige solid. The solid was tritiated with diethyl ether. This mixture was then dried to afford the desired THP-hydroxamate as a tan oil (0.18 g, 37% yield). ¹H NMR and LCMS confirmed the presence of the desired THP-hydroxamate.

Part D. Preparation of 4-{[2-(1,3-benzodioxol-5-yl)-1,3-benzothiazol-6-yl]sulfonyl}-N-hydroxytetrahydro-2H-pyran-4-carboxamide:

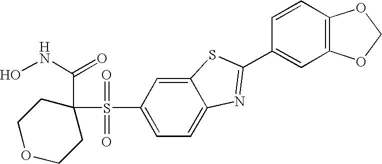

To the THP-hydroxamate product from Part C (0.18 g, MW 546.61, 1.0 eq) was added methanol (0.5 ml) and 4 N HCl in dioxane (5 ml). The resulting mixture was stirred for 1 hr at room temperature. The solvent was then concentrated to one-third volume, and diethyl ether was added. The resulting solid was dried to afford the desired hydroxamic acid as a yellow solid (0.15 g, 100+% yield). ¹H NMR confirmed the presence of the desired hydroxamic acid. HRMS for C₂₀H₁₈N₂O₇S₂ showed M^{+H}_{found}=463.0653 (M^{+H}_{calc}=463.0628).

### Example 24

### Preparation of 4-{[2-(4-ethoxyphenyl)-1,3-benzothiazol-6-yl]sulfonyl}-N-hydroxytetrahydro-2H-pyran-4-carboxamide

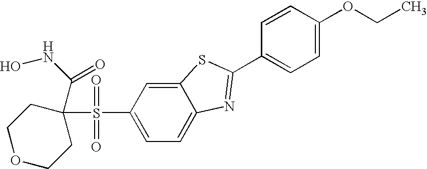

Part A. Preparation of tert-butyl 4-{[2-(4-ethoxyphenyl)-1,3-benzothiazol-6-yl]sulfonyl}tetrahydro-2H-pyran-4-carboxylate:

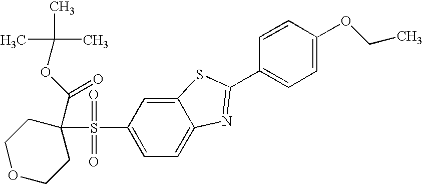

Tert-butyl-4-[(2-bromo-1,3-benzothiazol-6-yl)sulfonyl]tetrahydro-2H-pyran-4-carboxylate (1.0 g; MW 465.63; prepared in accordance with Part C, Example 9), 4-ethoxy boronic acid (from Aldrich, 0.43 g, MW 165.98, 1.2 eq), (1,1′bis-(diphenylphosphino)-ferrocene) palladium dichloride (from Aldrich, 0.09 g, MW 816.64, 0.05 eq), and 2 M sodium carbonate (aqueous, 3.3 ml, 3.0 eq) were slurried in ethylene glycol dimethylether (15 ml). The resulting mixture was heated at 55° C. for 3 hr. Afterward, the mixture was cooled to room temperature and then filtered through a Celite plug. The filtrate was diluted with water (20 ml). The diluted mixture was extracted with ethyl acetate (3×25 ml). The organics were combined and then washed with water (2×30 ml), washed with brine (1×30 ml), dried over sodium sulfate, filtered, and concentrated to form a black oily solid. Recrystallization from methanol afforded the desired ester as a white solid (0.45 g, 44% yield). ¹H NMR and LCMS confirmed the presence of the desired ester. The “equivalents” above indicate equivalents relative to the charged amount of tert-butyl-carboxylate.

Part B. Preparation of 4-{[2-(4-ethoxyphenyl)-1,3-benzothiazol-6-yl]sulfonyl}tetrahydro-2H-pyran-4-carboxylic Acid:

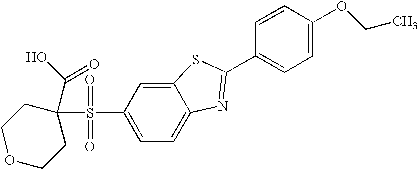

To a solution of the ester product from Part A (0.45 g, MW 503.63, 1.0 eq) in dichloromethane (4 ml) was added trifluoroacetic acid (from Aldrich, 8 ml). This mixture was stirred for 4 hr at room temperature. Afterward, the mixture was concentrated to one-third volume to form a residue, which, in turn, was dripped into stirring diethyl ether (10 ml). The resulting solid was collected, washed with diethyl ether, and dried to afford desired carboxylic acid as a tan solid (0.45 g, 100+% crude yield). LCMS confirmed the presence of the desired carboxylic acid.

Part C. Preparation of 4-{[2-(4-ethoxyphenyl)-1,3-benzothiazol-6-yl]sulfonyl}-N-(tetrahydro-2H-pyran-2-yloxy)tetrahydro-2H-pyran-4-carboxamide:

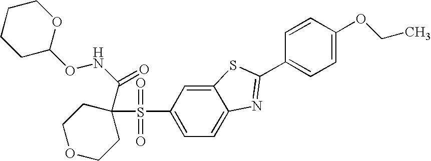

To the carboxylic acid product from Part B (0.44 g, MW 447.48, 1.0 eq) in N,N-dimethylacetamide (5 ml) was added triethylamine (from Aldrich, 0.19 ml, MW 101.19, 3.0 eq), followed by N-hydroxybenzotriazole hydrate (from Aldrich, 0.24 g, MW 135.13, 2.0 eq), O-(tetrahydro-2H-pyran-2-yl) hydroxylamine (0.16 g, MW 117.16, 1.5 eq), and, lastly, 1-(3-dimethylaminopropyl)-3-ethylcarbodiimide hydrochloride (from Sigma, 0.44 g, MW 191.76, 2.5 eq). The resulting mixture was stirred at room temperature for 15 hr. Afterward, the mixture was diluted with water (1 ml) and ethyl acetate (10 ml). The organic layer was separated, and the aqueous layer was further extracted with ethyl acetate (2×15 ml). The organics were combined and then washed with saturated aqueous NaHCO₃ (2×15 ml), washed with water (2×10 ml), washed with brine (1×20 ml), dried over sodium sulfate, and concentrated to form a crude product in the form of a beige solid. The solid was tritiated with diethyl ether and then dried to afford the desired THP-hydroxamate as a tan oil (0.41 g, 84% yield). ¹H NMR and LCMS confirmed the presence of the desired THP-hydroxamate.

Part D. Preparation of 4-{[2-(4-ethoxyphenyl)-1,3-benzothiazol-6-yl]sulfonyl}-N-hydroxytetrahydro-2H-pyran-4-carboxamide:

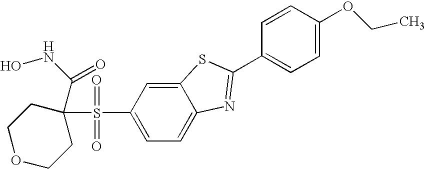

To the THP-hydroxamate product from Part C (0.41 g, MW 546.66, 1.0 eq) was added methanol (0.5 ml) and 4 N HCl in dioxane (5 ml). The resulting mixture was stirred for 1 hr at room temperature. The solvent was then concentrated to one-third volume, and diethyl ether was added. The resulting solid was dried to afford the desired hydroxamic acid as a white solid (0.25 g, 68% yield). ¹H NMR confirmed the presence of the desired hydroxamic acid. HRMS for C₂₁H₂₂N₂O₆S₂ showed M^{+H}_{found}=463.1015 (M^{+H}_{calc}=463.0992).

### Example 25

### Preparation of N-hydroxy-4-{(2-{4-[(trifluoromethyl)thio]phenyl}-1,3-benzothiazol-6-yl)sulfonyl}tetrahydro-2H-pyran-4-carboxamide

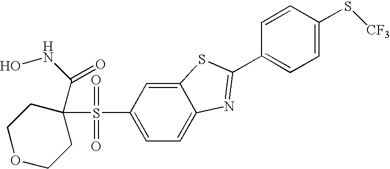

Part A. Preparation of tert-butyl 4-[(2-{4-[(trifluoromethyl)thio]phenyl}-1,3-benzothiazol-6-yl)sulfonyl]tetrahydro-2H-pyran-4-carboxylate:

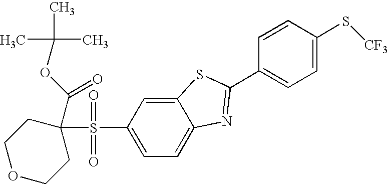

4-(Trifluoromethylthio)bromobenzene (from Lancaster, 0.67 g, MW 257.07, 1.2 eq), bis-pinacol diborane (from Aldrich, 0.73 g, MW 253.95, 1.3 eq), potassium acetate (from Aldrich, 0.86 g, MW 98.14, 4.0 eq), and (1,1′bis-(diphenylphosphino)-ferrocene) palladium dichloride (from Aldrich, 0.09 g, MW 816.64, 0.03 eq) were slurried in N,N-dimethylacetamide (5 ml). The resulting mixture was heated at 80° C. for 2 hr. At this point no bromide was detected by HPLC. Additional (1,1′bis-(diphenylphosphino)-ferrocene) palladium dichloride (from Aldrich, 0.09 g, MW 816.64, 0.03 eq) was added, along with aqueous sodium carbonate (2 M, 3.3 ml, 3.0 eq) and tert-butyl-4-[(2-bromo-1,3-benzothiazol-6-yl)sulfonyl]tetrahydro-2H-pyran-4-carboxylate (1.0 g, MW 462.38, 1.0 eq, prepared in accordance with Part C, Example 9). Stirring was continued at 80° C. for an additional 2 hr. Afterward, the reaction was quenched with water (5 ml). The mixture was then filtered through a Celite pad. The filtrate was extracted with ethyl acetate (3×15 ml). The organics were combined and then washed with water (2×30 ml), washed with brine (1×30 ml), dried over sodium sulfate, filtered, and concentrated to form a black residue. The residue was chromatographed on silica gel (ethyl acetate/hexanes) to afford the desired ester as a white solid (0.25 g, 21% yield). ¹H NMR and LCMS confirmed the presence of the desired ester. The “equivalents” above indicate equivalents relative to the charged amount of tert-butyl-4-[(2-bromo-1,3-benzothiazol-6-yl)sulfonyl]tetrahydro-2H-pyran-4-carboxylate.

Part B. Preparation of 4-[(2-{4-[(trifluoromethyl)thio]phenyl}-1,3-benzothiazol-6-yl)sulfonyl]tetrahydro-2H-pyran-4-carboxylic Acid:

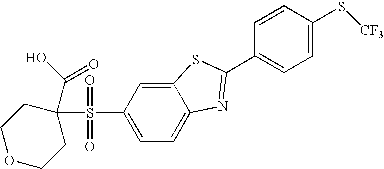

To a solution of the ester product from Part A (0.24 g, MW 559.64) in dichloromethane (4 ml) was added trifluoroacetic acid (from Aldrich, 8 ml). This mixture was stirred for 4 hr at room temperature. Afterward, the mixture was concentrated to one-third volume to form a residue, which, in turn, was dripped into stirring diethyl ether (10 ml). The resulting solid was collected, washed with diethyl ether, and dried to afford the desired carboxylic acid as a white solid (0.22 g, 100% crude yield). LCMS confirmed the presence of the desired carboxylic acid.

Part C. Preparation of N-(tetrahydro-2H-pyran-2-yloxy)-4-[(2-{4-[(trifluoromethyl)thio]phenyl}-1,3-benzothiazol-6-yl)sulfonyl]tetrahydro-2H-pyran-4-carboxamide:

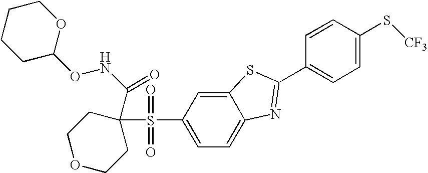

To the carboxylic acid product from Part B (0.22 g, MW 503.54) in N,N-dimethylacetamide (5 ml) was added triethylamine (from Aldrich, 0.12 ml, MW 101.19, 3.0 eq), followed by N-hydroxybenzotriazole hydrate (from Aldrich, 0.12 g, MW 135.13, 2.0 eq), O-(tetrahydro-2H-pyran-2-yl) hydroxylamine (0.08 g, MW 117.16, 1.5 eq), and, lastly, 1-(3-dimethylaminopropyl)-3-ethylcarbodiimide hydrochloride (from Sigma, 0.22 g, MW 191.76, 2.5 eq). The resulting mixture was stirred at room temperature for 15 hr. Afterward, the mixture was diluted with water (1 ml) and ethyl acetate (10 ml). The organic layer was separated, and the aqueous layer was further extracted with ethyl acetate (2×15 ml). The organics were combined and then washed with saturated aqueous NaHCO₃ (2×15 ml), washed with water (2×10 ml), washed with brine (1×20 ml), dried over sodium sulfate, and concentrated to form a crude product in the form of a beige solid. The solid was tritiated with diethyl ether and then dried to afford the desired THP-hydroxamate as a tan oil (0.28 g, 100+% yield). ¹H NMR and LCMS confirmed the presence of the desired THP-hydroxamate. The “equivalents” above indicate equivalents relative to the charged amount of product from Part B.

Part D. Preparation of N-hydroxy-4-[(2-{4-[(trifluoromethyl)thio]phenyl}-1,3-benzothiazol-6-yl)sulfonyl]tetrahydro-2H-pyran-4-carboxamide:

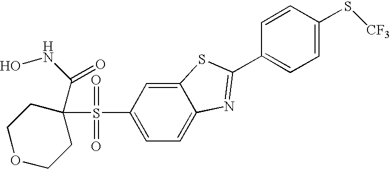

To the THP-hydroxamate product from Part C (0.26 g, MW 602.67) was added methanol (0.5 ml) and 4 N HCl in dioxane (5 ml). The resulting mixture was stirred for 1 hr at room temperature. The solvent was then concentrated to one-third volume, and diethyl ether was added. The resulting solid was dried to the desired hydroxamic acid as a white solid (0.16 g, 73% yield). ¹H NMR confirmed the presence of the desired hydroxamic acid. HRMS for C₂₀H₁₇₂N₂O₅S₃ showed M^{+H}_{found}=519.5619 (M^{+H}_{calc}=519.5607).

### Example 26

### Preparation of N-hydroxy-4-({6-[4-(3,3,3-trifluoropropyl)phenyl]pyridin-3-yl}sulfonyl)tetrahydro-2H-pyran-4-carboxamide Hydrochloride

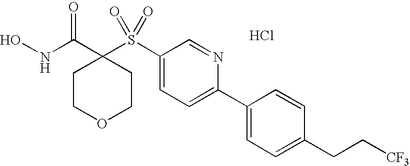

Part A. Preparation of 2-bromo-5-methanesulfonyl-pyridine:

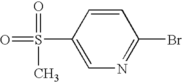

2,5-Dibromopyridine (from Aldrich, 10.0 g, MW 236.89) was dissolved in anhydrous diethyl ether (from Aldrich, 200 ml) and cooled to −78° C. n-Butyllithium (from Aldrich, 1.6 M in hexanes, 28 ml, 1.05 eq) was slowly dripped into the resulting mixture while maintaining temperature at less than −60° C. After complete lithium-bromide exchange, a solution of methyl disulfide (from Aldrich, 4.0 ml, MW 94.2, 1.05 eq) in diethyl ether (80 ml) was added to the mixture while continuing to maintain the temperature at less than -60° C. After stirring for 1 hr at −78° C., the reaction was quenched with water (100 ml). The mixture was then diluted with tetrahydrofuran (from Aldrich, 100 ml). With vigorous stirring, Oxone (from Aldrich, 77 g, MW 614 g, 3 eq) was added to the diluted mixture. The ice bath was removed and the mixture was stirred for 15 hr at room temperature. The mixture was then filtered through a Celite pad. After separating the filtrate, the organics were concentrated to form a residue, which, in turn, was taken up in ethyl acetate. The ethyl acetate was washed with water (3×), washed with brine (1×), dried over Na₂SO₄, and concentrated to afford the desired compound as a tan solid (9.2 g, 93% yield). ¹H, NOE, and HMBC NMR and LCMS confirmed the presence of desired compound. The “equivalents” above indicate equivalents relative to the charged amount of 2,5-dibromopyridine.

Part B. Preparation of (6-bromo-pyridine-3-sulfonyl)-acetic Acid Tert-Butyl Ester:

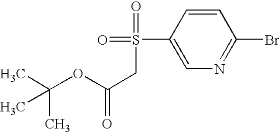

A solution of the product from Part A (9.2 g, MW 236.09) and t-butylcarboxlyate anhydride (from Aldrich, 10.5 g, MW 218.25, 1.2 eq) in tetrahydrofuran (from Aldrich, 80 ml) was cooled to −78° C. A solution of lithium bis(trimethylsilyl)amide (from Aldrich, 1.0 M in tetrahydrofuran, 116.9 ml, 3.0 eq) was slowly added to the cooled solution while maintaining the temperature at less than −65° C. After the addition, the mixture was warmed to 0° C. and stirred for 1 hr. The mixture was subsequently cooled back to −75° C. The reaction was then quenched with a saturated aqueous solution of ammonium chloride. The resulting mixture was warmed to room temperature and then separated. The aqueous layer was extracted with ethyl acetate (2×). The organics were combined and then washed with water (2×), washed with brine (2×), dried over Na₂SO₄, and concentrated to form a crude black oil. This oil was chromatographed (ethyl acetate:hexanes, 2:10) to afford the desired ester as a tan oil (7.9 g 59% yield). ¹H NMR confirmed the presence of the desired ester. The “equivalents” above indicate equivalents relative to the charged amount of the product from Part A.

Part C. Preparation of 4-(6-bromo-pyridine-3-sulfonyl)-tetrahydro-pyran-4-carboxylic Acid Tert-Butyl Ester:

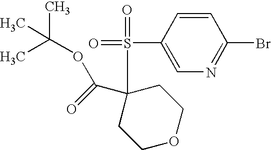

The ester product from Part B (4.37 g, MW 262.35), 18-crown-6 (Aldrich, 0.5 g, catalytic amount), potassium carbonate (from Aldrich, 7.39 g, MW 138.21, 5.3 eq), and bis(bromoethyl)ether (from Aldrich, 3.4 ml, MW 231.93, 2.1 eq) were slurried in N,N-dimethylformamide (25 ml). The resulting mixture was stirred at 65° C. for 15 hr (the reaction was complete at the end of this period). Afterward, the mixture was diluted with water (50 ml) and extracted with ethyl acetate (3×100 ml). The organics were combined and then washed with water (2×), washed with brine (1×), dried over Na₂SO₄, and concentrated to form an orange oily solid. The oil was slurried with hexanes, filtered, and dried to afford the desired ester as a yellow solid (3.8 g, 72% yield). ¹H NMR and LCMS confirmed the presence of the desired ester. The “equivalents” above indicate equivalents relative to the charged amount of the product from Part B.

Part D. Preparation of 4-[6-(4-hydroxy-phenyl)-pyridine-3-sulfonyl]-tetrahydro-pyran-4-carboxylic Acid Tert-Butyl Ester:

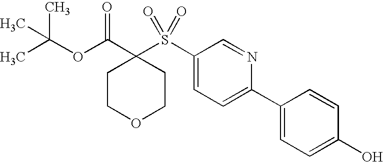

An N,N-dimethylformamide (212 mL) suspension of the ester product from Part C (14.62 g, 36.0 mmol), 4-(4, 4, 5, 5-tetramethyl-1, 3, 2-dioxaborolan-2-yl)phenol (from Aldrich, 9.50 g, 43.2 mmol), and [1,1′-bis(diphenylphosphino)ferrocene] dichloropalladium(II), complex with CH₂Cl₂, (from Aldrich, 1:1, 0.88 g, 1.08 mmol) was treated under N₂ with 2 M NaHCO₃ (90 mL, 180 mmol). The resulting orange suspension exothermed to 34° C. initially, and then was stirred while being heated at 80° C. for 4 hr. Afterward, the mixture was cooled to ambient temperature and diluted with 1:1 ethyl acetate/diethyl ether (200 mL). The diluted mixture was partitioned further with de-ionized water (150 mL). The layers separated very slowly. The aqueous layer was separated, saturated with NaCl (s), and extracted with ethyl acetate (5×100 mL). Because the resulting aqueous layer still had product, it was extracted with methylene chloride (2×100 mL). The combined organic layers were concentrated on the rotovap to about half the original total volume for ease of manipulation. The concentrated organics were then washed with saturated NaHCO₃ (50 mL), washed with brine (2×25 mL), dried overnight over MgSO₄, and concentrated in vacuo. The resulting brown oil was diluted with diethyl ether (ca. 15 mL), which, in turn, caused precipitation. The precipitate was filtered, washed with diethyl ether (ca. 5 mL), dried in a vacuum oven to afford the desired phenol product as a brown solid powder. The filtrate from the filtration was concentrated and then subjected again to the precipitation procedure to afford a second crop of product. The total amount of product was 10.94 g (72% yield). The presence of the desired phenol was confirmed by ¹H-NMR. LC/MS m/z -420 [M+H], 442 [M+Na].

Part E. Preparation of 4-[6-(4-trifluoromethanesulfonyloxy-phenyl)-pyridine-3-sulfonyl]-tetrahydro-pyran-4-carboxylic Acid Tert-Butyl Ester:

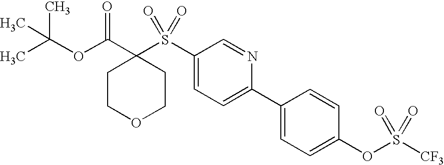

A pyridine (4.0 mL) solution of the product from Part E was treated under N₂ at 0° C. with trifluoromethanesulfonic anhydride (from Aldrich, 1.06 mL, 6.32 mmol). This mixture was stirred at 0° C. for 30 min, and then warmed to ambient temperature and stirred overnight. The reaction was driven to completion by cooling to 0° C., adding more trifluoromethanesulfonic anhydride (from Aldrich, 1.00 mL, 5.94 mmol), and then allowing the mixture to warm to ambient temperature overnight. The reaction was subsequently stopped by diluting with 1:1 diethyl ether/ethyl acetate (25 mL), and then partitioning with de-ionized water. The aqueous layer was extracted with ethyl acetate (10 mL). The organic layers were combined and washed with 1:1 brine/de-ionized water, washed with brine, dried over Na₂SO₄, filtered, and concentrated in vacuo. Because the resulting amber/yellow oil contained residual pyridine, it was dissolved in ethyl acetate, washed with 2 M aqueous HCl (2×25 mL), washed with brine (2×25 mL), dried over Na₂SO₄, filtered, and concentrated in vacuo. This afforded the desired ester as a yellow solid (2.77 g, 95% yield). The presence of the desired ester was confirmed by ¹H-NMR and ¹⁹F-NMR. LC/MS m/z=552 [M+H], 574 [M+Na].

Part F. Preparation of 4-{6-[4-(3,3,3-trifluoro-propyl)-phenyl]-pyridine-3-sulfonyl}-tetrahydro-pyran-4-carboxylic Acid Tert-Butyl Ester:

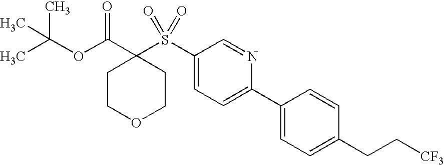

A THF (75 mL) suspension of Zn (from Aldrich, dust, 325 mesh, 30.0 g, 461 mmol) was stirred under N₂ at ambient temperature for 10 min. Afterward, 1,2-dibromoethane (from Aldrich, 4.75 g, 25.3 mmol) was added. The resulting mixture was brought to reflux times with a heat gun under N₂, and then cooled to ambient temperature in a water bath. These reflux and cooling steps were repeated two more times. The mixture was then cooled to 0° C. in an ice bath. Chlorotrimethylsilane (from Aldrich, 3.42 mL, 26.9 mmol) was slowly added to the cooled mixture over a period of a few minutes. The resulting mixture was stirred at 0° C. for 5 min, and then allowed to warm to ambient temperature over 15 min while continuing to be stirred. Afterward, the mixture was cooled to 0° C., and then slowly treated with 1,1,1-trifluoro-3-iodopropane causing an exothermic reaction. The mixture was warmed to ambient temperature and stirred for 1 hr. The mixture was then diluted with N,N-dimethylacetamide (10 mL) to afford an organozinc reagent. Separately, an N,N-dimethylacetamide (40 mL) solution of the product from Part E (2.0 g, 3.3 mmol) was treated with bis(benzonitrile)dichloropalladium(II) (from Aldrich, 0.08 g, 0.208 mmol) and 2-(dicyclohexylphosphino)-2′-methylbiphenyl (0.127 g, 0.349 mmol) under N₂. The organozinc reagent (2.2 mL of stock solution, 9.78 mmol) was then added to the mixture. The resulting mixture was stirred at 55° C. for 4 hr, and then allowed to cool to ambient temperature overnight. Subsequently, the reaction was quenched with saturated aqueous NaHCO₃ (20 mL). The mixture was then partitioned further with ethyl acetate (100 mL) and de-ionized water (50 mL). The resulting biphasic mixture was filtered through Celite (pre-washed with ethyl acetate). The filter cake, in turn, was washed with ethyl acetate. The organic layer was separated, and the aqueous layer was extracted with ethyl acetate (50 mL). The combined organic layers were washed with saturated aqueous NaHCO₃ (2×25 mL), washed with 1:1 brine/de-ionized water (2×25 mL), washed with brine (2×25 mL), dried over Na₂SO₄, filtered, and concentrated in vacuo. The resulting solid was diluted in diethyl ether, and then concentrated in vacuo, forming a glassy solid. This solid was triturated with 1:1 diethyl ether/hexanes. The solids were then filtered, washed with hexanes, and dried in a vacuum oven to afford the desired ester as a brown solid (1.25 g, 76% yield). The presence of the desired ester was confirmed by ¹H-NMR and ¹⁹F-NMR. LC/MS m/z=500 [M+H], 522 [M+Na].

Part G. Preparation of 4-{6-[4-(3,3,3-trifluoro-propyl)-phenyl]-pyridine-3-sulfonyl}-tetrahydro-pyran-4-carboxylic Acid:

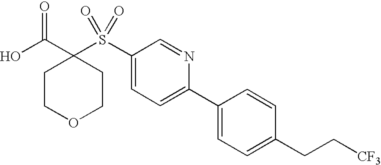

A methylene chloride (3.0 mL) solution of the ester product from Part F (1.22 g, 2.44 mmol) was treated with triethylsilane (from Aldrich, 1.0 mL, 6.26 mmol) and trifluoroacetic acid (from Aldrich, 3.0 mL, 38.9 mmol). The resulting solution was stirred at ambient temperature under N₂ for 3.5 days. Afterward, the mixture was concentrated in vacuo. The concentrated mixture was diluted with diethyl ether and then concentrated in vacuo, forming a glassy solid. The solid was triturated in 1:1 diethyl ether/hexanes, filtered, washed with 1:1 diethyl ether/hexanes, and dried in a vacuum oven to afford the desired carboxylic acid as a brown solid (0.95 g, >87% yield). The presence of the desired carboxylic acid was confirmed by ¹H-NMR and ¹⁹F-NMR. LC/MS m/z=444 [M+H].

Part H. Preparation of 4-{6-[4-(3,3,3-trifluoro-propyl)-phenyl]-pyridine-3-sulfonyl}-tetrahydro-pyran-4-carboxylic acid (tetrahydro-pyran-2-yloxy)-amide:

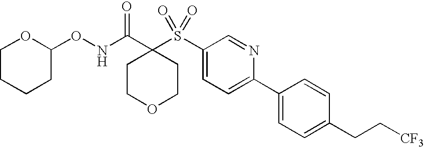

An N,N-dimethylformamide (4.2 mL) solution of the carboxylic acid product from Part D (0.93 g, 2.1 mmol) was treated with 1-[3-dimethylamino)propyl]-3-ethylcarbodiimide hydrochloride (from Aldrich, 0.60 g, 3.15 mmol) and 1-hydroxybenzotriazole (from Aldrich, 0.43 g, 3.15 mmol), followed by the addition of 4-N-methylmorpholine (from Aldrich, 0.69 mL, 6.30 mmol) and O-(tetrahydropyranyl) hydroxylamine (from Carbogen, 0.37 g, 3.15 mmol). The resulting solution was stirred at ambient temperature for 3 days. Afterward, the mixture was partitioned with ethyl acetate (20 mL) and de-ionized water (20 mL). The resulting layers were separated, and the aqueous layer was extracted with ethyl acetate (10 mL). The organic layers were combined and then washed with saturated aqueous NaHCO₃ (15 mL), washed with 1:1 brine/de-ionized water (2×15 mL), washed with brine (2×15 mL), dried over Na₂SO₄, filtered, and concentrated in vacuo. The resulting brown glassy solid was purified by silica chromatography (eluting with 7:3 hexanes/ethyl acetate (with 10% methanol)) to afford the desired THP-hydroxamate as a yellow glassy solid (0.86 g, 75% yield). The presence of the desired THP-hydroxamate was confirmed by ¹H-NMR and ¹⁹F-NMR. LC/MS m/z=543 [M+H], 565 [M+Na].

Part I. Preparation of N-hydroxy-4-({6-[4-(3,3,3-trifluoropropyl)phenyl]pyridin-3-yl}sulfonyl)tetrahydro-2H-pyran-4-carboxamide Hydrochloride:

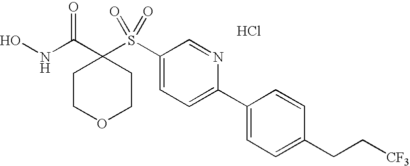

An ethyl acetate (9.2 mL) solution of the THP-hydroxamate of Part E (0.75 g, 1.38 mmol) was treated with 1.25 N HCl in methanol (from Fluka, 2.43 mL). This mixture was stirred at ambient temperature for 24 hr. The mixture was then diluted with diethyl ether (30 mL), resulting in the formation of a white precipitate. The solids were filtered, washed with diethyl ether, and dried in a vacuum oven to afford the desired hydroxamic acid as a white solid (0.41 g, 60% yield). The presence of the desired hydroxamic acid was confirmed by ¹H-NMR and ¹⁹F-NMR. LC/MS m/z=459 [M+H], 481 [M+Na]. HR-MS: M+H calculated for C₂₀H₂₂F₃N₂O₅S: 459.1196, found: 459.1172.

### Example 27

### Preparation of N-hydroxy-4-({6-[4-(3,3,4,4,4-pentafluorobutyl)phenyl]pyridin-3-yl}sulfonyl)tetrahydro-2H-pyran-4-carboxamide Hydrochloride

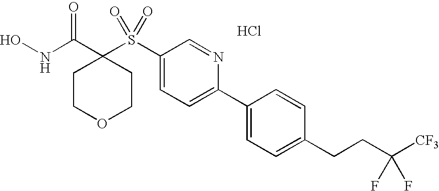

Part A. Preparation of 4-{6-[4-(3,3,4,4,4-pentafluoro-butyl)-phenyl]-pyridine-3-sulfonyl}-tetrahydro-pyran-4-carboxylic Acid Tert-Butyl Ester:

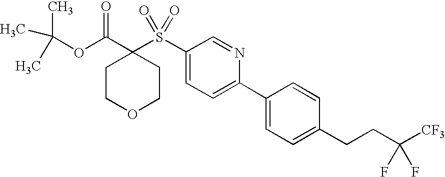

A THF (12 mL) suspension of Zn (from Aldrich, dust, 325 mesh, 3.98 g, 61.2 mmol) was stirred under N₂ at ambient temperature for 10 min. To this suspension was added 1,2-dibromoethane (from Aldrich, 0.42 mL, 4.9 mmol). The resulting mixture was brought to reflux with a heat gun under N₂, and then cooled to ambient temperature in a water bath. These reflux and cooling steps were repeated two more times. The mixture was then cooled to 0° C. in an ice bath. Chlorotrimethylsilane (from Aldrich, 0.69 mL, 5.4 mmol) was slowly added to the cooled mixture over a period of a few minutes. The resulting mixture was stirred at ambient temperature for 30 min, and then cooled to 0° C. The cooled mixture was slowly treated with 1,1,1,2,2-pentafluoro-4-iodobutane, which caused an exothermic reaction. The mixture was warmed to ambient temperature and then stirred for 2 hr at 50° C. Afterward, the mixture was cooled to ambient temperature resulting in an organozinc reagent. Separately, an N,N-dimethylacetamide (33 mL) solution of 4-[6-(4-trifluoromethanesulfonyloxy-phenyl)-pyridine-3-sulfonyl]-tetrahydro-pyran-4-carboxylic acid tert-butyl ester (1.5 g, 2.7 mmol, prepared in accordance with Example 26, Part E) was treated with bis(benzonitrile)dichloropalladium(II) (from Aldrich, 0.067 g, 0.174 mmol) and 2-(dicyclohexylphosphino)-2′-methylbiphenyl (from Strem Chemical, 0.11 g, 0.291 mmol) under N₂. The organozinc reagent (4.7 mL of stock solution, 8.23 mmol) was added to this mixture. The resulting mixture was stirred at 55° C. for 2 hr, and then allowed to cool to ambient temperature. Subsequently, the reaction was quenched with saturated aqueous NH₄Cl (12 mL). The mixture was then partitioned further with ethyl acetate (50 mL) and de-ionized water (50 mL). The biphasic mixture was filtered through Celite (pre-washed with ethyl acetate). The filter cake, in turn, was washed with ethyl acetate and de-ionized water. The organic layer was separated, and the aqueous layer was extracted with ethyl acetate (2×50 mL). The organic layers were combined and then washed with saturated aqueous NaHCO₃ (50 mL), washed with 1:1 brine/de-ionized water (2×50 mL), washed with brine (2×50 mL), dried over Na₂SO₄, filtered, and concentrated in vacuo. The resulting amber oil was purified by silica chromatography (eluting with 3:1 hexanes/ethyl acetate) to afford the desired ester as a yellow solid (0.67 g (clean) and 0.69 g (4:1 product/starting material), total 54% yield). The presence of the desired ester was confirmed by ¹H-NMR and ¹⁹F-NMR. LC/MS m/z=550 [M+H], 572 [M+Na].

Part B. Preparation of 4-{6-[4-(3,3,4,4,4-pentafluoro-butyl)-phenyl]-pyridine-3-sulfonyl}-tetrahydro-pyran-4-carboxylic Acid:

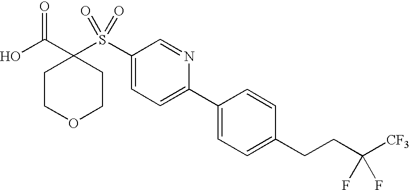

A methylene chloride (6.0 ml) solution of the ester product of Part A (2.16 g, 3.93 mmol) was treated with triethylsilane (from Aldrich, 2.0 ml, 12.5 mmol) and trifluoroacetic acid (from Aldrich, 5.0 ml, 64.9 mmol). The resulting solution was stirred at ambient temperature under N₂ for 3 days. Afterward, the mixture was concentrated in vacuo. The concentrated mixture was diluted with diethyl ether, and then concentrated in vacuo to form a glassy solid. These dilution and concentration steps were repeated two more times. The solid was then triturated in diethyl ether. Afterward, the mixture was filtered, and the resulting solids were washed with diethyl ether and dried in a vacuum oven to afford the desired carboxylic acid as a white solid (1.73 g, 89% yield). The presence of the desired carboxylic acid was confirmed by ¹H-NMR, and ¹⁹F-NMR also confirmed structure was not a trifluoroacetic acid “TFA” salt). LC/MS m/z=494 [M+H], 516 [M+Na].

Part C. Preparation of 4-{6-[4-(3,3,4,4,4-pentafluoro-butyl)-phenyl]-pyridine-3-sulfonyl}-tetrahydro-pyran-4-carboxylic acid (tetrahydro-pyran-2-yloxy)-amide:

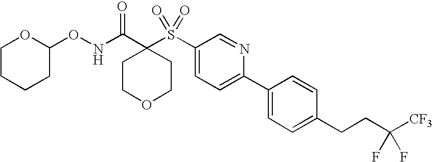

An N,N-dimethylformamide (“DMF”, 7.0 mL) solution of the carboxylic acid product from Part B (1.67 g, 3.38 mmol) was treated with 1-[3-dimethylamino)propyl]-3-ethylcarbodiimide hydrochloride (from Aldrich, 0.97 g, 5.08 mmol) and 1-hydroxybenzotriazole (from Aldrich, 0.69 g, 5.08 mmol). After stirring the mixture at ambient temperature for 15 min, 4-N-methylmorpholine (from Aldrich, 1.12 mL, 10.2 mmol) and O-(tetrahydropyranyl) hydroxylamine (from Carbogen, 0.59 g, 5.08 mmol) were added. The resulting mixture was stirred at ambient temperature under N₂ overnight. Afterward, the mixture was partitioned with ethyl acetate (25 mL) and de-ionized water (25 mL). The layers were separated, and the aqueous layer was extracted with ethyl acetate (2×25 mL). The organic layers were combined and then washed with saturated aqueous NaHCO₃ (2×15 mL), washed with 1:1 brine/de-ionized water (2×15 mL), washed with brine (2×15 mL), dried over Na₂SO₄, filtered, and concentrated in vacuo to afford the desired THP-hydroxamate as a yellow glassy solid (2.18 g, 108% mass recovery (the sample had residual DMF)). The presence of the desired THP-hydroxamate was confirmed by ¹H-NMR and ¹⁹F-NMR. LC/MS m/z=593 [M+H], 615 [M+Na].

Part D. Preparation of N-hydroxy-4-({6-[4-(3,3,4,4,4-pentafluorobutyl)phenyl]pyridin-3-yl}sulfonyl)tetrahydro-2H-pyran-4-carboxamide Hydrochloride:

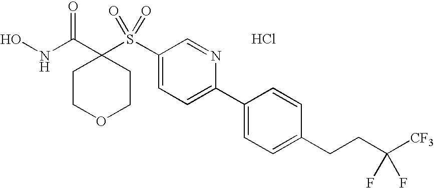

An ethyl acetate (22.6 mL) solution of the THP-hydroxamate product from Part C (2.01 g, 3.39 mmol) was treated with 1.25 N HCl in ethanol (from Fluka, 6.0 mL). This mixture was stirred at ambient temperature for 1.5 hr, during which the reaction formed a white suspension. After another 2 hr, the suspension was diluted with 4:1 diethyl ether/hexanes (50 mL). The diluted mixture was stirred for 1 hr. Afterward, the suspension was filtered, and the resulting solids were washed with diethyl ether (20 mL) and then dried in a vacuum oven to afford the desired hydroxamic acid as a white solid (1.77 g, >95% yield). The presence of the desired hydroxamic acid was confirmed by ¹H-NMR and ¹⁹F-NMR. LC/MS m/z=509 [M+H], 531 [M+Na]. HR-MS: M+H calculated for C₂₁H₂₂F₅N₂O₅S: 509.1164, found: 509.1145.

### Example 28

### Preparation of 4-[4-(5-butyl-thiophene-2-carbonyl)-3,4,5,6-tetrahydro-2H-[1,2′]bipyridinyl-5′-sulfonyl]-tetrahydro-pyran-4-carboxylic Acid Hydroxyamide Hydrochloride

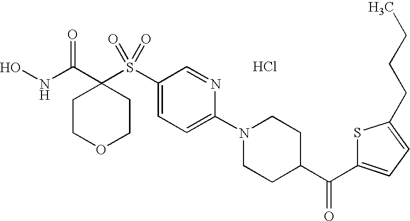

Part A. Preparation of 4-(5-butyl-thiophene-2-carbonyl)-piperidine-1-carboxylic Acid Tert-Butyl Ester:

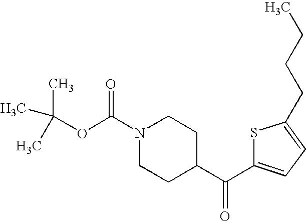

A solution of the n-butylthiophene (from Lancaster, 5.0 g, MW 140.26, 1.1 eq) in tetrahydrofuran (80 ml) at 0° C. was dripped into 1.6 M n-butyllithium in hexanes (from Aldrich, 24 ml, 1.2 eq). The resulting mixture was stirred at 0° C. for 0.5 hr under N₂. The reaction vessel was then cooled to −78° C. Afterward, a solution of 4-(methoxy-methyl-carbamoyl)-piperidine-1-carboxylic acid tert-butyl ester (8.7 g, MW 272.34, 1.0 eq) in tetrahydrofuran (30 ml) was slowly added. The dry ice bath was removed, and the mixture was allowed to warm to ambient temperature. After 3 hr, the conversion was complete. The reaction was quenched with water (50 ml). The organic was then removed in vacuo. More water (100 ml) was added. The resulting mixture was extracted with diethylether (3×100 ml). Afterward, the organic layers were combined and then washed with water (2×), washed with brine (1×), dried over Na₂SO₄, and concentrated to afford a brown oil. The oil was chromatographed (ethylacetate:hexanes , 1:9) to afford 7.5 g of the desired ester as a pale yellow solid (67% crude yield). ¹H NMR confirmed the presence of the desired ester. The “equivalents” above indicate equivalents relative to charged amount of 4-(methoxy-methyl-carbamoyl)-piperidine-1-carboxylic acid tert-butyl ester.

Part B. Preparation of the hydrochloride salt of (5-butyl-thiophen-2-yl)-piperidin-4-yl-methanone:

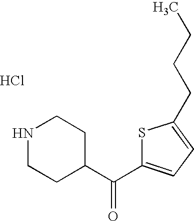

To a solution of the ester product from Part A (7.4 g, MW 351.50) in acetonitrile (10 ml) was added 4 N HCl in dioxane (40 ml, from Pierce). After 1 hr, the solvent was evaporated, and the residue was slurried in diethylether to afford the desired piperidine as white solid that was collected and dried (5.8 g, 97% yield). ¹H NMR confirmed the presence of the desired piperidine.

Part C. Preparation of 4-[4-(5-Butyl-thiophene-2-carbonyl)-3,4,5,6-tetrahydro-2H-[1,2′]bipyridinyl-5′-sulfonyl]-tetrahydro-pyran-4-carboxylic acid tert-butyl Ester:

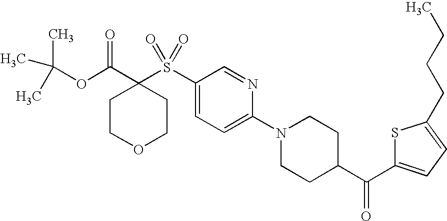

To a solution of the piperidine product of Part B (1.0 g, MW 287.85) in N,N-dimethylformamide (from Aldrich, 10 ml) was added K₂CO₃ (from Aldrich, 1.2 g, MW 138.2, 2.5 eq). After stirring the mixture for 5 min, 4-(6-bromo-pyridine-3-sulfonyl)-tetrahydro-pyran-4-carboxylic acid tert-butyl ester (1.4 g, MW 406.29, 1.0 eq, prepared in accordance with Example 26, Part C) was added. The resulting mixture was stirred at 80° C. for 2 hr. The mixture was then diluted with water (15 ml). The diluted mixture was extracted with ethylacetate (3×100 ml). The organics were combined and then washed with water (1×), washed with brine (2×), dried over Na₂SO₄, and concentrated to form a crude brown solid. This solid was recrystallized from hot methanol to afford the desired ester as a yellow solid (1.7 g, 85% yield). ¹H NMR confirmed the presence of the desired ester. The “equivalents” above indicate equivalents relative to the charged amount of the product from Part B.

Part D. Preparation of the trifluoroacetic acid salt of 4-[4-(5-butyl-thiophene-2-carbonyl)-3,4,5,6-tetrahydro-2H-[1,2]bipyridinyl-5′-sulfonyl]-tetrahydro-pyran-4-carboxylic Acid:

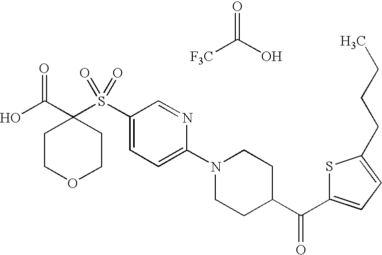

To a solution of the ester product from Part C (1.6 g, MW 576.77) in methylene chloride (5 ml) was added trifluoroacetic acid (10 ml). The resulting mixture was stirred 4 hr at ambient temperature. The mixture was then concentrated to one-third volume. Diethylether was added to the concentrated mixture. The resulting solid was collected and dried to afford the desired carboxylic acid as a tan solid (1.4 g, 82% yield). ¹H NMR and LCMS confirmed the presence of the desired carboxylic acid.

Part E. Preparation of 4-[4-(5-butyl-thiophene-2-carbonyl)-3,4,5,6-tetrahydro-2H-[1,2′]bipyridinyl-5′-sulfonyl]-tetrahydro-pyran-4-carboxylic acid (tetrahydro-pyran-2-yloxy)-amide:

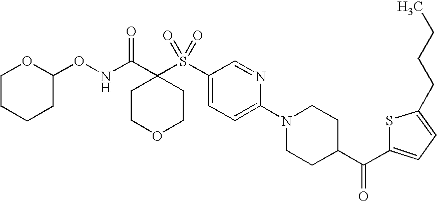

To a solution of the carboxylic acid product from Part D (1.3 g, MW 634.68) in N,N-dimethylacetamide (6 ml) was added triethylamine (from Aldrich, 0.9 ml, 3.0 eq), followed by N-hydroxybenzotriazole hydrate (from Aldrich, 0.5 g, 2.0 eq), O-(tetrahydro-2H-pyran-2-yl) hydroxylamine (0.4 g, 1.5 eq), and, lastly, 1-(3-dimethylaminopropyl)-3-ethylcarbodiimide hydrochloride (from Sigma, 1.0 g, 2.5 eq). The resulting mixture was stirred for 16 hr at ambient temperature. Afterward, the mixture was diluted with water (10 ml). The diluted mixture was extracted with ethylacetate (3×75 ml). The organics were combined and then washed with a saturated sodium bicarbonate solution (1×150 ml), washed with brine (1×150 ml), dried over Na₂SO₄, and concentrated to afford the desired THP-hydroxamate as a tan, foamy oil (1.3 g, 100+% yield). ¹H NMR and LCMS confirmed the presence of the desired THP-hydroxamate. The “equivalents” above indicate equivalents relative to the charged amount of the product from Part D.

Part F. Preparation of 4-[4-(5-butyl-thiophene-2-carbonyl)-3,4,5,6-tetrahydro-2H-[1,2′]bipyridinyl-5′-sulfonyl]-tetrahydro-pyran-4-carboxylic Acid Hydroxyamide Hydrochloride:

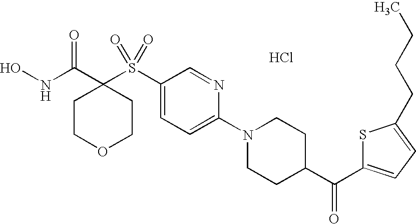

The THP-hydroxamate product from Part E (1.3 g, MW 619.79) was treated with methanol (0.5 ml) and 4 N HCl in dioxane (5 ml). The resulting mixture was stirred for 1 hr at room temperature. The solvents were concentrated to one third the volume using an N₂ stream. Diethylether was then added to the resulting residue to form a solid. The solid was collected and dried to afford the desired hydroxamic acid as a white solid (1.1 g, 100% yield). ¹H NMR confirmed the presence of the desired hydroxamic acid. HRMS confirmed (theo. M+H 535.1884; obs. M+H 535.1893).

### Example 29

### Preparation of N-hydroxy-4-{[6-(4-{2-[isobutyl(methyl)amino]-2-oxoethyl}phenyl)pyridin-3-yl]sulfonyl}tetrahydro-2H-pyran-4-carboxamide Hydrochloride

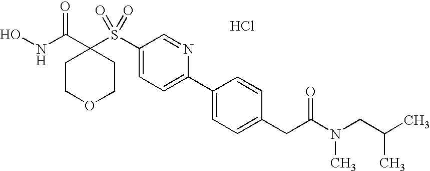

Part A. Preparation of Methyl (4-bromophenyl)acetate:

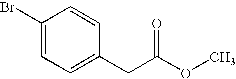

To a solution of 4-bromophenylacetic acid (10 g, 46.5 mmol) in methanol (70 mL) was slowly added thionyl chloride (4.0 mL, 55.8 mmol). The resulting mixture was heated to reflux. After 1.5 hr, the reaction mixture was concentrated in vacuo, and then partitioned between ethyl acetate and water. The organic layer was washed with saturated sodium bicarbonate, washed with brine, dried over sodium sulfate, filtered, and concentrated in vacuo to afford 10.5 g of the desired methyl ester as an oil. ESMS m/z=229 [M+H]⁺.

Part B. Preparation of methyl [4-(4,4,5,5-tetramethyl-1,3,2-dioxaborolan-2-yl)phenyl]acetate:

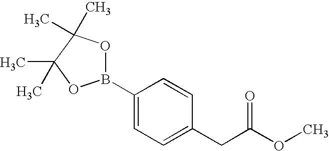

To a degassed suspension of the methyl ester product from Part A (5.0 g, 21.8 mmol), bis(pinacolato)diboron (5.8 g, 22.9 mmol), and potassium acetate (6.9 g, 69.9 mmol) in N,N-dimethylformamide (73 mL) was added bis(diphenylphosphinoferrocene)dichloro palladium II (562 mg, 0.69 mmol). The resulting mixture was heated to 80° C. for 16 hr, and then concentrated in vacuo. The concentrated mixture was then partitioned between ethyl acetate and brine. After filtering away the solids, the organic layer was dried over sodium sulfate, filtered, and concentrated in vacuo. The crude product was purified by flash chromatography (using 5-95% ethyl acetate/hexanes) to afford 3.4 g of the desired boronate as an oil. ESMS m/z=277 [M+H]⁺.

Part C. Preparation of tert-butyl 4-({6-[4-(2-methoxy-2-oxoethyl)phenyl]pyridin-3-yl}sulfonyl)tetrahydro-2H-pyran-4-carboxylate:

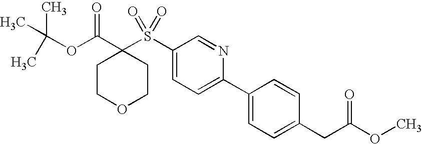

To a degassed solution of tert-butyl 4-[(6-bromopyridin-3-yl)sulfonyl]tetrahydro-2H-pyran-4-carboxylate (4.8 g, 11.8 mmol, prepared in accordance with Part C of Example 26) and the boronate product from Part B (3.4 g, 12.4 mmol) in toluene (58 mL) and ethanol (19 mL) was added a 2M solution of sodium carbonate (30 mL, 59 mmol) and bis(diphenylphosphinoferrocene)dichloro palladium II (290 mg, 0.035 mmol). The resulting mixture was heated to 75° C. for 1.5 hr, and then concentrated in vacuo. Afterward, the concentrated mixture was re-dissolved in ethyl acetate, and washed with saturated sodium bicarbonate and brine. The organic layer was dried over sodium sulfate, filtered, and concentrated in vacuo to afford 7.0 g of the desired product as a thick syrup.

ESMS m/z=476 [M+H]⁺.

Part D. Preparation of 4-[6-(4-carboxymethyl-phenyl)-pyridine-3-sulfonyl]-tetrahydro-pyran-4-carboxylic Acid Tert-Butyl Ester:

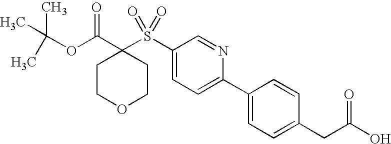

To a solution of the crude product from Part C (6.3 g, 13.3 mmol) in 1:1 mixture of tetrahydrofuran and water (40 mL) was added lithium hydroxide (1.7 g, 39.8 mmol). After 1 hr, the mixture was washed with diethyl ether. The aqueous layer was acidified to a pH of 3, and then extracted with ethyl acetate. The combined organic layers were washed with brine, dried over sodium sulfate, filtered, and concentrated in vacuo to afford 5.0 g of the desired acid as a tan solid. ESMS m/z=462 [M+H]⁺.

Part E. Preparation of 4-(6-{4-[(isobutyl-methyl-carbamoyl)-methyl]-phenyl}-pyridine-3-sulfonyl)-tetrahydro-pyran-4-carboxylic Acid Tert-Butyl Ester:

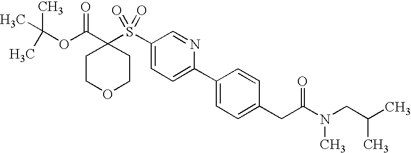

To a solution of the acid product from Part D (403 mg, 0.81 mmol) in N,N-dimethylformamide were added the following in the following order: 1-hydroxybenzotriazole (153 mg, 1.13 mmol), triethylamine (340 μL, 2.43 mmol), methylisobutylamine (0.71 mg, 1.94 mmol), and 1-(3-dimethyaminopropyl)-3-ethylcarbodiimide hydrochloride (217 mg, 1.13 mmol). The resulting mixture was heated to 40° C. After 8 hr, the mixture was diluted with ethyl acetate, washed with saturated sodium bicarbonate (2×), washed with brine (5×), dried over sodium sulfate, filtered, and concentrated in vacuo to afford 524 mg of the desired product as a brown oil. ESMS m/z=531 [M+H]⁺. The crude material was carried forward with no further purification.

Part F. Preparation of 4-(6-{4-[(isobutyl-methyl-carbamoyl)-methyl]-phenyl}-pyridine-3-sulfonyl)-tetrahydro-pyran-4-carboxylic Acid:

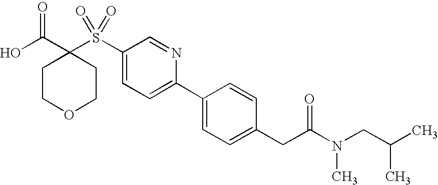

The crude product from Part E (534 mg, 0.99 mmol) was dissolved in trifluoroacetic acid (5 mL). After 2.5 hr, the resulting mixture was diluted with methylene chloride, and then concentrated in vacuo (3×) to afford 780 mg of the desired acid as a brown oil. ESMS m/z=475 [M+H]⁺.

Part G. Preparation of 4-(6-{4-[(isobutyl-methyl-carbamoyl)-methyl]-phenyl}-pyridine-3-sulfonyl)-tetrahydro-pyran-4-carboxylic acid (tetrahydro-pyran-2-yloxy)-amide:

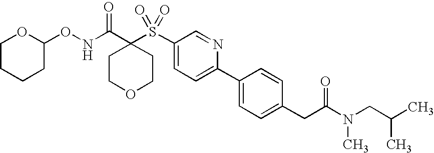

To a solution of the crude acid product from Part F (780 mg, 1.32 mmol) in N,N-dimethylformamide (5 ml) were added the following in the following order: 1-hydroxybenzotriazole (251 mg, 1.86 mmol), triethyl amine (0.55 mL, 3.96 mmol), tetrahydropyranhydroxylamine (463 mg, 3.96 mmol), and 1-(3-dimethyaminopropyl)-3-ethylcarbodiimide hydrochloride (357 mg, 1.86 mmol). The resulting mixture was heated at 40° C. for 10 hr, after which HPLC indicated complete consumption of the acid starting material (i.e., the acid from Part F). The mixture was then diluted with ethyl acetate, washed with saturated sodium bicarbonate solution (2×), washed with brine (5×), dried over sodium sulfate, filtered, and concentrated in vacuo. The crude solid was purified by reverse phase column chromatography using a gradient eluant of 10-50% acetonitrile/water to afford 292 mg of the desired THP-protected hydroxamate as a white solid. ESMS m/z=490 [M+H]⁺.

Part H. Preparation of N-hydroxy-4-{[6-(4-{2-[isobutyl(methyl)amino]-2-oxoethyl}phenyl)pyridin-3-yl]sulfonyl}tetrahydro-2H-pyran-4-carboxamide Hydrochloride:

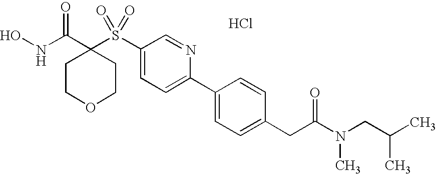

To a solution of the hydroxamate product from Part G (292 mg, 0.51 mmol) in ethyl acetate (4 mL) was added 1.25 M HCl in ethanol (0.94 mL, 1.17 mmol). After approximately 40 min, the resulting solid was isolated by filtration and trituration with hexanes to afford 83 mg of the desired hydroxamic acid as an off-white solid. HRMS calcd. for C₂₄H₃₁N₃O₆S: 490.2006 [M+H]⁺, found: 490.2027.

Examples 30-40. Additional compounds may prepared by one skilled in the art using methods similar to those described in Example 29 (either alone or in combination with techniques shown in the other examples above and/or techniques known in the art) with either the above described intermediate acid or a similarly prepared variant. Examples of such compounds prepared by Applicants include those shown in Table 1 corresponding in structure to Formula III.

TABLE 1

| | |
|---|---|
| | (III) |
| 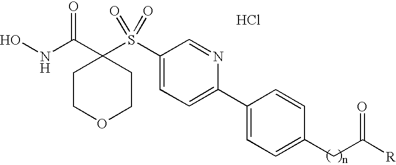 | |

| Ex- | | | | |
|---|---|---|---|---|
| am- | | | | |
| ple | | | Calculated | Observed |
| No. | n | R | Mass | Mass |
| 30 | 1 | piperidine | 488.1850 | 488.1861 |
| 31 | 1 | butylamine | 476.1850 | 476.1843 |
| 32 | 1 | N-methyl butylamine | 490.2006 | 490.1999 |
| 33 | 1 | N-methyl 4-trifluoromethoxyaniline | 594.1516 | 594.1517 |
| 34 | 0 | butylamine | 462.1693 | 462.1682 |
| 35 | 0 | piperidine | 474.1693 | 474.1690 |
| 36 | 0 | N-methyl aniline | 496.1537 | 496.1541 |
| 37 | 0 | N,N-diethylamine | 462.1693 | 462.1681 |
| 38 | 0 | morpholine | 476.1486 | 476.1458 |
| 39 | 0 | N-methyl 4-trifluoromethoxyaniline | 580.1360 | 580.1361 |
| 40 | 0 | N-methyl isobutylamine | 476.1850 | 476.1846 |

Examples 41-42. Additional compounds may prepared by one skilled in the art using methods similar to those shown in the above Examples, either alone or in combination with other techniques known in the art. Examples of such compounds prepared by Applicants include those shown in Table 2.

TABLE 2

| Example | | Calculated | Observed |
|---|---|---|---|
| No. | Structure | Mass | Mass |
| 41 | 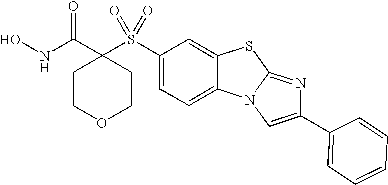 | | |
| 42 | 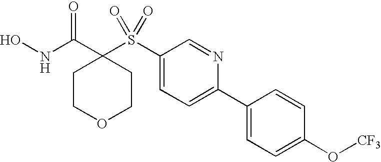 | 447.0832 | 447.0826 |

### Examples 43-84

### In Vitro MMP Inhibition Analysis

Several compounds and salts were analyzed in an in vitro assay to determine their ability to inhibit the MMP cleavage of peptide substrates. Inhibition constant (Kᵢ) were calculated from the assayed compound-MMP interactions.

Human recombinant MMP-1, MMP-2, MMP-9, MMP-13, and MMP-14 were used in this assay. All enzymes were prepared in Assignee's laboratories following usual laboratory procedures. Protocols for the preparation and use of these enzymes are available in the scientific literature. See, e.g., Enzyme Nomenclature (Academic Press, San Diego, Calif., 1992) (and the citations therein). See also, Freije et al., J. Biol. Chem., 269(24), 16766-16773 (1994).

The MMP-1 proenzyme was purified from the spent media of MMP-1-transfected HT-1080 cells provided by Dr. Harold Welgus of Washington University (St. Louis, Mo.). The protein was purified on a zinc chelating column.

The MMP-2 proenzyme was purified by gelatin Sepharose chromatography from MMP-2- transfected p2AHT2 cells provided by Dr. Gregory Goldberg of Washington University (St. Louis, Mo.).

The MMP-9 proenzyme was purified by gelatin Sepharose chromatography from spent media of MMP-9-transfected HT1080 cells provided by Dr. Howard Welgus of Washington University (St. Louis, Mo.).

The MMP-13 was obtained as a proenzyme from a full-length cDNA clone using baculovirus, as described by V. A. Luckow, “Insect Cell Expression Technology,” Protein Engineering: Principles and Practice, pp. 183-218 (edited by J. L. Cleland et al., Wiley-Liss, Inc., 1996). The expressed proenzyme was first purified over a heparin agarose column, and then over a chelating zinc chloride column. The proenzyme was then activated by APMA for use in the assay. Further details on baculovirus expression systems may be found in, for example, Luckow et al., J. Virol., 67(8):4566-79 (1993). See also, O'Reilly et al, Baculovirus Expression Vectors: A Laboratory Manual (W. H. Freeman and Co., New York, N.Y., 1992). See also, King et al., The Baculovirus Expression System: A Laboratory Guide (Chapman & Hall, London, England, 1992).

The MMP-14 full length cDNA was provided by Dr. Gregory Goldberg of Washington University (St. Louis, Mo.). The catalytic domain enzyme was expressed in E. Coli inclusion bodies, solubilized in urea, purified on a preparative C-14 reverse phase HPLC column, and then refolded in the presence of zinc acetate and purified for use.

All MMPs were activated using 4-aminophenylmercuric acetate (“APMA”, Sigma Chemical, St. Louis, Mo.) or trypsin. MMP-9 also was activated using human recombinant MMP-3 (purified in Assignee's laboratory following standard cloning and purification techniques).

The following fluorogenic, methoxycoumarin-containing polypeptide substrate (A) was used in the MMP inhibition assays:

MCA-ArgProLeuGlyLeuDpaAlaArgGluArgNH₂ (A)

“MCA” is 7-methoxycoumarin-4-yl acetyl. Substrate (A) was prepared Assignee's laboratory. In the absence of MMP inhibitory activity, the substrate is cleaved at the Gly-Leu peptide bond. This cleavage separates the highly fluorogenic peptide from the 2,4-dinitrophenyl quencher, thus resulting in increase of fluorescent intensity.

The stock solutions of the assayed compounds and salts were prepared in 1% dimethyl sulfoxide (DMSO). These stock solutions were diluted in Buffer A (100 mM Tris-HCl, 100 mM NaCl, 10 mM CaCl₂, 0.05% polyoxyethylene 23 lauryl ether, pH 7.5) to obtain solutions with different compound concentrations, i.e., assay solutions with different concentrations of the assayed MMP inhibitory compound. The experiment controls contained the same amount of Buffer A/DMSO as the assayed sample, but contained none of the tested compound or salt.

The assays from which the Kᵢ determinations were made were performed as follows. The assayed compound samples were incubated in separate wells of untreated white polystyrene plates (Nunc Nalgene International, Rochester, N.Y.), and analyzed on a Tecan SpectraFlour Plus plate reader. The excitation wavelength was 330 nm, and the emission wavelength -420 nm. All samples (assayed compounds and controls) were incubated in separate plate wells at room temperature for 1 hr in the presence of 4 μM of MMP substrate (A). In the absence of MMP inhibitory activity, substrate (A) was cleaved at the Gly-Leu bond resulting in an increase of relative fluorescence. Inhibition was observed as a reduced rate of this increase in relative fluorescence. The various compounds were analyzed using a single low enzyme concentration with a single substrate concentration fixed at or below the Kₘ. This protocol is a modification of method by Knight et al., FEBS Lett., 296(3), 263-266 (1992). Apparent inhibitory constants were determined by non-linear regression of reaction velocity as a function of inhibitor and enzyme concentration using Morrison's equation, as described by Kuzmic, P., et al., Anal. Biochem., 286(1):45-50 (2000). Modifications were made in the non-linear regression method to allow a common control reaction rate and effective enzyme concentration to be shared between all dose-response relationships on a given assay plate. Since the substrate concentration was chosen to be at or below the Kₘ, the apparent Kᵢ's from this analysis were reported as Kᵢ's without correction for the influence of substrate.

The above protocols were used to determine MMP inhibition Kᵢ constants for the compounds in Examples 1, 2, and 4-42 above. All Kᵢ values in Table 3 are given in nM units.

TABLE 3

| Ex- | | | | | | |
|---|---|---|---|---|---|---|
| am- | | | | | | |
| ple | | MMP-1 | MMP-2 | MMP-9 | MMP-13 | MMP-14 |
| No. | Compound | Kᵢ | Kᵢ | Kᵢ | Kᵢ | Kᵢ |
| 43 | 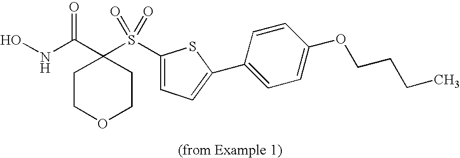 | >1250 | 0.483 | 0.806 | 0.127 | 466 |
| 44 | 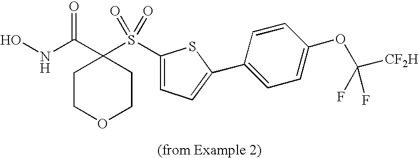 | 1120 | 0.173 | 0.354 | 0.134 | 429 |
| 45 | 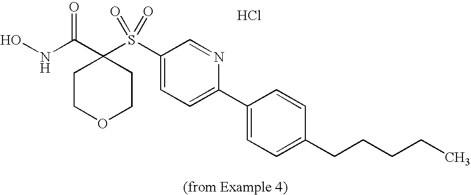 | >10000 | 2.08 | 8.96 | 1.19 | 3490 |
| 46 | 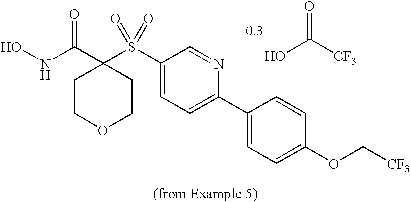 | 4340 | 0.367 | 0.988 | 0.169 | 852 |
| 47 | 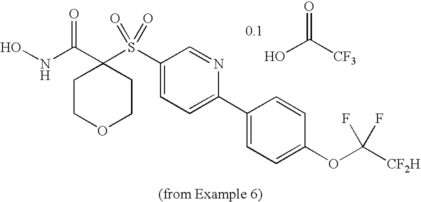 | 1490 | 0.274 | 1.53 | 0.306 | 2130 |
| 48 | 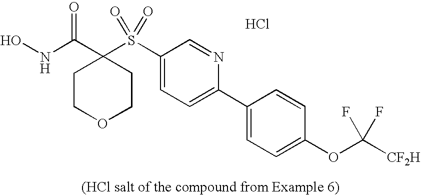 | 1240 | 0.222 | 1.06 | 0.192 | 1850 |
| 49 | 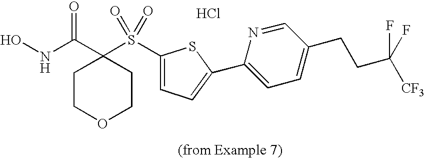 | >10000 | 3.14 | 10.2 | 0.822 | 9250 |
| 50 | 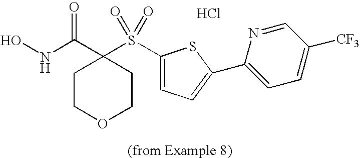 | >10000 | 29.4 | 133 | 10.3 | 2820 |
| 51 | 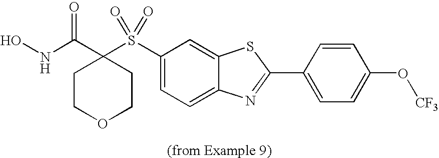 | 6530 | 2.07 | 0.636 | 0.275 | 3330 |
| 52 | 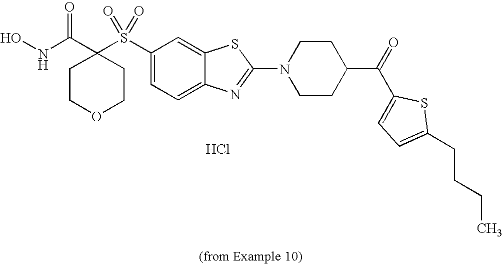 | >10000 | 76.3 | 837 | 1.58 | >10000 |
| 53 | 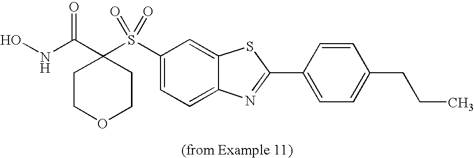 | >10000 | 3.52 | 12.8 | 0.314 | >10000 |
| 54 | 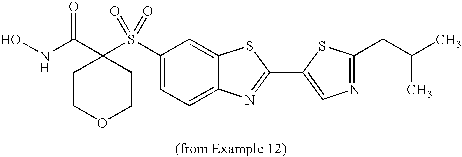 | >10000 | 35.4 | 99 | 2.73 | >10000 |
| 55 | 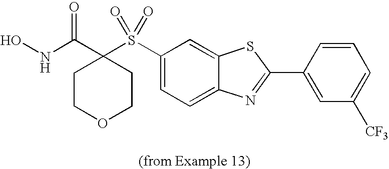 | >10000 | 621 | 3310 | 177 | >10000 |
| 56 | 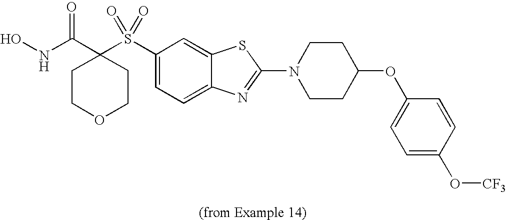 | >10000 | 40.3 | 68.5 | 3.43 | 798 |
| 57 | 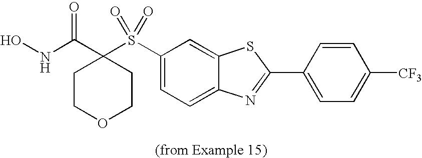 | >10000 | 1.86 | 0.964 | 0.384 | 2160 |
| 58 | 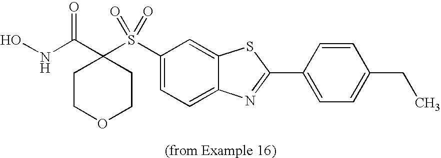 | >10000 | 1.51 | 1.06 | 0.336 | 2500 |
| 59 | 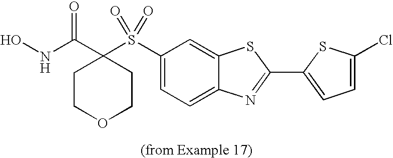 | >1250 | 2.01 | 5.09 | 0.588 | 927 |
| 60 | 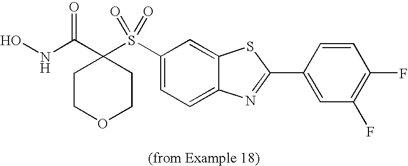 | 2840 | 10.3 | 14.1 | 1.54 | 707 |
| 61 | 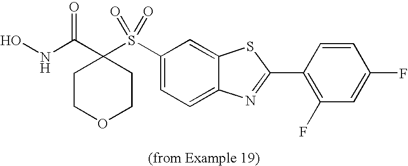 | >10000 | 14.8 | 21.5 | 2.94 | >10000 |
| 62 | 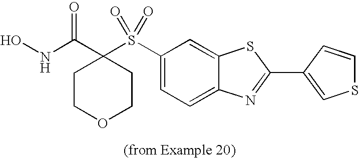 | 2020 | 4.83 | 40.5 | 1.36 | 355 |
| 63 | 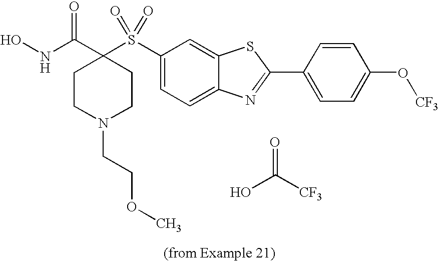 | 9160 | 2.25 | 0.624 | 0.309 | 4230 |
| 64 | 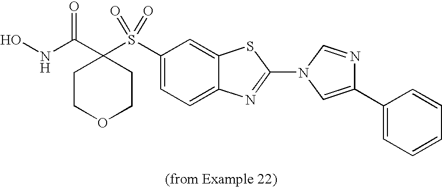 | >10000 | 151 | 799 | 22.1 | 3830 |
| 65 | 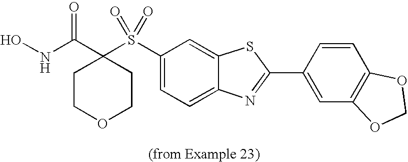 | >1250 | 4.43 | 7.2 | 1.05 | 686 |
| 66 | 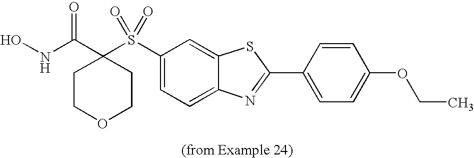 | >312 | 2.21 | 3.37 | 0.082 | >312 |
| 67 | 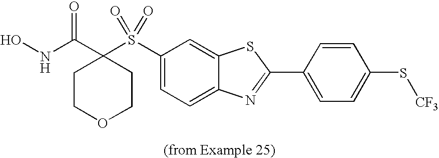 | >10000 | 2.76 | 0.471 | 0.293 | 3605 |
| 68 | 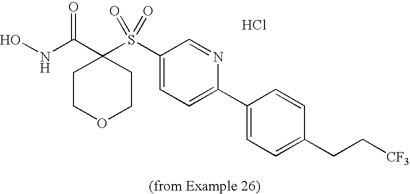 | >10000 | 0.665 | 2.82 | 0.721 | 2450 |
| 69 | 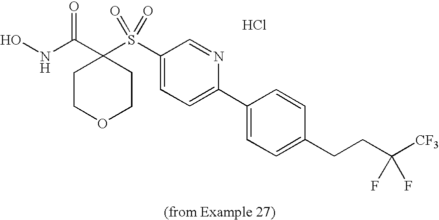 | >10000 | 0.766 | 8.93 | 0.531 | 5070 |
| 70 | 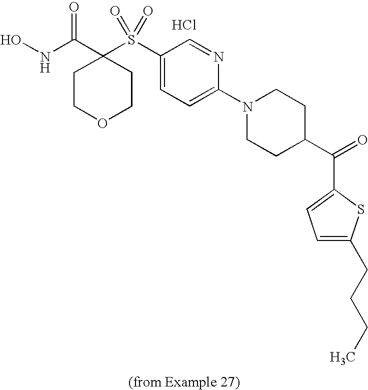 | >10000 | 1840 | >10000 | 1.8 | >10000 |
| 71 | 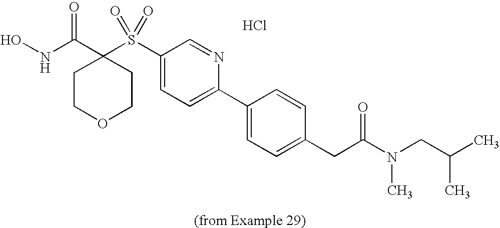 | >10000 | 553 | >10000 | 1.93 | >10000 |
| 72 | 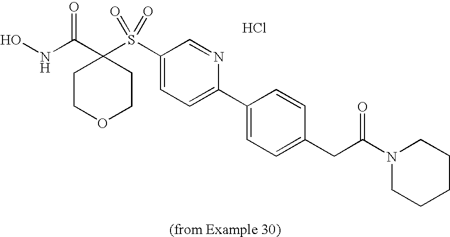 | >10000 | 305 | 7770 | 1.59 | >10000 |
| 73 | 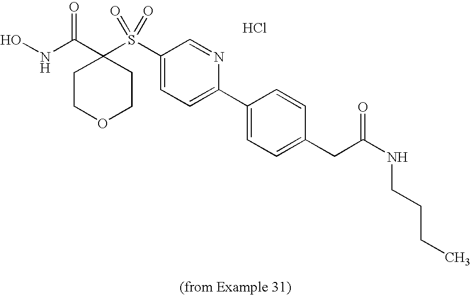 | >10000 | 91.8 | 2220 | 18.8 | >10000 |
| 74 | 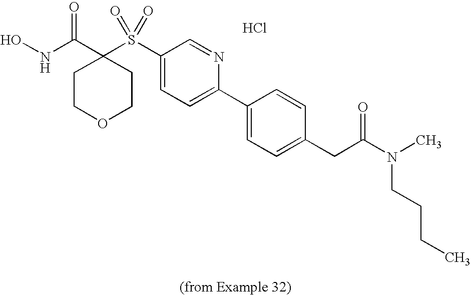 | >10000 | 387 | 4930 | 1.91 | >10000 |
| 75 | 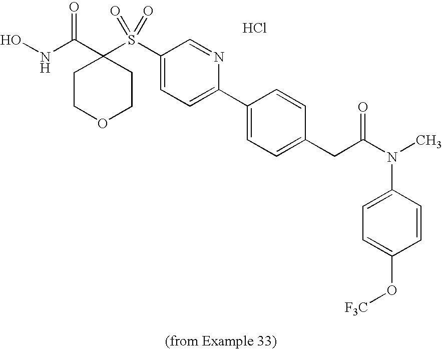 | >10000 | 0.218 | 0.159 | 140 | >10000 |
| 76 | 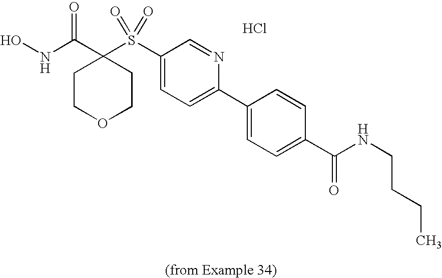 | 4200 | 26.3 | 428 | 5.59 | >10000 |
| 77 | 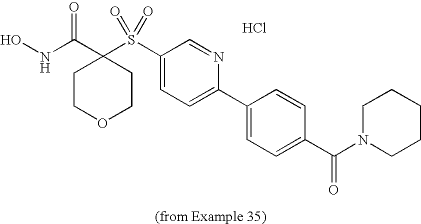 | >10000 | 4970 | >10000 | 477 | >10000 |
| 78 | 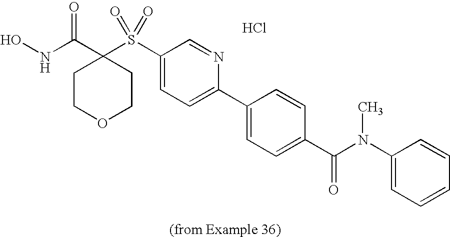 | >10000 | 895 | 1290 | 447 | >10000 |
| 79 | 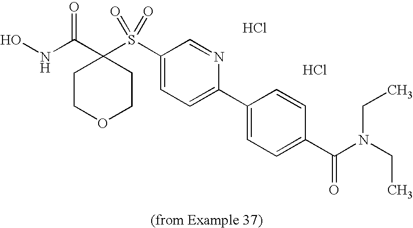 | >10000 | 8200 | >10000 | 607 | >10000 |
| 80 | 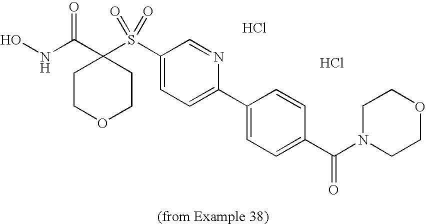 | >10000 | 1760 | >10000 | 469 | >10000 |
| 81 | 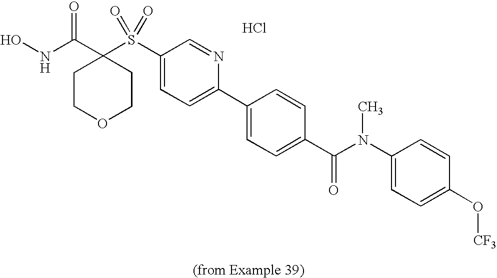 | >10000 | 2060 | 3400 | 264 | >10000 |
| 82 | 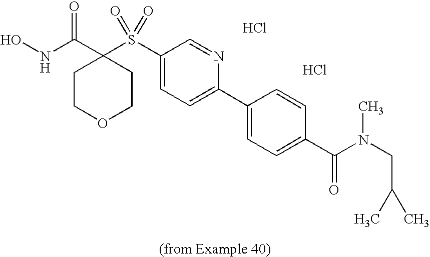 | >10000 | 668 | 894 | 229 | >10000 |
| 83 | 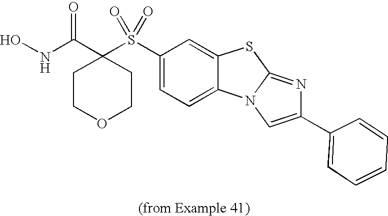 | >10000 | 46.6 | 2040 | 577 | 3150 |
| 84 | 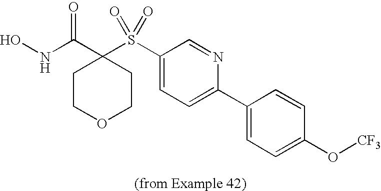 | 4480 | 0.819 | 4.26 | 0.79 | 671 |

### Example 85

### In Vivo Angiogenesis Assay

The study of angiogenesis depends on a reliable and reproducible model for the stimulation and inhibition of a neovascular response. The corneal micropocket assay provides such a model of angiogenesis in the cornea of a mouse. See, Kenyon, BM, et al., “A Model of Angiogenesis in the Mouse Cornea”, Investigative Ophthalmology & Visual Science, Vol. 37(8):1625-1632 (July 1996).

In this assay, uniformly sized Hydron™ pellets containing bFGF and sucralfate are prepared and surgically implanted into the stroma mouse cornea adjacent to the temporal limbus. The pellets are formed by making a suspension of 20 μL sterile saline containing 10 μg recombinant bFGF, 10 mg of sucralfate and 10 μL of 12 percent Hydron™ in ethanol. The slurry is then deposited on a 10×10 mm piece of sterile nylon mesh. After drying, the nylon fibers of the mesh are separated to release the pellets.

The corneal pocket is made by anesthetizing a 7 week old C57B1/6 female mouse, then proptosing the eye with a jeweler's forceps. Using a dissecting microscope, a central, intrastromal linear keratotomy of approximately 0.6 mm in length is performed with a #15 surgical blade, parallel to the insertion of the lateral rectus muscle. Using a modified cataract knife, a lamellar micropocket is dissected toward the temporal limbus. The pocket is extended to within 1.0 mm of the temporal limbus. A single pellet is placed on the corneal surface at the base of the pocket with a jeweler's forceps. The pellet is then advanced to the temporal end of the pocket. Antibiotic ointment is then applied to the eye.

Mice are dosed on a daily basis for the duration of the assay. Dosing of the animals is based on bioavailability and overall potency of the compound. An exemplary dose is 10 or 50 mg/kg (mpk) bid, po. Neovascularization of the corneal stroma is permitted to continue under the influence of the assayed compound for 2 days. At that point, the degree of angiogenic inhibition is scored by viewing the neovascular progression with a slit lamp microscope.

The mice are anesthetized and the studied eye is once again proptosed. The maximum vessel length of neovascularization, extending from the limbal vascular plexus toward the pellet is measured. In addition, the contiguous circumferential zone of neovascularization is measured as clock hours, where 30 degrees of arc equals one clock hour. The area of angiogenesis is calculated as follows.

$$area = \frac{( 0.4 \times clock hours \times 3.14 \times vessel length ( in mm ) )}{2}$$

Five to six mice should be utilized for each compound in each study. The studied mice are thereafter compared to control mice and the difference in the area of neovascularization is recorded as an averaged value. A contemplated compound typically exhibits about 25 to about 75 percent inhibition, whereas the vehicle control exhibits zero percent inhibition.

### Example 86

### Tumor Necrosis Factor Assays

Cell Culture.

The cells used in the assay are the human monocytic line U-937 (ATCC CRL-1593). The cells are grown in RPMI w/10% FCS and PSG supplement (R-10) and are not permitted to overgrow. The assay is carried out as follows:

1. Count, then harvest cells by centrifugation. Resuspend the pellet in R-10 supplement to a concentration of 1.540×10⁶ cells/mL.

2. Add test compound in 65 uL R-10 to the appropriate wells of a 96-well flat bottom tissue culture plate. The initial dilution from a DMSO stock (100 mM compound) provides a 400 uM solution, from which five additional three-fold serial dilutions are made. Each dilution of 65 ul (in triplicate) yields final compound test concentrations of 100 μM, 33.3 μM, 11.1 μM, 3.7 μM, 1.2 μM and 0.4 μM.

3. The counted, washed and resuspended cells (200,000 cells/well) in 130 μL are added to the wells.

4. Incubation is for 45 min to 1 hr at 37° C. in 5% CO₂ in a water saturated container.

5. R-10 (65 uL)containing 160 ng/mL PMA (Sigma) is added to each well.

6. The test system is incubated at 37° C. in 5% CO₂ overnight (18-20 hr) under 100% humidity.

7. Supernatant, 150 μL, is carefully removed from each well for use in the ELISA assay.

8. For toxicity, a 50 μL aliquot of working solution containing 5 mL R-10, 5 mL MTS solution [CellTiter 96 AQueous One Solution Cell Proliferation Assay Cat.#G358/0,1 (Promega Biotech)] and 250 ul PMS solution are added to each well containing the remaining supernatant and cells and the cells incubated at 37° C. in 5% CO₂ until the color develops. The system is excited at 570 nm and read at 630 nm.

TNF Receptor II ELISA Assay

1. Plate 100 μL/well 2 ug/mL mouse anti-human TNFrII antibody (R&D Systems #MAB226) in 1×PBS (pH 7.1, Gibco) on NUNC-Immuno Maxisorb plate. Incubate the plate at 4° C. overnight (about 18-20 hr).

2. Wash the plate with PBS-Tween (1×PBS w/0.05% Tween).

3. Add 200 1L 5% BSA in PBS and block at 37° C. in a water saturated atmosphere for 2 hr.

4. Wash the plate with PBS-Tween.

5. Add sample and controls (100 ul of each) to each well. The standards are 0, 50, 100, 200, 300 and 500 pg recombinant human TNFrII (R&D Systems #226-B2) in 100 μL 0.5% BSA in PBS. The assay is linear to between 400-500 pg of standard.

6. Incubate at 37° C. in a saturated atmosphere for 1.5 hr.

7. Wash the plate with PBS-Tween.

8. Add 100 μL goat anti-human TNFrII polyclonal (1.5 μg/mL R&D Systems #AB226-PB in 0.5% BSA in PBS).

9. Incubate at 37° C. in a saturated atmosphere for 1 hr.

10. Wash the plate with PBS-Tween.

11. Add 100 μL anti-goat IgG-peroxidase (1:50,000 in 0.5% BSA in PBS, Sigma #A5420).

12. Incubate at 37° C. in a saturated atmosphere for 1 hr.

13. Wash the plate with PBS-Tween.

14. Add 10 μL KPL TMB developer, develop at room temperature (usually about 10 min), then terminate with phosphoric acid and excite at 450 nm and read at 570 nm.

TNFα ELISA Assay.

Coat Immulon® 2 plates with 0.1 mL/well of lug/mL Genzyme mAb in 0.1 M NaHCO3 pH 8.0 buffer overnight (about 18-20 hr) at 4° C., wrapped tightly in Saran® wrap.

Flick out coating solution and block plates with 0.3 mL/well blocking buffer overnight at 4° C., wrapped in Saran® wrap.

Wash wells thoroughly 4× with wash buffer and completely remove all wash buffer. Add 0.1 mL/well of either samples or rhTNFα standards. Dilute samples if necessary in appropriate diluant (e.g. tissue culture medium). Dilute standard in same diluant. Standards and samples should be in triplicates.

Incubate at 37° C. for 1 hr in humidified container.

Wash plates as above. Add 0.1 mL/well of 1:200 dilution of Genzyme rabbit anti-hTNFa.

Repeat incubation.

Repeat wash. Add 0.1 mL/well of 1 μg/mL Jackson goat anti-rabbit IgG (H+L)-peroxidase.

Incubate at 37° C. for 30 min.

Repeat wash. Add 0.1 mL/well of peroxide-ABTS solution.

Incubate at room temperature for 5-20 min.

Read OD at 405 nm.

Reagents are:

Genzyme mouse anti-human TNF monoclonal (Cat.# 80-3399-01)

Genzyme rabbit anti-human TNF polyclonal (Cat.#IP-300)

Genzyme recombinant human TNF (Cat.#TNF-H).

Jackson Immunoresearch peroxide-conjugated goat anti-rabbit IgG (H+L) (Cat.#111-035-144).

Kirkegaard/Perry peroxide ABTS solution (Cat#50-66-01).

Immulon 2 96-well microtiter plates.

Blocking solution is 1 mg/mL gelatin in PBS with 1× thimerasol.

Wash buffer is 0.5 mL Tween® 20 in 1 liter of PBS.

### Example 87

### In Vitro Aggrecanase Inhibition Analysis

Assays for measuring the potency (IC₅₀) of a compound toward inhibiting aggrecanase are known in the art.

One such assay, for example, is reported in European Patent Application Publ. No. EP 1 081 137 A1. In that assay, primary porcine chondrocytes from articular joint cartilage are isolated by sequential trypsin and collagenase digestion followed by collagenase digestion overnight and are plated at 2×10⁵ cells per well into 48 well plates with 5 μCi/ml³⁵S (1000 Ci/mmol) sulfur in type 1 collagen coated plates. Cells are allowed to incorporate label into their proteoglycan matrix (approximately 1 week) at 37° C. under an atmosphere of 5% CO₂. The night before initiating the assay, chondrocyte monolayers are washed 2 times in DMEM/1% PSF/G and then allowed to incubate in fresh DMEM/1% FBS overnight. The next morning, chondrocytes are washed once in DMEM/1% PSF/G. The final wash is allowed to sit on the plates in the incubator while making dilutions. Media and dilutions are made as described in the following Table 4:

TABLE 4

| | |
|---|---|
| control media | DMEM alone |
| IL-1 media | DMEM + IL-1 (5 ng/ml) |
| drug dilutions | Make all compound stocks at 10 mM in DMSO. |
| | Make a 100 μM stock of each compound in DMEM in |
| | 96-well plate. Store in freezer overnight. |
| | The next day, perform serial dilutions in DMEM with |
| | IL-1 to 5 μM, 500 nM, and 50 nM. |
| | Aspirate final wash from wells and add 50 μM of |
| | compound from above dilutions to 450 μL of IL-1 media |
| | in appropriate wells of the 48 well plates. |
| | Final compound concentrations equal 500 nM, |
| | 50 nM, and 5 nM. All samples completed in triplicate |
| | with control and IL-1 alone on each plate. |

Plates are labeled and only the interior 24 wells of the plate are used. On one of the plates, several columns are designated as IL-1 (no drug) and control (no IL-1, no drug). These control columns are periodically counted to monitor ³⁵S-proteoglycan release. Control and IL-1 media are added to wells (450 μL) followed by compound (50 μL) so as to initiate the assay. Plates are incubated at 37° C. with 5% CO₂ atmosphere. At 40-50% release (when CPM from IL-1 media is 4-5 times control media) as assessed by liquid scintillation counting (LSC) of media samples, the assay is terminated (about 9 to about 12 hours). Media is removed from all wells and placed into scintillation tubes. Scintillate is added and radioactive counts are acquired (LSC). To solubilize cell layers, 500 μL of papain digestion buffer (0.2 M Tris, pH 7.0, 5 mM DTT, and 1 mg/ml papain) is added to each well. Plates with digestion solution are incubated at 60° C. overnight. The cell layer is removed from the plates the next day and placed in scintillation tubes. Scintillate is then added, and samples counted (LSC). The percent of released counts from the total present in each well is determined. Averages of the triplicates are made with control background subtracted from each well. The percent of compound inhibition is based on IL-1 samples as 0% inhibition (100% of total counts).

Another assay for measuring aggrecanase inhibition is reported in WIPO Int'l Publ. No. WO 00/59874. That assay reportedly uses active aggrecanase accumulated in media from stimulated bovine cartilage (BNC) or related cartilage sources and purified cartilage aggrecan monomer or a fragment thereof as a substrate. Aggrecanase is generated by stimulation of cartilage slices with interleukin-1 (IL-1), tumor necrosis factor alpha (TNF-α), or other stimuli. To accumulate BNC aggrecanase in culture media, cartilage reportedly is first depleted of endogenous aggrecan by stimulation with 500 ng/ml human recombinant IL-β for 6 days with media changes every 2 days. Cartilage is then stimulated for an additional 8 days without media change to allow accumulation of soluble, active aggrecanase in the culture media. To decrease the amounts of matrix metalloproteinases released into the media during aggrecanase accumulation, agents which inhibit MMP-1, -2, -3, and -9 biosynthesis are included during stimulation. This BNC conditioned media containing aggrecanase activity is then used as the source of aggrecanase for the assay. Aggrecanase enzymatic activity is detected by monitoring production of aggrecan fragments produced exclusively by cleavage at the Glu373-Ala374 bond within the aggrecan core protein by Western analysis using the monoclonal antibody, BC-3 (Hughes, et al., Biochem J, 305:799-804 (1995)). This antibody reportedly recognizes aggrecan fragments with the N-terminus, 374ARGSVIL, generated upon cleavage by aggrecanase. The BC-3 antibody reportedly recognizes this neoepitope only when it is at the N-terminus and not when it is present internally within aggrecan fragments or within the aggrecan protein core. Only products produced upon cleavage by aggrecanase reportedly are detected. Kinetic studies using this assay reportedly yield a Km of 1.5+/−0.35 μM for aggrecanase. To evaluate inhibition of aggrecanase, compounds are prepared as 10 mM stocks in DMSO, water, or other solvents and diluted to appropriate concentrations in water. Drug (50 μL) is added to 50 μL of aggrecanase-containing media and 50 μL of 2 mg/ml aggrecan substrate and brought to a final volume of 200 μL in 0.2 M Tris, pH 7.6, containing 0.4 M NaCl and 40 mM CaCl₂. The assay is run for 4 hr at 37° C., quenched with 20 mM EDTA, and analyzed for aggrecanase-generated products. A sample containing enzyme and substrate without drug is included as a positive control and enzyme incubated in the absence of substrate serves as a measure of background. Removal of the glycosaminoglycan side chains from aggrecan reportedly is necessary for the BC-3 antibody to recognize the ARGSVIL epitope on the core protein. Therefore, for analysis of aggrecan fragments generated by cleavage at the Glu373-Ala374 site, proteoglycans and proteoglycan fragments are enzymatically deglycosylated with chondroitinase ABC (0.1 units/10 μg GAG) for 2 hr at 37° C. and then with keratanase (0.1 units/10 μg GAG) and keratanase II (0.002 units/10 μg GAG) for 2 hr at 37° C. in buffer containing 50 mM sodium acetate, 0.1 M Tris/HCl, pH 6.5. After digestion, aggrecan in the samples is precipitated with 5 volumes of acetone and resuspended in 30 μL of Tris glycine SDS sample buffer (Novex) containing 2.5% beta mercaptoethanol. Samples are loaded and then separated by SDS-PAGE under reducing conditions with 4-12% gradient gels, transferred to nitrocellulose and immunolocated with 1:500 dilution of antibody BC3. Subsequently, membranes are incubated with a 1:5000 dilution of goat anti-mouse IgG alkaline phosphatase second antibody and aggrecan catabolites visualized by incubation with appropriate substrate for 10-30 minutes to achieve optimal color development. Blots are quantitated by scanning densitometry and inhibition of aggrecanase determined by comparing the amount of product produced in the presence versus absence of compound.

The above detailed description of preferred embodiments is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

We claim:

1. A compound or a salt thereof, wherein:
the compound corresponds in structure to Formula 1-1: 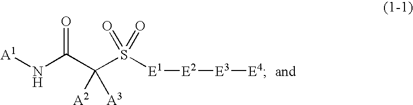
A¹ is selected from the group consisting of hydrogen, hydroxyl, carbocyclyloxy, and hete rocyclyloxy; and
as to A² and A³:
A² and A³, together with the carbon to which they are both bonded, form heterocyclyl or carbocyclyl, wherein:
the heterocyclyl or carbocyclyl optionally is substituted with up to 3 independently selected R^{x} substituents, and
the heterocyclyl or carbocyclyl optionally is substituted with two substituents such that the two substituents, together with the atom(s) to which they are bonded, form a carbocyclyl or heterocyclyl, wherein:
the optional heterocyclyl or carbocyclyl is, in turn, optionally substituted with up to 3 independently selected Rx substituents, or
A² and A³ are independently selected from the group consisting of hydrogen, alkoxyalkyl, alkylthioalkyl, alkenyl, alkynyl, carbocyclyl, carbocyclylalkyl, carbocyclylalkenyl, carbocyclylalkynyl, carbocyclyloxyalkyl, carbocyclylalkoxyalkyl, carbocyclylalkylthio, carbocyclylthioalkyl, carbocyclylalkylthioalkyl, heterocyclyl, heterocyclylalkyl, heterocyclylalkenyl, heterocyclylalkynyl, heterocyclyloxyalkyl, heterocyclylalkoxyalkyl, heterocyclylalkylthio, heterocyclylthioalkyl, and heterocyclylalkylthioalkyl, wherein:
any member of such group optionally is substituted with up to 3 independently selected R^{x} substituents, and
any member of such group optionally is substituted with two substituents such that the two substituents, together with the atom(s) to which they are bonded, form a carbocyclyl or heterocyclyl, wherein:
the heterocyclyl and carbocyclyl optionally are substituted with up to 3 independently selected R^{x} substituents; and
E¹ is heteroaryl, wherein the heteroaryl optionally is substituted with one or more independently selected R^{x} substituents; and
E² is carbocyclyl, wherein the carbocyclyl optionally is substituted with one or more independently selected R^{x} substituents; and
E³ is selected from the group consisting of —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, and a bond, wherein:
any alkyl or alkenyl portion of a substituent in such group optionally is substituted with one or more independently selected Rc substituents; and
E⁴ is selected from the group consisting of hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, and heterocyclylalkoxyalkyl, wherein:
any member of such group optionally is substituted with one or more independently selected R^{d} substituents; and
each R^{X} is independently selected from the group consisting of halogen, cyano, hydroxy, nitro, nitroso, oxo, alkyl, alkenyl, alkynyl, alkoxy, alkoxyalkoxy, R^{b}-oxyalkyl, alkenyloxy, alkynyloxy, alkylthio, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, carbocyclylthio, heterocyclyl, heterocyclylalkyl, heterocyclyloxy, heterocyclyloxyalkoxy, heterocyclylthio, alkyliminocarbonyl, alkylthioalkyl, alkylsulfonylalkyl, alkylsulfoxidoalkyl, alkylthioalkenyl, alkylsulfoxidoalkenyl, alkylsulfonylalkenyl, carbocyclylalkoxyalkyl, carbocyclyliminocarbonyl, carbocyclylthioalkyl, carbocyclylsulfoxidoalkyl, carbocyclylsulfonylalkyl, carbocyclylthioalkenyl, carbocyclylsulfoxidoalkenyl, carbocyclylsulfonylalkenyl, heterocyclylalkoxyalkyl, heterocyclylthioalkyl, heterocyclylsulfoxidoalkyl, heterocyclylsulfonylalkyl, heterocyclylthioalkenyl, heterocyclylsulfoxidoalkenyl, heterocyclylsulfonylalkenyl, heterocyclyliminocarbonyl, aminosulfonylalkyl, and —R^{x1}—R^{x2}, wherein:
any member of such group optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkoxyalkyl, and alkoxyalkoxy, wherein:
any member of such group optionally is substituted with one or more substituents independently selected from the group consisting of halogen, hydroxy, and alkyl; and
each R^{x1} is selected from the group consisting of —C(O)—, —C(S)—, —C(NRY)—, —S(O)—, and —S(O)₂—; and
each R^{y} is selected from the group consisting of hydrogen and hydroxy; and
each R^{x2} is selected from the group consisting of hydrogen, hydroxy, alkyl, alkenyl, alkynyl, alkoxy, alkoxyalkyl, alkoxyalkoxy, R^{b}-oxyalkyl, alkenyloxy, alkynyloxy, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, heterocyclyl, heterocyclylalkyl, heterocyclyloxy, and heterocyclyloxyalkoxy, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkoxy, alkoxyalkyl, and alkoxyalkoxy, wherein:
any member of such group optionally is substituted with one or more substituents independently selected from the group consisting of halogen and hydroxy; and
each R^{b} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl; and
each R^{c} is independently selected from the group consisting of halogen, hydroxy, cyano, carboxy, —C(H)(NH), —C(H)(NOH), thiol, sulfo, nitro, nitroso, oxo, thioxo, imino, amino, alkyl, alkoxy, alkenyl, alkynyl, alkoxyalkyl, mono-alkylamino, di-alkylamino, alkylthio, carbocyclyl, carbocyclylalkyl, carbocyclyloxy, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, amino, alkyl, and carbocyclylalkyl; and
each R^{d} is independently selected from the group consisting of halogen, hydroxy, cyano, sulfo, nitro, nitroso, oxo, thioxo, imino, alkyl, alkoxy, alkoxyalkyl, —N(R^{e})₂, —C(O)(R^{f}), —S—R^{e}, —S(O)₂—R^{e}, carbocyclyl, alkylcarbocyclyl, alkoxycarbocyclyl, carbocyclylalkyl, heterocyclyl, alkylheterocyclyl, alkoxyheterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino; and
each R^{e} is independently selected from the group consisting of hydrogen alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino; and
each R^{f} is independently selected from the group consisting of hydrogen, alkyl, —O—R^{e}, —N(R^{e})₂, carbocyclylalkyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino.

2. A compound or salt thereof according to claim 1, wherein A¹ is tetrahydropyranyloxy.

3. A compound or salt thereof according to claim 1, wherein A¹ is hydrogen.

4. A compound or salt thereof according to claim 1, wherein A¹ is hydroxy.

5. A compound or salt thereof according to claim 4, wherein A² and A³, together with the carbon to which they are both bonded, form heterocyclyl or carbocyclyl, wherein:
the heterocyclyl or carbocyclyl optionally is substituted with up to 3 independently selected R^{X} substituents, and
the heterocyclyl or carbocyclyl optionally is substituted with two substituents such that the two substituents, together with the atom(s) to which they are bonded, form a carbocyclyl or heterocyclyl, wherein:
the optional heterocyclyl or carbocyclyl is, in turn, optionally substituted with up to 3 independently selected R^{X} substituents.

6. A compound or salt thereof according to claim 5, wherein 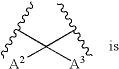
selected from the group consisting of:

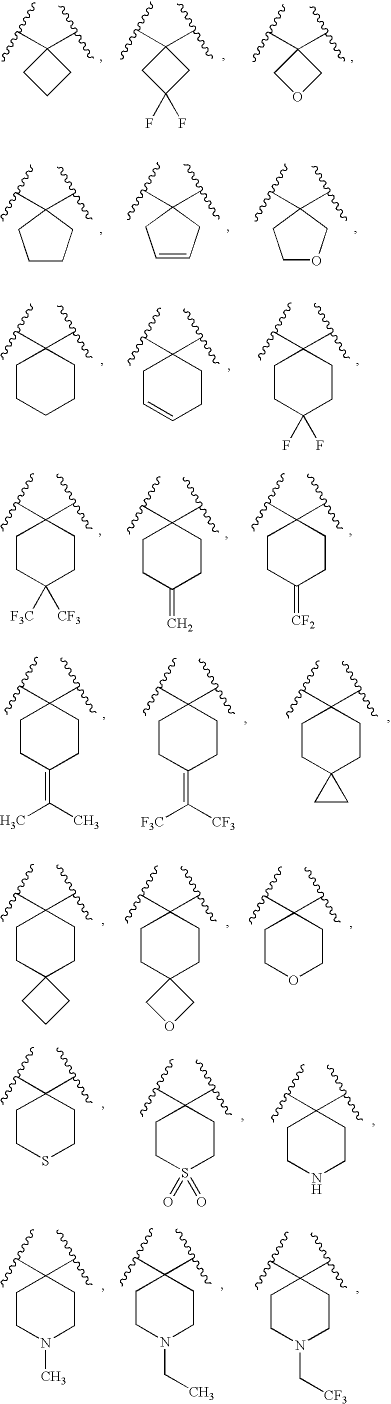

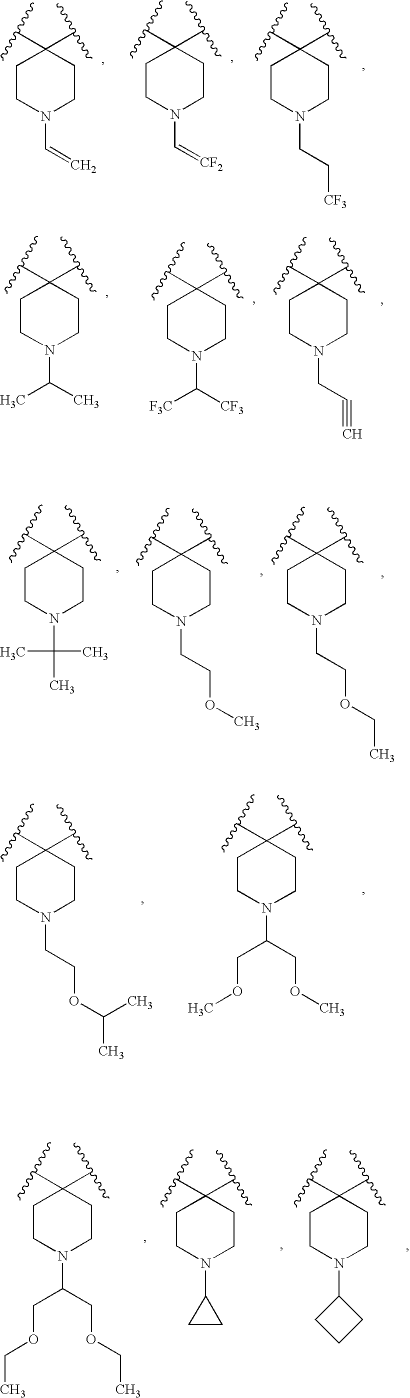

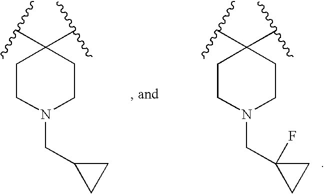

7. A compound or salt thereof according to claim 5, wherein:
the compound corresponds in structure to Formula (7-1): 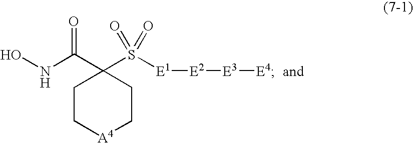
A⁴ is selected from the group consisting of —C(H)₂—, —C(Rx)(H)—, —C(R^{x})₂—, —O—, —N(H)—, —N(R^{x})—, —S—, —S(O)—, and —S(O)₂—.

8. A compound or salt thereof according to claim 7, wherein the compound corresponds in structure to Formula (8-1):

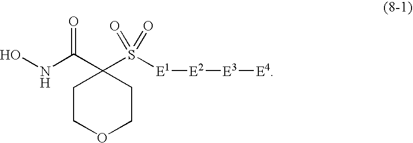

9. A compound or salt thereof according to claim 7, wherein the compound corresponds in structure to Formula (9-1):

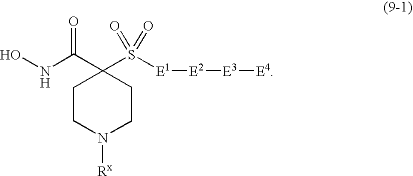

10. A compound or salt thereof according to claim 7, wherein E² is phenyl, wherein the phenyl optionally is substituted with one or more independently selected R^{x} substituents.

11. A compound or salt thereof according to claim 7, wherein E¹ is selected from the group consisting of furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, and acridinyl, wherein:
any member of such group optionally is substituted with one or more independently selected R^{x} substituents.

12. A compound or salt thereof according to claim 11, wherein E¹ is a 5-member ring.

13. A compound or salt thereof according to claim 11, wherein E¹ is a 6-member ring.

14. A compound or salt thereof according to claim 13, wherein the compound corresponds in structure to Formula (14-1):

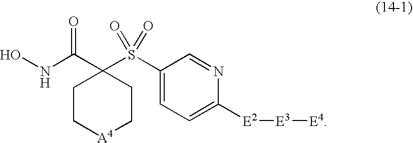

15. A compound or salt thereof according to claim 11, wherein E¹ is a 9-member fused-ring structure.

16. A compound or salt thereof according to claim 15, wherein:
the compound corresponds in structure to Formula (16-1):

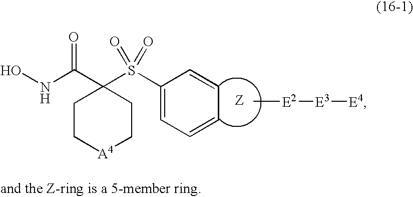

17. A compound or salt thereof according to claim 16, wherein the compound corresponds in structure to a formula selected from the group consisting of:

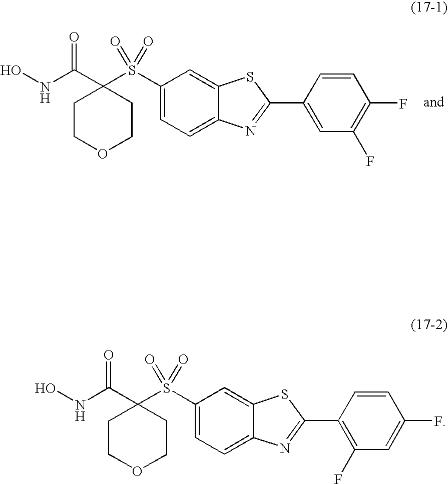

18. A compound or salt thereof according to claim 11, wherein E¹ is a 12-member fused-ring structure.

19. A compound or salt thereof according to claim 18, wherein the compound corresponds in structure to Formula (19-1):

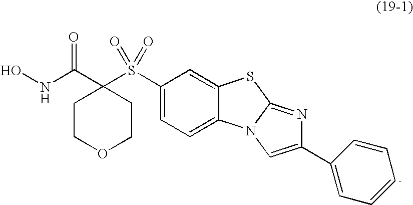

20. A compound or salt thereof according to claim 7, wherein E⁴ is carbocyclyl optionally substituted with one or more independently selected R^{d} substituents.

21. A compound or salt thereof according to claim 20, wherein the compound corresponds in structure to a formula selected from the group consisting of:

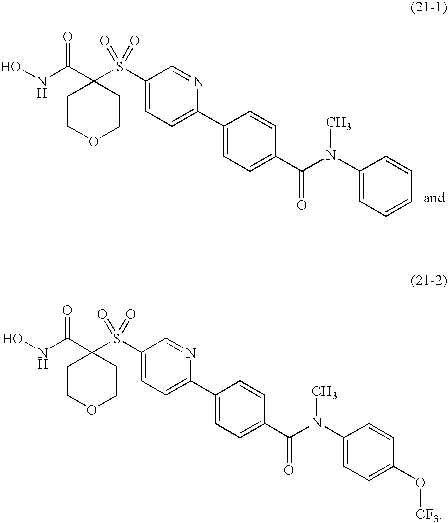

22. A compound or salt thereof according to claim 7, wherein E⁴ is heterocyclyl optionally substituted with one or more independently selected R^{d} substituents.

23. A compound or salt thereof according to claim 22, wherein the compound corresponds in structure to a formula selected from the group consisting of:

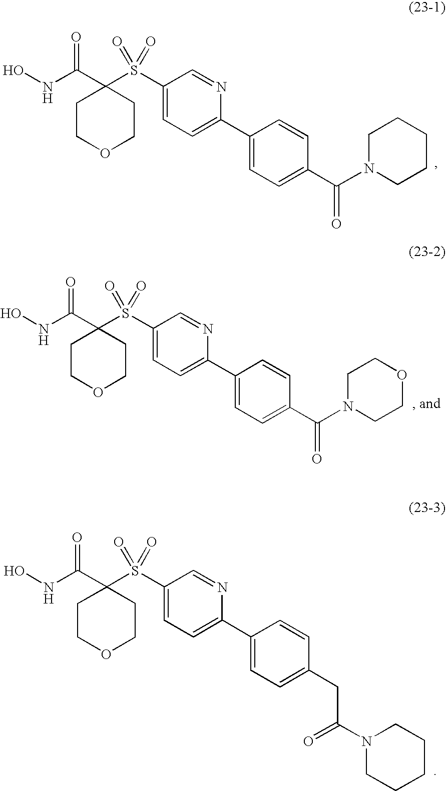

24. A compound or salt thereof according to claim 7, wherein E⁴ is aminoalkyl optionally substituted with one or more independently selected R^{d} substituents.

25. A compound or salt thereof according to claim 24, wherein the compound corresponds in structure to a formula selected from the group consisting of:

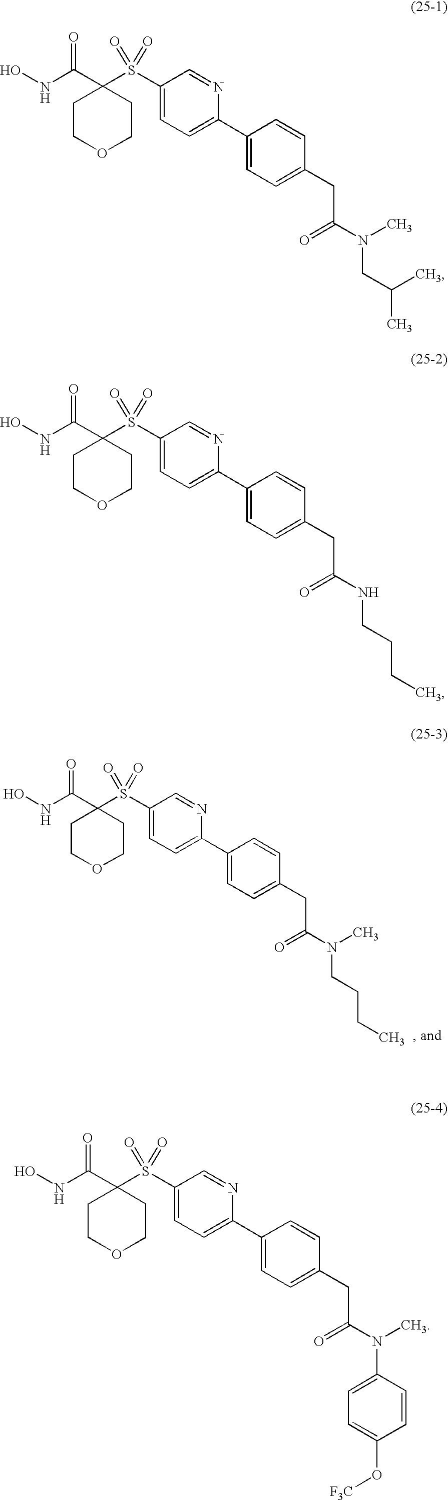

26. A compound or salt thereof according to claim 7, wherein E⁴ is selected from the group consisting of alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, and aminoalkyl, wherein:
any member of such group optionally is substituted with one or more independently selected halogen.

27. A compound or salt thereof according to claim 26, wherein E³ is selected from the group consisting of a bond, —O—, —C(O)—N(H)—, —C(O)—N(CH₃)—, and —C(O)—N(CH₂CH₃)—.

28. A compound or salt thereof according to claim 27, wherein the compound corresponds in structure to a formula selected from the group consisting of:

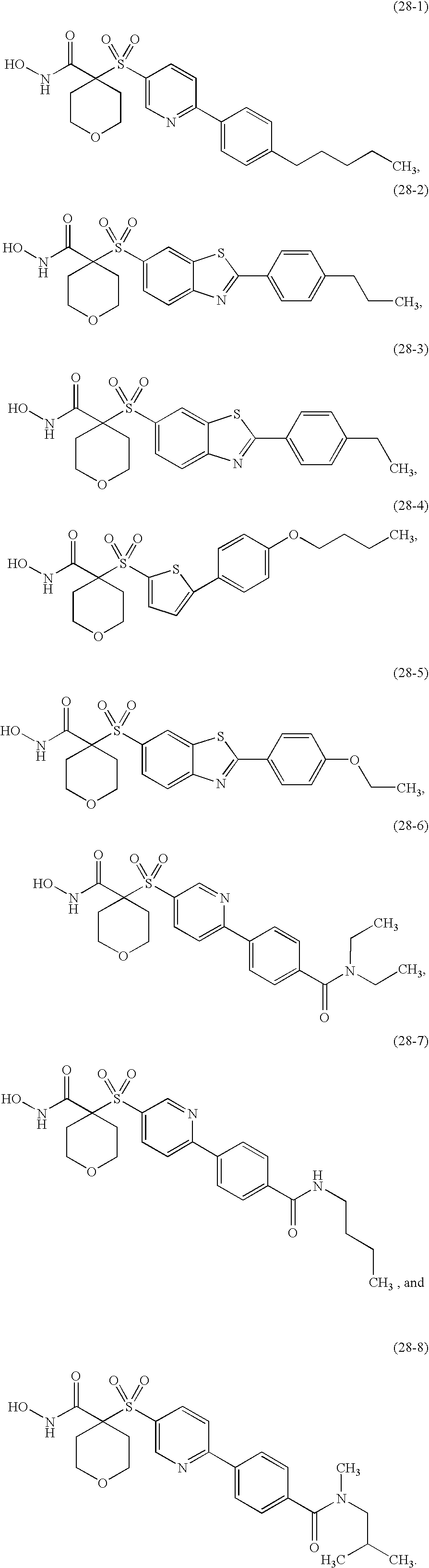

29. A compound or salt thereof according to claim 26, wherein E⁴ is C₁-C₆-alkyl substituted with one or more fluoro.

30. A compound or salt thereof according to claim 29, wherein E ⁴ is selected from the group consisting of:
trifluoromethyl, and
C₁-C₅-alkyl substituted with trifluoromethyl.

31. A compound or salt thereof according to claim 30, wherein E³ is selected from the group consisting of a bond, —O—, and —S—.

32. A compound or salt thereof according to claim 31, wherein the compound is selected from the group consisting of:

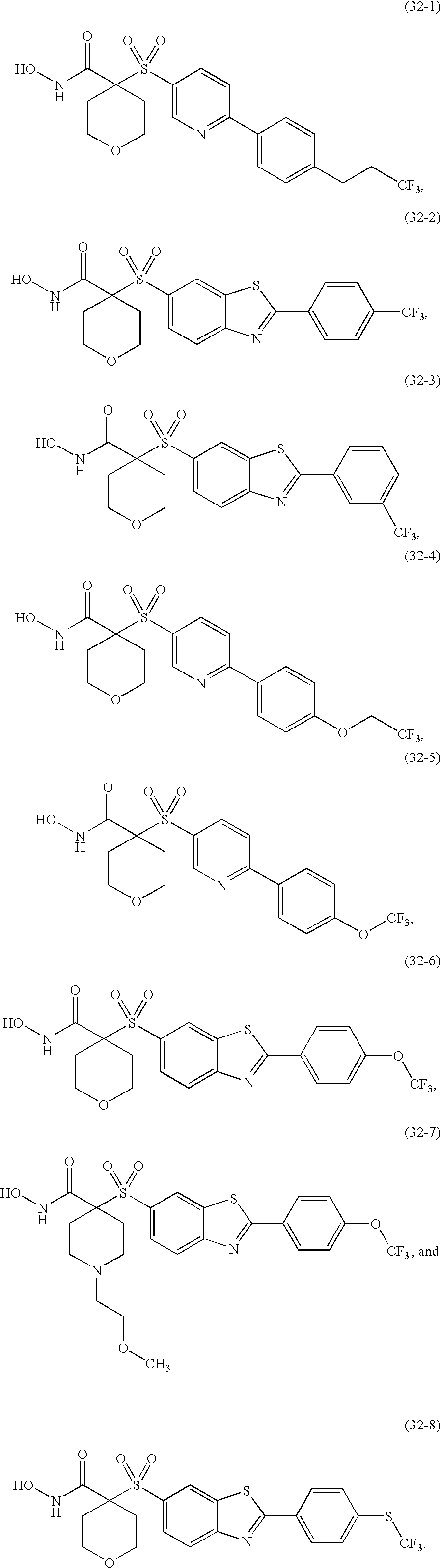

33. A compound or salt thereof according to claim 29, wherein E is selected from the group consisting of:
pentafluoroethyl, and
C₁-C₄-alkyl substituted with pentafluoroethyl.

34. A compound or salt thereof according to claim 33, wherein the compound corresponds in structure to Formula (34-1):

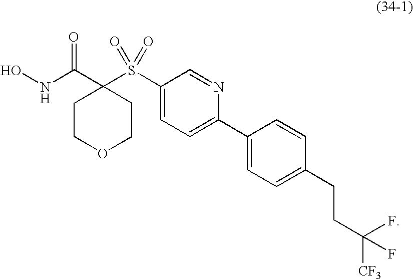

35. A compound or salt thereof according to claim 29, wherein E⁴ is C₁-C₆-alkyl comprising a carbon atom bonded to at least one hydrogen and at least one fluoro.

36. A compound or salt thereof according to claim 35, wherein the compound corresponds in structure to a formula selected from the group consisting of:

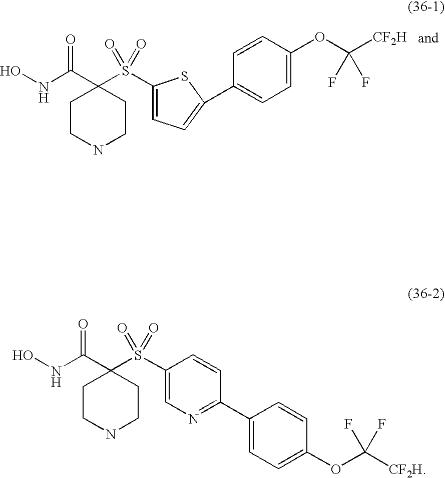

37. A compound or a salt thereof, wherein:
the compound corresponds in structure to Formula 37-1: 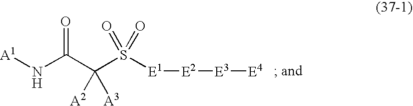
A¹ is selected from the group consisting of hydrogen, hydroxyl, carbocyclyloxy, and heterocyclyloxy; and
as to A² and A³:
A² and A³, together with the carbon to which they are both bonded, form heterocyclyl or carbocyclyl, wherein:
the heterocyclyl or carbocyclyl optionally is substituted with up to 3 independently selected R^{x} substituents, and
the heterocyclyl or carbocyclyl optionally is substituted with two substituents such that the two substituents, together with the atom(s) to which they are bonded, form a carbocyclyl or heterocyclyl, wherein:
the optional heterocyclyl or carbocyclyl substituent is, in turn, optionally substituted with up to 3 independently selected R^{x} substituents, or
A² and A³ are independently selected from the group consisting of hydrogen, alkyl, alkoxyalkyl, alkylthioalkyl, alkenyl, alkynyl, carbocyclyl, carbocyclylalkyl, carbocyclylalkenyl, carbocyclylalkynyl, carbocyclyloxyalkyl, carbocyclylalkoxyalkyl, carbocyclylalkylthio, carbocyclylthioalkyl, carbocyclylalkylthioalkyl, heterocyclyl, heterocyclylalkyl, heterocyclylalkenyl, heterocyclylalkynyl, heterocyclyloxyalkyl, heterocyclylalkoxyalkyl, heterocyclylalkylthio, heterocyclylthioalkyl, and heterocyclylalkylthioalkyl, wherein:
any member of such group optionally is substituted with up to 3 independently selected R^{x} substituents, and
any member of such group optionally is substituted with two substituents such that the two substituents, together with the atom(s) to which they are bonded, form a carbocyclyl or heterocyclyl, wherein:
the optional heterocyclyl or carbocyclyl is, in turn, optionally substituted with up to 3 independently selected Rx substituents; and
E¹ is selected from the group consisting of furanyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, and acridinyl, wherein:
any member of such group optionally is substituted with one or more independently selected R^{x} substituents; and
E² is heterocyclyl, wherein the heterocyclyl optionally is substituted with one or more independently selected R^{x} substituents; and
E³ is absent or selected from the group consisting of —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, and a bond, wherein:
any alkyl or alkenyl portion of a substituent in such group optionally is substituted with one or more independently selected RC substituents; and
E⁴ is absent or selected from the group consisting of hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, and heterocyclylalkoxyalkyl, wherein:
any member of such group optionally is substituted with one or more independently selected R^{d} substituents; and
each R^{X} is independently selected from the group consisting of halogen, cyano, hydroxy, nitro, nitroso, oxo, alkyl, alkenyl, alkynyl, alkoxy, alkoxyalkoxy, R^{b}-oxyalkyl, alkenyloxy, alkynyloxy, alkylthio, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, carbocyclylthio, heterocyclyl, heterocyclylalkyl, heterocyclyloxy, heterocyclyloxyalkoxy, heterocyclylthio, alkyliminocarbonyl, alkylthioalkyl, alkylsulfonylalkyl, alkylsulfoxidoalkyl, alkylthioalkenyl, alkylsulfoxidoalkenyl, alkylsulfonylalkenyl, carbocyclylalkoxyalkyl, carbocyclyliminocarbonyl, carbocyclylthioalkyl, carbocyclylsulfoxidoalkyl, carbocyclylsulfonylalkyl, carbocyclylthioalkenyl, carbocyclylsulfoxidoalkenyl, carbocyclylsulfonylalkenyl, heterocyclylalkoxyalkyl, heterocyclylthioalkyl, heterocyclylsulfoxidoalkyl, heterocyclylsulfonylalkyl, heterocyclylthioalkenyl, heterocyclylsulfoxidoalkenyl, heterocyclylsulfonylalkenyl, heterocyclyliminocarbonyl, aminosulfonylalkyl, and —R^{x1}—R^{x2}, wherein:
any member of such group optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkoxyalkyl, and alkoxyalkoxy, wherein:
any member of such group optionally is substituted with one or more substituents independently selected from the group consisting of halogen, hydroxy, and alkyl; and
each R^{X1} is selected from the group consisting of —C(O)—, —C(S)—, —C(NR^{y})—, —S(O)—, and —S(O)₂—; and
each R^{y} is selected from the group consisting of hydrogen and hydroxy; and
each R^{x2} is selected from the group consisting of hydrogen, hydroxy, alkyl, alkenyl, alkynyl, alkoxy, alkoxyalkyl, alkoxyalkoxy, R^{b}-oxyalkyl, alkenyloxy, alkynyloxy, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, heterocyclyl, heterocyclylalkyl, heterocyclyloxy, and heterocyclyloxyalkoxy, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkoxy, alkoxyalkyl, and alkoxyalkoxy, wherein:
any member of such group optionally is substituted with one or more substituents independently selected from the group consisting of halogen and hydroxy; and
each R^{b} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl; and
each R^{c} is independently selected from the group consisting of halogen, hydroxy, cyano, carboxy, —C(H)(NH), —C(H)(NOH), thiol, sulfo, nitro, nitroso, oxo, thioxo, imino, amino, alkyl, alkoxy, alkenyl, alkynyl, alkoxyalkyl, mono-alkylamino, di-alkylamino, alkylthio, carbocyclyl, carbocyclylalkyl, carbocyclyloxy, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, amino, alkyl, and carbocyclylalkyl; and
each R^{d} is independently selected from the group consisting of halogen, hydroxy, cyano, sulfo, nitro, nitroso, oxo, thioxo, imino, alkyl, alkoxy, alkoxyalkyl, —N(R^{e})₂, —C(O)(R^{f}), —S—R^{e}, —S(O)₂—R^{e}, carbocyclyl, alkylcarbocyclyl, alkoxycarbocyclyl, carbocyclylalkyl, heterocyclyl, alkylheterocyclyl, alkylheterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino; and
each R^{e} is independently selected from the group consisting of hydrogen alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino; and
each R^{f} is independently selected from the group consisting of hydrogen, alkyl, —O—R^{e}, —N(R^{e})₂, carbocyclylalkyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino.

38. A compound or salt thereof according to claim 37, wherein:
the compound corresponds in structure to Formula (38-1): 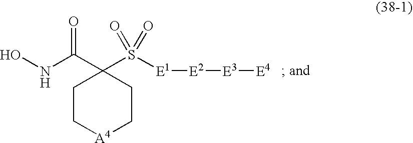
A⁴ is selected from the group consisting of C(H)₂—, —C(Rx)(H)—, —C(Rx)₂—, —O—, —N(H)—, —N(R^{x})—, —S—, —S(O)—, and —S(O)₂—.

39. A compound or salt thereof according to claim 38, wherein E¹ is selected from the group consisting of oxazolyl, isoxazolyl, isothiazolyl, thiodiazolyl, oxadiazolyl, pyrrolyl, pyrazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoisoxazolyl, anthranilyl, benzothienyl, isobenzothienyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, and acridinyl, wherein:
any member of such group optionally is substituted with one or more independently selected R^{x} substituents.

40. A compound or salt thereof according to claim 38, wherein E¹ is 5-member heteroaryl, wherein the heteroaryl optionally is substituted with one or more independently selected R^{x} substituents.

41. A compound or salt thereof according to claim 38, wherein E¹ is 6-member heteroaryl, wherein the heteroaryl optionally is substituted with one or more independently selected R^{x} substituents.

42. A compound or salt thereof according to claim 41, wherein -E¹-E²-E³-E⁴ corresponds in structure to a formula selected from the group consisting of:

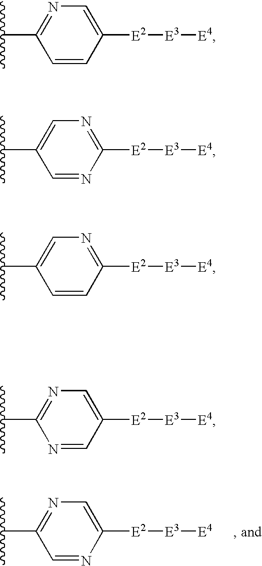

43. A compound or salt thereof according to claim 42, wherein the compound corresponds in structure to Formula (43-1):

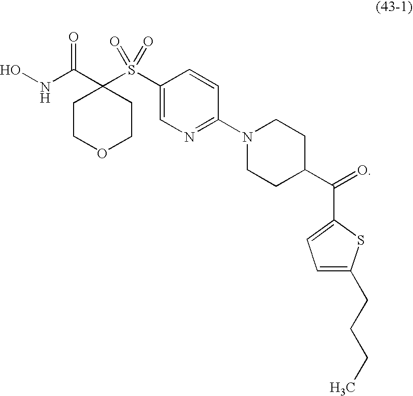

44. A compound or salt thereof according to claim 38, wherein -E¹-E²-E³-E⁴ corresponds in structure to the following formula:

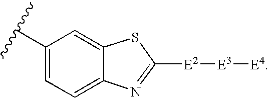

45. A compound or salt thereof according to claim 44, wherein the compound corresponds in structure to a formula selected from the group consisting of:

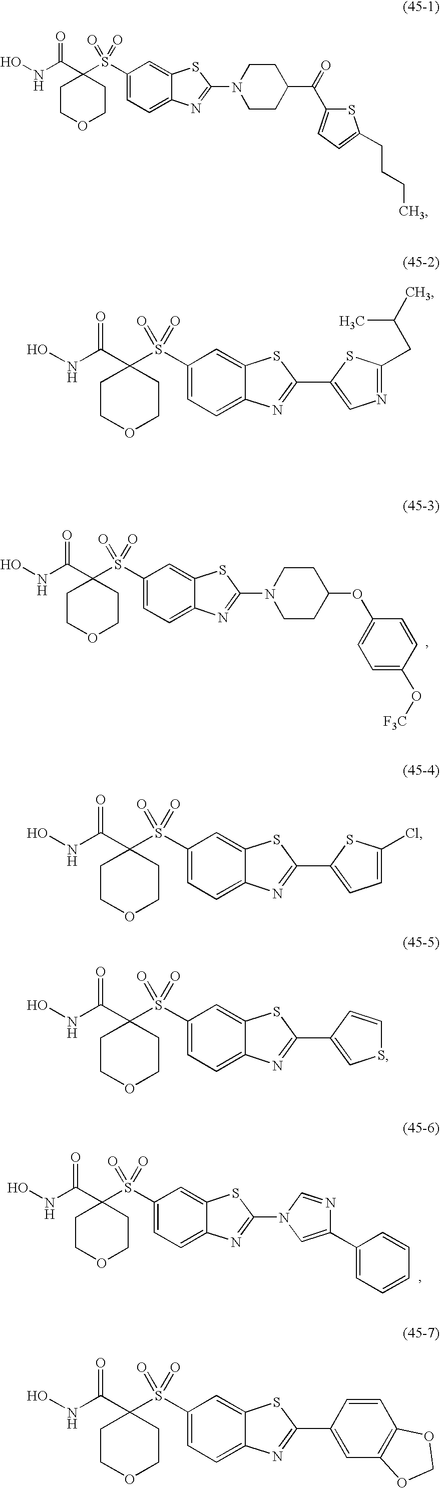

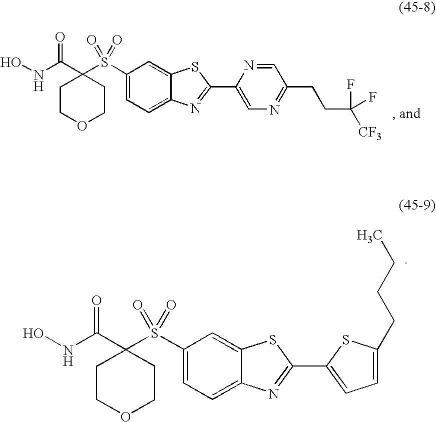

46. A compound or salt thereof according to claim 38, wherein E² is selected from the group consisting of furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, acridinyl, oxatriazolyl, dihydrofuranyl, tetrahydrofuranyl, dihydrothienyl, tetrahydrothienyl, isopyrrolyl, pyrrolinyl, pyrrolidinyl, isoimidazolyl, imidazolinyl, imidazolidinyl, pyrazolinyl, pyrazolidinyl, dithiolyl, oxathiolyl, oxathiolanyl, oxazolidinyl, isoxazolidinyl, thiazolinyl, isothiazolinyl, thiazolidinyl, isothiazolidinyl, dioxazolyl, pyranyl, dihydropyranyl, tetrahydropyranyl, piperidinyl, piperazinyl, oxazinyl, isoxazinyl, oxadiazinyl, morpholinyl, azepinyl, diazepinyl, pyrindinyl, isoindolyl, indoleninyl, pyrazolopyrimidinyl, pyrazolopyrazinyl, pyrazolopyridazyl, benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, 4H-quinolizinyl, benzoxazinyl, benzoisoxazinyl, benzoxadiazinyl, and xanthenyl, wherein:
any member of such group is optionally substituted with one or more independently selected R^{x} substituents.

47. A compound or salt thereof according to claim 46, wherein -E²-E³-E⁴ is selected from the group consisting of:

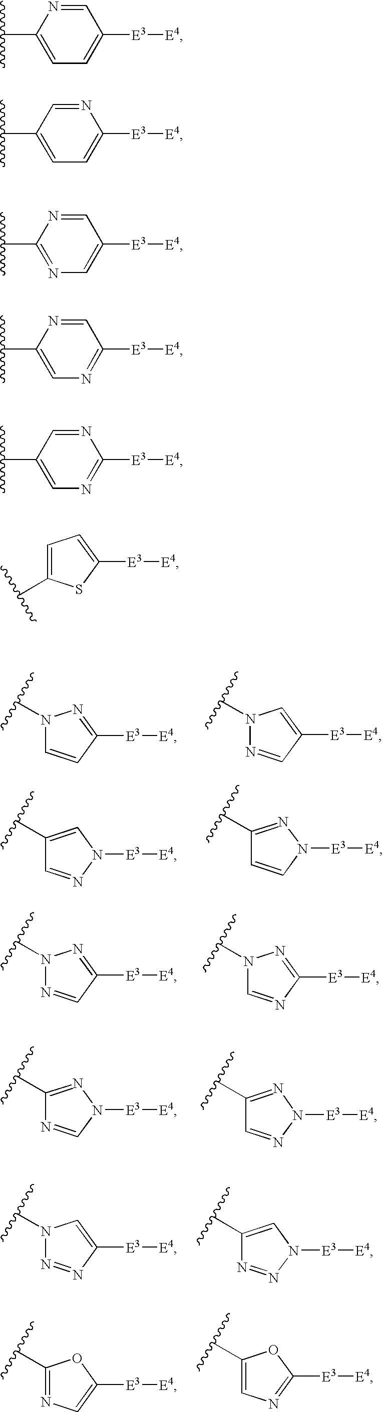

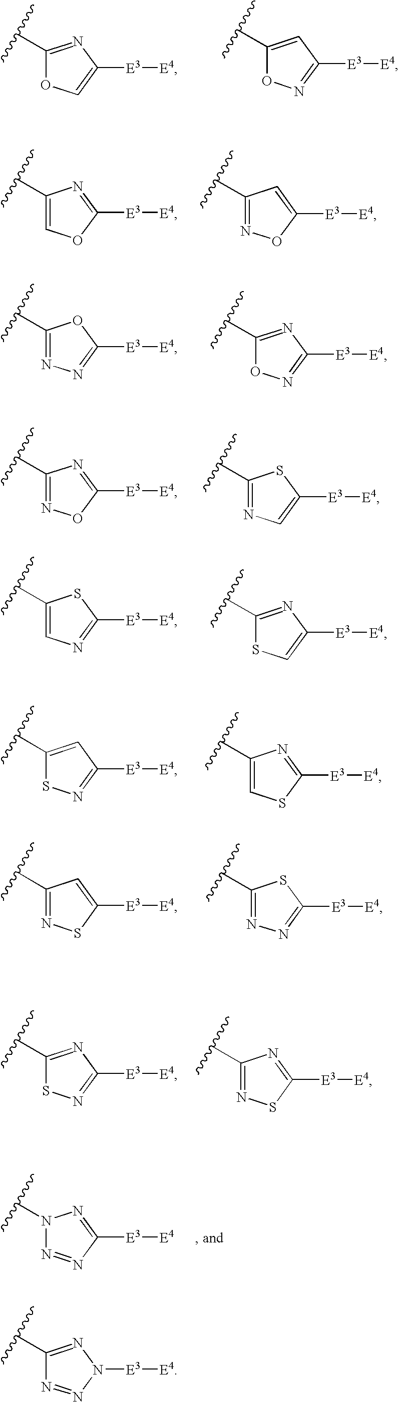

48. A compound or salt thereof according to claim 38, wherein E² is 5-member heterocyclyl, wherein the heterocyclyl optionally is substituted with one or more independently selected R^{x} substituents.

49. A compound or salt thereof according to claim 38, wherein E² is 6-member heterocyclyl, wherein the heterocyclyl optionally is substituted with one or more independently selected R^{x} substituents.

50. A compound or a salt thereof, wherein:
the compound corresponds in structure to Formula 50-1: 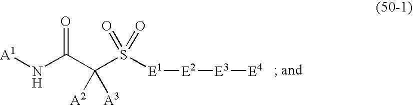
A¹ is selected from the group consisting of hydrogen, hydroxyl, carbocyclyloxy, and heterocyclyloxy; and
as to A² and A³:
A² and A³, together with the carbon to which they are both bonded, form heterocyclyl or carbocyclyl, wherein:
the heterocyclyl or carbocyclyl optionally is substituted with up to 3 independently selected R^{x} substituents, and
the heterocyclyl or carbocyclyl optionally is substituted with two substituents such that the two substituents, together with the atom(s) to which they are bonded, form a carbocyclyl or heterocyclyl, wherein:
the optional heterocyclyl or carbocyclyl substituent is, in turn, optionally substituted with up to 3 independently selected R^{x} substituents, or
A² and A³ are independently selected from the group consisting of hydrogen, alkyl, alkoxyalkyl, alkylthioalkyl, alkenyl, alkynyl, carbocyclyl, carbocyclylalkyl, carbocyclylalkenyl, carbocyclylalkynyl, carbocyclyloxyalkyl, carbocyclylalkoxyalkyl, carbocyclylalkylthio, carbocyclylthioalkyl, carbocyclylalkylthioalkyl, heterocyclyl, heterocyclylalkyl, heterocyclylalkenyl, heterocyclylalkynyl, heterocyclyloxyalkyl, heterocyclylalkoxyalkyl, heterocyclylalkylthio, heterocyclylthioalkyl, and heterocyclylalkylthioalkyl, wherein:
any member of such group optionally is substituted with up to 3 independently selected R^{X} substituents, and
any member of such group optionally is substituted with two substituents such that the two substituents, together with the atom(s) to which they are bonded, form a carbocyclyl or heterocyclyl, wherein:
the optional heterocyclyl or carbocyclyl is, in turn, optionally substituted with up to 3 independently selected Rx substituents; and
E¹ is heteroaryl, wherein the heteroaryl optionally is substituted with one or more independently selected R^{x} substituents; and
E² is selected from the group consisting of carbocyclyl and heterocyclyl, wherein:
the carbocyclyl and heterocyclyl optionally are substituted with one or more independently selected R^{x} substituents; and
E³ is selected from the group consisting of —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})C(NH)—, N(R^{b})—C(NOH)—, —C(NH)—N(R^{b})—, —C(NOH)—N(R^{b})—, alkyl, alkenyl, carbonylalkyl, alkylcarbonyl, and a bond, wherein:
any alkyl or alkenyl portion of a substituent in such group optionally is substituted with one or more independently selected RC substituents; and
E⁴ is selected from the group consisting of halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, and heterocyclylalkoxyalkyl, wherein:
any member of such group optionally is substituted with one or more independently selected R^{d} substituents; and
each R^{X} is independently selected from the group consisting of halogen, cyano, hydroxy, nitro, nitroso, oxo, alkyl, alkenyl, alkynyl, alkoxy, alkoxyalkoxy, R^{b}-oxyalkyl, alkenyloxy, alkynyloxy, alkylthio, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, carbocyclylthio, heterocyclyl, heterocyclylalkyl, heterocyclyloxy, heterocyclyloxyalkoxy, heterocyclylthio, alkyliminocarbonyl, alkylthioalkyl, alkylsulfonylalkyl, alkylsulfoxidoalkyl, alkylthioalkenyl, alkylsulfoxidoalkenyl, alkylsulfonylalkenyl, carbocyclylalkoxyalkyl, carbocyclyliminocarbonyl, carbocyclylthioalkyl, carbocyclylsulfoxidoalkyl, carbocyclylsulfonylalkyl, carbocyclylthioalkenyl, carbocyclylsulfoxidoalkenyl, carbocyclylsulfonylalkenyl, heterocyclylalkoxyalkyl, heterocyclylthioalkyl, heterocyclylsulfoxidoalkyl, heterocyclylsulfonylalkyl, heterocyclylthioalkenyl, heterocyclylsulfoxidoalkenyl, heterocyclylsulfonylalkenyl, heterocyclyliminocarbonyl, aminosulfonylalkyl, and —Rₓ₁—R^{x2}, wherein:
any member of such group optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkoxyalkyl, and alkoxyalkoxy, wherein:
any member of such group optionally is substituted with one or more substituents independently selected from the group consisting of halogen, hydroxy, and alkyl; and
each R^{x1} is selected from the group consisting of —C(O)—, —C(S)—, —C(NR^{y})—, —S(O)—, and —S(O)₂—; and
each R^{y} is selected from the group consisting of hydrogen and hydroxy; and
each R^{x2} is selected from the group consisting of hydrogen, hydroxy, alkyl, alkenyl, alkynyl, alkoxy, alkoxyalkyl, alkoxyalkoxy, R^{b}-oxyalkyl, alkenyloxy, alkynyloxy, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, heterocyclyl, heterocyclylalkyl, heterocyclyloxy, and heterocyclyloxyalkoxy, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkoxy, alkoxyalkyl, and alkoxyalkoxy, wherein:
any member of such group optionally is substituted with one or more substituents independently selected from the group consisting of halogen and hydroxy; and
each R^{b} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl; and
each R^{c} is independently selected from the group consisting of halogen, hydroxy, cyano, carboxy, —C(H)(NH), —C(H)(NOH), thiol, sulfo, nitro, nitroso, oxo, thioxo, imino, amino, alkyl, alkoxy, alkenyl, alkynyl, alkoxyalkyl, mono-alkylamino, di-alkylamino, alkylthio, carbocyclyl, carbocyclylalkyl, carbocyclyloxy, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, amino, alkyl, and carbocyclylalkyl; and
each R^{d} is independently selected from the group consisting of halogen, hydroxy, cyano, sulfo, nitro, nitroso, oxo, thioxo, imino, alkyl, alkoxy, alkoxyalkyl, —N(R^{e})₂, —C(O)(R^{f}), —S—R^{e}, —S(O)₂—R^{e}, carbocyclyl, alkylcarbocyclyl, alkylcarbocyclyl, carbocyclylalkyl, heterocyclyl, alkylheterocyclyl, alkoxyheterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino; and
each R^{e} is independently selected from the group consisting of hydrogen alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino; and
each R^{f} is independently selected from the group consisting of hydrogen, alkyl, —O—R^{e}, —N(R^{e})₂, carbocyclylalkyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino.

51. A compound or salt thereof according to claim 50, wherein E¹ is selected from the group consisting of furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, and acridinyl, wherein:
any member of such group optionally is substituted with one or more independently selected R^{x} substituents.

52. A compound or salt thereof according to claim 50, wherein:
the compound corresponds in structure to Formula (52-1): 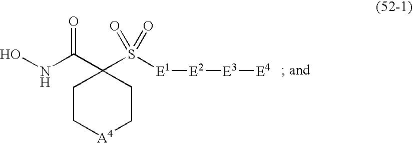
A⁴ is selected from the group consisting of —C(H)₂—, —C(R^{x})(H)—, —C(R^{x})₂—, —O—, —N(H)—, —N(R^{x})—, —S—, —S(O)—, and —S(O)₂—.

53. A compound or salt thereof according to claim 52, wherein E² is selected from the group consisting of furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, acridinyl, dihydrofuranyl, tetrahydrofuranyl, dihydrothienyl, tetrahydrothienyl, isopyrrolyl, pyrrolinyl, pyrrolidinyl, isoimidazolyl, imidazolinyl, imidazolidinyl, pyrazolinyl, pyrazolidinyl, dithiolyl, oxathiolyl, oxathiolanyl, oxazolidinyl, isoxazolidinyl, thiazolinyl, isothiazolinyl, thiazolidinyl, isothiazolidinyl, dioxazolyl, pyranyl, dihydropyranyl, tetrahydropyranyl, piperidinyl, piperazinyl, oxazinyl, isoxazinyl, oxadiazinyl, morpholinyl, azepinyl, diazepinyl, pyrindinyl, isoindolyl, indoleninyl, pyrazolopyrimidinyl, pyrazolopyrazinyl, pyrazolopyridazyl, benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, 4H-quinolizinyl, benzoxazinyl, benzoisoxazinyl, benzoxadiazinyl, and xanthenyl, wherein:
any member of such group optionally is substituted with one or more independently selected R^{x} substituents.

54. A compound or salt thereof according to claim 53, wherein -E¹-E²-E³-E⁴ corresponds in structure to a formula selected from the group consisting of:

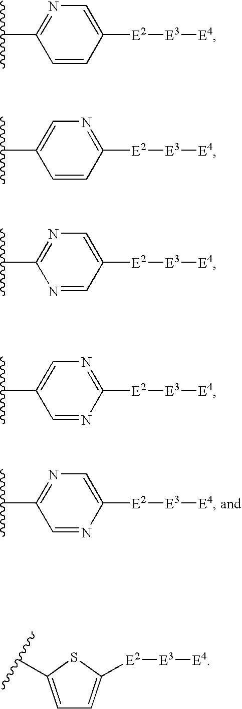

55. A compound or salt thereof according to claim 54, wherein the compound corresponds in structure to a formula selected from the group consisting of:

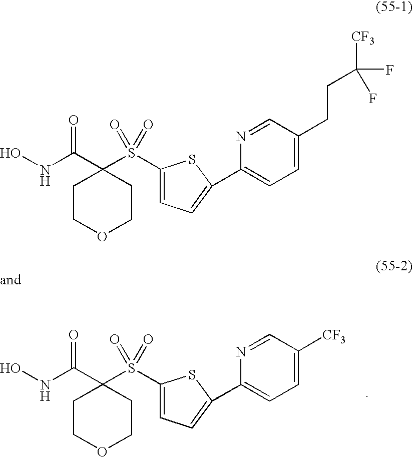

56. A method for treating a condition associated with pathologically excessive matrix metalloprotease, TNF-α convertase, or aggrecanase activity in a mammal, wherein the method comprises administering a compound (or a pharmaceutically acceptable salt thereof) recited in claim 1 to the mammal in an amount that is therapeutically effective to treat the condition.

57. A method according to claim 56, wherein A¹ is hydrogen.

58. A method according to claim 56, wherein A¹ is hydroxy.

59. A method for treating a pathological condition in a mammal, wherein:
the pathological condition is selected from the group consisting of tissue destruction, a fibrotic disease, matrix weakening, defective injury repair, a cardiovascular disease, a pulmonary disease, a kidney disease, a liver disease, an ophthalmologic disease, and a central nervous system disease; and
the method comprises administering a compound (or a pharmaceutically acceptable salt thereof) recited in claim 1 to the mammal in an amount that is therapeutically effective to treat the pathological condition.

60. A method for treating a pathological condition in a mammal, wherein:
the pathological condition is selected from the group consisting of osteoarthritis, rheumatoid arthritis, septic arthritis, tumor invasion, tumor metastasis, tumor angiogenesis, a decubitis ulcer, a gastric ulcer, a corneal ulcer, periodontal disease, liver cirrhosis, fibrotic lung disease, otosclerosis, atherosclerosis, multiple sclerosis, dilated cardiomyopathy, epidermal ulceration, epidermolysis bullosa, aortic aneurysm, defective injury repair, an adhesion, scarring, congestive heart failure, post myocardial infarction, coronary thrombosis, emphysema, proteinuria, Alzheimer's disease, bone disease, chronic obstructive pulmonary disease, and a disease of the central nervous system; and
the method comprises administering a compound (or a pharmaceutically acceptable salt thereof) recited in claim 1 to the mammal in an amount that is therapeutically effective to treat the pathological condition.

61. A method for treating a pathological condition of the central nervous system in a mammal, wherein the method comprises administering a compound (or a pharmaceutically acceptable salt thereof) recited in claim 1 to the mammal in an amount that is therapeutically effective to treat the pathological condition.

62. A pharmaceutical composition, wherein the composition comprises a therapeutically-effective amount of a compound (or a pharmaceutically acceptable salt thereof) recited in claim 1.

63. A method for treating a condition associated with pathologically excessive matrix metalloprotease, TNF-α convertase, or aggrecanase activity in a mammal, wherein the method comprises administering a compound (or a pharmaceutically acceptable salt thereof) recited in claim 37 to the mammal in an amount that is therapeutically effective to treat the condition.

64. A method according to claim 63, wherein A¹ is hydrogen.

65. A method according to claim 63, wherein A¹ is hydroxy.

66. A method for treating a pathological condition in a mammal, wherein:
the pathological condition is selected from the group consisting of tissue destruction, a fibrotic disease, matrix weakening, defective injury repair, a cardiovascular disease, a pulmonary disease, a kidney disease, a liver disease, an ophthalmologic disease, and a central nervous system disease; and
the method comprises administering a compound (or a pharmaceutically acceptable salt thereof) recited in claim 37 to the mammal in an amount that is therapeutically effective to treat the pathological condition.

67. A method for treating a pathological condition in a mammal, wherein:
the pathological condition is selected from the group consisting of osteoarthritis, rheumatoid arthritis, septic arthritis, tumor invasion, tumor metastasis, tumor angiogenesis, a decubitis ulcer, a gastric ulcer, a corneal ulcer, periodontal disease, liver cirrhosis, fibrotic lung disease, otosclerosis, atherosclerosis, multiple sclerosis, dilated cardiomyopathy, epidermal ulceration, epidermolysis bullosa, aortic aneurysm, defective injury repair, an adhesion, scarring, congestive heart failure, post myocardial infarction, coronary thrombosis, emphysema, proteinuria, Alzheimer's disease, bone disease, chronic obstructive pulmonary disease, and a disease of the central nervous system; and
the method comprises administering a compound (or a pharmaceutically acceptable salt thereof) recited in claim 37 to the mammal in an amount that is therapeutically effective to treat the pathological condition.

68. A method for treating a pathological condition of the central nervous system in a mammal, wherein the method comprises administering a compound (or a pharmaceutically acceptable salt thereof) recited in claim 37 to the mammal in an amount that is therapeutically effective to treat the pathological condition.

69. A pharmaceutical composition, wherein the composition comprises a therapeutically-effective amount of a compound (or a pharmaceutically acceptable salt thereof) recited in claim 37.

70. A method for treating a condition associated with pathologically excessive matrix metalloprotease, TNF-α convertase, or aggrecanase activity in a mammal, wherein the method comprises administering a compound (or a pharmaceutically acceptable salt thereof) recited in claim 50 to the mammal in an amount that is therapeutically effective to treat the condition.

71. A method according to claim 70, wherein A¹ is hydrogen.

72. A method according to claim 70, wherein A¹ is hydroxy.

73. A method for treating a pathological condition in a mammal, wherein:
the pathological condition is selected from the group consisting of tissue destruction, a fibrotic disease, matrix weakening, defective injury repair, a cardiovascular disease, a pulmonary disease, a kidney disease, a liver disease, an ophthalmologic disease, and a central nervous system disease; and
the method comprises administering a compound (or a pharmaceutically acceptable salt thereof) recited in claim 50 to the mammal in an amount that is therapeutically effective to treat the pathological condition.

74. A method for treating a pathological condition in a mammal, wherein:
the pathological condition is selected from the group consisting of osteoarthritis, rheumatoid arthritis, septic arthritis, tumor invasion, tumor metastasis, tumor angiogenesis, a decubitis ulcer, a gastric ulcer, a corneal ulcer, periodontal disease, liver cirrhosis, fibrotic lung disease, otosclerosis, atherosclerosis, multiple sclerosis, dilated cardiomyopathy, epidermal ulceration, epidermolysis bullosa, aortic aneurysm, defective injury repair, an adhesion, scarring, congestive heart failure, post myocardial infarction, coronary thrombosis, emphysema, proteinuria, Alzheimer's disease, bone disease, chronic obstructive pulmonary disease, and a disease of the central nervous system; and
the method comprises administering a compound (or a pharmaceutically acceptable salt thereof) recited in claim 50 to the mammal in an amount that is therapeutically effective to treat the pathological condition.

75. A method for treating a pathological condition of the central nervous system in a mammal, wherein the method comprises administering a compound (or a pharmaceutically acceptable salt thereof) recited in claim 50 to the mammal in an amount that is therapeutically effective to treat the pathological condition.

76. A pharmaceutical composition, wherein the composition comprises a therapeutically-effective amount of a compound (or a pharmaceutically acceptable salt thereof) recited in claim 50.

77. A compound or a salt thereof, wherein:
the compound corresponds in structure to Formula 77-1: 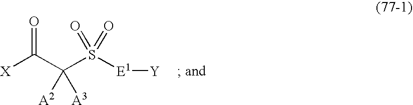
X is selected from the group consisting of —O—R¹, —NH—O—R², —NH—O—R³, and —NR⁴R⁵; and
R¹ is selected from the group consisting of hydrogen, C₁-C₆-alkyl, aryl, and aryl-C₁-C₆-alkyl; and
R² is a selectively removable protecting group; and
R³ is selected from the group consisting of hydrogen and C(W)R⁶; and
W is selected from the group consisting of O and S; and
R⁶ is selected from the group consisting of C₁-C₆-alkyl, aryl, heteroaryl-C₁-C₆-alkyl, C₃-C₈-cycloalkyl-C₁-C₆-alkyl, aryl-C₁-C₆-alkyl, heteroaryl, and amino-C₁-C₆-alkyl, wherein the amino-C₁-C₆-alkyl nitrogen optionally is substituted with:
up to two substituents independently selected from the group consisting of C₁-C₆-alkyl, aryl, aryl-C₁-C₆-alkyl, C₃-C₈-cycloalkyl-C₁-C₆-alkyl, aryl-C₁-C₆-alkoxycarbonyl, C₁-C₆-alkoxycarbonyl, and C₁-C₆-alkylcarbonyl, or
two substituents such that the amino-C₁-C₆-alkyl nitrogen and two substituents together form a 5- to 8-member heterocyclyl; and as to R⁴ and R⁵:
R⁴ is selected from the group consisting of hydrogen, C₁-C₆-alkyl, C₁-C₆-alkoxy, amino-C₁-C₆-alkyl, hydroxy-C₁-C₆-alkyl, aryl, aryloxy, and aryl-C₁-C₆-alkyl; and R⁵ is selected from the group consisting of hydrogen, C₁-C₆-alkyl, amino-C₁-C₆-alkyl, hydroxy-C₁-C₆-alkyl, aryl, and aryl-C₁-C₆-alkyl, or
R⁴ and R⁵, together with the nitrogen atom to which they are both bonded, form a 5- to 8-member ring optionally comprising up to one additional heteroatom selected from the group consisting of oxygen, nitrogen, and sulfur; and
as to A² and A³:
A² and A³, together with the carbon to which they are both bonded, form heterocyclyl or carbocyclyl, wherein:
the heterocyclyl or carbocyclyl optionally is substituted with up to 3 independently selected R^{x} substituents, and
the heterocyclyl or carbocyclyl optionally is substituted with two substituents such that the two substituents, together with the atom(s) to which they are bonded, form a carbocyclyl or heterocyclyl, wherein:
the optional heterocyclyl or carbocyclyl is, in turn, optionally substituted with up to 3 independently selected Rx substituents, or
A² and A³ are independently selected from the group consisting of hydrogen, alkyl, alkoxyalkyl, alkylthioalkyl, alkenyl, alkynyl, carbocyclyl, carbocyclylalkyl, carbocyclylalkenyl, carbocyclylalkynyl, carbocyclyloxyalkyl, carbocyclylalkoxyalkyl, carbocyclylalkylthio, carbocyclylthioalkyl, carbocyclylalkylthioalkyl, heterocyclyl, heterocyclylalkyl, heterocyclylalkenyl, heterocyclylalkynyl, heterocyclyloxyalkyl, heterocyclylalkoxyalkyl, heterocyclylalkylthio, heterocyclylthioalkyl, and heterocyclylalkylthioalkyl, wherein:
any member of such group optionally is substituted with up to 3 independently selected R^{x} substituents, and
any member of such group optionally is substituted with two substituents such that the two substituents, together with the atom(s) to which they are bonded, form a carbocyclyl or heterocyclyl, wherein:
the optional heterocyclyl or carbocyclyl is, in turn, optionally substituted with up to 3 independently selected Rx substituents; and
E¹ is heteroaryl, wherein the heteroaryl optionally substituted with one or more independently selected R^{x} substituents; and
Y is selected from the group consisting of halogen, nitro, azido, phenylsulfoxido, aryloxy, C₂-C₆-alkoxy, C₁-C₆-alkylsulfonate, arylsulfonate, and trisubstituted ammonium, wherein:
the trisubstituted ammonium substituents are independently selected from the group consisting of aryl, aryl-C₁-C₆-alkyl, and C₁-C₆-alkyl; and
each R^{x} is independently selected from the group consisting of halogen, cyano, hydroxy, nitro, nitroso, oxo, alkyl, alkenyl, alkynyl, alkoxy, alkoxyalkoxy, R^{b}-oxyalkyl, alkenyloxy, alkynyloxy, alkylthio, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, carbocyclylthio, heterocyclyl, heterocyclylalkyl, heterocyclyloxy, heterocyclyloxyalkoxy, heterocyclylthio, alkyliminocarbonyl, alkylthioalkyl, alkylsulfonylalkyl, alkylsulfoxidoalkyl, alkylthioalkenyl, alkylsulfoxidoalkenyl, alkylsulfonylalkenyl, carbocyclylalkoxyalkyl, carbocyclyliminocarbonyl, carbocyclylthioalkyl, carbocyclylsulfoxidoalkyl, carbocyclylsulfonylalkyl, carbocyclylthioalkenyl, carbocyclylsulfoxidoalkenyl, carbocyclylsulfonylalkenyl, heterocyclylalkoxyalkyl, heterocyclylthioalkyl, heterocyclylsulfoxidoalkyl, heterocyclylsulfonylalkyl, heterocyclylthioalkenyl, heterocyclylsulfoxidoalkenyl, heterocyclylsulfonylalkenyl, heterocyclyliminocarbonyl, aminosulfonylalkyl, and —R^{x1}—R^{x2}, wherein:
any member of such group optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkoxyalkyl, and alkoxyalkoxy, wherein:
any member of such group optionally is substituted with one or more substituents independently selected from the group consisting of halogen, hydroxy, and alkyl; and
each R^{x1} is selected from the group consisting of —C(O)—, —C(S)—, —C(NRY)—, —S(O)—, and —S(O)₂—; and
each R^{y} is selected from the group consisting of hydrogen and hydroxy; and
each R^{x2} is selected from the group consisting of hydrogen, hydroxy, alkyl, alkenyl, alkynyl, alkoxy, alkoxyalkyl, alkoxyalkoxy, R^{b}-oxyalkyl, alkenyloxy, alkynyloxy, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, heterocyclyl, heterocyclylalkyl, heterocyclyloxy, and heterocyclyloxyalkoxy, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkoxy, alkoxyalkyl, and alkoxyalkoxy, wherein:
any member of such group optionally is substituted with one or more substituents independently selected from the group consisting of halogen and hydroxy; and
each R^{b} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl.

78. A compound or salt thereof according to claim 77, wherein:
the compound corresponds in structure to Formula (78-1): 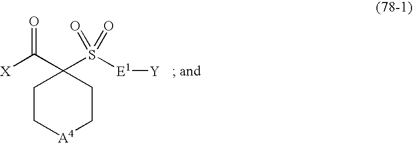
A⁴ is selected from the group consisting of —C(H)₂—, —C(R^{x})(H)—, —C(R^{x})₂—, —O—, —N(H)—, —N(R^{x})—, —S—, —S(O)—, and —S(O)₂—.

79. A compound or salt thereof according to claim 78, wherein Y is bromo.

80. A compound or salt thereof according to claim 78, wherein X is —NH— —R², and R² is 2-tetrahydropyranyl.

81. A compound or salt thereof according to claim 78, wherein X is —O—R¹, and R¹ is selected from the group consisting of hydrogen and t-butyl.

82. A compound or salt thereof according to claim 81, wherein the compound corresponds in structure to Formula (82-1):

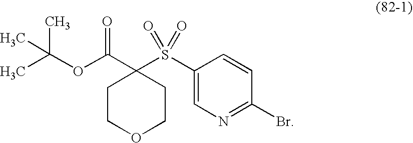

83. A compound or salt thereof according to claim 78, wherein E¹ is selected from the group consisting of furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, and acridinyl, wherein:
any member of such group is substituted with one or more independently selected R^{x} substituents.
